(19) United States
(12) Patent Application Publication
(10) Pub. No.: US 2026/0215069 A1
(43) Pub. Date: Jul. 23, 2026

(54) COMPOSITION FOR ORGANIC OPTOELECTRONIC DIODE, ORGANIC OPTOELECTRONIC DIODE, AND DISPLAY DEVICE

(71) Applicant: SAMSUNG SDI CO., LTD., Yongin-si, Gyeonggi-do (KR)

(72) Inventors: Byoungkwan LEE, Suwon-si, Gyeonggi-do (KR); Hyung Sun KIM, Suwon-si, Gyeonggi-do (KR); Ho Kuk JUNG, Suwon-si, Gyeonggi-do (KR); Dong Min KANG, Suwon-si, Gyeonggi-do (KR); Jiyun KWON, Suwon-si, Gyeonggi-do (KR); Jonghoon KIM, Suwon-si, Gyeonggi-do (KR); Changwoo KIM, Suwon-si, Gyeonggi-do (KR); Hansol SEO, Suwon-si, Gyeonggi-do (KR); Yunsoo KIM, Suwon-si, Gyeonggi-do (KR); Jinseok JANG, Suwon-si, Gyeonggi-do (KR); Kipo JANG, Suwon-si, Gyeonggi-do (KR); Youngkyoung JO, Suwon-si, Gyeonggi-do (KR)

(21) Appl. No.: 19/138,865

(22) PCT Filed: Jul. 24, 2024
(86) PCT No.: PCT/KR2024/010656

§ 371 Date: Jun. 13, 2025

(30) Foreign Application Priority Data
Jul. 24, 2023 (KR) 10-2023-0096302

(51) Int. Cl. H10K 50/11; C07D 403/14; C07D 487/04; H10K 85/30; H10K 85/40; H10K 85/60

(52) U.S. Cl. CPC H10K 50/11; C07D 403/14; C07D 487/04; H10K 85/342; H10K 85/40; H10K 85/622; H10K 85/626; H10K 85/633; H10K 85/636; H10K 85/654; H10K 85/6572; H10K 85/6574; H10K 85/6576

(57) ABSTRACT

Disclosed are a composition for an organic optoelectronic device including a first compound represented by Chemical Formula 1, and a second compound represented by a combination of Chemical Formula 2 and Chemical Formula 3, an organic optoelectronic device including the same, and a display device. The contents of Chemical Formula 1 to Chemical Formula 3 are as defined in the specification.

### TECHNICAL FIELD

A composition for an organic optoelectronic device, an organic optoelectronic device, and a display device are disclosed.

### BACKGROUND ART

An organic optoelectronic device (organic optoelectronic diode) is a device capable of converting electrical energy and optical energy to each other.

Organic optoelectronic devices may be largely divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device include an organic photoelectric device, an organic light emitting diode, an organic solar cell, and an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode is greatly influenced by an organic material between electrodes.

### DISCLOSURE

### Technical Problem

An embodiment provides a composition for an organic optoelectronic device that can implement a high-efficiency, and long life-span organic optoelectronic device.

Another embodiment provides an organic optoelectronic device including the composition for an organic optoelectronic device.

Another embodiment provides a display device including the organic optoelectronic device.

### Technical Solution

According to an embodiment, a first compound represented by Chemical Formula 1, and a second compound represented by a combination of Chemical Formula 2 and Chemical Formula 3.

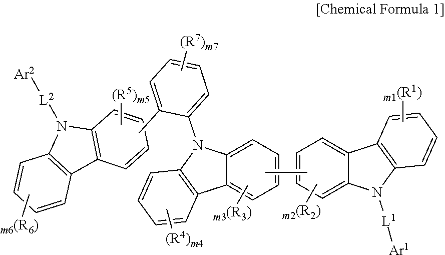

In Chemical Formula 1,
R¹ to R⁶ are each independently hydrogen, deuterium, a cyano group, a halogen, a substituted or unsubstituted C1 to C30 alkyl group, a substituted or unsubstituted C6 to C30 aryl group, a substituted or unsubstituted C2 to C30 heterocyclic group, or a combination thereof,
R⁷s are each independently hydrogen, deuterium, or a substituted or unsubstituted C6 to C12 aryl group,
L¹ and L² are each independently a single bond, a substituted or unsubstituted C6 to C20 arylene group, a substituted or unsubstituted C2 to C20 heterocyclic group, or a combination thereof,
Ar¹ and Ar² are each independently a substituted or unsubstituted C6 to C30 aryl group, or a substituted or unsubstituted C2 to C30 heterocyclic group,
m1, m4, m6, and m7 are each independently one of integers of 1 to 4, and
m2, m3, and m5 are each independently one of integers of 1 to 3;

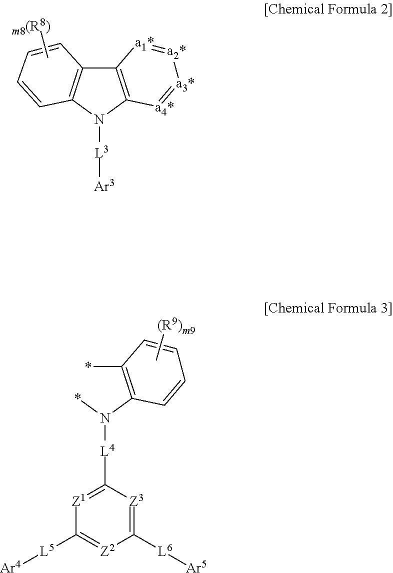

wherein, in Chemical Formula 2 and Chemical Formula 3,
Ar³ to Ar⁵ are each independently a substituted or unsubstituted C6 to C30 aryl group, or a substituted or unsubstituted C2 to C30 heterocyclic group,
among a1* to a4* of Chemical Formula 2, two adjacent ones are linking points linked to * of Chemical Formula 3, and among a1* to a4* of Chemical Formula 2, the remaining two that are not linked to * of Chemical Formula 3 are each independently C-L^{a}-R^{a},
Z¹ to Z³ are each independently N or C-L^{b}-R^{b},
at least two of Z¹ to Z³ are N,
L^{a}, L^{b}, and L³ to L⁶ are each independently a single bond or a substituted or unsubstituted C6 to C20 arylene group,
R^{a}, R^{b}, R⁸, and R⁹ are each independently hydrogen, deuterium, a cyano group, a halogen, a substituted or unsubstituted amine group, a substituted or unsubstituted C1 to C30 alkyl group, a substituted or unsubstituted silyl group, a substituted or unsubstituted C6 to C30 aryl group, or a substituted or unsubstituted C2 to C30 heterocyclic group, and
m8 and m9 are each independently one of integers of 1 to 4.

According to another embodiment, an organic optoelectronic device includes an anode and a cathode facing each other, and at least one organic layer between the anode and the cathode, wherein the organic layer includes the composition for an organic optoelectronic device.

According to another embodiment, a display device including the organic optoelectronic device is provided.

### Advantageous Effects

Low-driving, high-efficiency, and long life-span organic optoelectronic devices can be implemented.

### DESCRIPTION OF THE DRAWINGS

FIG. 1 is a cross-sectional view illustrating an organic light emitting diode according to an embodiment.

### DESCRIPTION OF SYMBOLS

100: organic light emitting diode
105: organic layer
110: cathode
120: anode
130: light emitting layer
140: hole transport region
150: electron transport region

### BEST MODE

Hereinafter, embodiments of the present invention are described in detail. However, these embodiments are exemplary, and this disclosure is not limited thereto.

In the present specification, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present invention, “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In addition, in a specific example of the present invention, “substituted” refers to replacement of at least one hydrogen of a substituent or a compound by deuterium, a C1 to C20 alkyl group, a C6 to C30 aryl group, or a cyano group. In addition, in a specific example of the present invention, “substituted” refers to replacement of at least one hydrogen of a substituent or a compound by deuterium, a C1 to C5 alkyl group, a C6 to C18 aryl group, or a cyano group. In addition, in a specific example of the present invention, “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

In the present specification, “unsubstituted” refers to non-replacement of a hydrogen atom by another substituent and remaining of the hydrogen atom.

In the present specification, “hydrogen substitution (—H) may include “deuterium substitution (-D) or “tritium substitution (-T).

In the present specification, when a definition is not otherwise provided, “hetero” refers to one including one to three heteroatoms selected from N, O, S, P, and Si, and remaining carbons in one functional group.

In the present specification, “aryl group” refers to a group including at least one hydrocarbon aromatic moiety, and may include a group in which all elements of the hydrocarbon aromatic moiety have p-orbitals which form conjugation, for example a phenyl group, a naphthyl group, and the like, a group in which two or more hydrocarbon aromatic moieties may be linked by a sigma bond, for example a biphenyl group, a terphenyl group, a quaterphenyl group, and the like, and a group in which two or more hydrocarbon aromatic moieties are fused directly or indirectly to provide a non-aromatic fused ring, for example a fluorenyl group, and the like.

The aryl group may include a monocyclic, polycyclic or fused ring polycyclic (i.e., rings sharing adjacent pairs of carbon atoms) functional group.

In the present specification, “heterocyclic group” has a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” refers to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted benzophenanthrenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, a substituted or unsubstituted benzonaphthofuranyl group, a substituted or unsubstituted benzonaphthothiophenyl group, a substituted or unsubstituted benzofuranofluorenyl group, a substituted or unsubstituted benzothiophenefluorenyl group, or a combination thereof, but is not limited thereto.

In the present specification, hole characteristics refer to an ability to donate an electron to form a hole when an electric field is applied and that a hole formed in the anode may be easily injected into the light emitting layer and transported in the light emitting layer due to conductive characteristics according to the highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to the lowest unoccupied molecular orbital (LUMO) level.

Hereinafter, a composition for an organic optoelectronic device according to an embodiment will be described.

The composition for an organic optoelectronic device according to an embodiment includes a first compound and a second compound.

The first compound is represented by Chemical Formula 1 and the second compound is represented by a combination of Chemical Formula 2 and Chemical Formula 3.

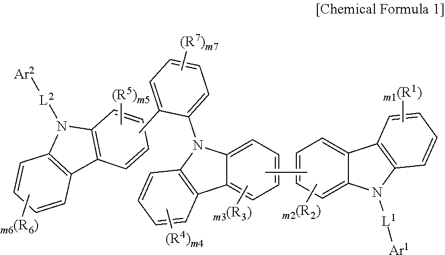

In Chemical Formula 1,
R¹ to R⁶ are each independently hydrogen, deuterium, a cyano group, a halogen, a substituted or unsubstituted C1 to C30 alkyl group, a substituted or unsubstituted C6 to C30 aryl group, a substituted or unsubstituted C2 to C30 heterocyclic group, or a combination thereof,
R⁷s are each independently hydrogen, deuterium, or a substituted or unsubstituted C6 to C12 aryl group,
L¹ and L² are each independently a single bond, a substituted or unsubstituted C6 to C20 arylene group, a substituted or unsubstituted C2 to C20 heterocyclic group, or a combination thereof,
Ar¹ and Ar² are each independently a substituted or unsubstituted C6 to C30 aryl group, or a substituted or unsubstituted C2 to C30 heterocyclic group,
m1, m4, m6, and m7 are each independently one of integers of 1 to 4, and
m2, m3, and m5 are each independently one of integers of 1 to 3;

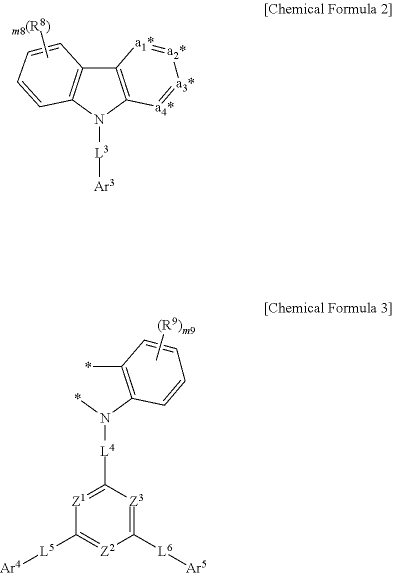

wherein, in Chemical Formula 2 and Chemical Formula 3,
Ar³ to Ar⁵ are each independently a substituted or unsubstituted C6 to C30 aryl group, or a substituted or unsubstituted C2 to C30 heterocyclic group,
among a1* to a4* of Chemical Formula 2, two adjacent ones are linking points linked to * of Chemical Formula 3, and among a1* to a4* of Chemical Formula 2, the remaining two that are not linked to * of Chemical Formula 3 are each independently C-L^{a}-R^{a},
Z¹ to Z³ are each independently N or C-L^{b}-R^{b},
at least two of Z¹ to Z³ are N,
L^{a}, L^{b}, and L³ to L⁶ are each independently a single bond or a substituted or unsubstituted C6 to C20 arylene group,
R^{a}, R^{b}, R⁸, and R⁹ are each independently hydrogen, deuterium, a cyano group, a halogen, a substituted or unsubstituted amine group, a substituted or unsubstituted C1 to C30 alkyl group, a substituted or unsubstituted silyl group, a substituted or unsubstituted C6 to C30 aryl group, or a substituted or unsubstituted C2 to C30 heterocyclic group,
m8 and m9 are each independently one of integers of 1 to 4.

The compound represented by Chemical Formula 1 may have a shallower HOMO energy level by substituting carbazole in the N-direction of bicarbazole, and Chemical Formula 1 may have a faster hole mobility by having three carbazoles in total.

In particular, carbazole substituted in the N-direction of bicarbazole increases steric hindrance by including ortho-phenylene as a linking group, and thus the efficiency of an organic light emitting diode to which it is applied may be further improved.

In addition, when the second compound represented by a combination of Chemical Formula 2 and Chemical Formula 3 is used in combination, hole injection may be made smoother, and a balance between electrons and holes may be well maintained to implement stable device characteristics. In particular, if the first compound and the second compound having similar deposition temperatures are introduced together, it can have the advantage of not significantly changing the device characteristics during the deposition process.

When m1 is 2 or more, each R¹ may be the same or different from each other.

When m2 is 2 or more, each R² may be the same or different from each other.

When m3 is 2 or more, each R³ may be the same or different from each other.

When m4 is 2 or more, each R⁴ may be the same or different from each other.

When m5 is 2 or more, each R⁵ may be the same or different from each other.

When m6 is 2 or more, each R⁶ may be the same or different from each other.

When m7 is 2 or more, each R⁷ may be the same or different from each other.

As an example, the first compound may be represented by any one of Chemical Formula 1-1 to Chemical Formula 1-8.

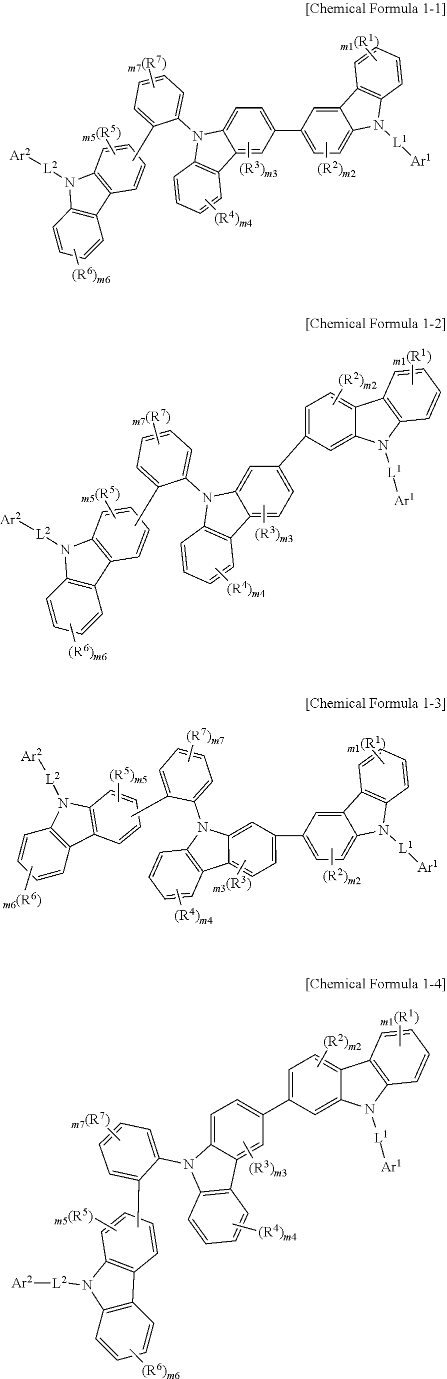

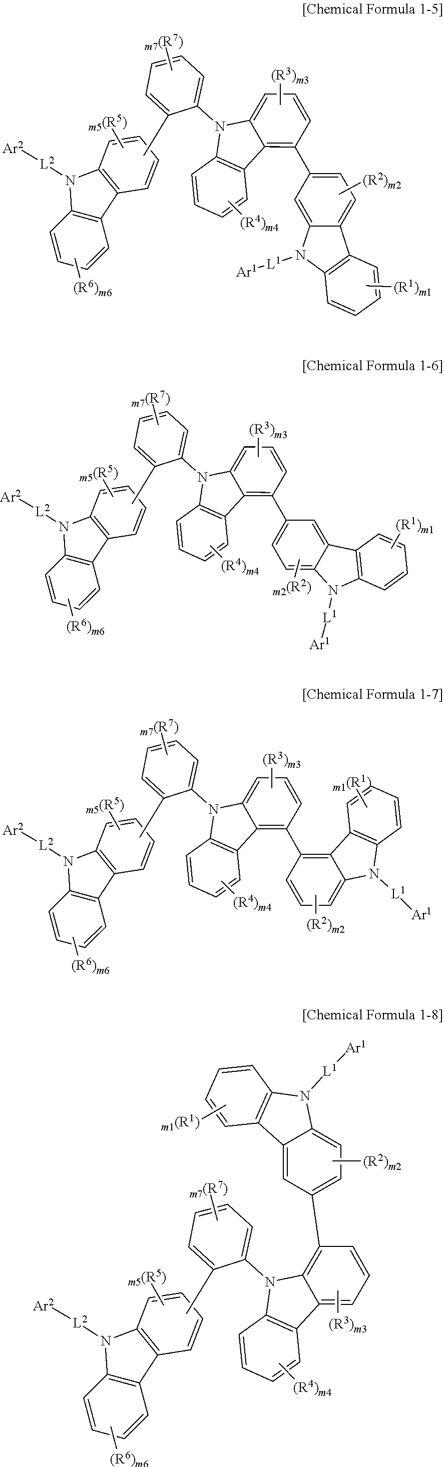

In Chemical Formula 1-1 to Chemical Formula 1-8,
R¹ to R⁷, L¹ and L², Ar¹ and Ar², and m1 to m7 are the same as defined in Chemical Formula 1.

As a specific example, the first compound may be represented by Chemical Formula 1-1 or Chemical Formula 1-7.

For example, Ar¹ and Ar² may each independently be a substituted or unsubstituted phenyl group, a substituted or unsubstituted biphenyl group, a substituted or unsubstituted terphenyl group, a substituted or unsubstituted fluorenyl group, a substituted or unsubstituted dibenzofuranyl group, a substituted or unsubstituted dibenzothiophenyl group, or a substituted or unsubstituted dibenzosilolyl group.

In an embodiment, Ar¹ and Ar² may each independently be a substituted or unsubstituted phenyl group, a substituted or unsubstituted biphenyl group or a substituted or unsubstituted terphenyl group.

For example, L¹ and L² may each independently be a single bond or a substituted or unsubstituted phenylene group.

In a specific embodiment, L¹-Ar¹ and L²-Ar² of Chemical Formula 1 may each independently be selected from the substituents listed in Group I.

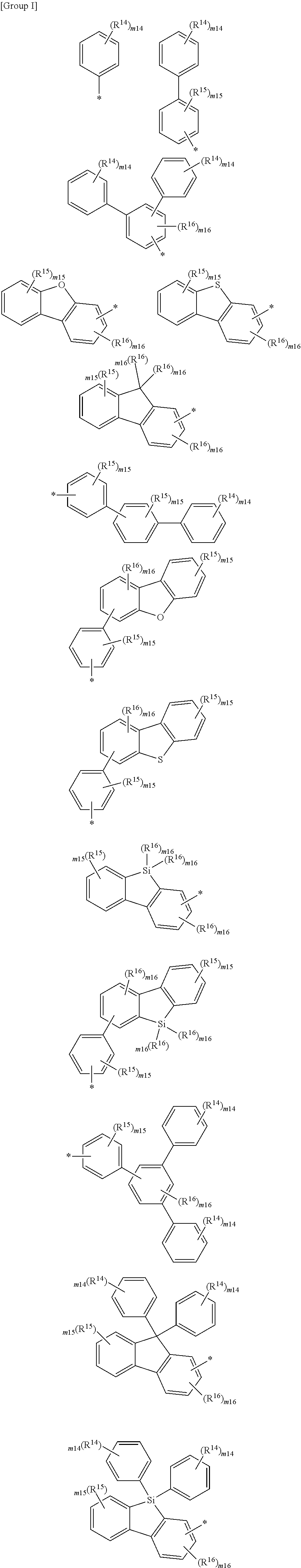

In Group I,
R¹⁴ to R¹⁶, R^{16a}, R^{16b}, R^{16c}, R^{16d}, R^{16e}, and R^{16f} are each independently hydrogen, deuterium, a substituted or unsubstituted C1 to C4 alkyl group, a substituted or unsubstituted C6 to C18 aryl group, or a substituted or unsubstituted C2 to C30 heteroaryl group,
m14 is one of integers of 1 to 5,
m15 is one of integers of 1 to 4,
m16 is one of integers of 1 to 3, and
* is a linking point.

When m14 is 2 or more, each R¹⁴ may be the same or different from each other. When m15 is 2 or more, each R¹⁵ may be the same or different from each other. When m16 is 2 or more, each R¹⁶ may be the same or different from each other.

As an example, R¹ to R⁶ may each independently be hydrogen, deuterium, a cyano group, a halogen, a substituted or unsubstituted C1 to C10 alkyl group, a substituted or unsubstituted phenyl group, a substituted or unsubstituted biphenyl group, a substituted or unsubstituted terphenyl group, or a substituted or unsubstituted naphthyl group.

For example, R¹ to R⁶ may each independently be hydrogen, deuterium, a cyano group, a substituted or unsubstituted C1 to C6 alkyl group, a substituted or unsubstituted phenyl group, a substituted or unsubstituted biphenyl group, or a substituted or unsubstituted naphthyl group.

As an example, R⁷ may be hydrogen, deuterium, a substituted or unsubstituted phenyl group, or a substituted or unsubstituted biphenyl group.

For example, the first compound may be one selected from the compounds listed in Group 1, but is not limited thereto.

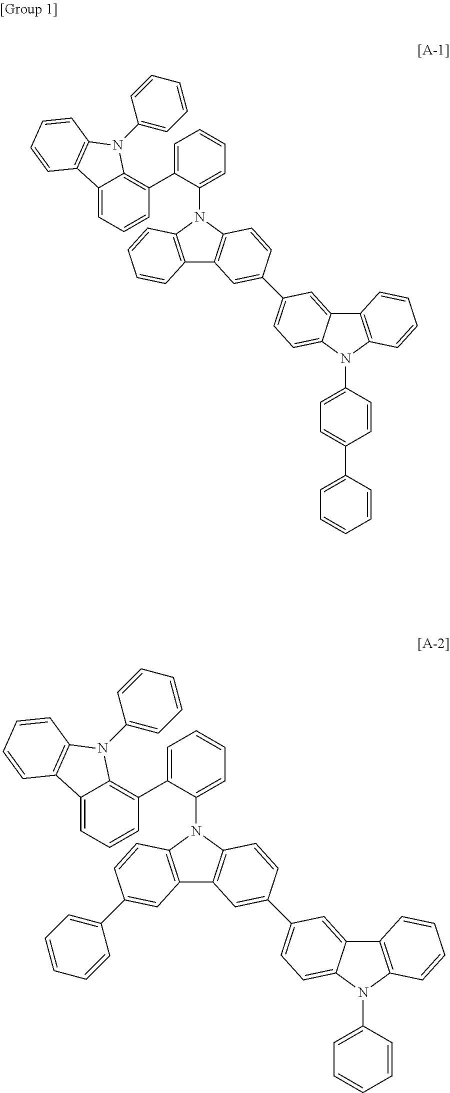 [SMILES: c1ccc(-c2ccc(-n3c4ccccc4c4cc(-c5ccc6c(c5)c5ccccc5n6-c5ccccc5-c5cccc6c7ccccc7n(-c7ccccc7)c56)ccc43)cc2)cc1 ; c1ccc(-c2ccc3c(c2)c2cc(-c4ccc5c(c4)c4ccccc4n5-c4ccccc4)ccc2n3-c2ccccc2-c2cccc3c4ccccc4n(-c4ccccc4)c23)cc1]

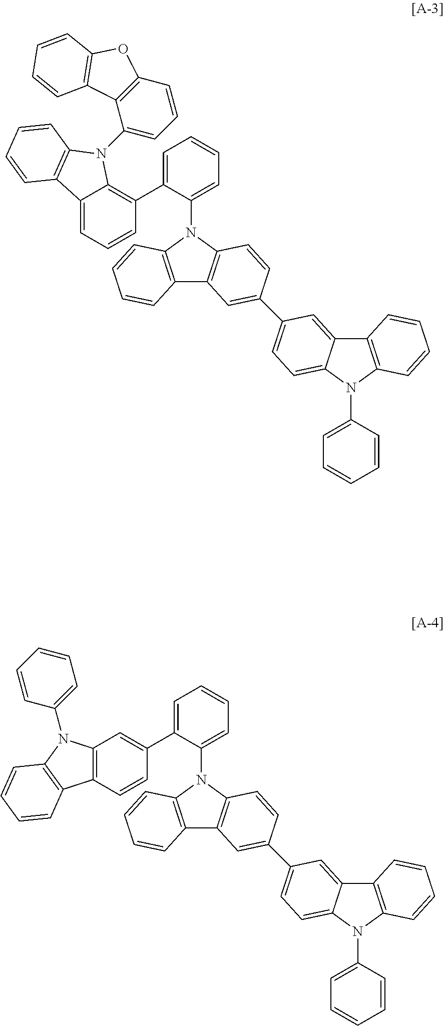 [SMILES: c1ccc(-n2c3ccccc3c3cc(-c4ccc5c(c4)c4ccccc4n5-c4ccccc4-c4cccc5c6ccccc6n(-c6cccc7oc8ccccc8c67)c45)ccc32)cc1 ; c1ccc(-n2c3ccccc3c3cc(-c4ccc5c(c4)c4ccccc4n5-c4ccccc4-c4ccc5c6ccccc6n(-c6ccccc6)c5c4)ccc32)cc1]

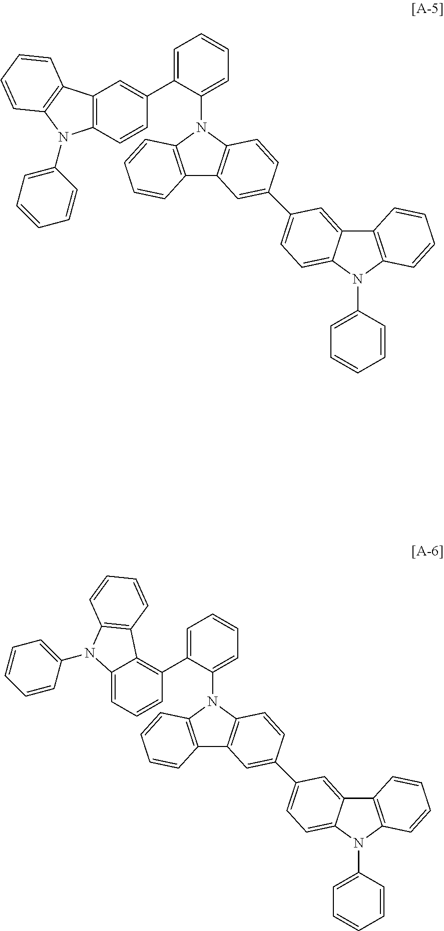 [SMILES: c1ccc(-n2c3ccccc3c3cc(-c4ccc5c(c4)c4ccccc4n5-c4ccccc4-c4ccc5c(c4)c4ccccc4n5-c4ccccc4)ccc32)cc1 ; c1ccc(-n2c3ccccc3c3cc(-c4ccc5c(c4)c4ccccc4n5-c4ccccc4-c4cccc5c4c4ccccc4n5-c4ccccc4)ccc32)cc1]

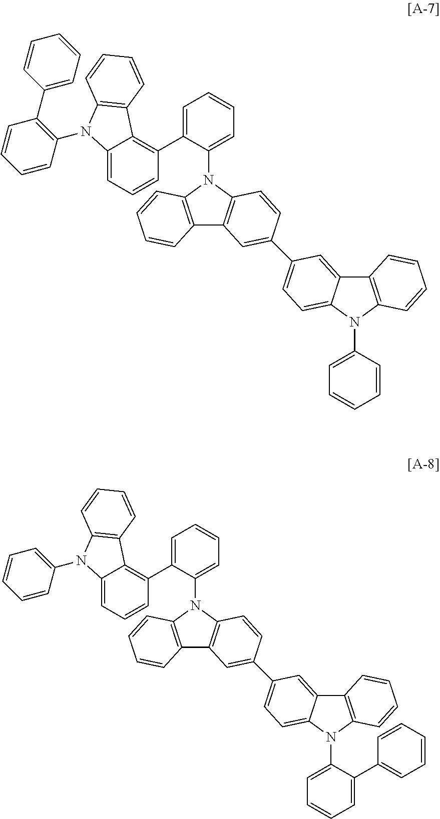 [SMILES: c1ccc(-c2ccccc2-n2c3ccccc3c3c(-c4ccccc4-n4c5ccccc5c5cc(-c6ccc7c(c6)c6ccccc6n7-c6ccccc6)ccc54)cccc32)cc1 ; c1ccc(-c2ccccc2-n2c3ccccc3c3cc(-c4ccc5c(c4)c4ccccc4n5-c4ccccc4-c4cccc5c4c4ccccc4n5-c4ccccc4)ccc32)cc1]

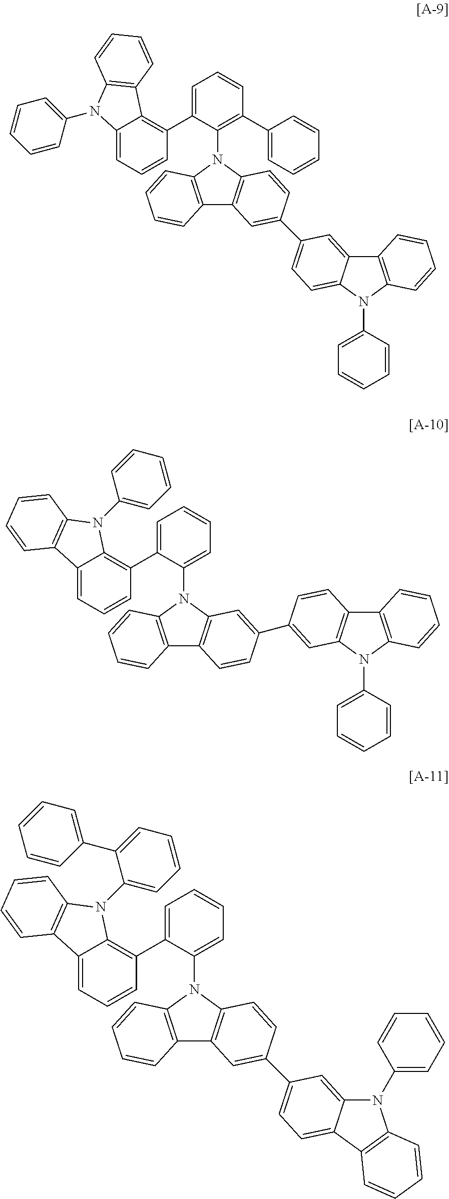 [SMILES: c1ccc(-c2cccc(-c3cccc4c3c3ccccc3n4-c3ccccc3)c2-n2c3ccccc3c3cc(-c4ccc5c(c4)c4ccccc4n5-c4ccccc4)ccc32)cc1 ; c1ccc(-n2c3ccccc3c3ccc(-c4ccc5c6ccccc6n(-c6ccccc6-c6cccc7c8ccccc8n(-c8ccccc8)c67)c5c4)cc32)cc1 ; c1ccc(-c2ccccc2-n2c3ccccc3c3cccc(-c4ccccc4-n4c5ccccc5c5cc(-c6ccc7c8ccccc8n(-c8ccccc8)c7c6)ccc54)c32)cc1]

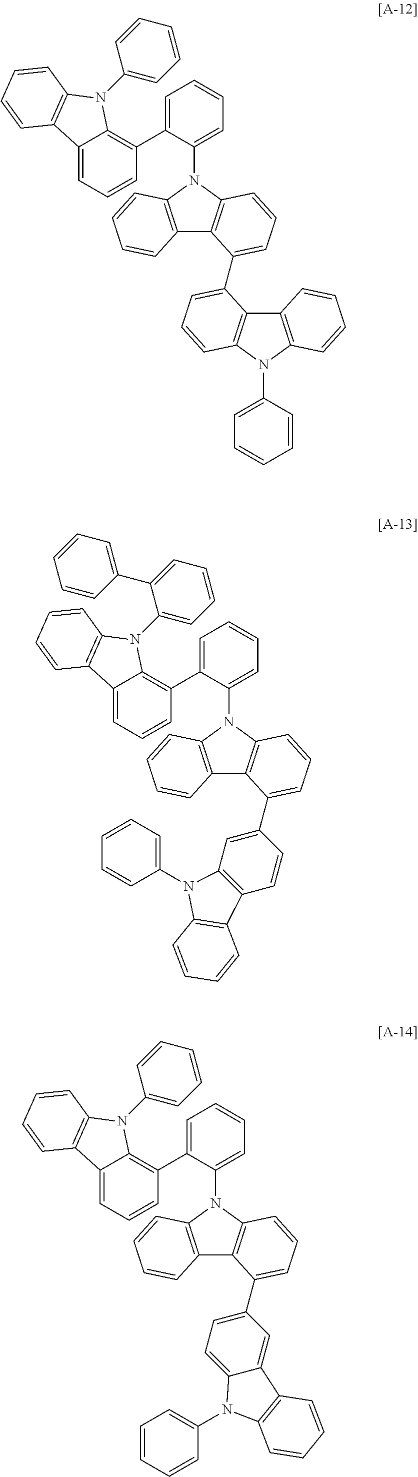 [SMILES: c1ccc(-n2c3ccccc3c3c(-c4cccc5c4c4ccccc4n5-c4ccccc4-c4cccc5c6ccccc6n(-c6ccccc6)c45)cccc32)cc1 ; c1ccc(-c2ccccc2-n2c3ccccc3c3cccc(-c4ccccc4-n4c5ccccc5c5c(-c6ccc7c8ccccc8n(-c8ccccc8)c7c6)cccc54)c32)cc1 ; c1ccc(-n2c3ccccc3c3cc(-c4cccc5c4c4ccccc4n5-c4ccccc4-c4cccc5c6ccccc6n(-c6ccccc6)c45)ccc32)cc1]

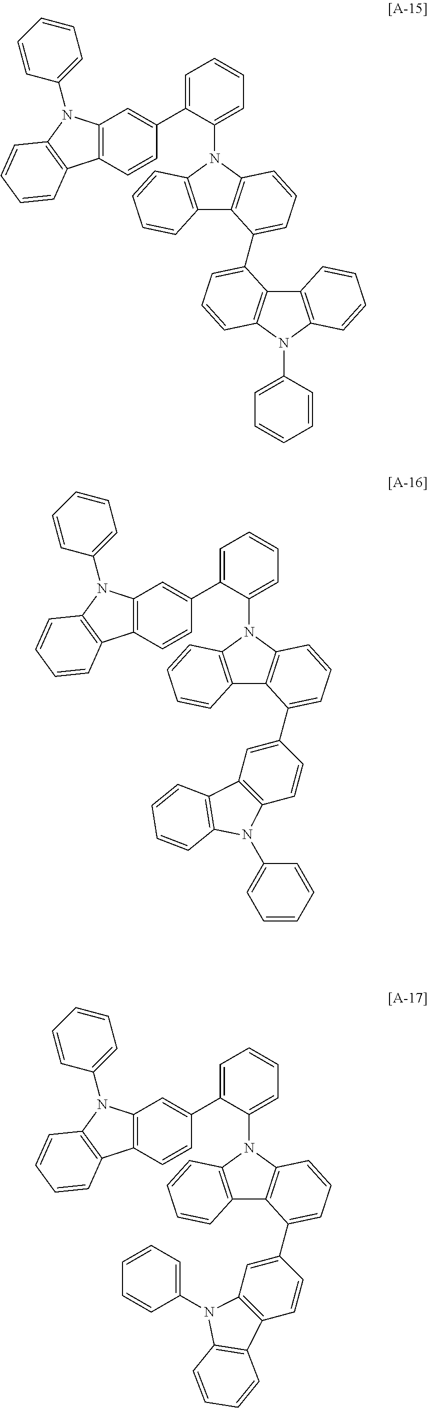 [SMILES: c1ccc(-n2c3ccccc3c3ccc(-c4ccccc4-n4c5ccccc5c5c(-c6cccc7c6c6ccccc6n7-c6ccccc6)cccc54)cc32)cc1 ; c1ccc(-n2c3ccccc3c3cc(-c4cccc5c4c4ccccc4n5-c4ccccc4-c4ccc5c6ccccc6n(-c6ccccc6)c5c4)ccc32)cc1 ; c1ccc(-n2c3ccccc3c3ccc(-c4ccccc4-n4c5ccccc5c5c(-c6ccc7c8ccccc8n(-c8ccccc8)c7c6)cccc54)cc32)cc1]

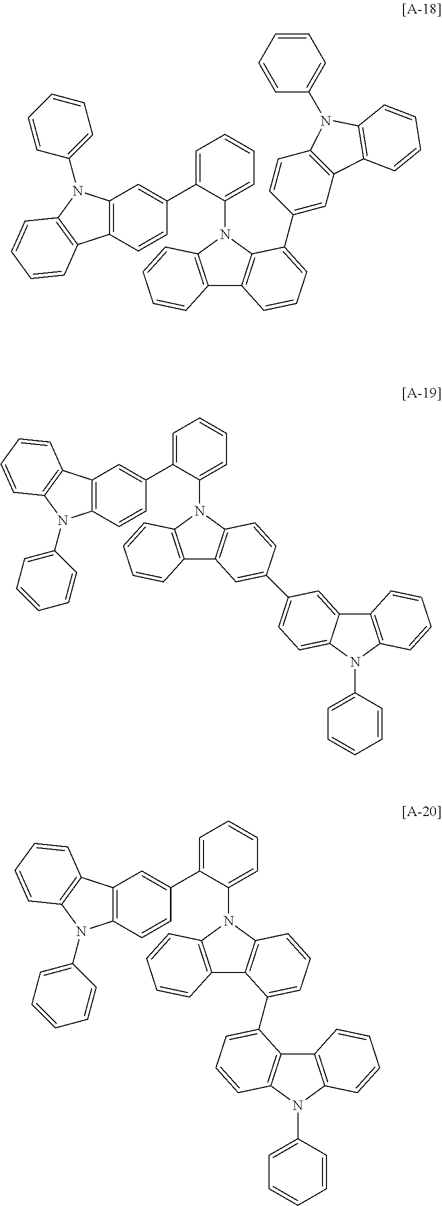 [SMILES: c1ccc(-n2c3ccccc3c3cc(-c4cccc5c6ccccc6n(-c6ccccc6-c6ccc7c8ccccc8n(-c8ccccc8)c7c6)c45)ccc32)cc1 ; c1ccc(-n2c3ccccc3c3cc(-c4ccc5c(c4)c4ccccc4n5-c4ccccc4-c4ccc5c(c4)c4ccccc4n5-c4ccccc4)ccc32)cc1 ; c1ccc(-n2c3ccccc3c3cc(-c4ccccc4-n4c5ccccc5c5c(-c6cccc7c6c6ccccc6n7-c6ccccc6)cccc54)ccc32)cc1]

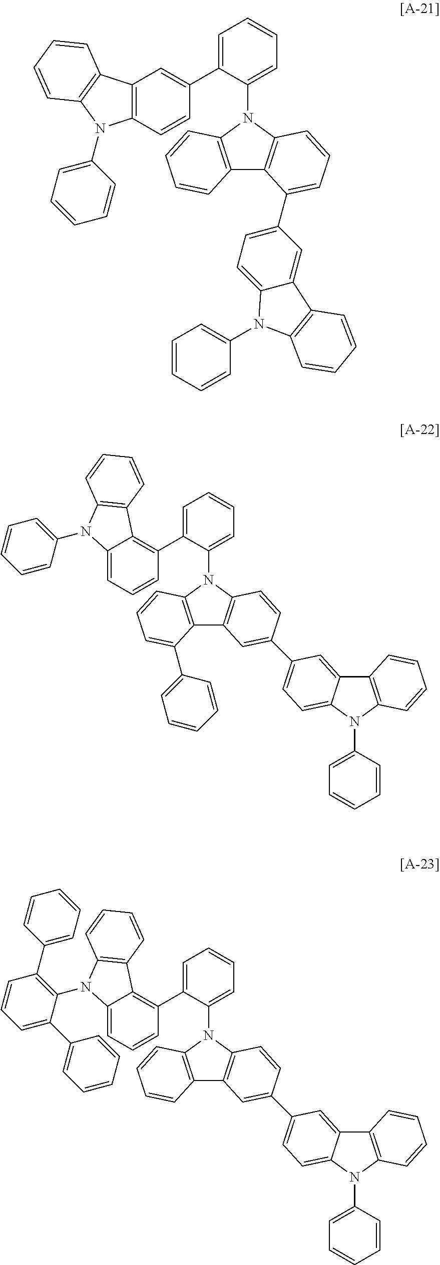 [SMILES: c1ccc(-n2c3ccccc3c3cc(-c4ccccc4-n4c5ccccc5c5c(-c6ccc7c(c6)c6ccccc6n7-c6ccccc6)cccc54)ccc32)cc1 ; c1ccc(-c2cccc3c2c2cc(-c4ccc5c(c4)c4ccccc4n5-c4ccccc4)ccc2n3-c2ccccc2-c2cccc3c2c2ccccc2n3-c2ccccc2)cc1 ; c1ccc(-c2cccc(-c3ccccc3)c2-n2c3ccccc3c3c(-c4ccccc4-n4c5ccccc5c5cc(-c6ccc7c(c6)c6ccccc6n7-c6ccccc6)ccc54)cccc32)cc1]

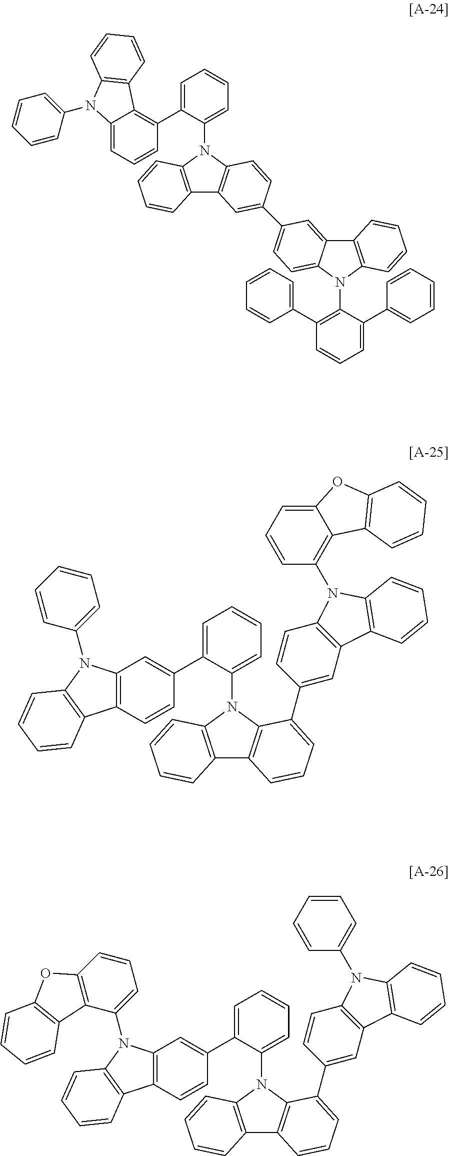 [SMILES: c1ccc(-c2cccc(-c3ccccc3)c2-n2c3ccccc3c3cc(-c4ccc5c(c4)c4ccccc4n5-c4ccccc4-c4cccc5c4c4ccccc4n5-c4ccccc4)ccc32)cc1 ; c1ccc(-n2c3ccccc3c3ccc(-c4ccccc4-n4c5ccccc5c5cccc(-c6ccc7c(c6)c6ccccc6n7-c6cccc7oc8ccccc8c67)c54)cc32)cc1 ; c1ccc(-n2c3ccccc3c3cc(-c4cccc5c6ccccc6n(-c6ccccc6-c6ccc7c8ccccc8n(-c8cccc9oc%10ccccc%10c89)c7c6)c45)ccc32)cc1]

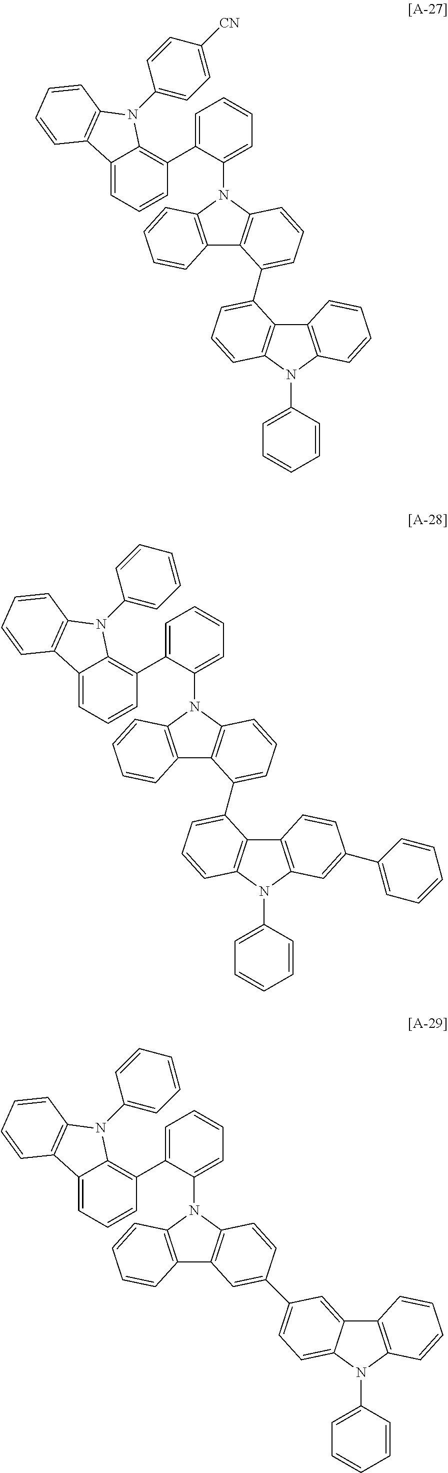 [SMILES: N#Cc1ccc(-n2c3ccccc3c3cccc(-c4ccccc4-n4c5ccccc5c5c(-c6cccc7c6c6ccccc6n7-c6ccccc6)cccc54)c32)cc1 ; c1ccc(-c2ccc3c4c(-c5cccc6c5c5ccccc5n6-c5ccccc5-c5cccc6c7ccccc7n(-c7ccccc7)c56)cccc4n(-c4ccccc4)c3c2)cc1 ; c1ccc(-n2c3ccccc3c3cc(-c4ccc5c(c4)c4ccccc4n5-c4ccccc4-c4cccc5c6ccccc6n(-c6ccccc6)c45)ccc32)cc1]

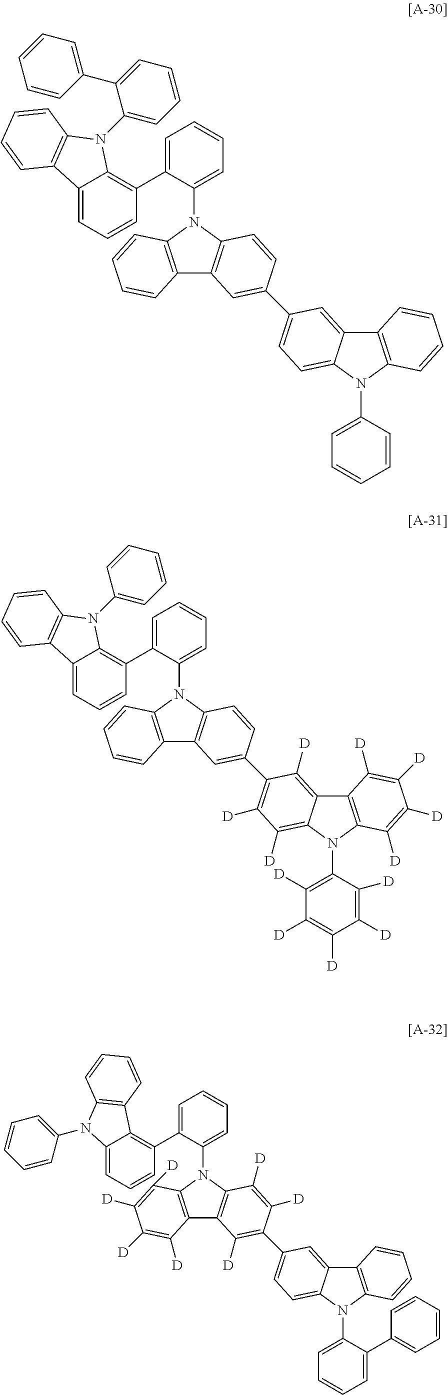 [SMILES: c1ccc(-c2ccccc2-n2c3ccccc3c3cccc(-c4ccccc4-n4c5ccccc5c5cc(-c6ccc7c(c6)c6ccccc6n7-c6ccccc6)ccc54)c32)cc1 ; [2H]c1c([2H])c([2H])c(-n2c3c([2H])c([2H])c([2H])c([2H])c3c3c([2H])c(-c4ccc5c(c4)c4ccccc4n5-c4ccccc4-c4cccc5c6ccccc6n(-c6ccccc6)c45)c([2H])c([2H])c32)c([2H])c1[2H] ; [2H]c1c([2H])c([2H])c2c(c1[2H])c1c([2H])c(-c3ccc4c(c3)c3ccccc3n4-c3ccccc3-c3ccccc3)c([2H])c([2H])c1n2-c1ccccc1-c1cccc2c1c1ccccc1n2-c1ccccc1]

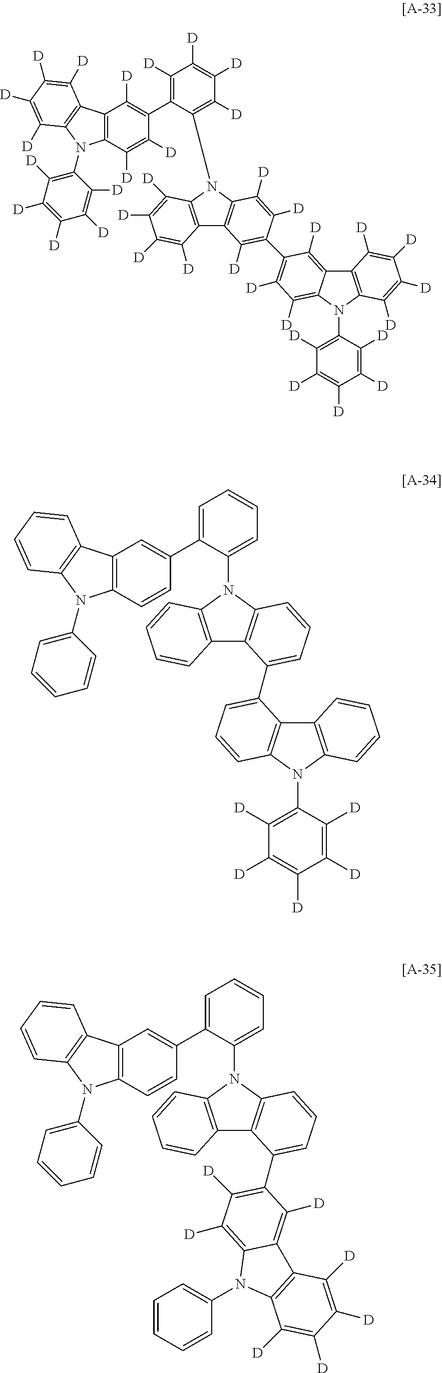

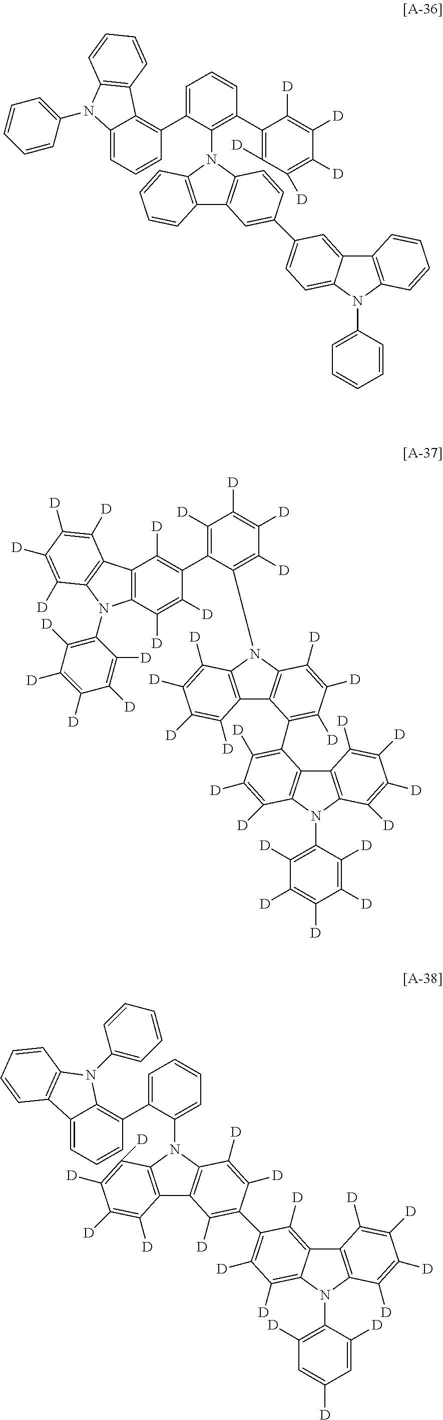

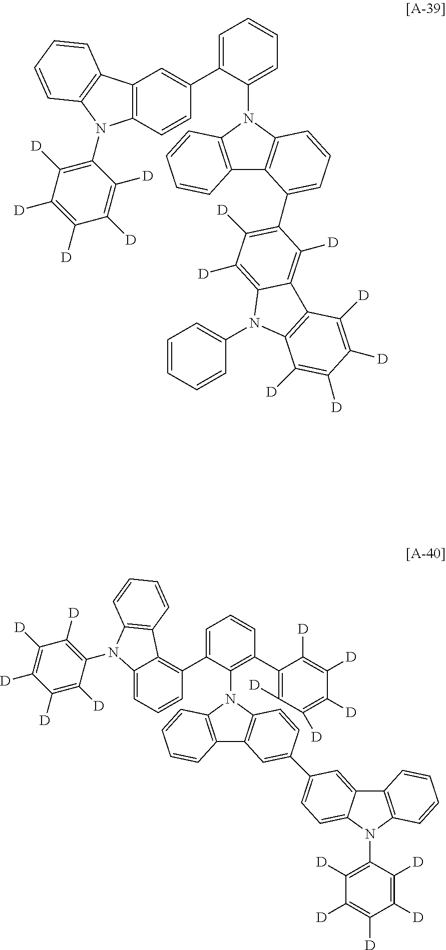 [SMILES: [2H]c1c([2H])c([2H])c(-n2c3ccccc3c3cc(-c4ccccc4-n4c5ccccc5c5c(-c6c([2H])c([2H])c7c(c6[2H])c6c([2H])c([2H])c([2H])c([2H])c6n7-c6ccccc6)cccc54)ccc32)c([2H])c1[2H] ; [2H]c1c([2H])c([2H])c(-c2cccc(-c3cccc4c3c3ccccc3n4-c3c([2H])c([2H])c([2H])c([2H])c3[2H])c2-n2c3ccccc3c3cc(-c4ccc5c(c4)c4ccccc4n5-c4c([2H])c([2H])c([2H])c([2H])c4[2H])ccc32)c([2H])c1[2H]]

For example, the second compound may be represented by any one of Chemical Formula 2A to Chemical Formula 2F.

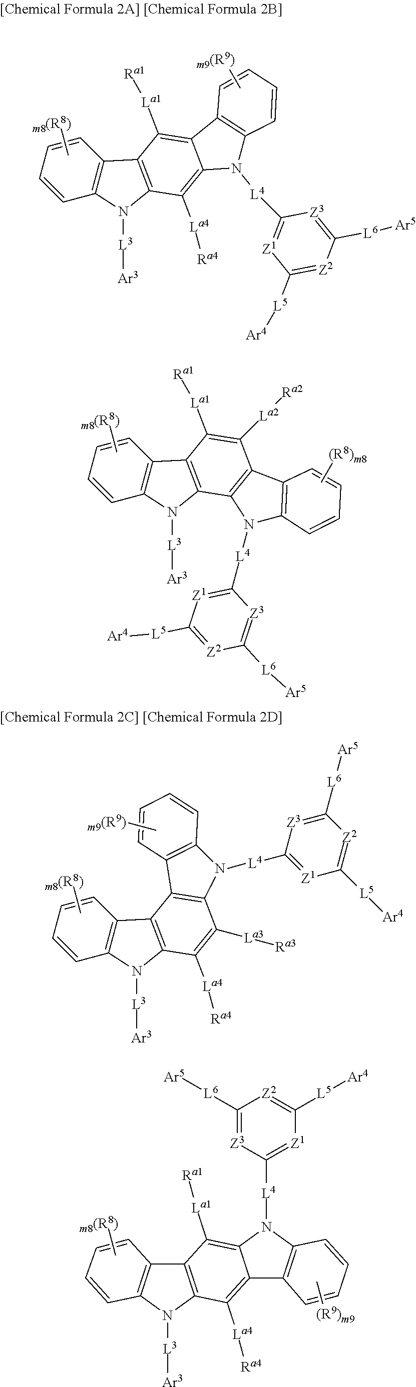

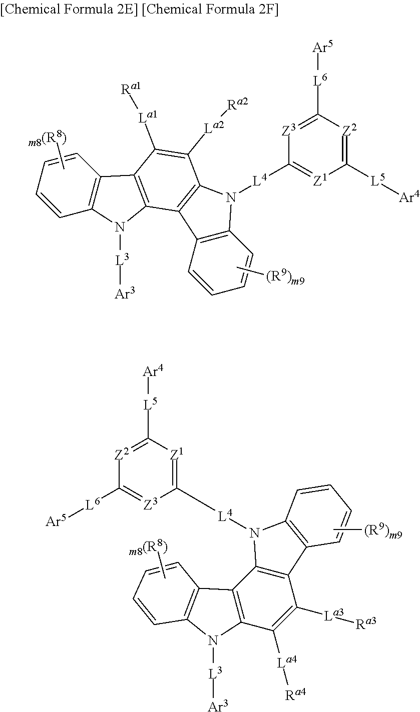

In Chemical Formula 2A to Chemical Formula 2F,
Z¹ to Z³, R⁸, R⁹, L³ to L⁶, Ar³ to Ar⁵, m8, and m9 are the same as defined in Chemical Formula 2 and Chemical Formula 3,
R^{a1} to R^{a4} are defined as R⁸ and R⁹, and
L^{a1} to L^{a4} are defined as L³ to L⁶.

As a specific example, the second compound may be represented by one of Chemical Formula 2A, Chemical Formula 2B, and Chemical Formula 2D.

As a more specific example, the second compound may be represented by Chemical Formula 2B or Chemical Formula 2D.

In an embodiment, the second compound may be represented by Chemical Formula 2B.

When m8 is 2 or more, each R⁸ may be the same or different from each other.

When m9 is 2 or more, each R⁹ may be the same or different from each other.

For example, Ar³ may be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, a substituted or unsubstituted dibenzofuranyl group, a substituted or unsubstituted dibenzothiophenyl group, a substituted or unsubstituted carbazolyl group, or a substituted or unsubstituted dibenzosilolyl group.

For example, Ar⁴ and Ar⁵ may each independently be a substituted or unsubstituted phenyl group, a substituted or unsubstituted biphenyl group, a substituted or unsubstituted terphenyl group, a substituted or unsubstituted fluorenyl group, a substituted or unsubstituted dibenzofuranyl group, a substituted or unsubstituted dibenzothiophenyl group, a substituted or unsubstituted carbazolyl group, or a substituted or unsubstituted dibenzosilolyl group.

As a specific example, Ar⁴ and Ar⁵ may each independently be a substituted or unsubstituted phenyl group, a substituted or unsubstituted biphenyl group, a substituted or unsubstituted terphenyl group, or a substituted or unsubstituted fluorenyl group.

For example, L⁴ to L⁶ may each independently be a single bond, a substituted or unsubstituted phenylene group, or a substituted or unsubstituted biphenylene group.

L⁵-Ar⁴ and L⁶-Ar⁵ may each independently be selected from substituents listed in Group II.

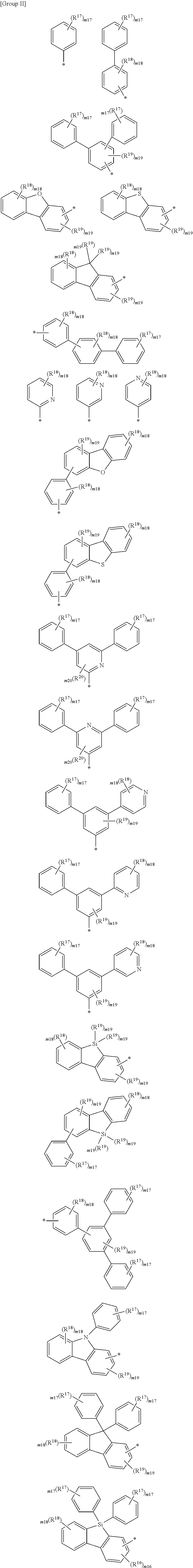

In Group II,
R¹⁷ to R²⁰, R^{19a}, R^{19b}, R^{19c}, R^{19d}, R^{19e}, and R^{19f} are each independently hydrogen, deuterium, a substituted or unsubstituted C1 to C4 alkyl group, a substituted or unsubstituted C6 to C18 aryl group, or a substituted or unsubstituted C2 to C30 heteroaryl group,
m17 is one of integers of 1 to 5,
m18 is one of integers of 1 to 4,
m19 is one of integers of 1 to 3,
m20 is an integer of 1 or 2, and
* is a linking point.

When m17 is 2 or more, each R¹⁷ may be the same or different from each other.

When m18 is 2 or more, each R¹⁸ may be the same or different from each other.

When m19 is 2 or more, each R¹⁹ may be the same or different from each other.

When m20 is 2 or more, each R²⁰ may be the same or different from each other.

As an example, R⁸ and R⁹ may each independently be hydrogen, deuterium, a cyano group, a halogen, a substituted or unsubstituted C1 to C10 alkyl group, a substituted or unsubstituted phenyl group, a substituted or unsubstituted biphenyl group, a substituted or unsubstituted terphenyl group, or a substituted or unsubstituted naphthyl group.

For example, R⁸ and R⁹ may each independently be hydrogen, deuterium, a cyano group, a substituted or unsubstituted C1 to C6 alkyl group, a substituted or unsubstituted phenyl group, a substituted or unsubstituted biphenyl group, or a substituted or unsubstituted terphenyl group.

As an example, R^{a1} to R^{a4} may each independently be hydrogen, deuterium, a cyano group, a halogen, a substituted or unsubstituted C1 to C10 alkyl group, a substituted or unsubstituted phenyl group, a substituted or unsubstituted biphenyl group, a substituted or unsubstituted terphenyl group, or a substituted or unsubstituted naphthyl group.

For example, R^{a1} to R^{a4} may each independently be hydrogen, deuterium, a cyano group, a substituted or unsubstituted C1 to C6 alkyl group, a substituted or unsubstituted phenyl group, a substituted or unsubstituted biphenyl group, or a substituted or unsubstituted terphenyl group.

For example, the second compound may be one selected from the compounds listed in Group 2, but is not limited thereto.

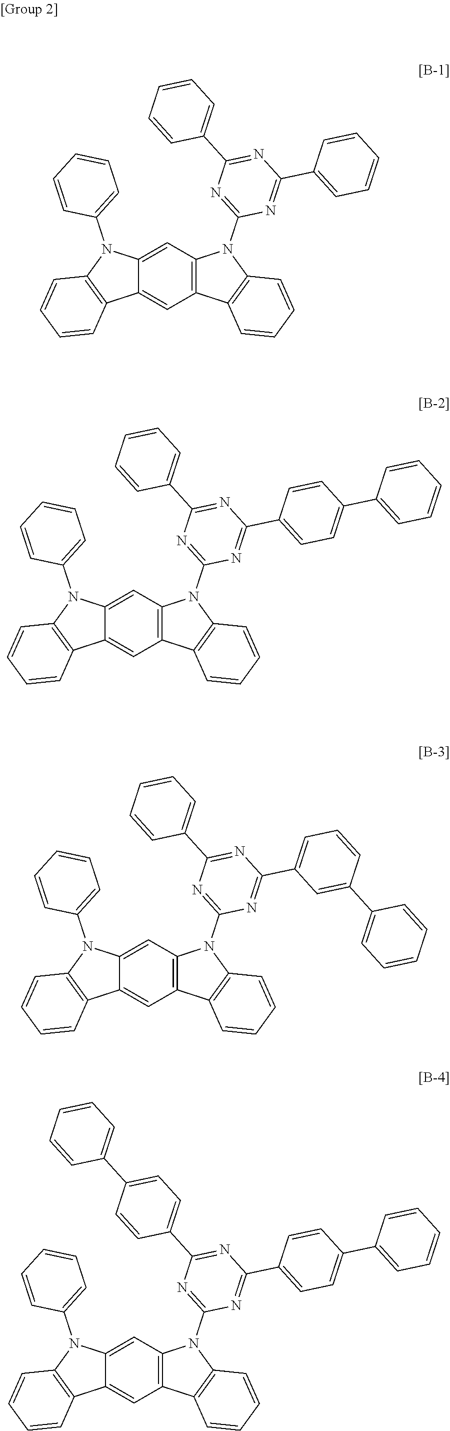 [SMILES: c1ccc(-c2nc(-c3ccccc3)nc(-n3c4ccccc4c4cc5c6ccccc6n(-c6ccccc6)c5cc43)n2)cc1 ; c1ccc(-c2ccc(-c3nc(-c4ccccc4)nc(-n4c5ccccc5c5cc6c7ccccc7n(-c7ccccc7)c6cc54)n3)cc2)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc(-c2ccc(-c3nc(-c4ccc(-c5ccccc5)cc4)nc(-n4c5ccccc5c5cc6c7ccccc7n(-c7ccccc7)c6cc54)n3)cc2)cc1]

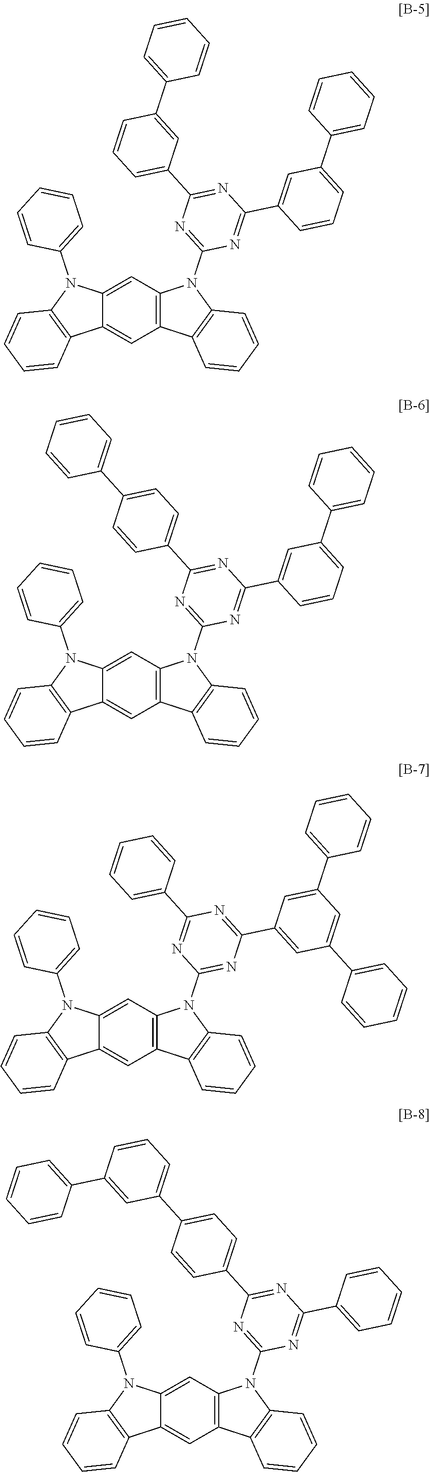 [SMILES: c1ccc(-c2cccc(-c3nc(-c4cccc(-c5ccccc5)c4)nc(-n4c5ccccc5c5cc6c7ccccc7n(-c7ccccc7)c6cc54)n3)c2)cc1 ; c1ccc(-c2ccc(-c3nc(-c4cccc(-c5ccccc5)c4)nc(-n4c5ccccc5c5cc6c7ccccc7n(-c7ccccc7)c6cc54)n3)cc2)cc1 ; c1ccc(-c2cc(-c3ccccc3)cc(-c3nc(-c4ccccc4)nc(-n4c5ccccc5c5cc6c7ccccc7n(-c7ccccc7)c6cc54)n3)c2)cc1 ; c1ccc(-c2cccc(-c3ccc(-c4nc(-c5ccccc5)nc(-n5c6ccccc6c6cc7c8ccccc8n(-c8ccccc8)c7cc65)n4)cc3)c2)cc1]

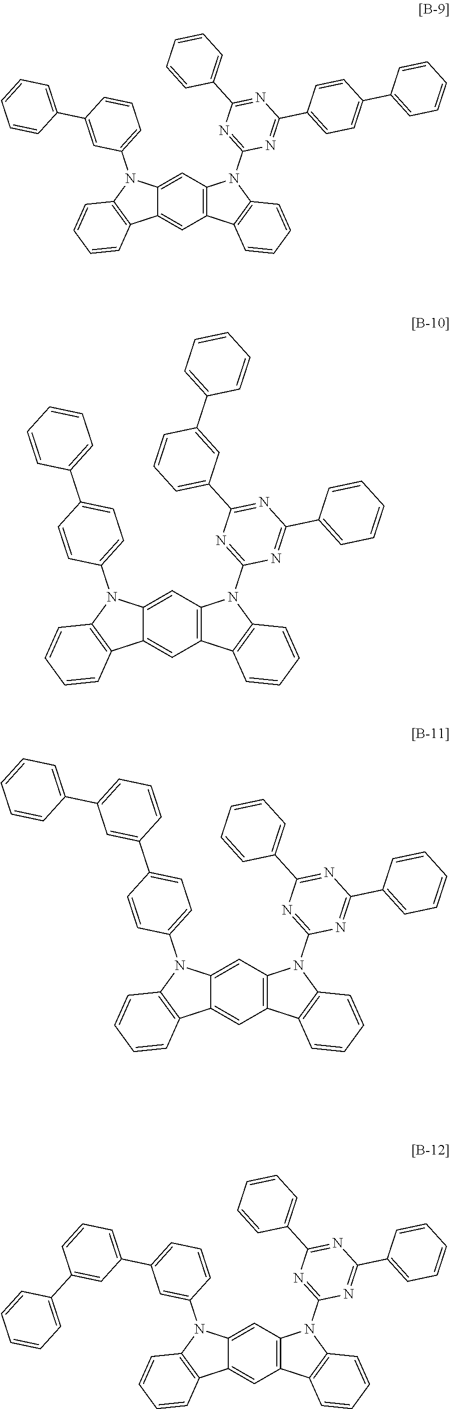 [SMILES: c1ccc(-c2ccc(-c3nc(-c4ccccc4)nc(-n4c5ccccc5c5cc6c7ccccc7n(-c7cccc(-c8ccccc8)c7)c6cc54)n3)cc2)cc1 ; c1ccc(-c2ccc(-n3c4ccccc4c4cc5c6ccccc6n(-c6nc(-c7ccccc7)nc(-c7cccc(-c8ccccc8)c7)n6)c5cc43)cc2)cc1 ; c1ccc(-c2cccc(-c3ccc(-n4c5ccccc5c5cc6c7ccccc7n(-c7nc(-c8ccccc8)nc(-c8ccccc8)n7)c6cc54)cc3)c2)cc1 ; c1ccc(-c2cccc(-c3cccc(-n4c5ccccc5c5cc6c7ccccc7n(-c7nc(-c8ccccc8)nc(-c8ccccc8)n7)c6cc54)c3)c2)cc1]

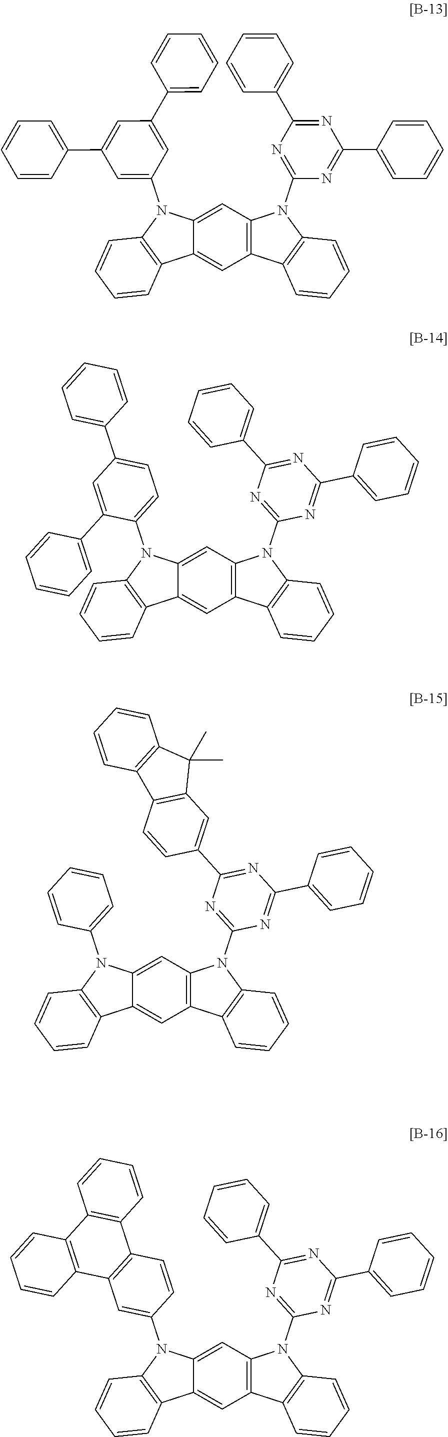 [SMILES: c1ccc(-c2cc(-c3ccccc3)cc(-n3c4ccccc4c4cc5c6ccccc6n(-c6nc(-c7ccccc7)nc(-c7ccccc7)n6)c5cc43)c2)cc1 ; c1ccc(-c2ccc(-n3c4ccccc4c4cc5c6ccccc6n(-c6nc(-c7ccccc7)nc(-c7ccccc7)n6)c5cc43)c(-c3ccccc3)c2)cc1 ; CC1(C)c2ccccc2-c2ccc(-c3nc(-c4ccccc4)nc(-n4c5ccccc5c5cc6c7ccccc7n(-c7ccccc7)c6cc54)n3)cc21 ; c1ccc(-c2nc(-c3ccccc3)nc(-n3c4ccccc4c4cc5c6ccccc6n(-c6ccc7c8ccccc8c8ccccc8c7c6)c5cc43)n2)cc1]

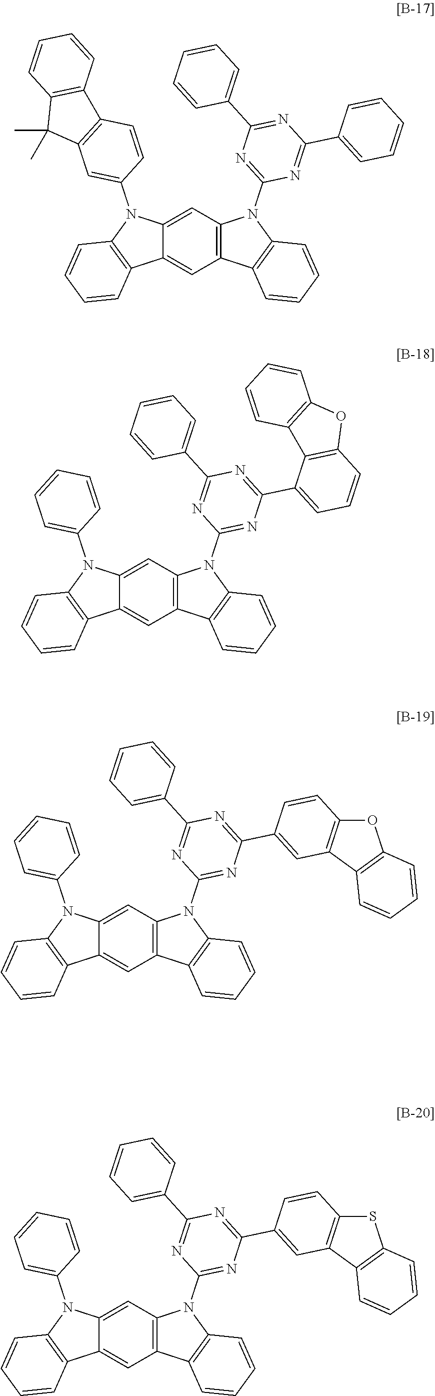 [SMILES: CC1(C)c2ccccc2-c2ccc(-n3c4ccccc4c4cc5c6ccccc6n(-c6nc(-c7ccccc7)nc(-c7ccccc7)n6)c5cc43)cc21 ; c1ccc(-c2nc(-c3cccc4oc5ccccc5c34)nc(-n3c4ccccc4c4cc5c6ccccc6n(-c6ccccc6)c5cc43)n2)cc1 ; c1ccc(-c2nc(-c3ccc4oc5ccccc5c4c3)nc(-n3c4ccccc4c4cc5c6ccccc6n(-c6ccccc6)c5cc43)n2)cc1 ; c1ccc(-c2nc(-c3ccc4sc5ccccc5c4c3)nc(-n3c4ccccc4c4cc5c6ccccc6n(-c6ccccc6)c5cc43)n2)cc1]

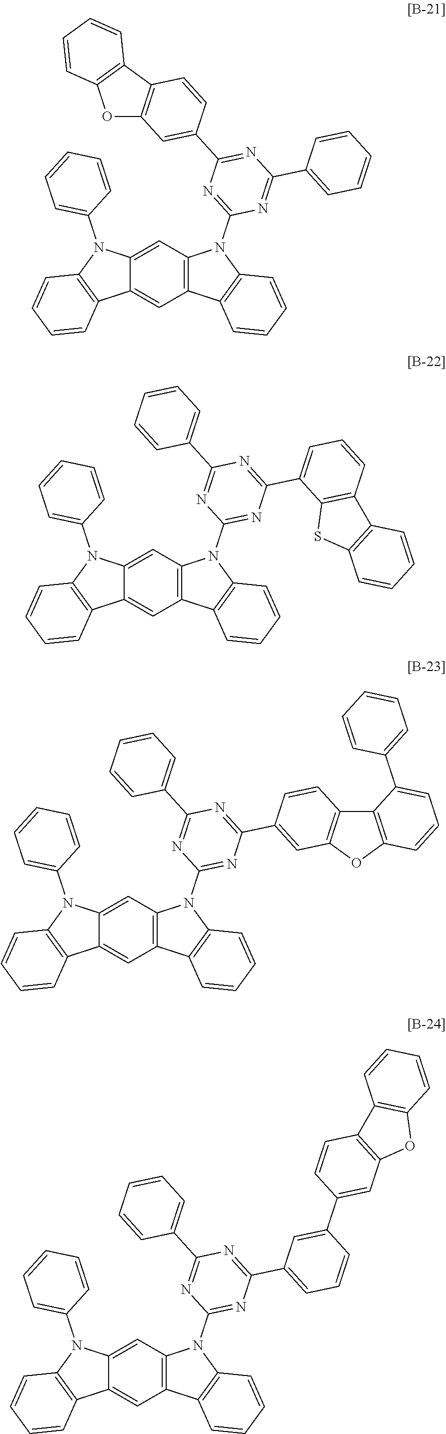 [SMILES: c1ccc(-c2nc(-c3ccc4c(c3)oc3ccccc34)nc(-n3c4ccccc4c4cc5c6ccccc6n(-c6ccccc6)c5cc43)n2)cc1 ; c1ccc(-c2nc(-c3cccc4c3sc3ccccc34)nc(-n3c4ccccc4c4cc5c6ccccc6n(-c6ccccc6)c5cc43)n2)cc1 ; c1ccc(-c2nc(-c3ccc4c(c3)oc3cccc(-c5ccccc5)c34)nc(-n3c4ccccc4c4cc5c6ccccc6n(-c6ccccc6)c5cc43)n2)cc1 ; c1ccc(-c2nc(-c3cccc(-c4ccc5c(c4)oc4ccccc45)c3)nc(-n3c4ccccc4c4cc5c6ccccc6n(-c6ccccc6)c5cc43)n2)cc1]

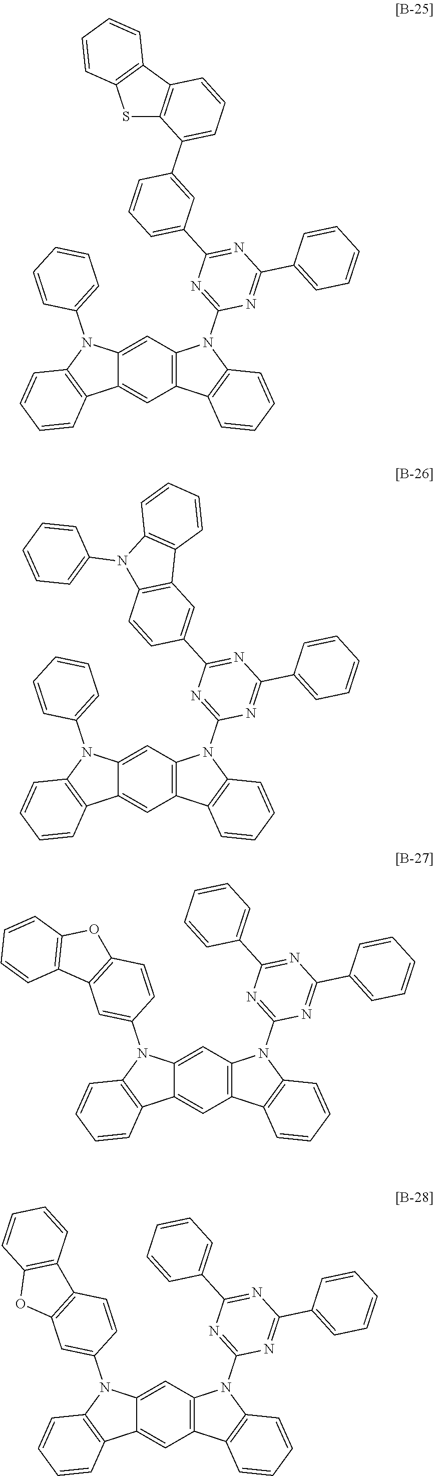 [SMILES: c1ccc(-c2nc(-c3cccc(-c4cccc5c4sc4ccccc45)c3)nc(-n3c4ccccc4c4cc5c6ccccc6n(-c6ccccc6)c5cc43)n2)cc1 ; c1ccc(-c2nc(-c3ccc4c(c3)c3ccccc3n4-c3ccccc3)nc(-n3c4ccccc4c4cc5c6ccccc6n(-c6ccccc6)c5cc43)n2)cc1 ; c1ccc(-c2nc(-c3ccccc3)nc(-n3c4ccccc4c4cc5c6ccccc6n(-c6ccc7oc8ccccc8c7c6)c5cc43)n2)cc1 ; c1ccc(-c2nc(-c3ccccc3)nc(-n3c4ccccc4c4cc5c6ccccc6n(-c6ccc7c(c6)oc6ccccc67)c5cc43)n2)cc1]

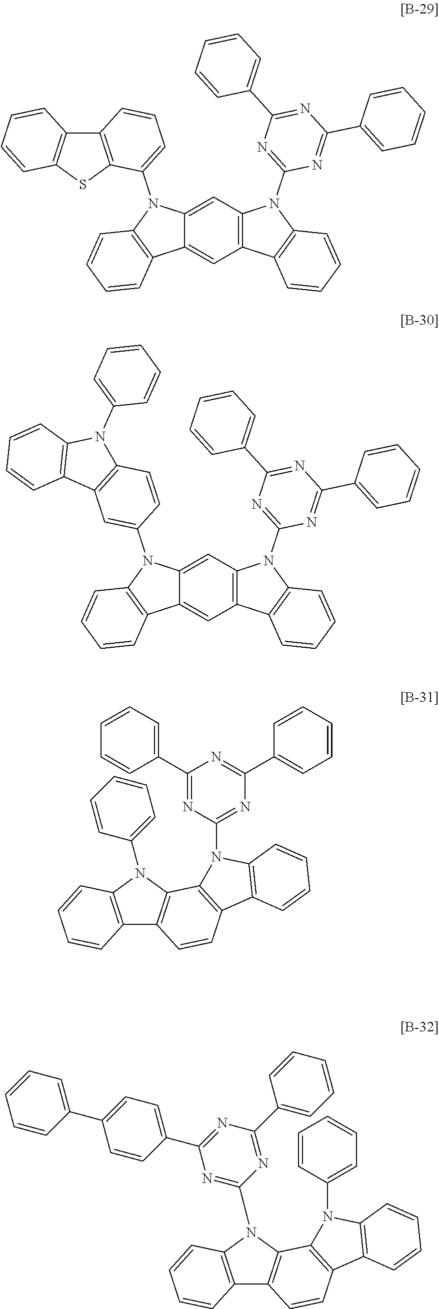 [SMILES: c1ccc(-c2nc(-c3ccccc3)nc(-n3c4ccccc4c4cc5c6ccccc6n(-c6cccc7c6sc6ccccc67)c5cc43)n2)cc1 ; c1ccc(-c2nc(-c3ccccc3)nc(-n3c4ccccc4c4cc5c6ccccc6n(-c6ccc7c(c6)c6ccccc6n7-c6ccccc6)c5cc43)n2)cc1 ; c1ccc(-c2nc(-c3ccccc3)nc(-n3c4ccccc4c4ccc5c6ccccc6n(-c6ccccc6)c5c43)n2)cc1 ; c1ccc(-c2ccc(-c3nc(-c4ccccc4)nc(-n4c5ccccc5c5ccc6c7ccccc7n(-c7ccccc7)c6c54)n3)cc2)cc1]

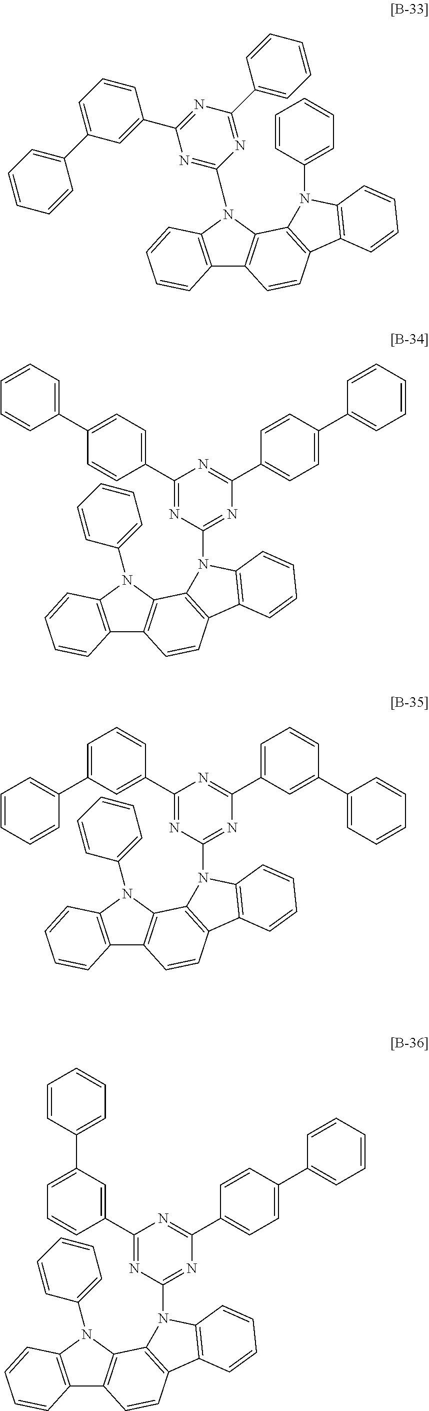 [SMILES: c1ccc(-c2cccc(-c3nc(-c4ccccc4)nc(-n4c5ccccc5c5ccc6c7ccccc7n(-c7ccccc7)c6c54)n3)c2)cc1 ; c1ccc(-c2ccc(-c3nc(-c4ccc(-c5ccccc5)cc4)nc(-n4c5ccccc5c5ccc6c7ccccc7n(-c7ccccc7)c6c54)n3)cc2)cc1 ; c1ccc(-c2cccc(-c3nc(-c4cccc(-c5ccccc5)c4)nc(-n4c5ccccc5c5ccc6c7ccccc7n(-c7ccccc7)c6c54)n3)c2)cc1 ; c1ccc(-c2ccc(-c3nc(-c4cccc(-c5ccccc5)c4)nc(-n4c5ccccc5c5ccc6c7ccccc7n(-c7ccccc7)c6c54)n3)cc2)cc1]

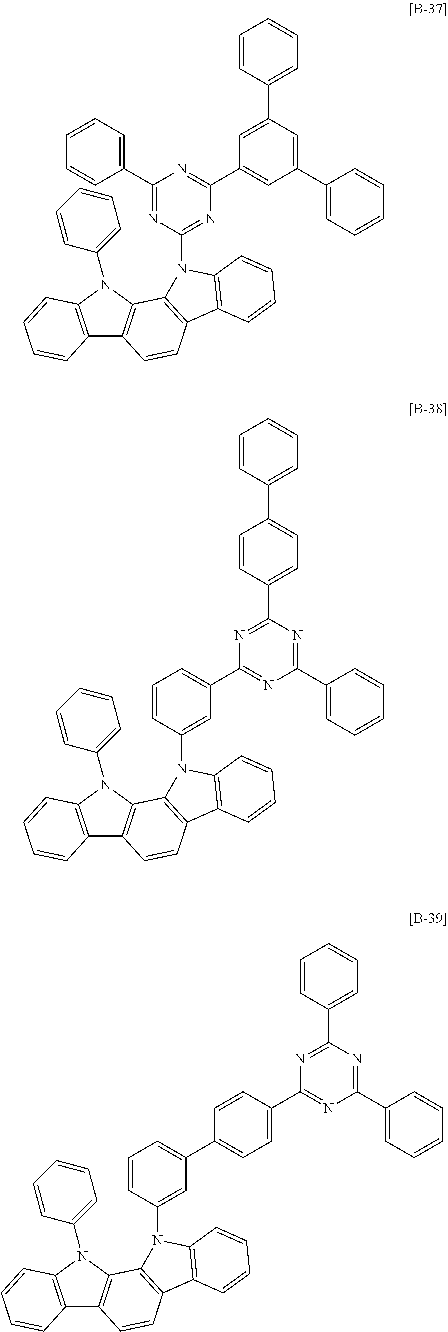 [SMILES: c1ccc(-c2cc(-c3ccccc3)cc(-c3nc(-c4ccccc4)nc(-n4c5ccccc5c5ccc6c7ccccc7n(-c7ccccc7)c6c54)n3)c2)cc1 ; c1ccc(-c2ccc(-c3nc(-c4ccccc4)nc(-c4cccc(-n5c6ccccc6c6ccc7c8ccccc8n(-c8ccccc8)c7c65)c4)n3)cc2)cc1 ; c1ccc(-c2nc(-c3ccccc3)nc(-c3ccc(-c4cccc(-n5c6ccccc6c6ccc7c8ccccc8n(-c8ccccc8)c7c65)c4)cc3)n2)cc1]

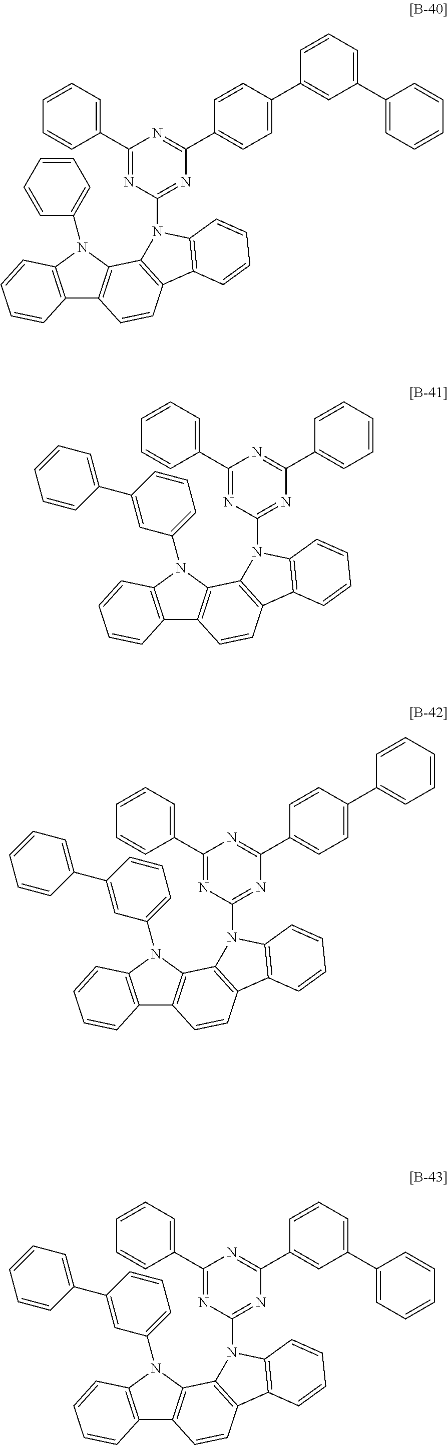 [SMILES: c1ccc(-c2cccc(-c3ccc(-c4nc(-c5ccccc5)nc(-n5c6ccccc6c6ccc7c8ccccc8n(-c8ccccc8)c7c65)n4)cc3)c2)cc1 ; c1ccc(-c2cccc(-n3c4ccccc4c4ccc5c6ccccc6n(-c6nc(-c7ccccc7)nc(-c7ccccc7)n6)c5c43)c2)cc1 ; c1ccc(-c2ccc(-c3nc(-c4ccccc4)nc(-n4c5ccccc5c5ccc6c7ccccc7n(-c7cccc(-c8ccccc8)c7)c6c54)n3)cc2)cc1 ; c1ccc(-c2cccc(-c3nc(-c4ccccc4)nc(-n4c5ccccc5c5ccc6c7ccccc7n(-c7cccc(-c8ccccc8)c7)c6c54)n3)c2)cc1]

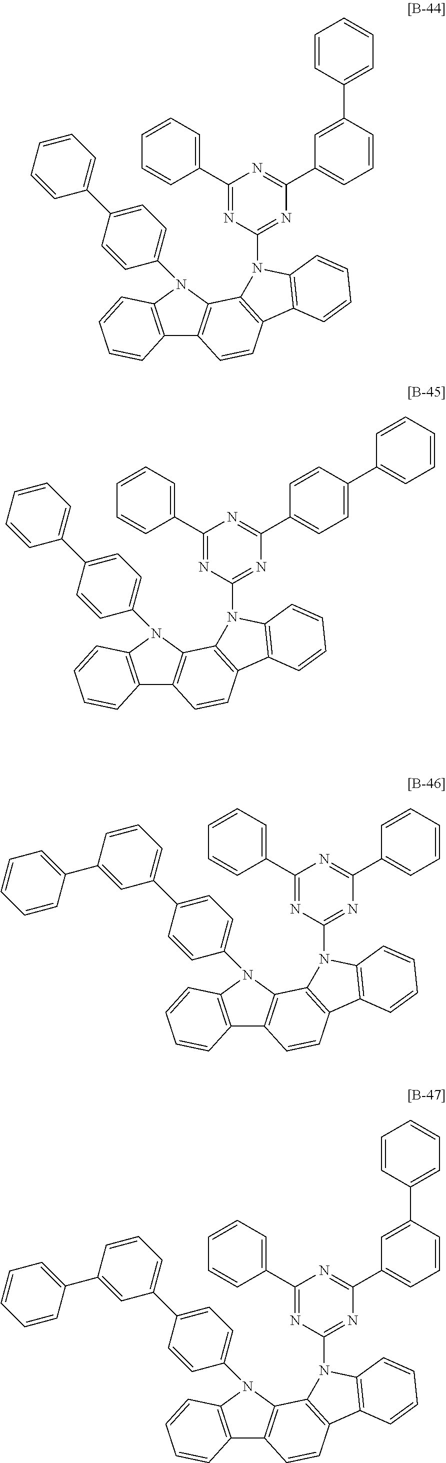 [SMILES: c1ccc(-c2ccc(-n3c4ccccc4c4ccc5c6ccccc6n(-c6nc(-c7ccccc7)nc(-c7cccc(-c8ccccc8)c7)n6)c5c43)cc2)cc1 ; c1ccc(-c2ccc(-c3nc(-c4ccccc4)nc(-n4c5ccccc5c5ccc6c7ccccc7n(-c7ccc(-c8ccccc8)cc7)c6c54)n3)cc2)cc1 ; c1ccc(-c2cccc(-c3ccc(-n4c5ccccc5c5ccc6c7ccccc7n(-c7nc(-c8ccccc8)nc(-c8ccccc8)n7)c6c54)cc3)c2)cc1 ; c1ccc(-c2cccc(-c3ccc(-n4c5ccccc5c5ccc6c7ccccc7n(-c7nc(-c8ccccc8)nc(-c8cccc(-c9ccccc9)c8)n7)c6c54)cc3)c2)cc1]

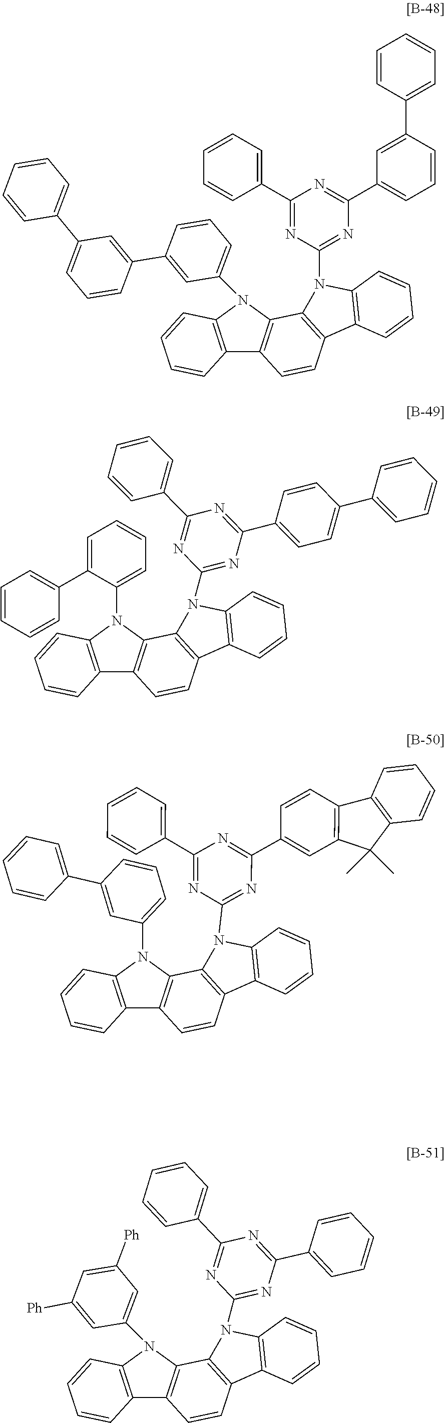 [SMILES: c1ccc(-c2cccc(-c3cccc(-n4c5ccccc5c5ccc6c7ccccc7n(-c7nc(-c8ccccc8)nc(-c8cccc(-c9ccccc9)c8)n7)c6c54)c3)c2)cc1 ; c1ccc(-c2ccc(-c3nc(-c4ccccc4)nc(-n4c5ccccc5c5ccc6c7ccccc7n(-c7ccccc7-c7ccccc7)c6c54)n3)cc2)cc1 ; CC1(C)c2ccccc2-c2ccc(-c3nc(-c4ccccc4)nc(-n4c5ccccc5c5ccc6c7ccccc7n(-c7cccc(-c8ccccc8)c7)c6c54)n3)cc21 ; c1ccc(-c2cc(-c3ccccc3)cc(-n3c4ccccc4c4ccc5c6ccccc6n(-c6nc(-c7ccccc7)nc(-c7ccccc7)n6)c5c43)c2)cc1]

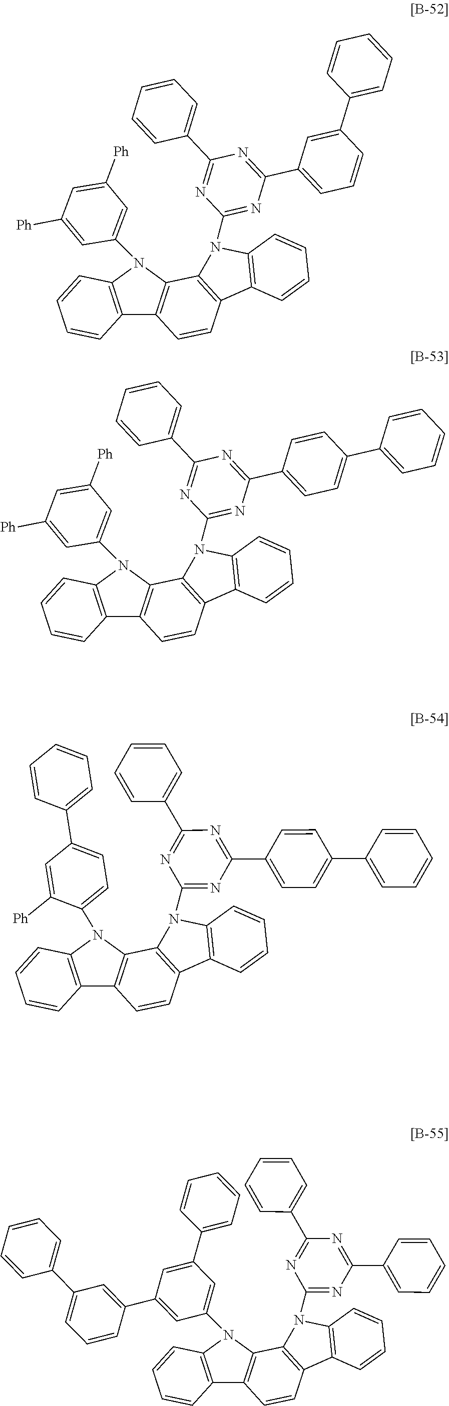 [SMILES: c1ccc(-c2cccc(-c3nc(-c4ccccc4)nc(-n4c5ccccc5c5ccc6c7ccccc7n(-c7cc(-c8ccccc8)cc(-c8ccccc8)c7)c6c54)n3)c2)cc1 ; c1ccc(-c2ccc(-c3nc(-c4ccccc4)nc(-n4c5ccccc5c5ccc6c7ccccc7n(-c7cc(-c8ccccc8)cc(-c8ccccc8)c7)c6c54)n3)cc2)cc1 ; c1ccc(-c2ccc(-c3nc(-c4ccccc4)nc(-n4c5ccccc5c5ccc6c7ccccc7n(-c7ccc(-c8ccccc8)cc7-c7ccccc7)c6c54)n3)cc2)cc1 ; c1ccc(-c2cccc(-c3cc(-c4ccccc4)cc(-n4c5ccccc5c5ccc6c7ccccc7n(-c7nc(-c8ccccc8)nc(-c8ccccc8)n7)c6c54)c3)c2)cc1]

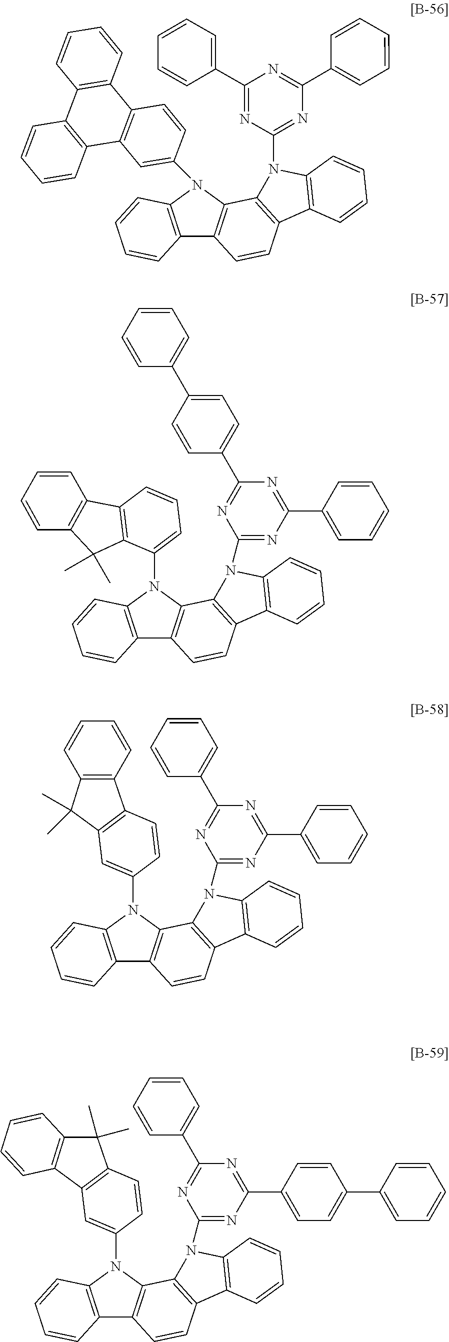 [SMILES: c1ccc(-c2nc(-c3ccccc3)nc(-n3c4ccccc4c4ccc5c6ccccc6n(-c6ccc7c8ccccc8c8ccccc8c7c6)c5c43)n2)cc1 ; CC1(C)c2ccccc2-c2cccc(-n3c4ccccc4c4ccc5c6ccccc6n(-c6nc(-c7ccccc7)nc(-c7ccc(-c8ccccc8)cc7)n6)c5c43)c21 ; CC1(C)c2ccccc2-c2ccc(-n3c4ccccc4c4ccc5c6ccccc6n(-c6nc(-c7ccccc7)nc(-c7ccccc7)n6)c5c43)cc21 ; CC1(C)c2ccccc2-c2cc(-n3c4ccccc4c4ccc5c6ccccc6n(-c6nc(-c7ccccc7)nc(-c7ccc(-c8ccccc8)cc7)n6)c5c43)ccc21]

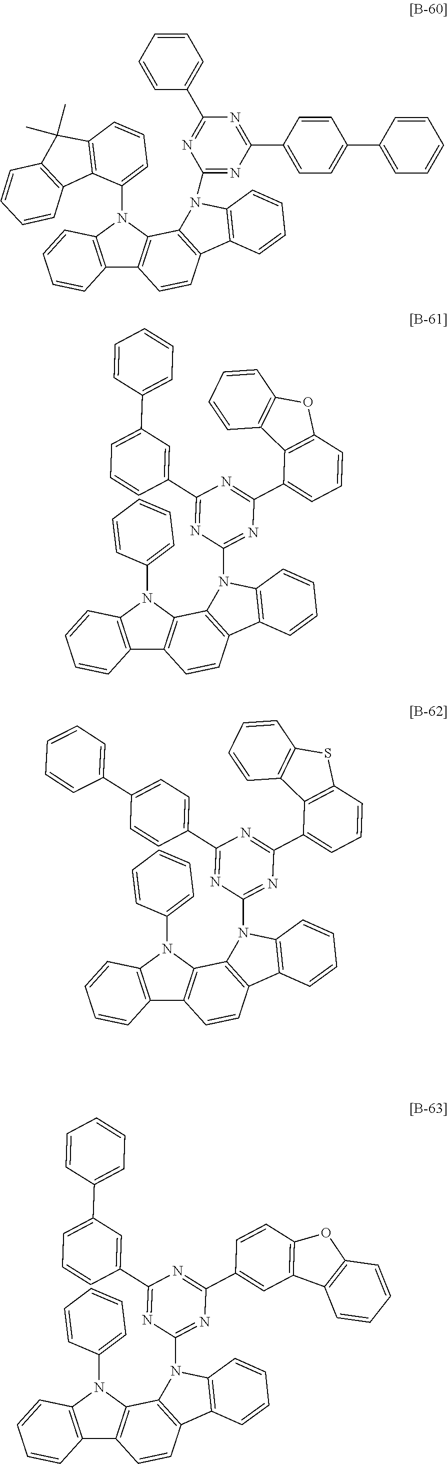

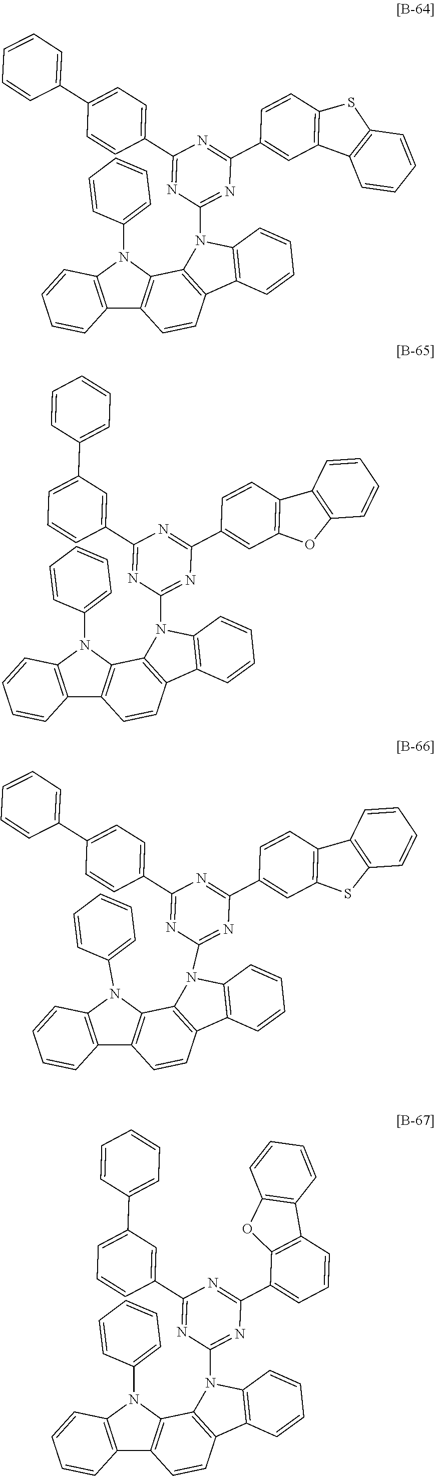

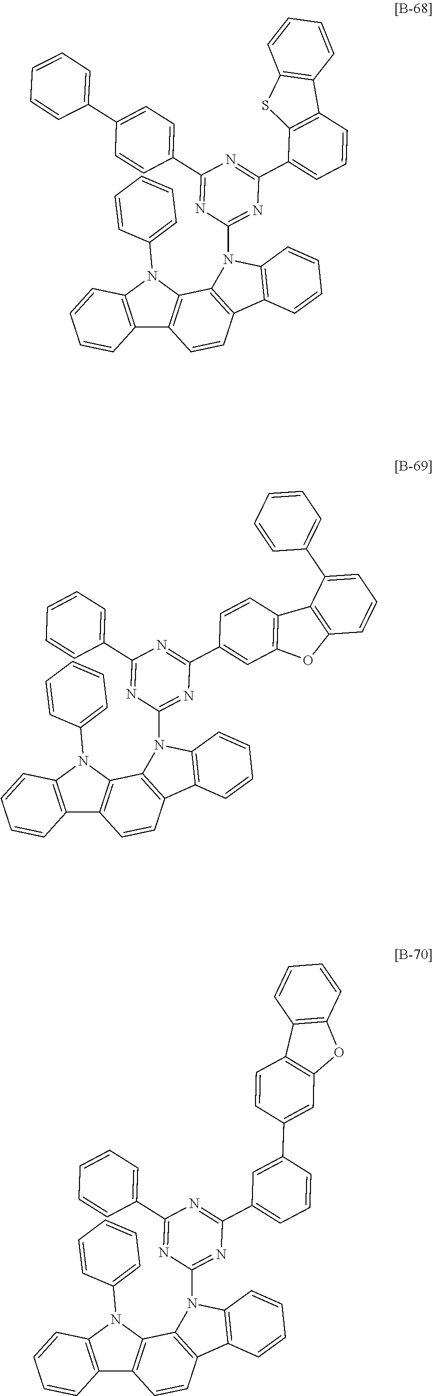 [SMILES: c1ccc(-c2ccc(-c3nc(-c4cccc5c4sc4ccccc45)nc(-n4c5ccccc5c5ccc6c7ccccc7n(-c7ccccc7)c6c54)n3)cc2)cc1 ; c1ccc(-c2nc(-c3ccc4c(c3)oc3cccc(-c5ccccc5)c34)nc(-n3c4ccccc4c4ccc5c6ccccc6n(-c6ccccc6)c5c43)n2)cc1 ; c1ccc(-c2nc(-c3cccc(-c4ccc5c(c4)oc4ccccc45)c3)nc(-n3c4ccccc4c4ccc5c6ccccc6n(-c6ccccc6)c5c43)n2)cc1]

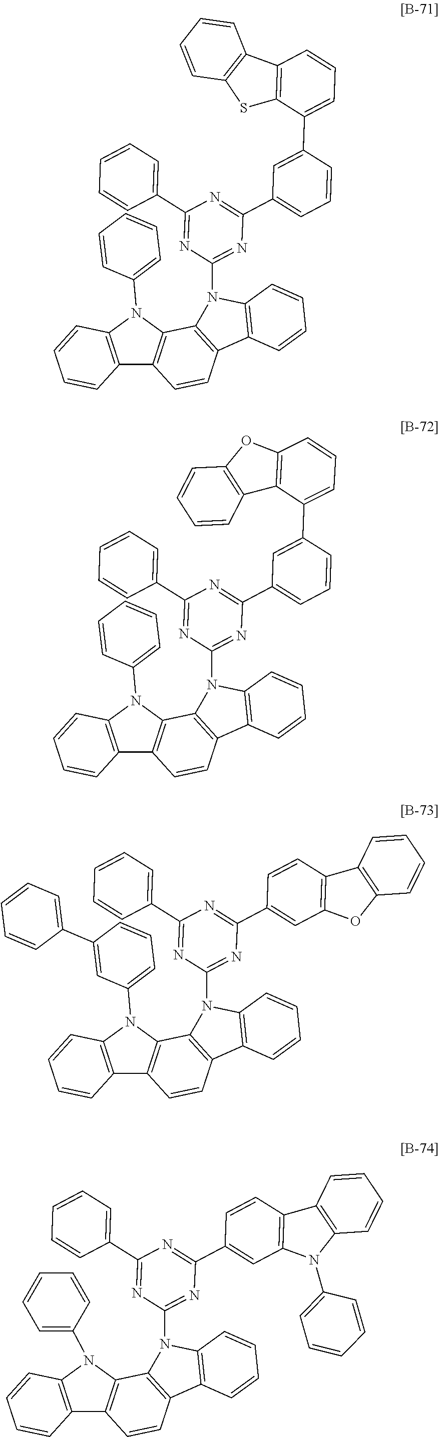 [SMILES: c1ccc(-c2nc(-c3cccc(-c4cccc5c4sc4ccccc45)c3)nc(-n3c4ccccc4c4ccc5c6ccccc6n(-c6ccccc6)c5c43)n2)cc1 ; c1ccc(-c2nc(-c3cccc(-c4cccc5oc6ccccc6c45)c3)nc(-n3c4ccccc4c4ccc5c6ccccc6n(-c6ccccc6)c5c43)n2)cc1 ; c1ccc(-c2cccc(-n3c4ccccc4c4ccc5c6ccccc6n(-c6nc(-c7ccccc7)nc(-c7ccc8c(c7)oc7ccccc78)n6)c5c43)c2)cc1 ; c1ccc(-c2nc(-c3ccc4c5ccccc5n(-c5ccccc5)c4c3)nc(-n3c4ccccc4c4ccc5c6ccccc6n(-c6ccccc6)c5c43)n2)cc1]

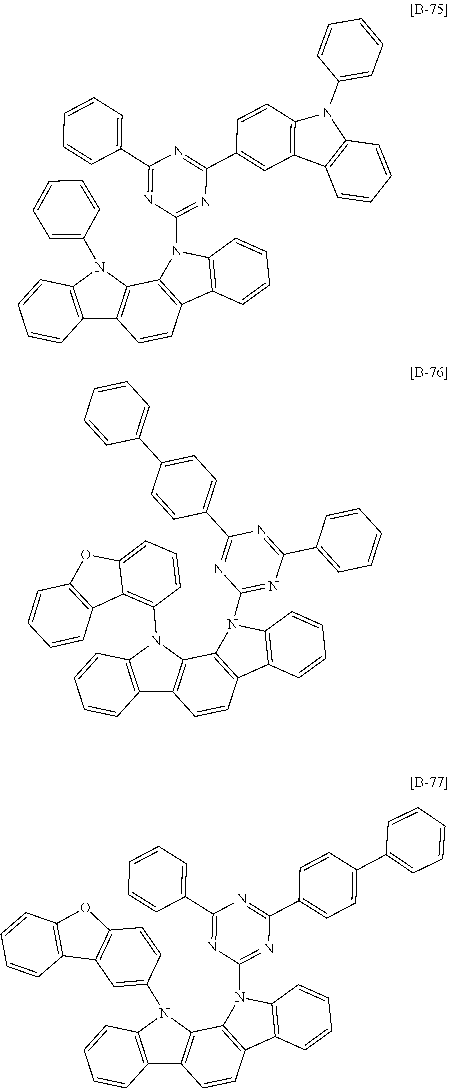

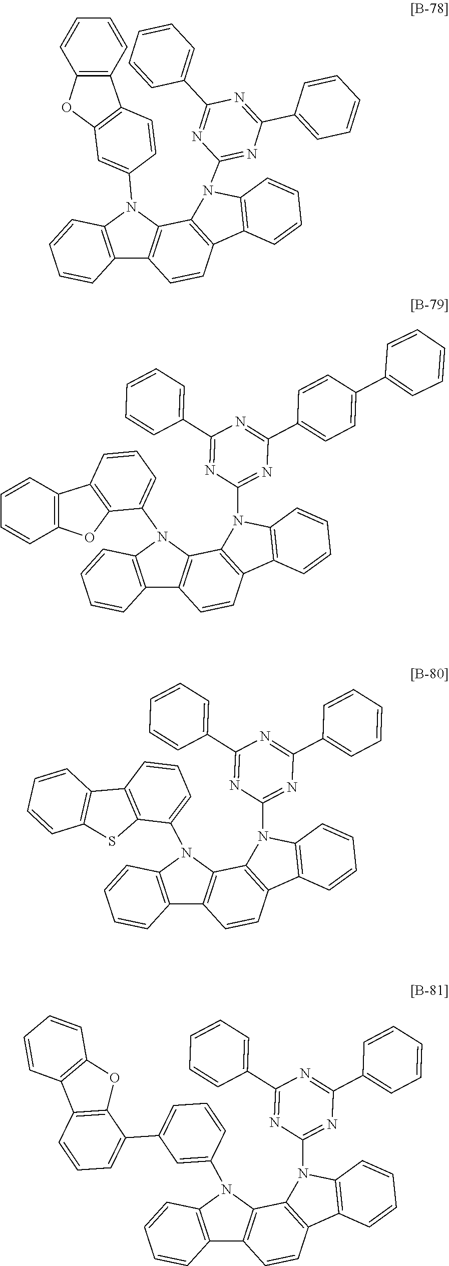 [SMILES: c1ccc(-c2nc(-c3ccccc3)nc(-n3c4ccccc4c4ccc5c6ccccc6n(-c6ccc7c(c6)oc6ccccc67)c5c43)n2)cc1 ; c1ccc(-c2ccc(-c3nc(-c4ccccc4)nc(-n4c5ccccc5c5ccc6c7ccccc7n(-c7cccc8c7oc7ccccc78)c6c54)n3)cc2)cc1 ; c1ccc(-c2nc(-c3ccccc3)nc(-n3c4ccccc4c4ccc5c6ccccc6n(-c6cccc7c6sc6ccccc67)c5c43)n2)cc1 ; c1ccc(-c2nc(-c3ccccc3)nc(-n3c4ccccc4c4ccc5c6ccccc6n(-c6cccc(-c7cccc8c7oc7ccccc78)c6)c5c43)n2)cc1]

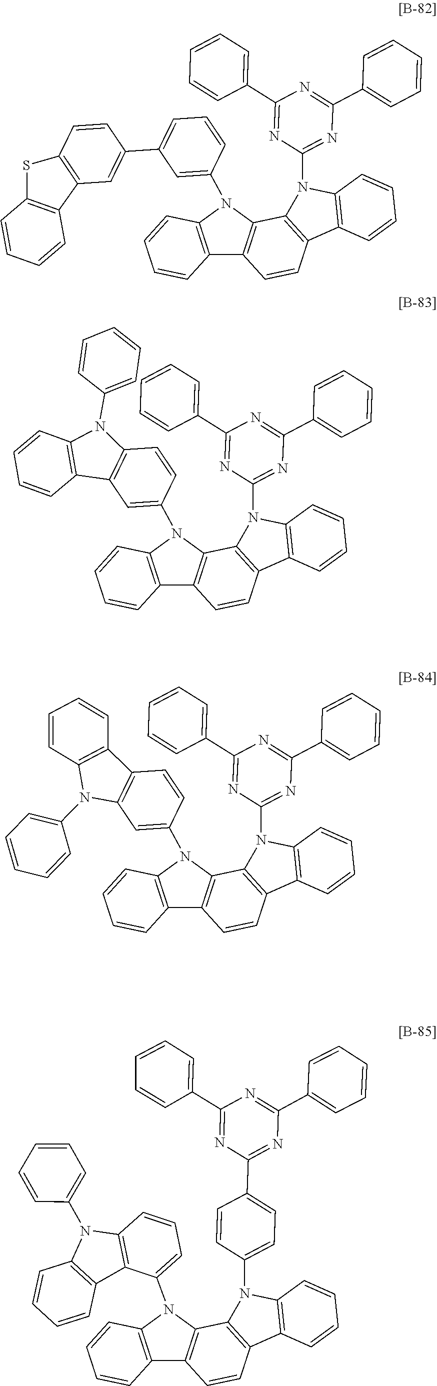 [SMILES: c1ccc(-c2nc(-c3ccccc3)nc(-n3c4ccccc4c4ccc5c6ccccc6n(-c6cccc(-c7ccc8sc9ccccc9c8c7)c6)c5c43)n2)cc1 ; c1ccc(-c2nc(-c3ccccc3)nc(-n3c4ccccc4c4ccc5c6ccccc6n(-c6ccc7c(c6)c6ccccc6n7-c6ccccc6)c5c43)n2)cc1 ; c1ccc(-c2nc(-c3ccccc3)nc(-n3c4ccccc4c4ccc5c6ccccc6n(-c6ccc7c8ccccc8n(-c8ccccc8)c7c6)c5c43)n2)cc1 ; c1ccc(-c2nc(-c3ccccc3)nc(-c3ccc(-n4c5ccccc5c5ccc6c7ccccc7n(-c7cccc8c7c7ccccc7n8-c7ccccc7)c6c54)cc3)n2)cc1]

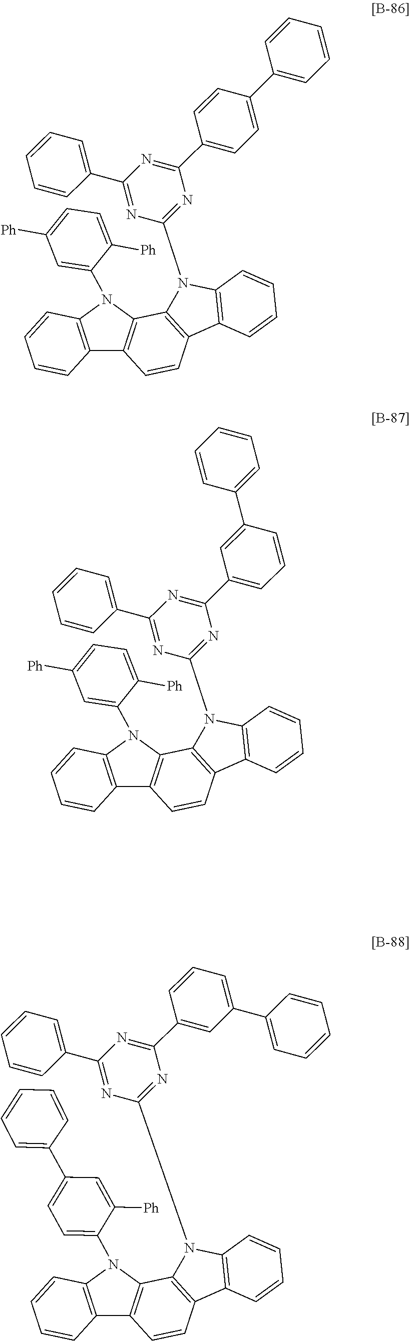 [SMILES: c1ccc(-c2ccc(-c3nc(-c4ccccc4)nc(-n4c5ccccc5c5ccc6c7ccccc7n(-c7cc(-c8ccccc8)ccc7-c7ccccc7)c6c54)n3)cc2)cc1 ; c1ccc(-c2cccc(-c3nc(-c4ccccc4)nc(-n4c5ccccc5c5ccc6c7ccccc7n(-c7cc(-c8ccccc8)ccc7-c7ccccc7)c6c54)n3)c2)cc1 ; c1ccc(-c2cccc(-c3nc(-c4ccccc4)nc(-n4c5ccccc5c5ccc6c7ccccc7n(-c7ccc(-c8ccccc8)cc7-c7ccccc7)c6c54)n3)c2)cc1]

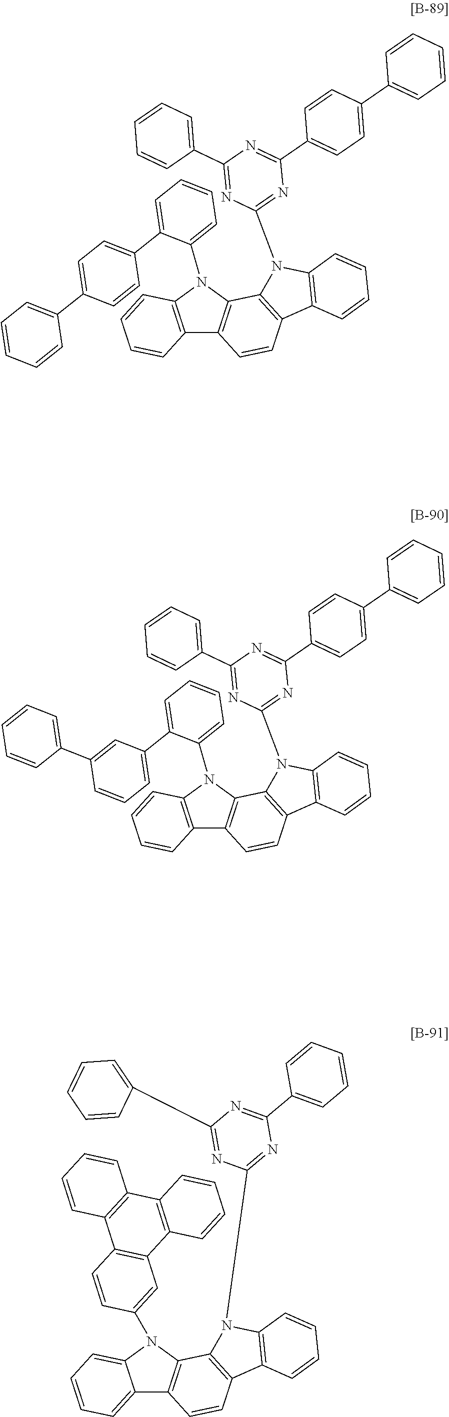 [SMILES: c1ccc(-c2ccc(-c3nc(-c4ccccc4)nc(-n4c5ccccc5c5ccc6c7ccccc7n(-c7ccccc7-c7ccc(-c8ccccc8)cc7)c6c54)n3)cc2)cc1 ; c1ccc(-c2ccc(-c3nc(-c4ccccc4)nc(-n4c5ccccc5c5ccc6c7ccccc7n(-c7ccccc7-c7cccc(-c8ccccc8)c7)c6c54)n3)cc2)cc1 ; c1ccc(-c2nc(-c3ccccc3)nc(-n3c4ccccc4c4ccc5c6ccccc6n(-c6ccc7c8ccccc8c8ccccc8c7c6)c5c43)n2)cc1]

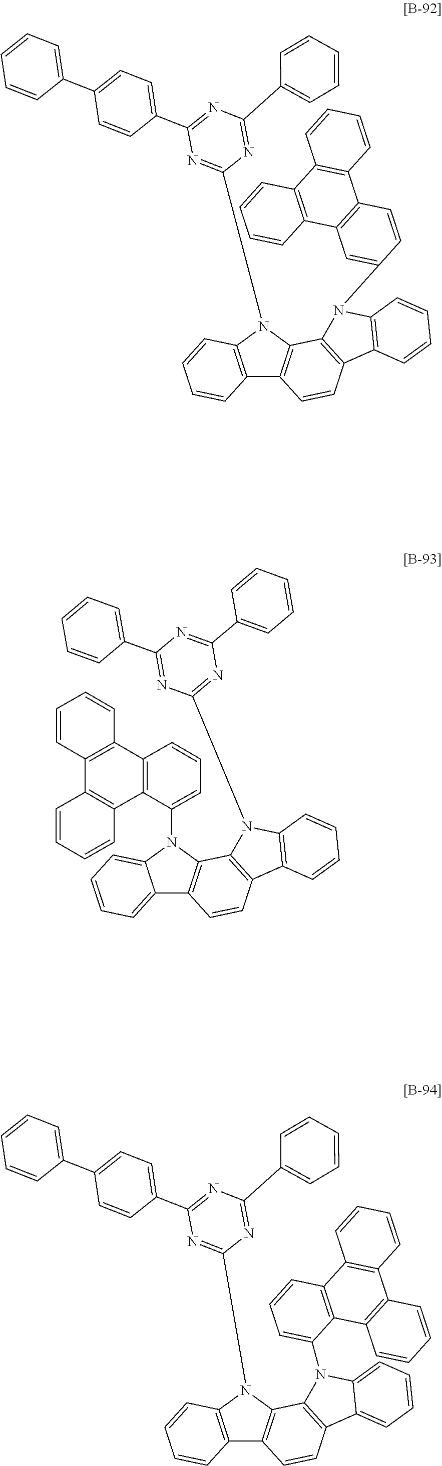 [SMILES: c1ccc(-c2ccc(-c3nc(-c4ccccc4)nc(-n4c5ccccc5c5ccc6c7ccccc7n(-c7ccc8c9ccccc9c9ccccc9c8c7)c6c54)n3)cc2)cc1 ; c1ccc(-c2nc(-c3ccccc3)nc(-n3c4ccccc4c4ccc5c6ccccc6n(-c6cccc7c8ccccc8c8ccccc8c67)c5c43)n2)cc1 ; c1ccc(-c2ccc(-c3nc(-c4ccccc4)nc(-n4c5ccccc5c5ccc6c7ccccc7n(-c7cccc8c9ccccc9c9ccccc9c78)c6c54)n3)cc2)cc1]

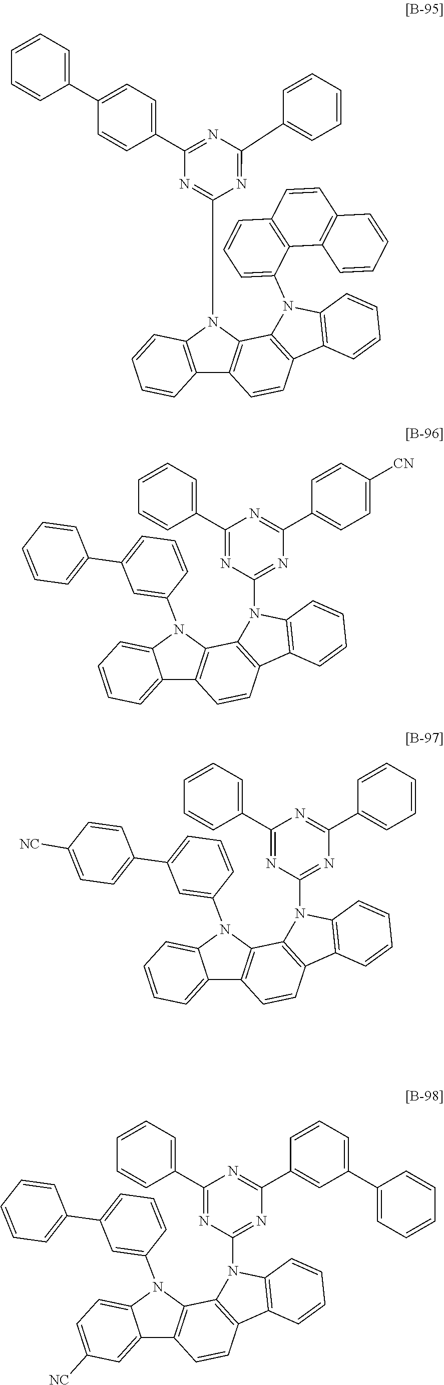 [SMILES: c1ccc(-c2ccc(-c3nc(-c4ccccc4)nc(-n4c5ccccc5c5ccc6c7ccccc7n(-c7cccc8ccc9ccccc9c78)c6c54)n3)cc2)cc1 ; N#Cc1ccc(-c2nc(-c3ccccc3)nc(-n3c4ccccc4c4ccc5c6ccccc6n(-c6cccc(-c7ccccc7)c6)c5c43)n2)cc1 ; N#Cc1ccc(-c2cccc(-n3c4ccccc4c4ccc5c6ccccc6n(-c6nc(-c7ccccc7)nc(-c7ccccc7)n6)c5c43)c2)cc1 ; N#Cc1ccc2c(c1)c1ccc3c4ccccc4n(-c4nc(-c5ccccc5)nc(-c5cccc(-c6ccccc6)c5)n4)c3c1n2-c1cccc(-c2ccccc2)c1]

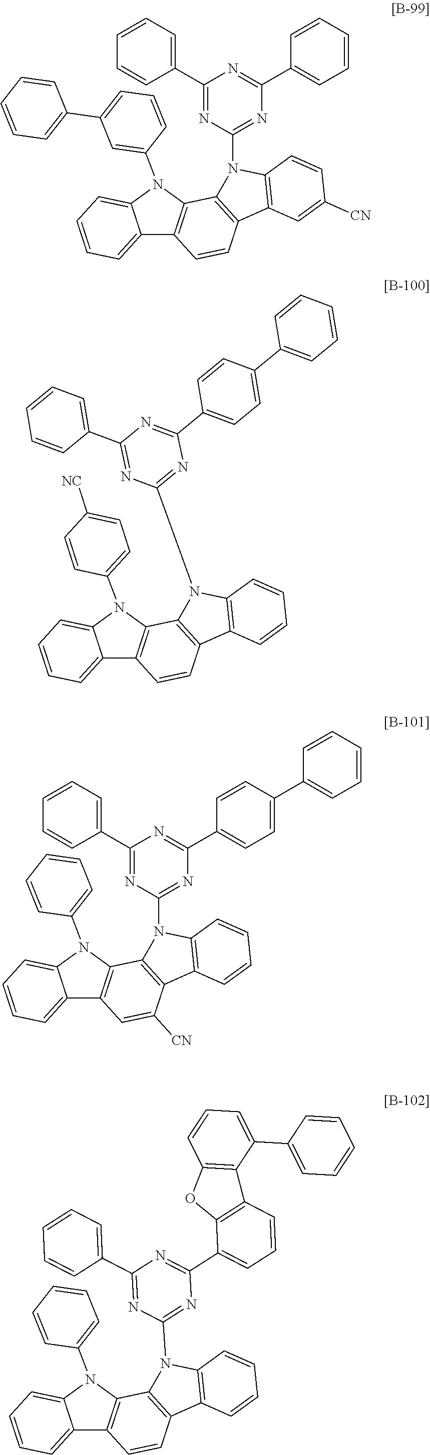 [SMILES: N#Cc1ccc2c(c1)c1ccc3c4ccccc4n(-c4cccc(-c5ccccc5)c4)c3c1n2-c1nc(-c2ccccc2)nc(-c2ccccc2)n1 ; N#Cc1ccc(-n2c3ccccc3c3ccc4c5ccccc5n(-c5nc(-c6ccccc6)nc(-c6ccc(-c7ccccc7)cc6)n5)c4c32)cc1 ; N#Cc1cc2c3ccccc3n(-c3ccccc3)c2c2c1c1ccccc1n2-c1nc(-c2ccccc2)nc(-c2ccc(-c3ccccc3)cc2)n1 ; c1ccc(-c2nc(-c3cccc4c3oc3cccc(-c5ccccc5)c34)nc(-n3c4ccccc4c4ccc5c6ccccc6n(-c6ccccc6)c5c43)n2)cc1]

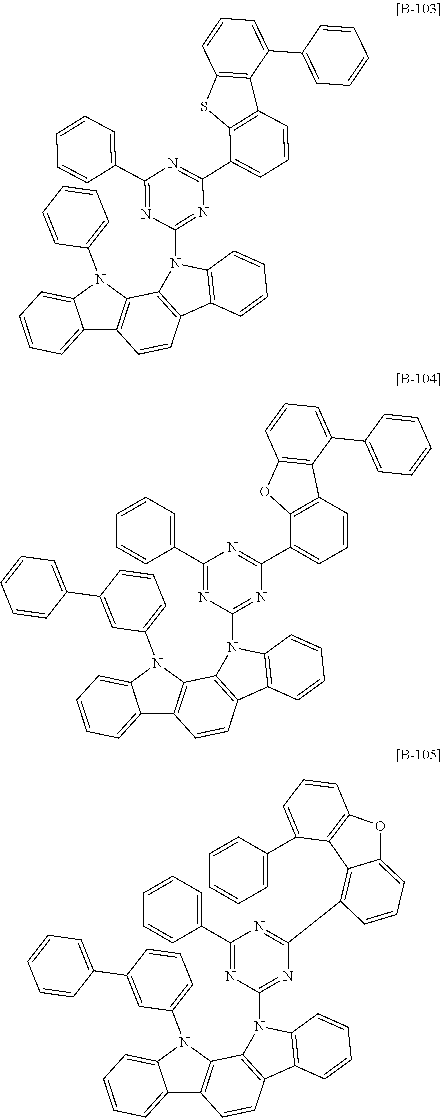 [SMILES: c1ccc(-c2nc(-c3cccc4c3sc3cccc(-c5ccccc5)c34)nc(-n3c4ccccc4c4ccc5c6ccccc6n(-c6ccccc6)c5c43)n2)cc1 ; c1ccc(-c2cccc(-n3c4ccccc4c4ccc5c6ccccc6n(-c6nc(-c7ccccc7)nc(-c7cccc8c7oc7cccc(-c9ccccc9)c78)n6)c5c43)c2)cc1 ; c1ccc(-c2cccc(-n3c4ccccc4c4ccc5c6ccccc6n(-c6nc(-c7ccccc7)nc(-c7cccc8oc9cccc(-c%10ccccc%10)c9c78)n6)c5c43)c2)cc1]

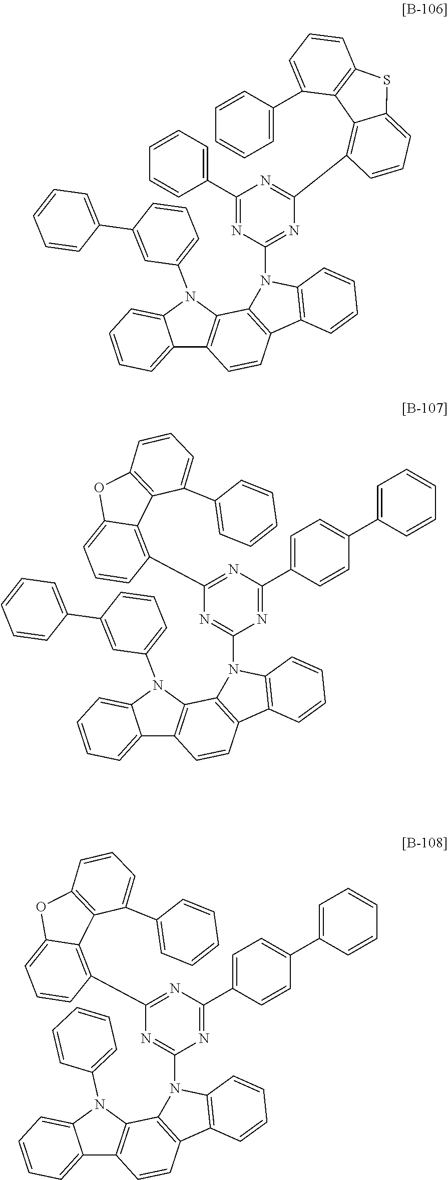 [SMILES: c1ccc(-c2cccc(-n3c4ccccc4c4ccc5c6ccccc6n(-c6nc(-c7ccccc7)nc(-c7cccc8sc9cccc(-c%10ccccc%10)c9c78)n6)c5c43)c2)cc1 ; c1ccc(-c2ccc(-c3nc(-c4cccc5oc6cccc(-c7ccccc7)c6c45)nc(-n4c5ccccc5c5ccc6c7ccccc7n(-c7cccc(-c8ccccc8)c7)c6c54)n3)cc2)cc1 ; c1ccc(-c2ccc(-c3nc(-c4cccc5oc6cccc(-c7ccccc7)c6c45)nc(-n4c5ccccc5c5ccc6c7ccccc7n(-c7ccccc7)c6c54)n3)cc2)cc1]

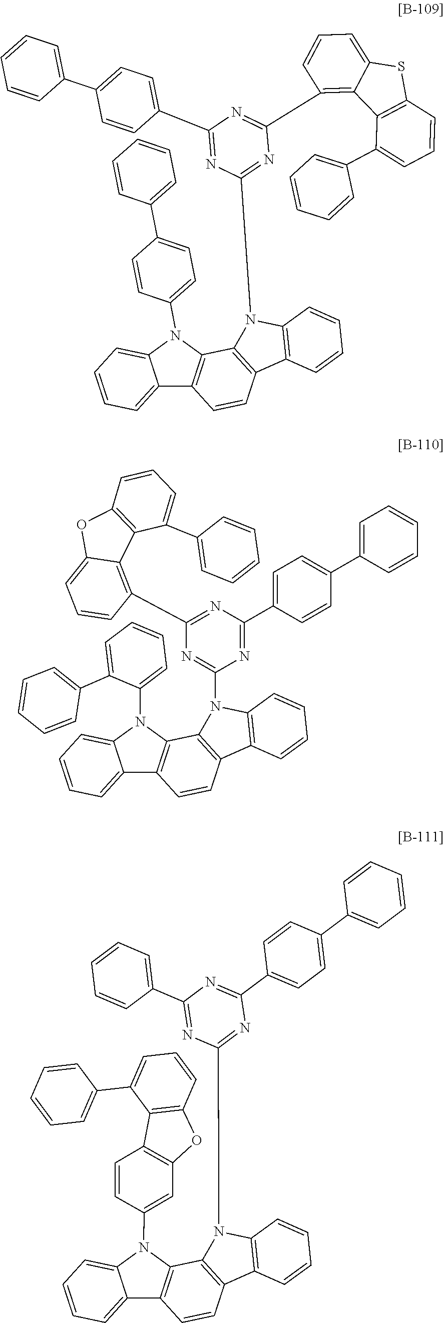 [SMILES: c1ccc(-c2ccc(-c3nc(-c4cccc5sc6cccc(-c7ccccc7)c6c45)nc(-n4c5ccccc5c5ccc6c7ccccc7n(-c7ccc(-c8ccccc8)cc7)c6c54)n3)cc2)cc1 ; c1ccc(-c2ccc(-c3nc(-c4cccc5oc6cccc(-c7ccccc7)c6c45)nc(-n4c5ccccc5c5ccc6c7ccccc7n(-c7ccccc7-c7ccccc7)c6c54)n3)cc2)cc1 ; c1ccc(-c2ccc(-c3nc(-c4ccccc4)nc(-n4c5ccccc5c5ccc6c7ccccc7n(-c7ccc8c(c7)oc7cccc(-c9ccccc9)c78)c6c54)n3)cc2)cc1]

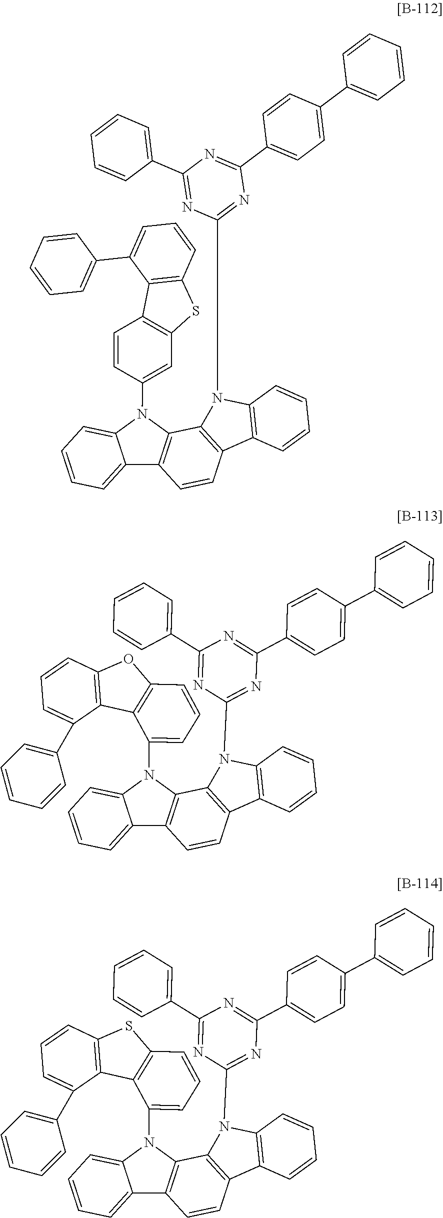 [SMILES: c1ccc(-c2ccc(-c3nc(-c4ccccc4)nc(-n4c5ccccc5c5ccc6c7ccccc7n(-c7ccc8c(c7)sc7cccc(-c9ccccc9)c78)c6c54)n3)cc2)cc1 ; c1ccc(-c2ccc(-c3nc(-c4ccccc4)nc(-n4c5ccccc5c5ccc6c7ccccc7n(-c7cccc8oc9cccc(-c%10ccccc%10)c9c78)c6c54)n3)cc2)cc1 ; c1ccc(-c2ccc(-c3nc(-c4ccccc4)nc(-n4c5ccccc5c5ccc6c7ccccc7n(-c7cccc8sc9cccc(-c%10ccccc%10)c9c78)c6c54)n3)cc2)cc1]

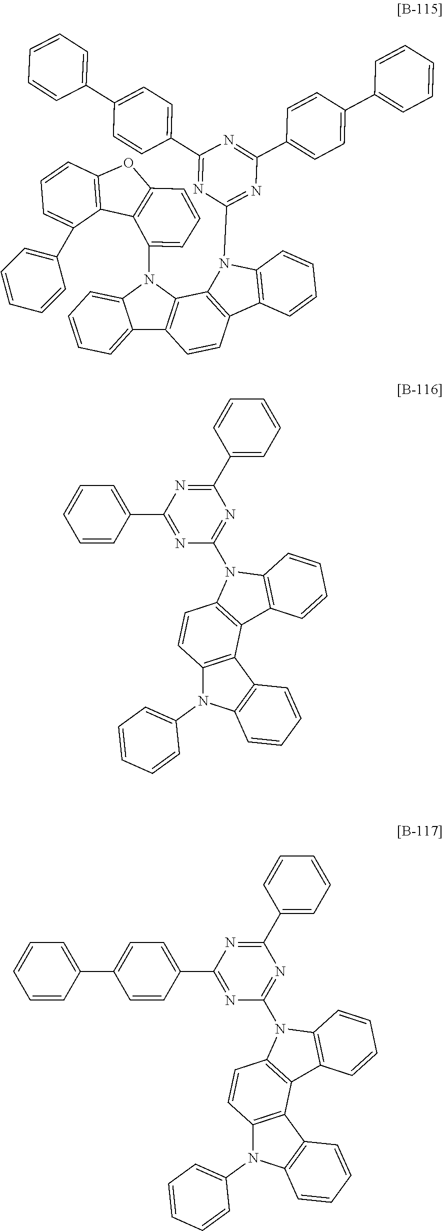 [SMILES: c1ccc(-c2ccc(-c3nc(-c4ccc(-c5ccccc5)cc4)nc(-n4c5ccccc5c5ccc6c7ccccc7n(-c7cccc8oc9cccc(-c%10ccccc%10)c9c78)c6c54)n3)cc2)cc1 ; c1ccc(-c2nc(-c3ccccc3)nc(-n3c4ccccc4c4c5c6ccccc6n(-c6ccccc6)c5ccc43)n2)cc1 ; c1ccc(-c2ccc(-c3nc(-c4ccccc4)nc(-n4c5ccccc5c5c6c7ccccc7n(-c7ccccc7)c6ccc54)n3)cc2)cc1]

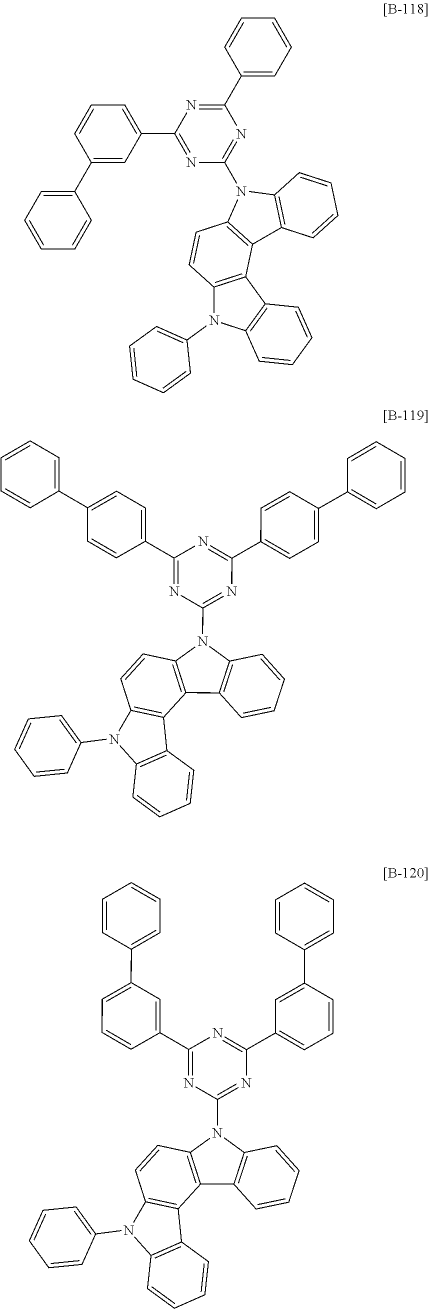 [SMILES: c1ccc(-c2cccc(-c3nc(-c4ccccc4)nc(-n4c5ccccc5c5c6c7ccccc7n(-c7ccccc7)c6ccc54)n3)c2)cc1 ; c1ccc(-c2ccc(-c3nc(-c4ccc(-c5ccccc5)cc4)nc(-n4c5ccccc5c5c6c7ccccc7n(-c7ccccc7)c6ccc54)n3)cc2)cc1 ; c1ccc(-c2cccc(-c3nc(-c4cccc(-c5ccccc5)c4)nc(-n4c5ccccc5c5c6c7ccccc7n(-c7ccccc7)c6ccc54)n3)c2)cc1]

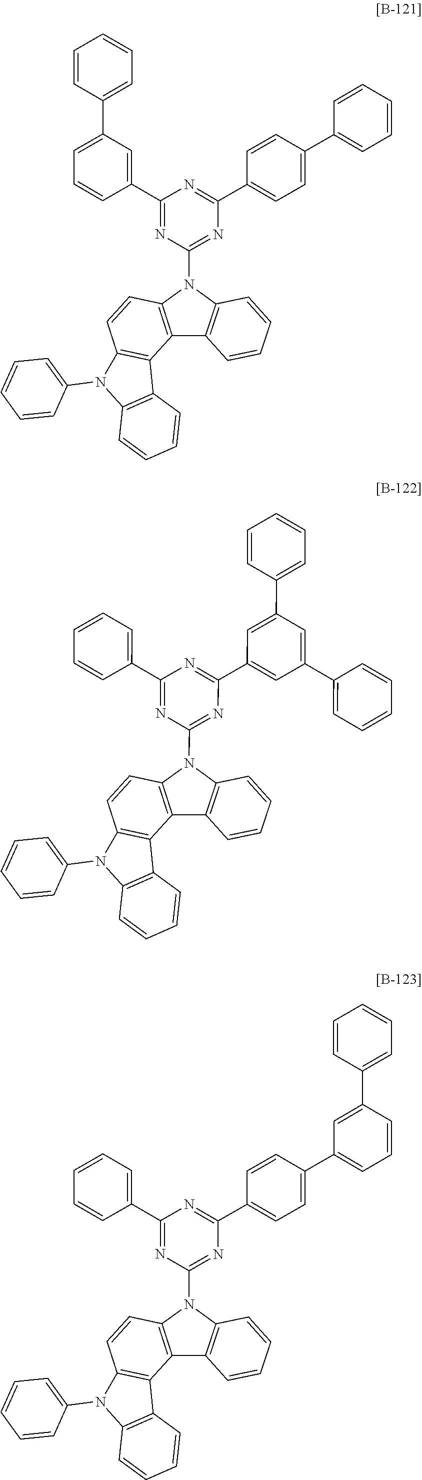 [SMILES: c1ccc(-c2ccc(-c3nc(-c4cccc(-c5ccccc5)c4)nc(-n4c5ccccc5c5c6c7ccccc7n(-c7ccccc7)c6ccc54)n3)cc2)cc1 ; c1ccc(-c2cc(-c3ccccc3)cc(-c3nc(-c4ccccc4)nc(-n4c5ccccc5c5c6c7ccccc7n(-c7ccccc7)c6ccc54)n3)c2)cc1 ; c1ccc(-c2cccc(-c3ccc(-c4nc(-c5ccccc5)nc(-n5c6ccccc6c6c7c8ccccc8n(-c8ccccc8)c7ccc65)n4)cc3)c2)cc1]

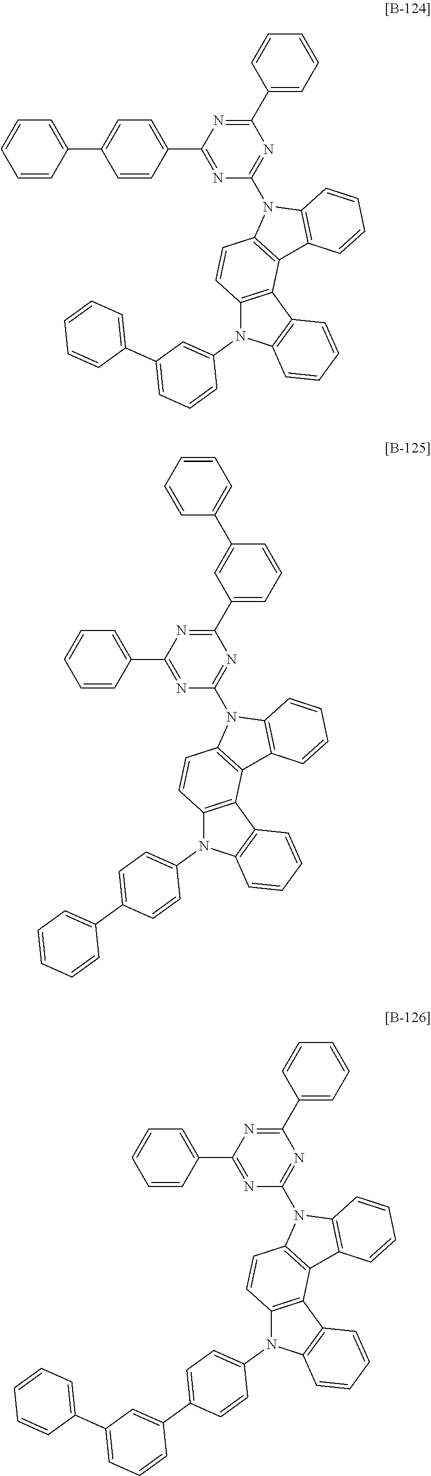 [SMILES: c1ccc(-c2ccc(-c3nc(-c4ccccc4)nc(-n4c5ccccc5c5c6c7ccccc7n(-c7cccc(-c8ccccc8)c7)c6ccc54)n3)cc2)cc1 ; c1ccc(-c2ccc(-n3c4ccccc4c4c5c6ccccc6n(-c6nc(-c7ccccc7)nc(-c7cccc(-c8ccccc8)c7)n6)c5ccc43)cc2)cc1 ; c1ccc(-c2cccc(-c3ccc(-n4c5ccccc5c5c6c7ccccc7n(-c7nc(-c8ccccc8)nc(-c8ccccc8)n7)c6ccc54)cc3)c2)cc1]

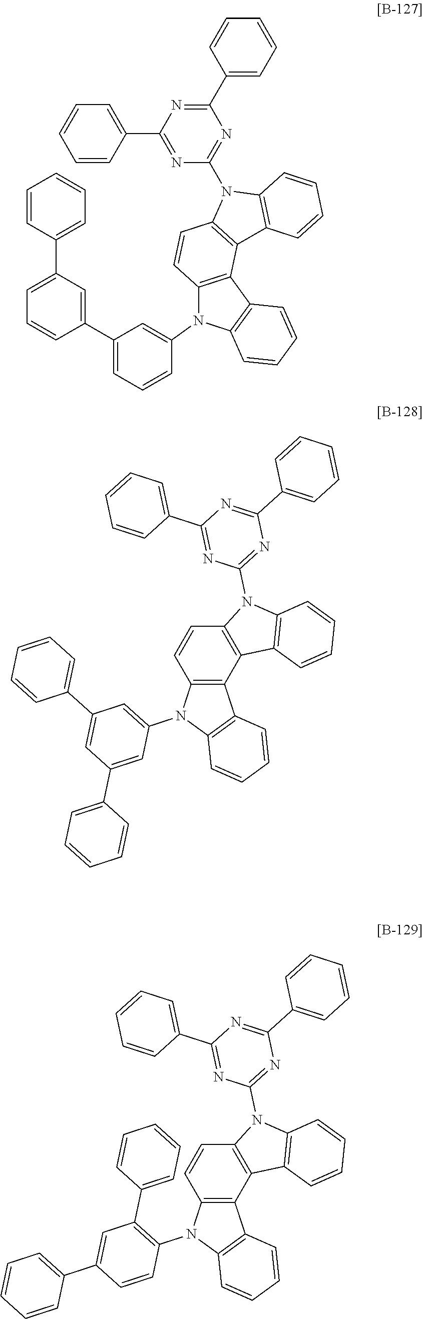 [SMILES: c1ccc(-c2cccc(-c3cccc(-n4c5ccccc5c5c6c7ccccc7n(-c7nc(-c8ccccc8)nc(-c8ccccc8)n7)c6ccc54)c3)c2)cc1 ; c1ccc(-c2cc(-c3ccccc3)cc(-n3c4ccccc4c4c5c6ccccc6n(-c6nc(-c7ccccc7)nc(-c7ccccc7)n6)c5ccc43)c2)cc1 ; c1ccc(-c2ccc(-n3c4ccccc4c4c5c6ccccc6n(-c6nc(-c7ccccc7)nc(-c7ccccc7)n6)c5ccc43)c(-c3ccccc3)c2)cc1]

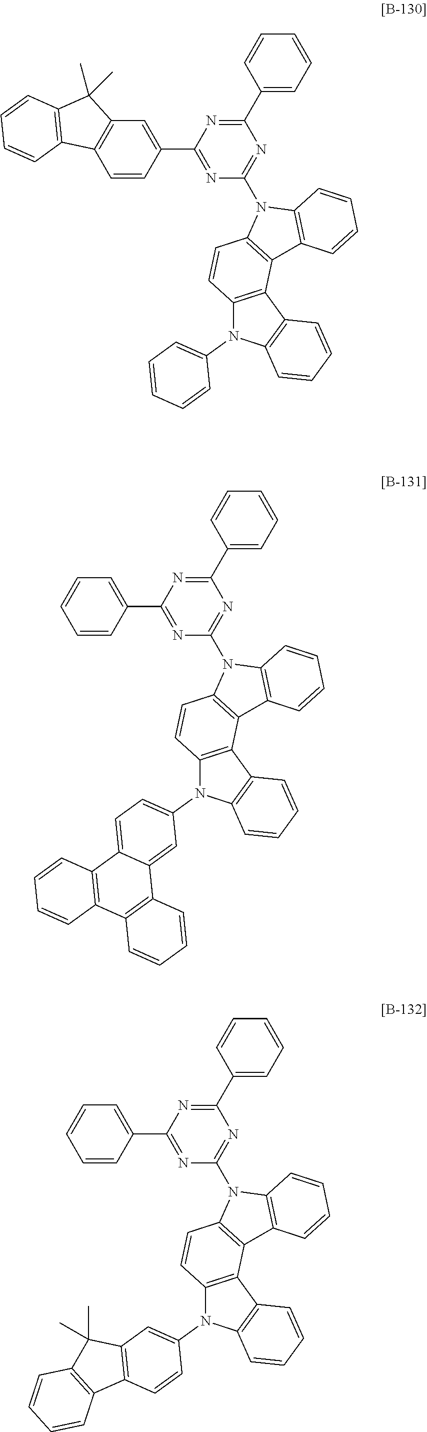 [SMILES: CC1(C)c2ccccc2-c2ccc(-c3nc(-c4ccccc4)nc(-n4c5ccccc5c5c6c7ccccc7n(-c7ccccc7)c6ccc54)n3)cc21 ; c1ccc(-c2nc(-c3ccccc3)nc(-n3c4ccccc4c4c5c6ccccc6n(-c6ccc7c8ccccc8c8ccccc8c7c6)c5ccc43)n2)cc1 ; CC1(C)c2ccccc2-c2ccc(-n3c4ccccc4c4c5c6ccccc6n(-c6nc(-c7ccccc7)nc(-c7ccccc7)n6)c5ccc43)cc21]

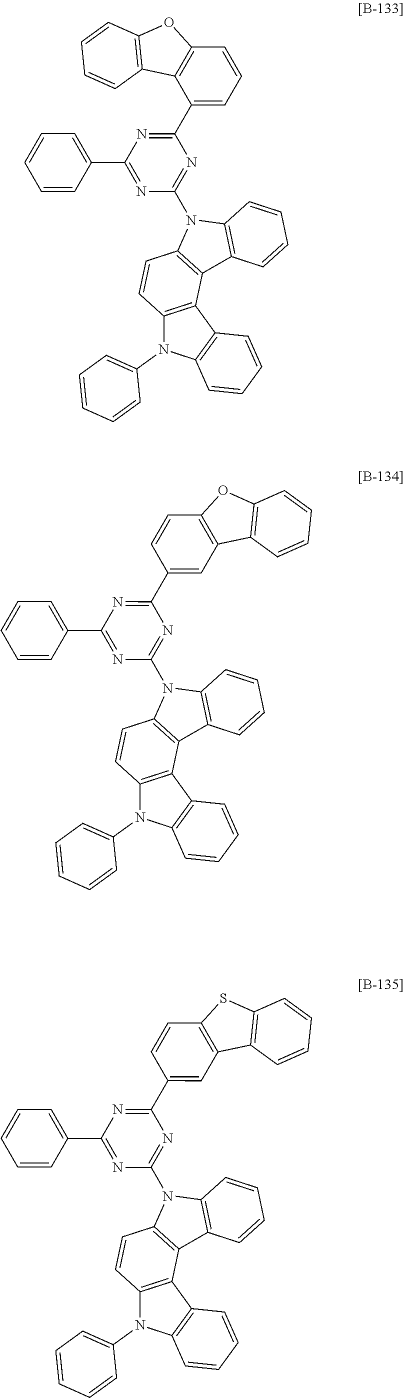 [SMILES: c1ccc(-c2nc(-c3cccc4oc5ccccc5c34)nc(-n3c4ccccc4c4c5c6ccccc6n(-c6ccccc6)c5ccc43)n2)cc1 ; c1ccc(-c2nc(-c3ccc4oc5ccccc5c4c3)nc(-n3c4ccccc4c4c5c6ccccc6n(-c6ccccc6)c5ccc43)n2)cc1 ; c1ccc(-c2nc(-c3ccc4sc5ccccc5c4c3)nc(-n3c4ccccc4c4c5c6ccccc6n(-c6ccccc6)c5ccc43)n2)cc1]

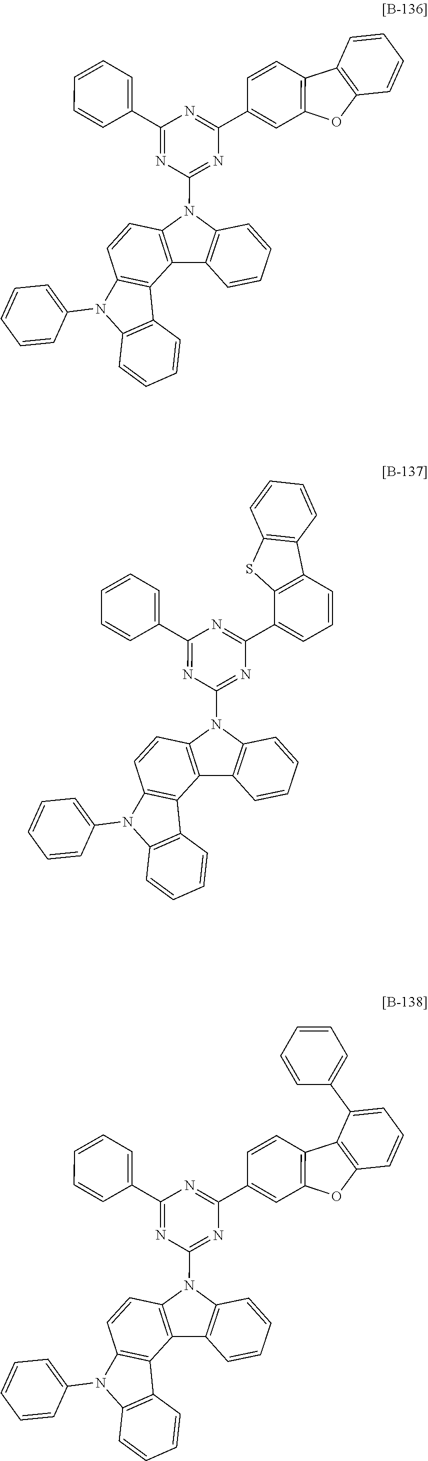 [SMILES: c1ccc(-c2nc(-c3ccc4c(c3)oc3ccccc34)nc(-n3c4ccccc4c4c5c6ccccc6n(-c6ccccc6)c5ccc43)n2)cc1 ; c1ccc(-c2nc(-c3cccc4c3sc3ccccc34)nc(-n3c4ccccc4c4c5c6ccccc6n(-c6ccccc6)c5ccc43)n2)cc1 ; c1ccc(-c2nc(-c3ccc4c(c3)oc3cccc(-c5ccccc5)c34)nc(-n3c4ccccc4c4c5c6ccccc6n(-c6ccccc6)c5ccc43)n2)cc1]

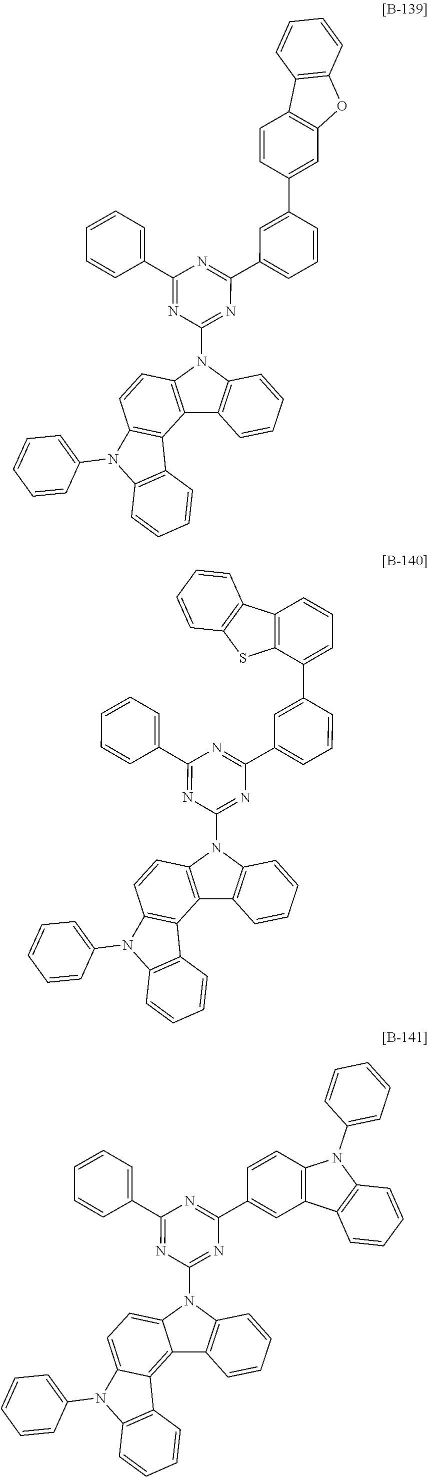 [SMILES: c1ccc(-c2nc(-c3cccc(-c4ccc5c(c4)oc4ccccc45)c3)nc(-n3c4ccccc4c4c5c6ccccc6n(-c6ccccc6)c5ccc43)n2)cc1 ; c1ccc(-c2nc(-c3cccc(-c4cccc5c4sc4ccccc45)c3)nc(-n3c4ccccc4c4c5c6ccccc6n(-c6ccccc6)c5ccc43)n2)cc1 ; c1ccc(-c2nc(-c3ccc4c(c3)c3ccccc3n4-c3ccccc3)nc(-n3c4ccccc4c4c5c6ccccc6n(-c6ccccc6)c5ccc43)n2)cc1]

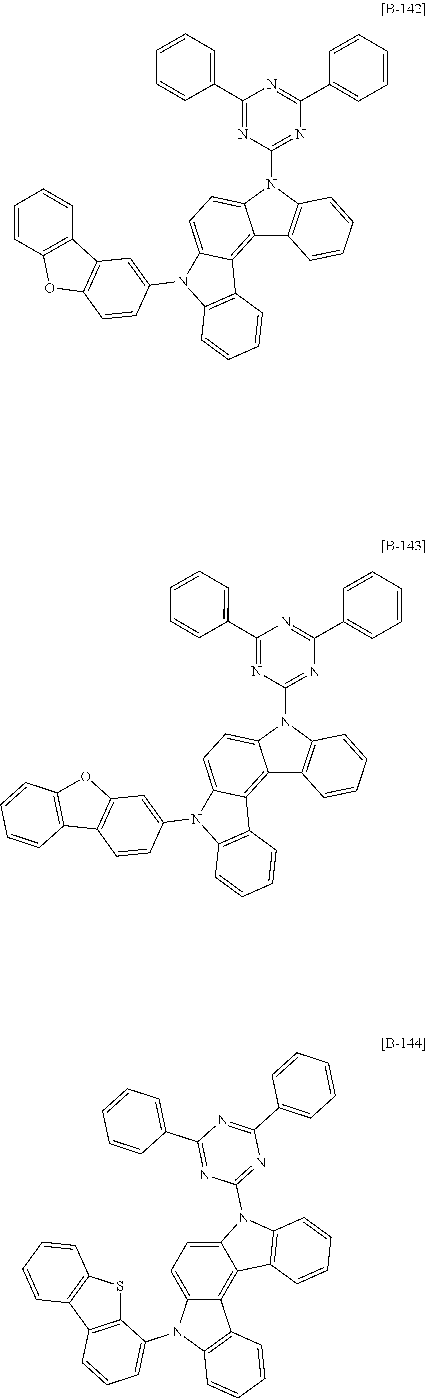 [SMILES: c1ccc(-c2nc(-c3ccccc3)nc(-n3c4ccccc4c4c5c6ccccc6n(-c6ccc7oc8ccccc8c7c6)c5ccc43)n2)cc1 ; c1ccc(-c2nc(-c3ccccc3)nc(-n3c4ccccc4c4c5c6ccccc6n(-c6ccc7c(c6)oc6ccccc67)c5ccc43)n2)cc1 ; c1ccc(-c2nc(-c3ccccc3)nc(-n3c4ccccc4c4c5c6ccccc6n(-c6cccc7c6sc6ccccc67)c5ccc43)n2)cc1]

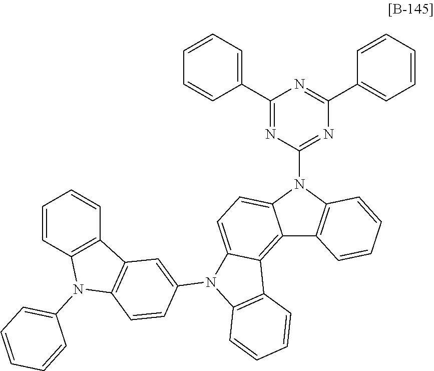 [SMILES: c1ccc(-c2nc(-c3ccccc3)nc(-n3c4ccccc4c4c5c6ccccc6n(-c6ccc7c(c6)c6ccccc6n7-c6ccccc6)c5ccc43)n2)cc1]

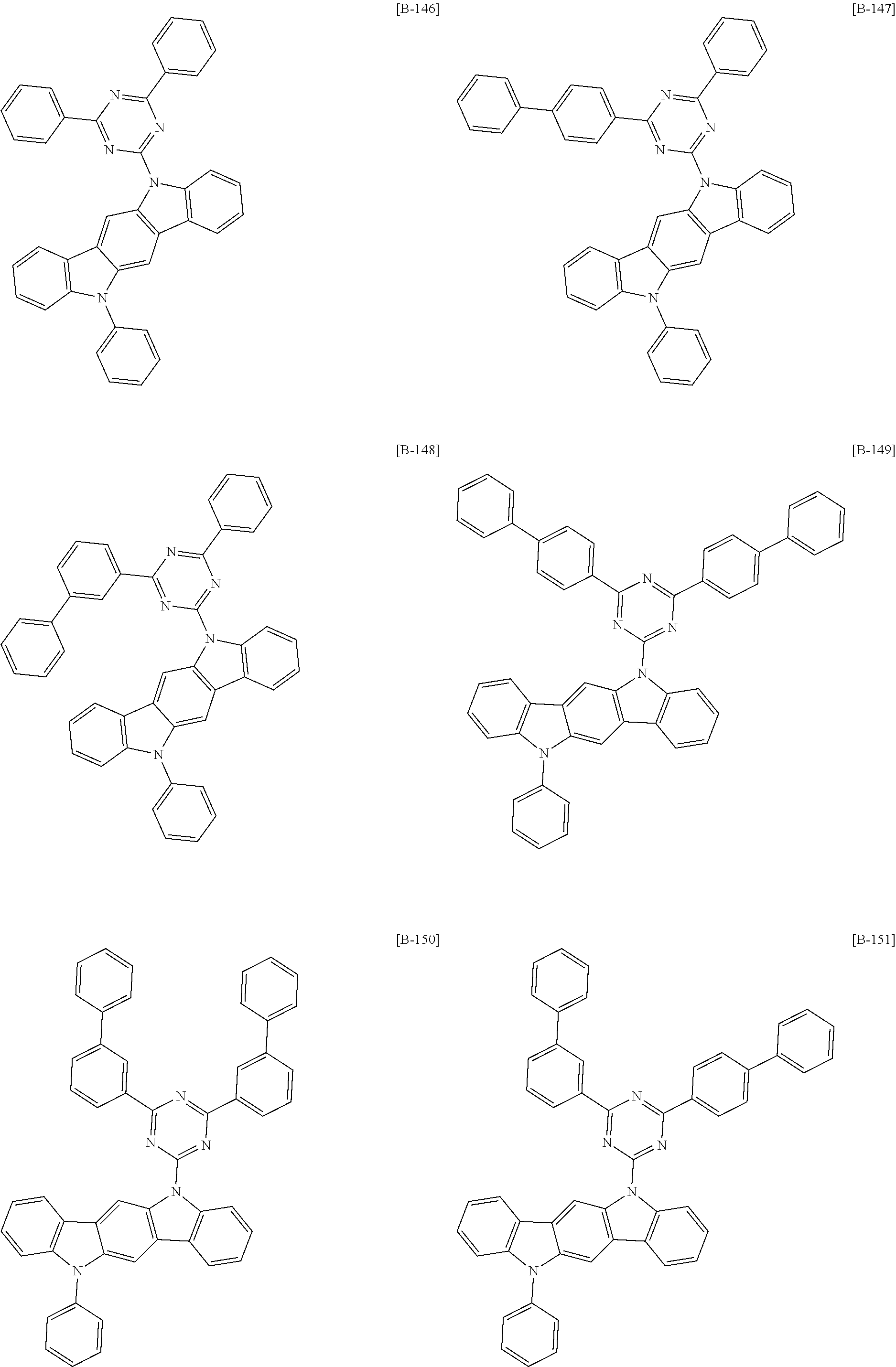

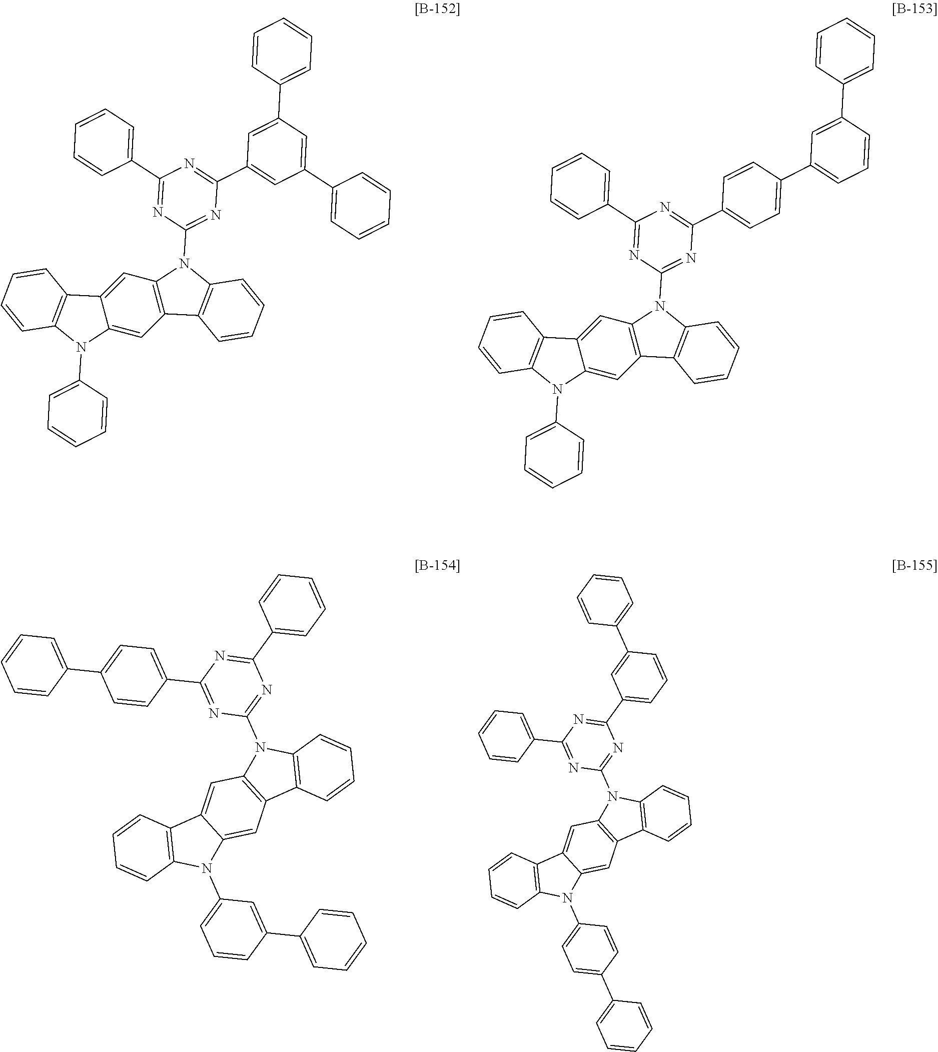 [SMILES: c1ccc(-c2cc(-c3ccccc3)cc(-c3nc(-c4ccccc4)nc(-n4c5ccccc5c5cc6c(cc54)c4ccccc4n6-c4ccccc4)n3)c2)cc1 ; c1ccc(-c2cccc(-c3ccc(-c4nc(-c5ccccc5)nc(-n5c6ccccc6c6cc7c(cc65)c5ccccc5n7-c5ccccc5)n4)cc3)c2)cc1 ; c1ccc(-c2ccc(-c3nc(-c4ccccc4)nc(-n4c5ccccc5c5cc6c(cc54)c4ccccc4n6-c4cccc(-c5ccccc5)c4)n3)cc2)cc1 ; c1ccc(-c2ccc(-n3c4ccccc4c4cc5c(cc43)c3ccccc3n5-c3nc(-c4ccccc4)nc(-c4cccc(-c5ccccc5)c4)n3)cc2)cc1]

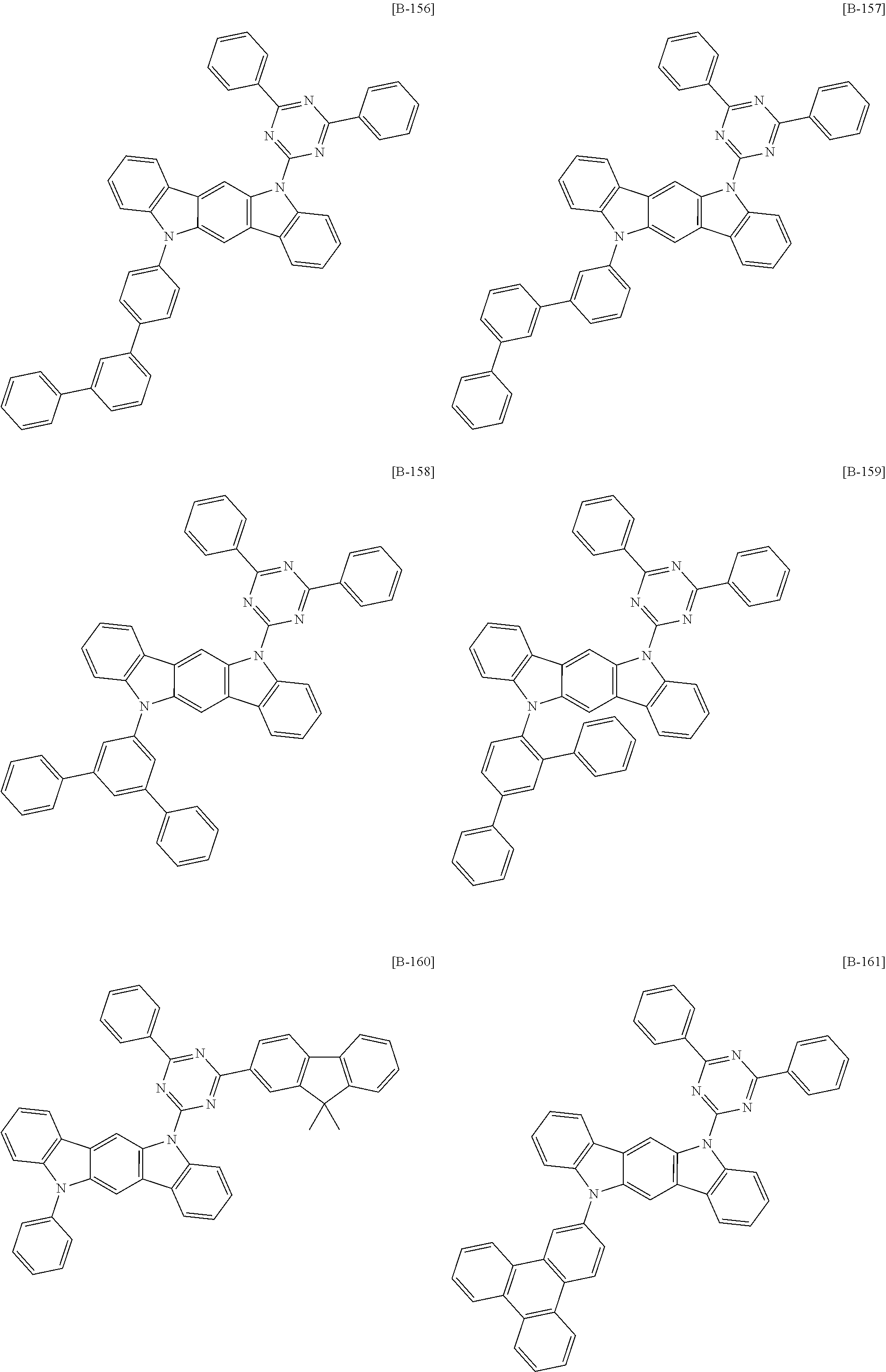

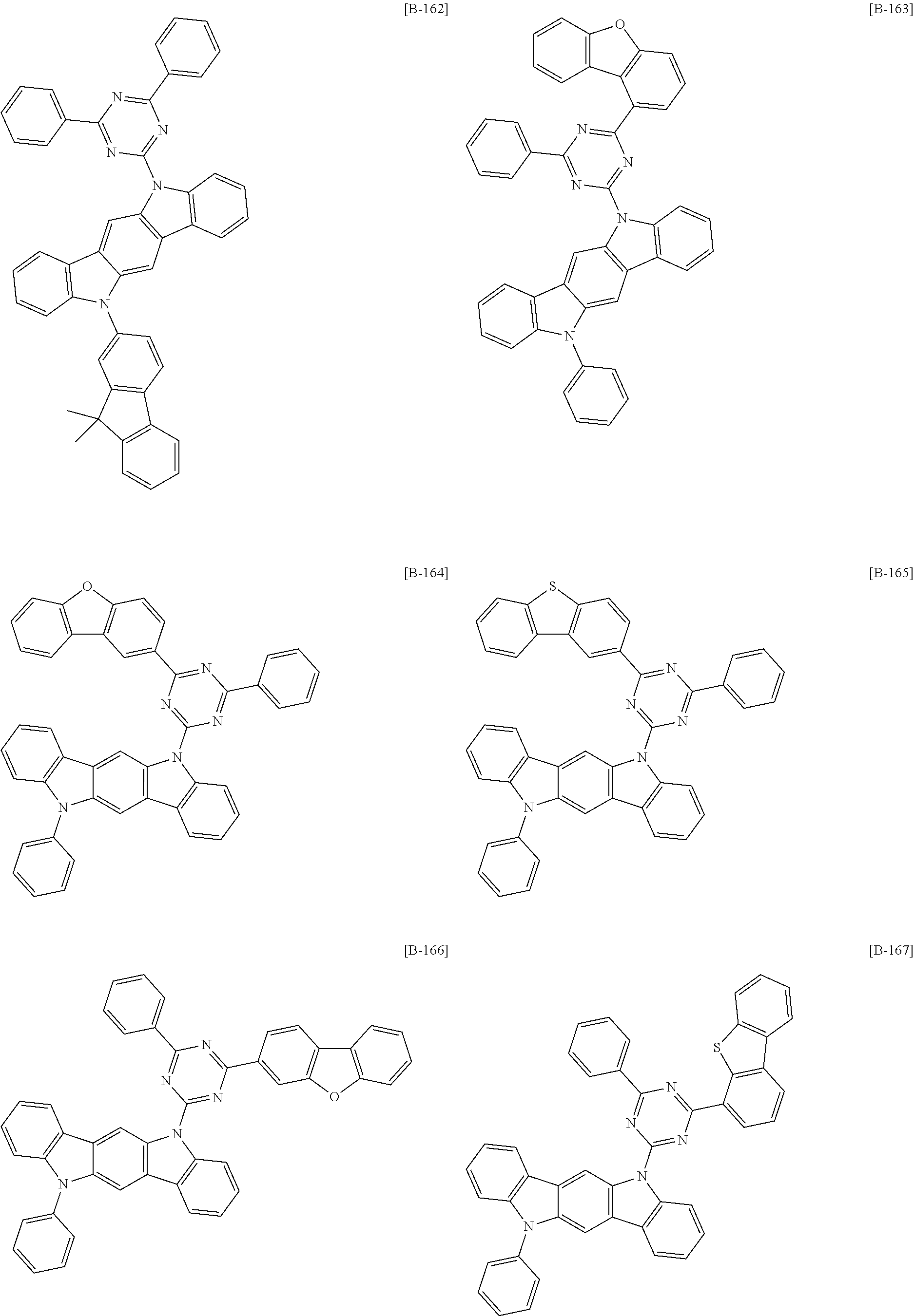

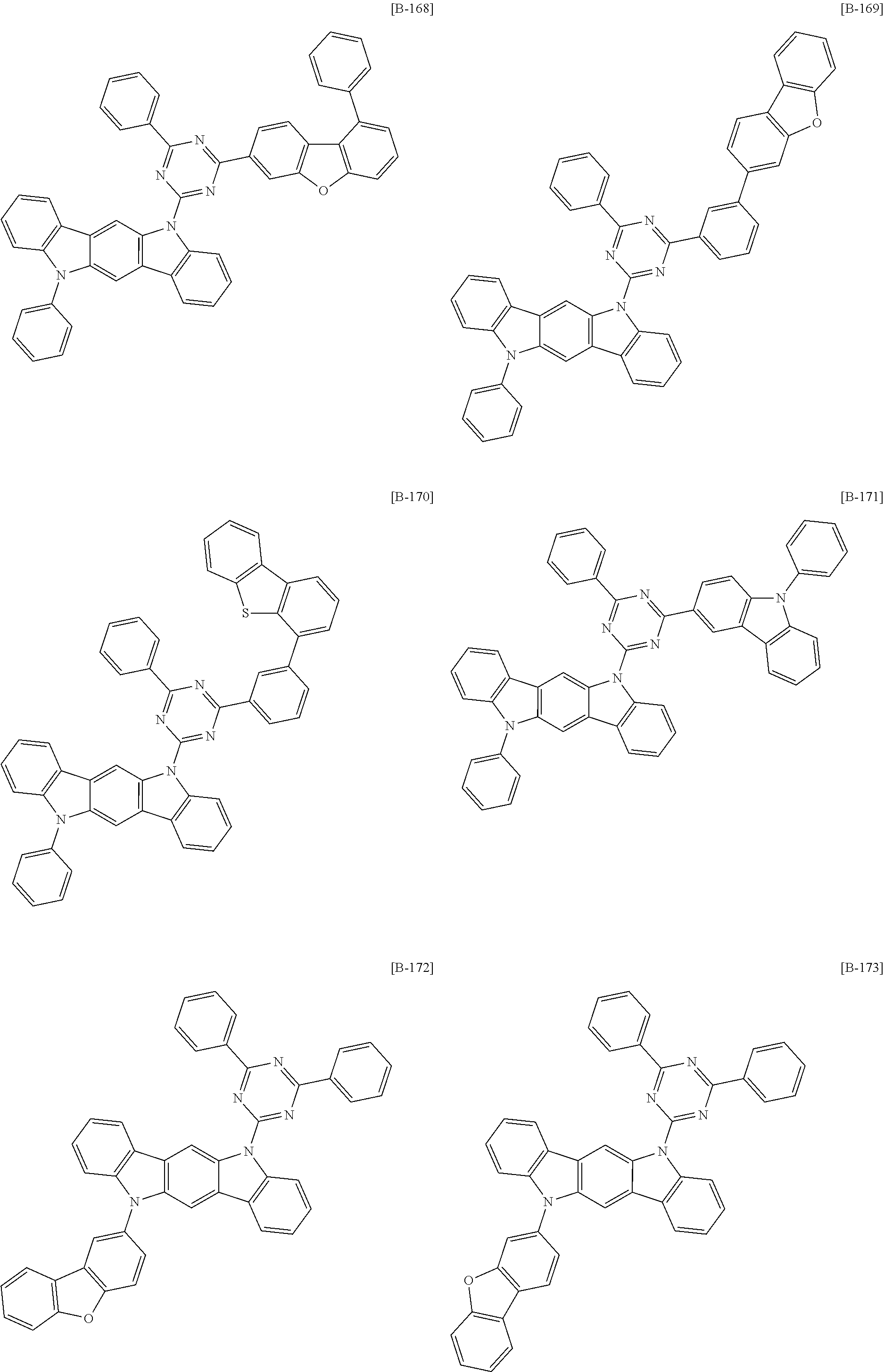

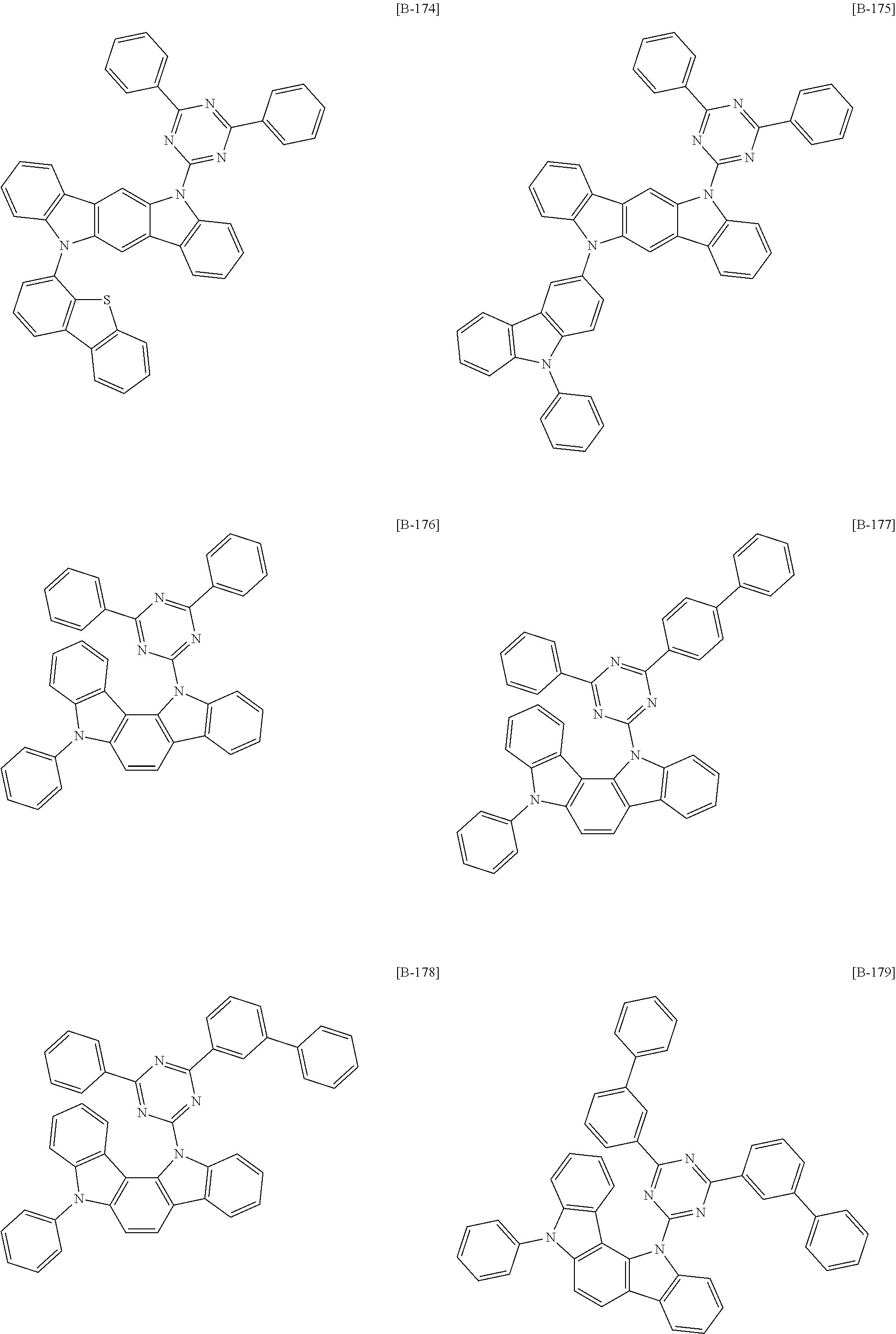

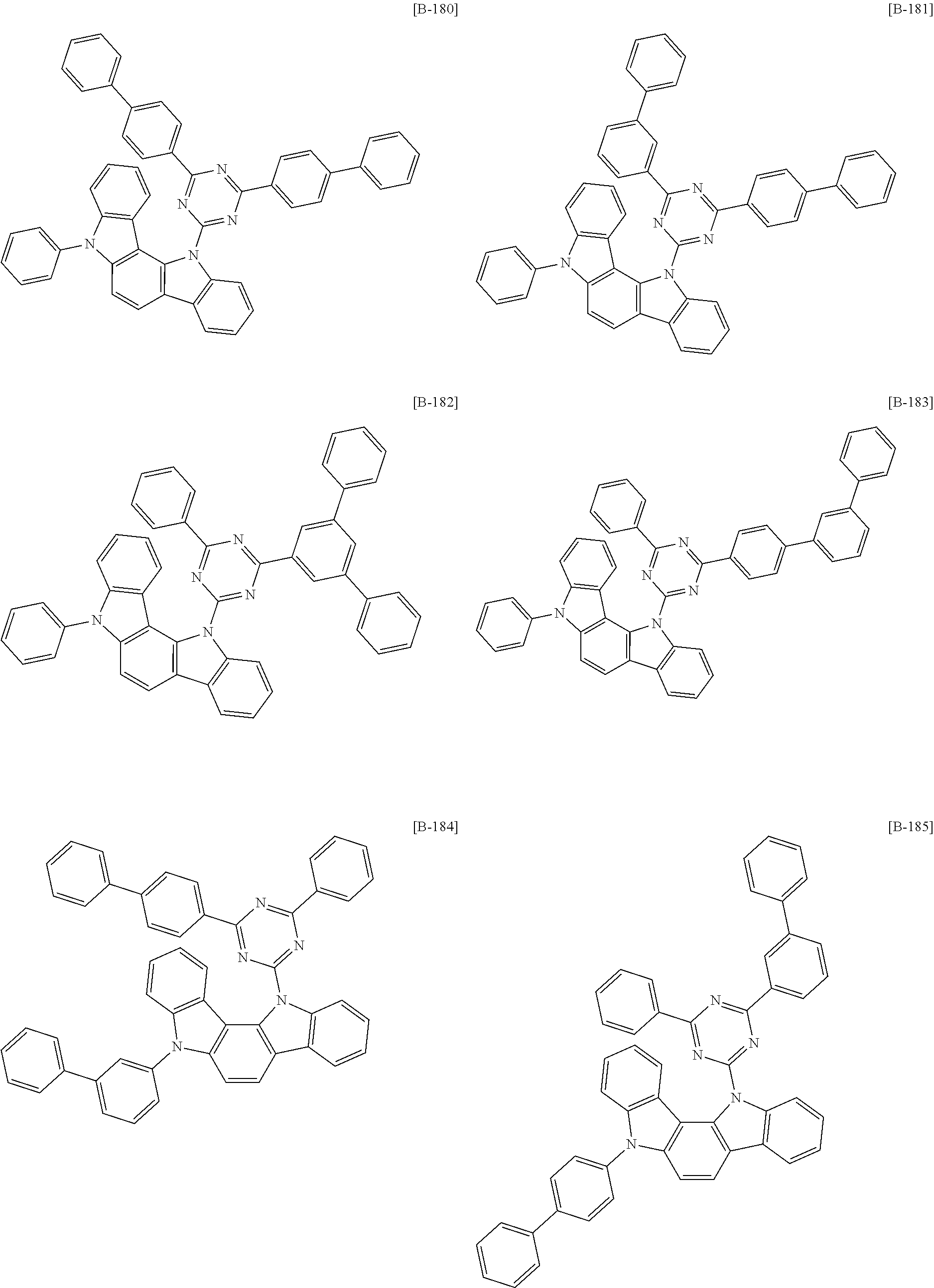

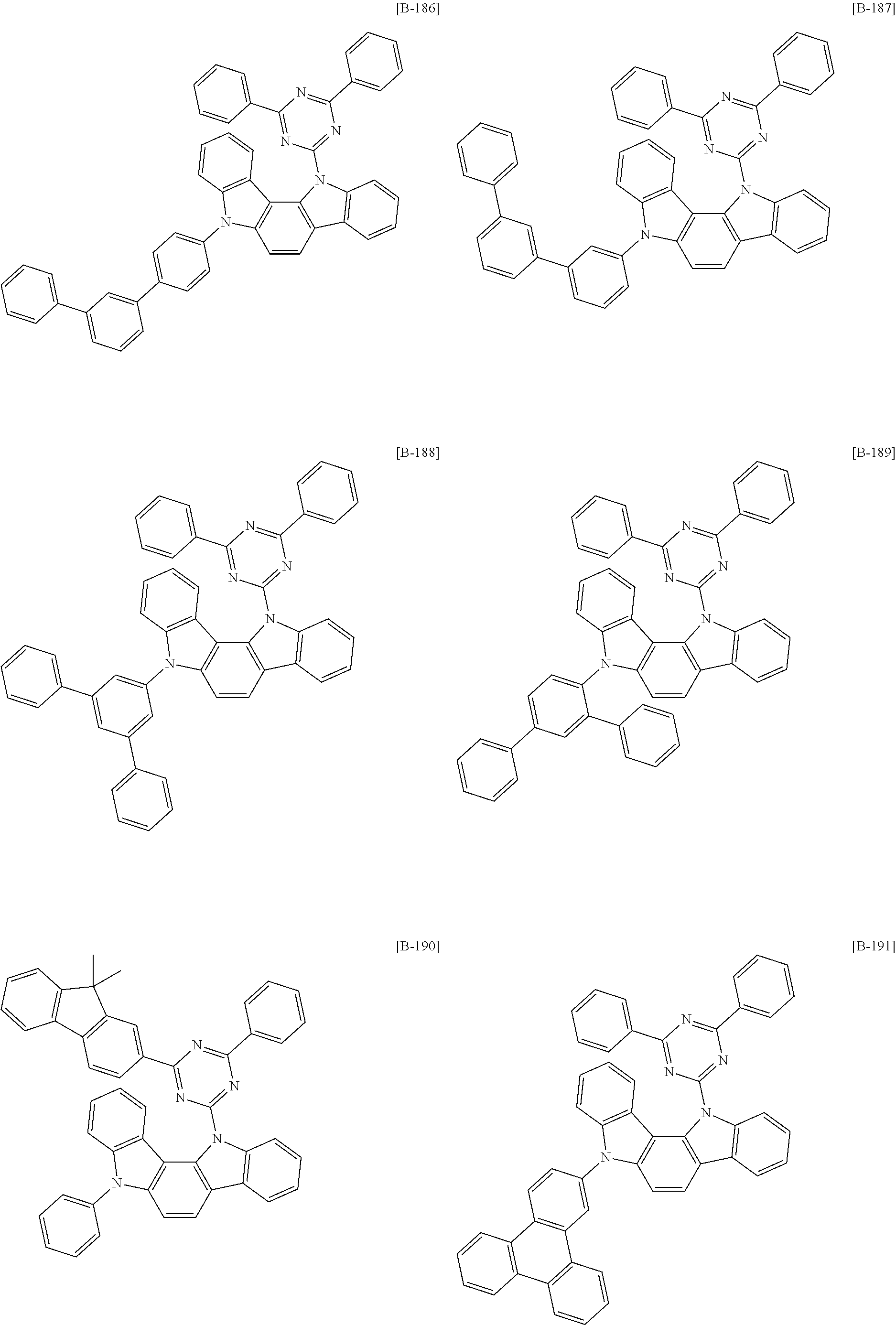

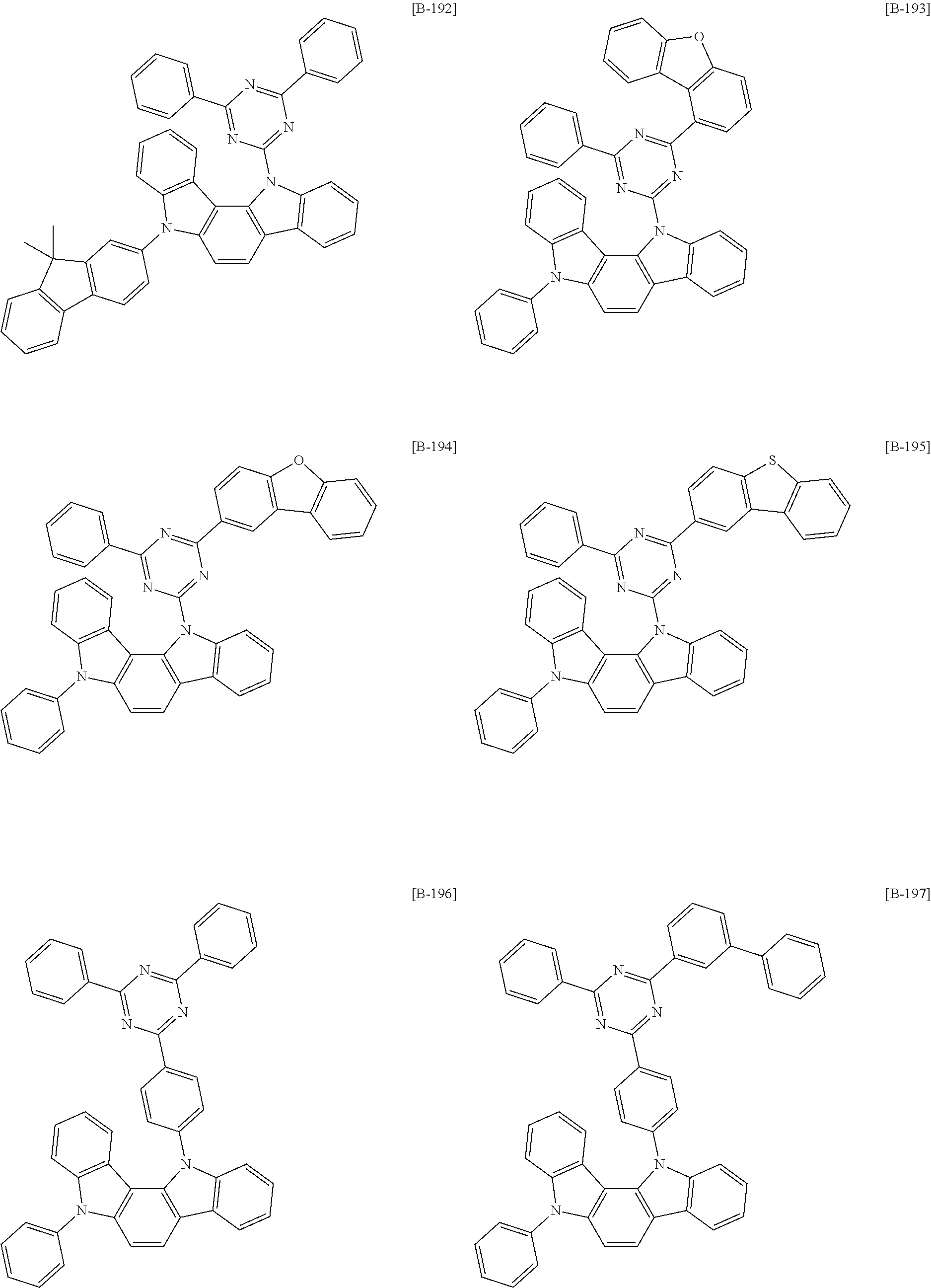

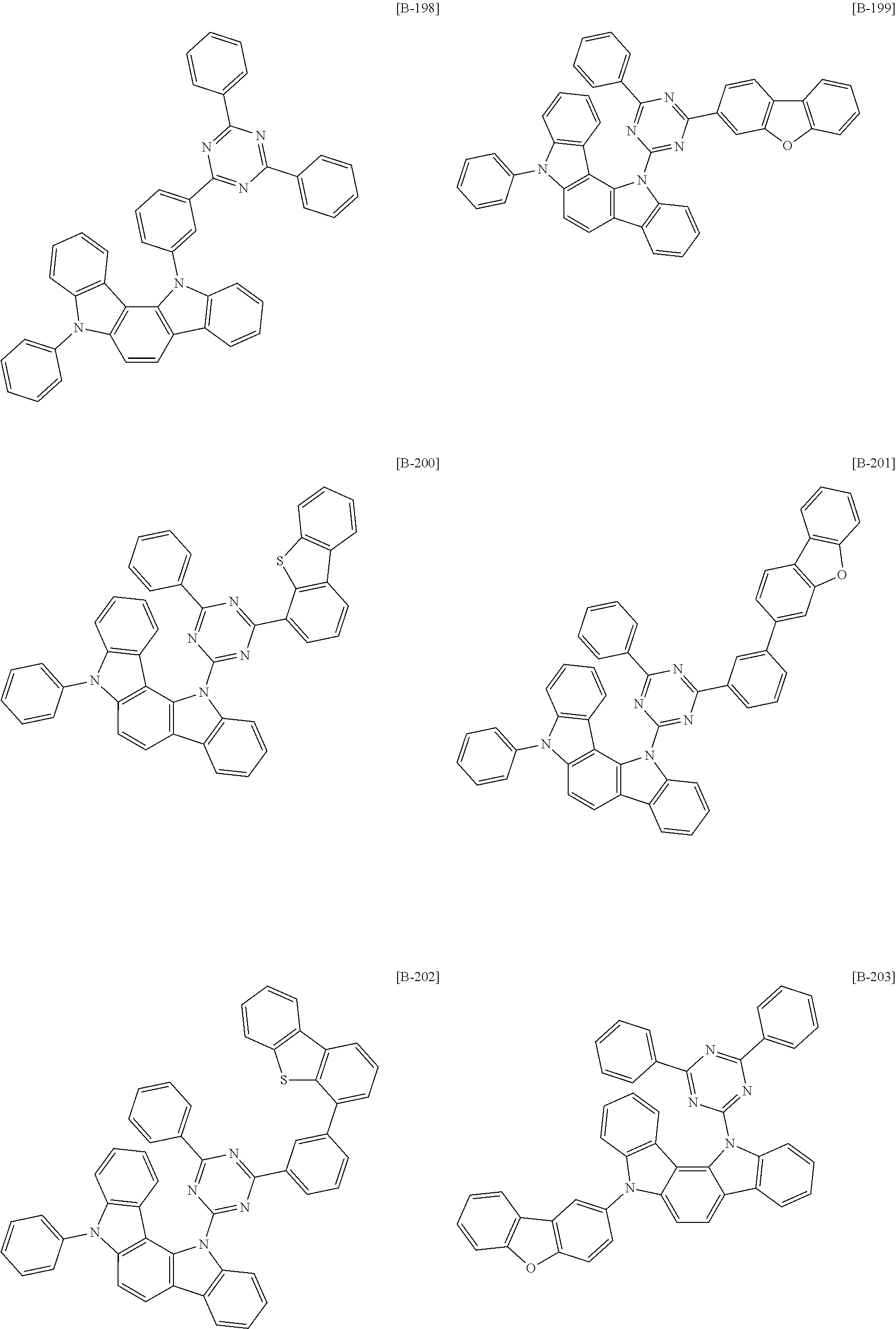

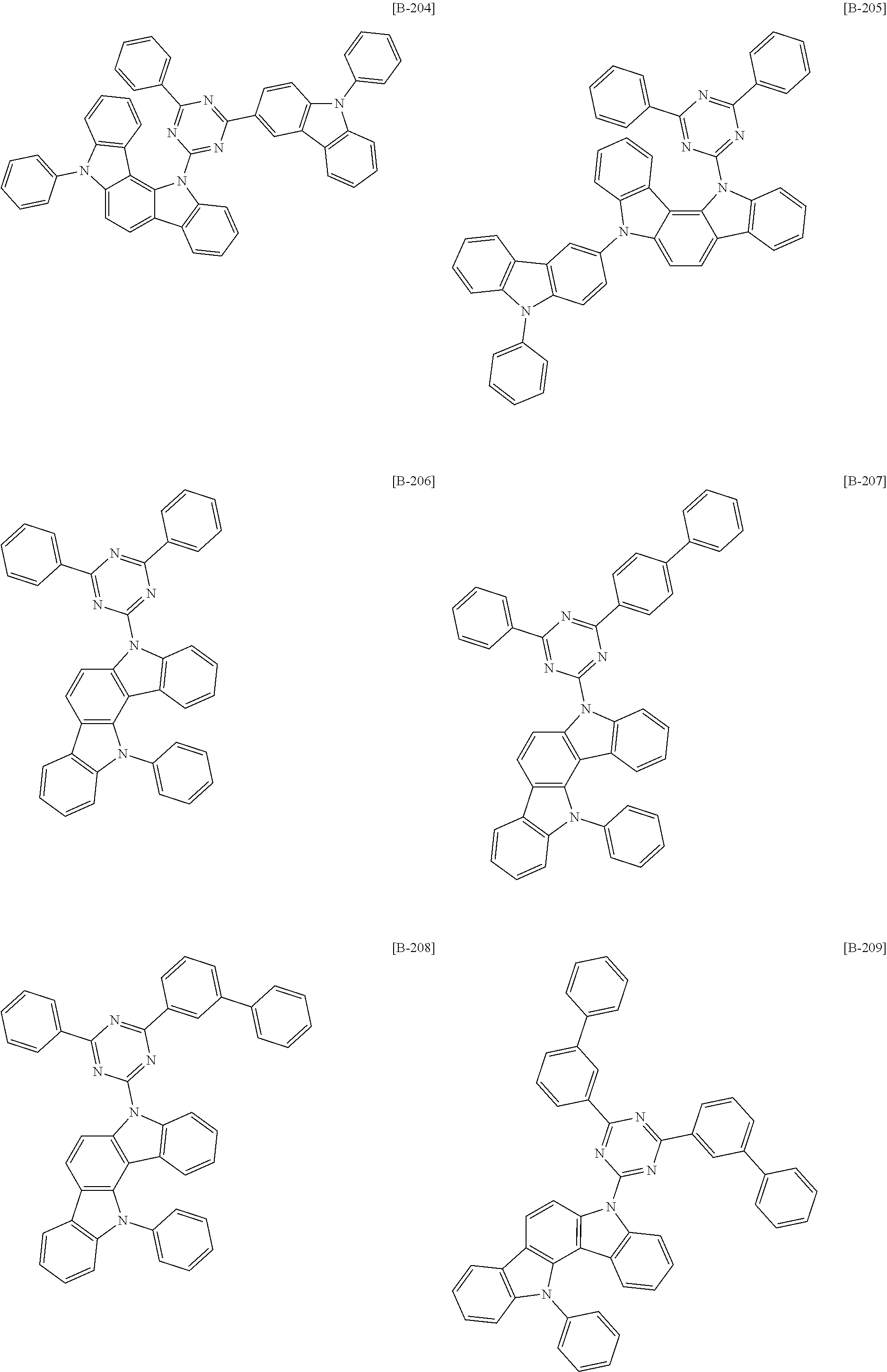

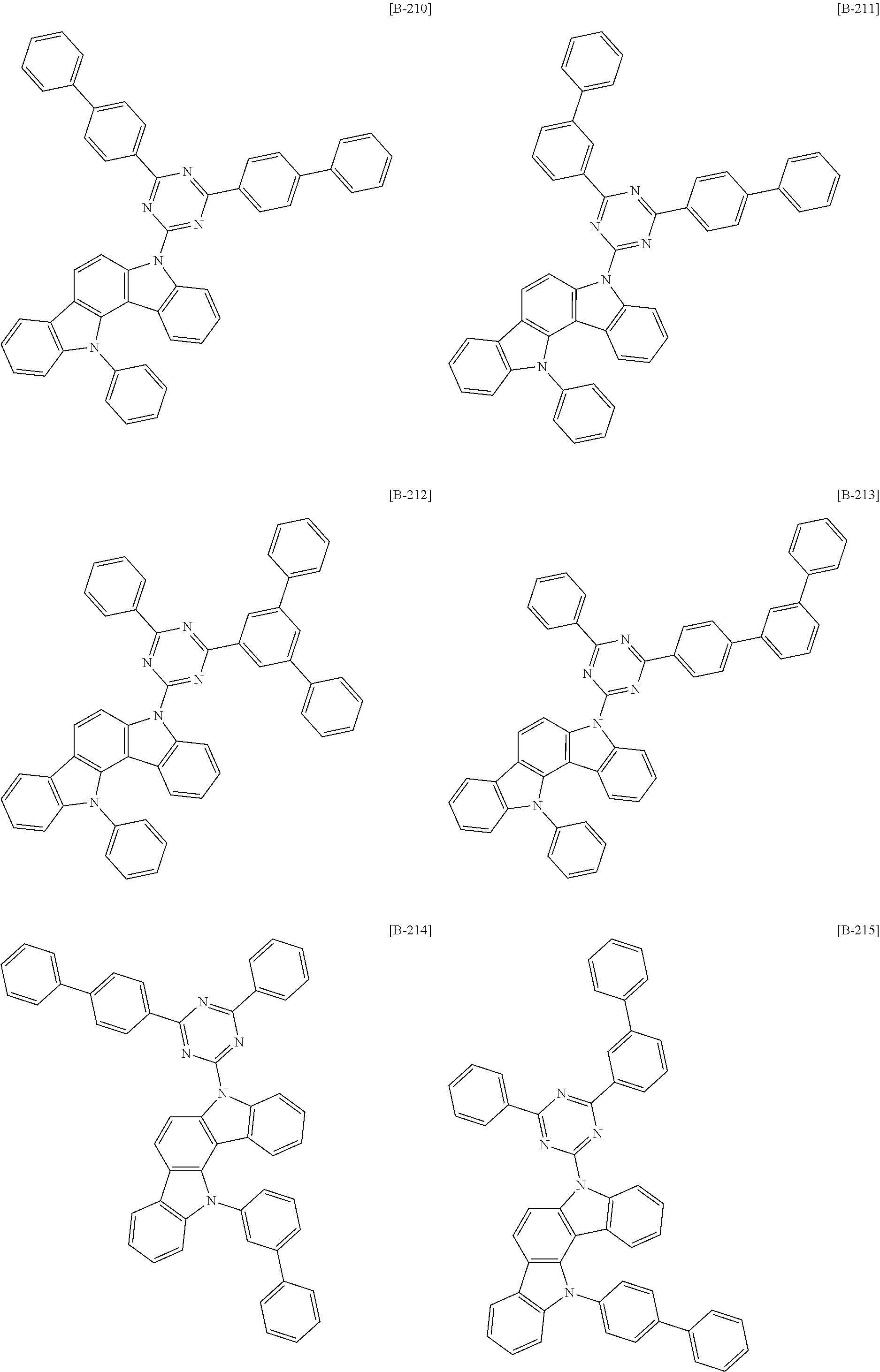

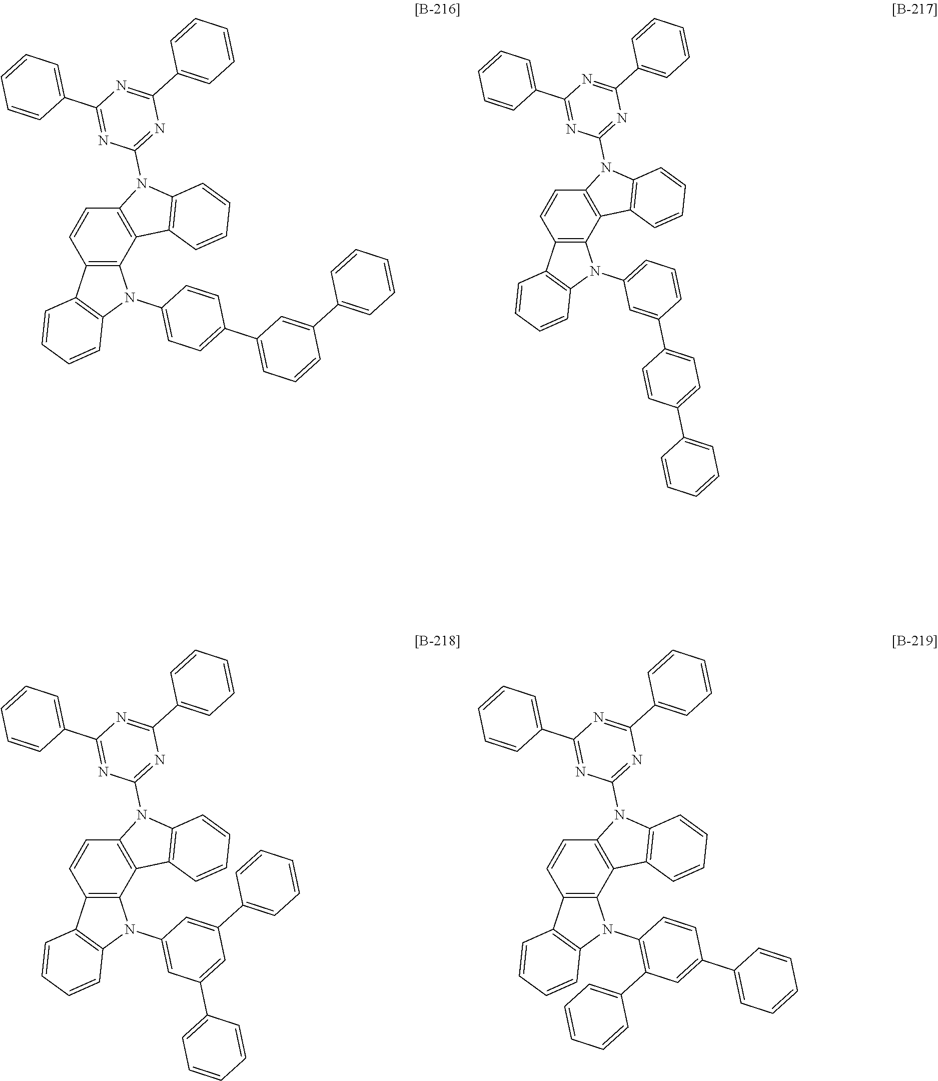 [SMILES: c1ccc(-c2cccc(-c3ccc(-n4c5ccccc5c5ccc6c(c7ccccc7n6-c6nc(-c7ccccc7)nc(-c7ccccc7)n6)c54)cc3)c2)cc1 ; c1ccc(-c2ccc(-c3cccc(-n4c5ccccc5c5ccc6c(c7ccccc7n6-c6nc(-c7ccccc7)nc(-c7ccccc7)n6)c54)c3)cc2)cc1 ; c1ccc(-c2cc(-c3ccccc3)cc(-n3c4ccccc4c4ccc5c(c6ccccc6n5-c5nc(-c6ccccc6)nc(-c6ccccc6)n5)c43)c2)cc1 ; c1ccc(-c2ccc(-n3c4ccccc4c4ccc5c(c6ccccc6n5-c5nc(-c6ccccc6)nc(-c6ccccc6)n5)c43)c(-c3ccccc3)c2)cc1]

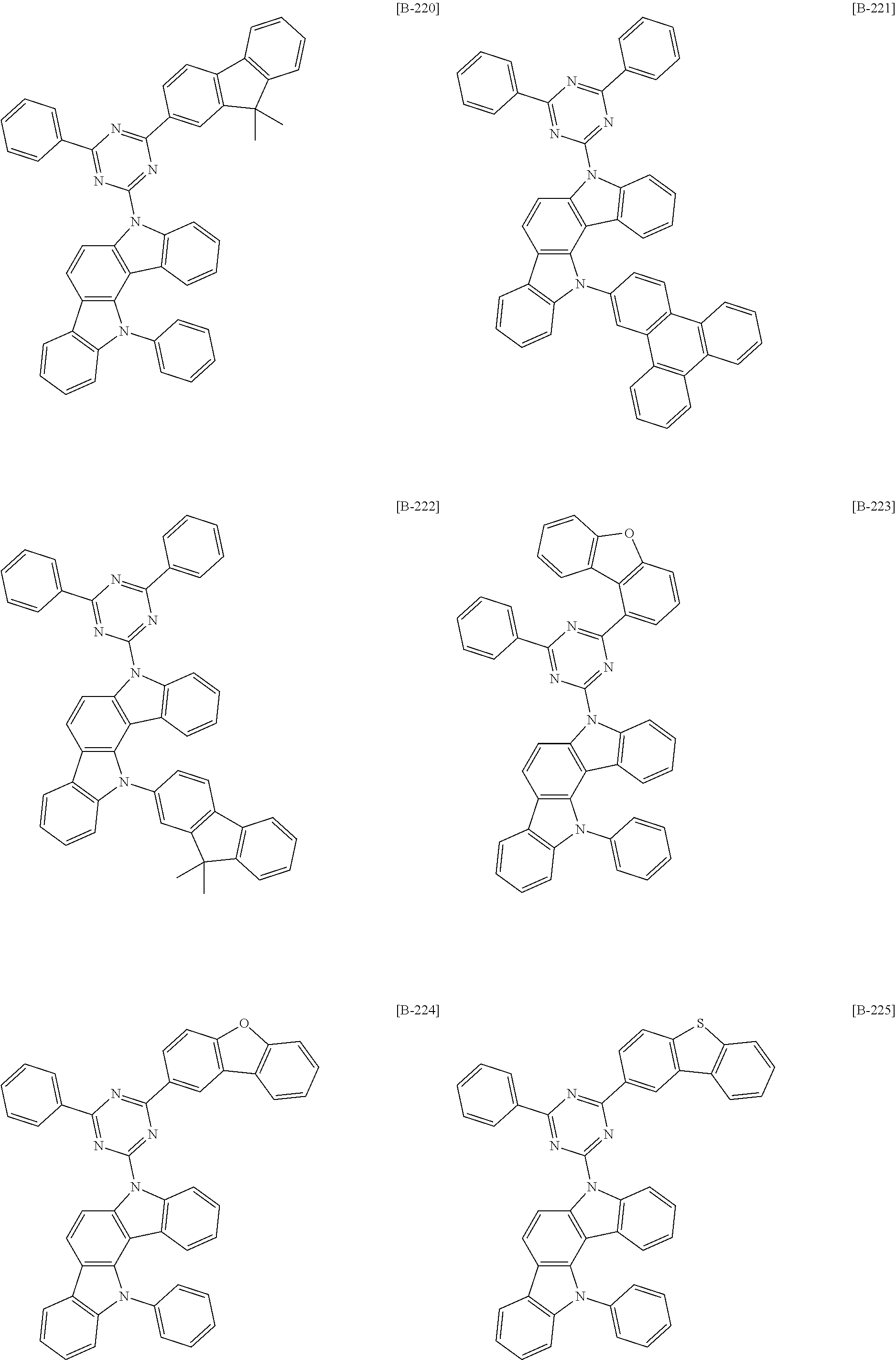

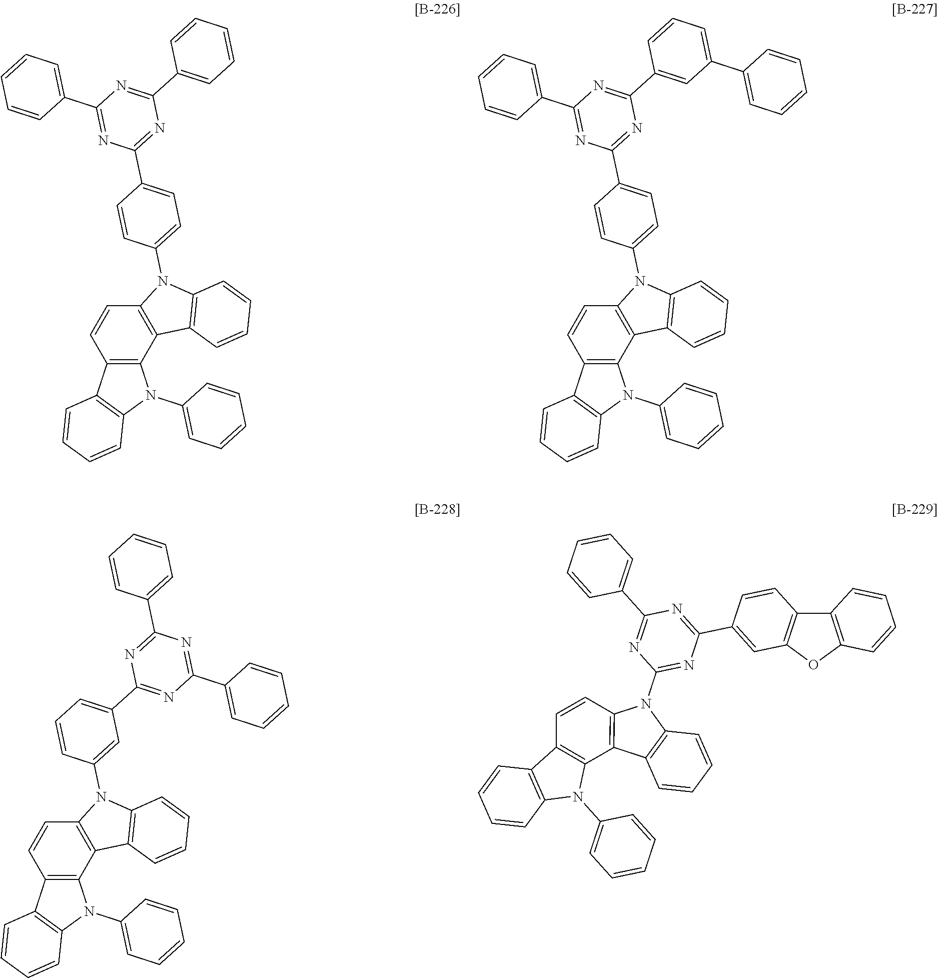 [SMILES: c1ccc(-c2nc(-c3ccccc3)nc(-c3ccc(-n4c5ccccc5c5c4ccc4c6ccccc6n(-c6ccccc6)c45)cc3)n2)cc1 ; c1ccc(-c2cccc(-c3nc(-c4ccccc4)nc(-c4ccc(-n5c6ccccc6c6c5ccc5c7ccccc7n(-c7ccccc7)c56)cc4)n3)c2)cc1 ; c1ccc(-c2nc(-c3ccccc3)nc(-c3cccc(-n4c5ccccc5c5c4ccc4c6ccccc6n(-c6ccccc6)c45)c3)n2)cc1 ; c1ccc(-c2nc(-c3ccc4c(c3)oc3ccccc34)nc(-n3c4ccccc4c4c3ccc3c5ccccc5n(-c5ccccc5)c34)n2)cc1]

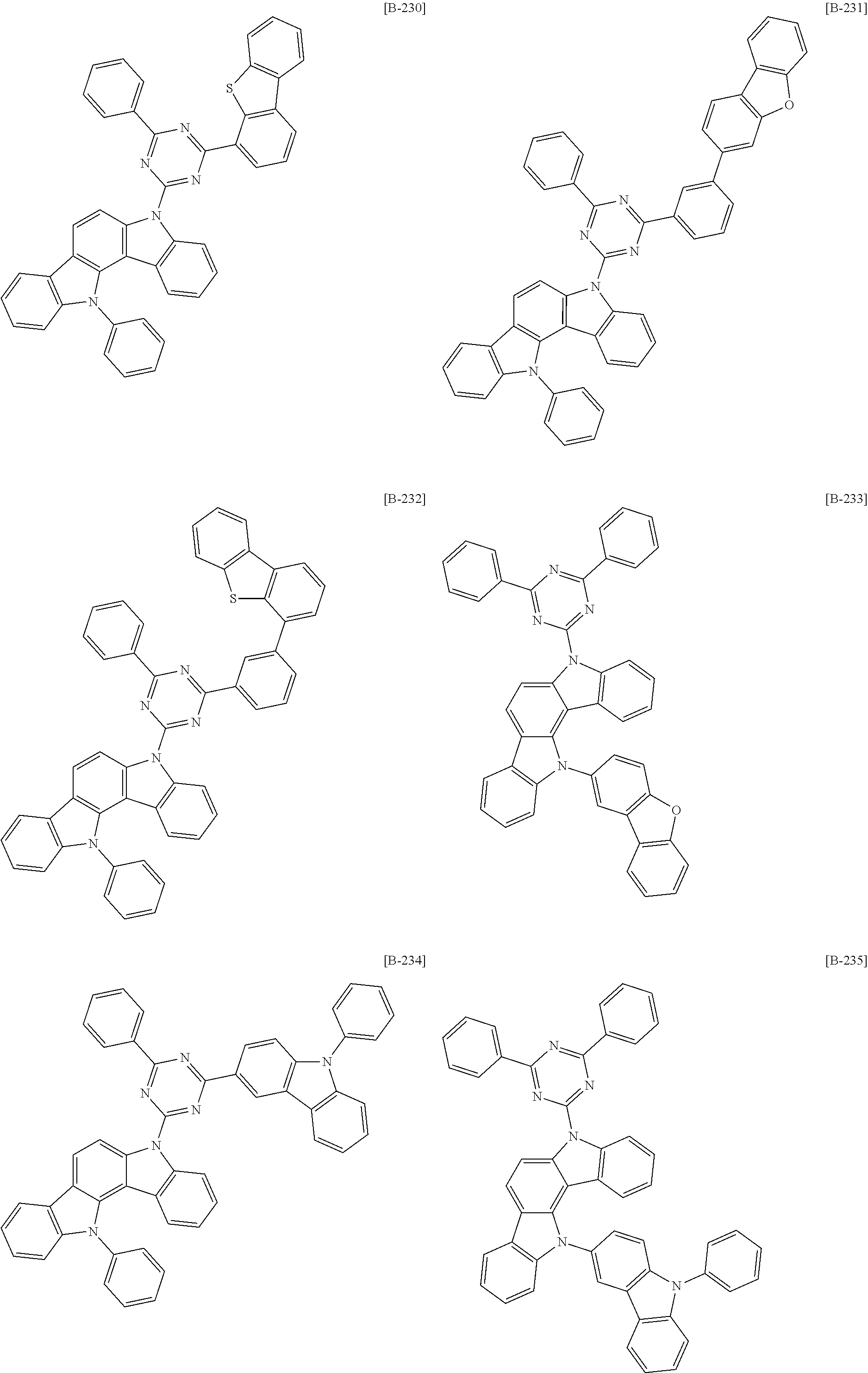

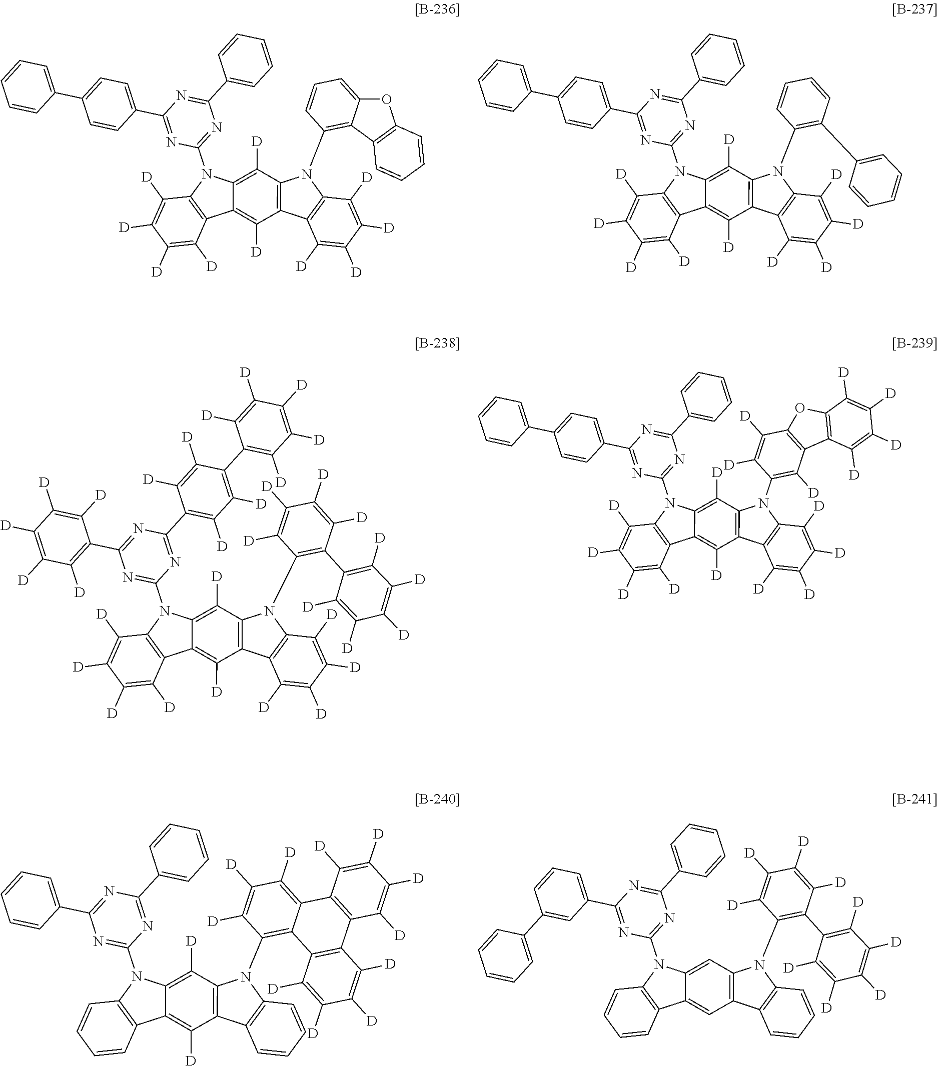

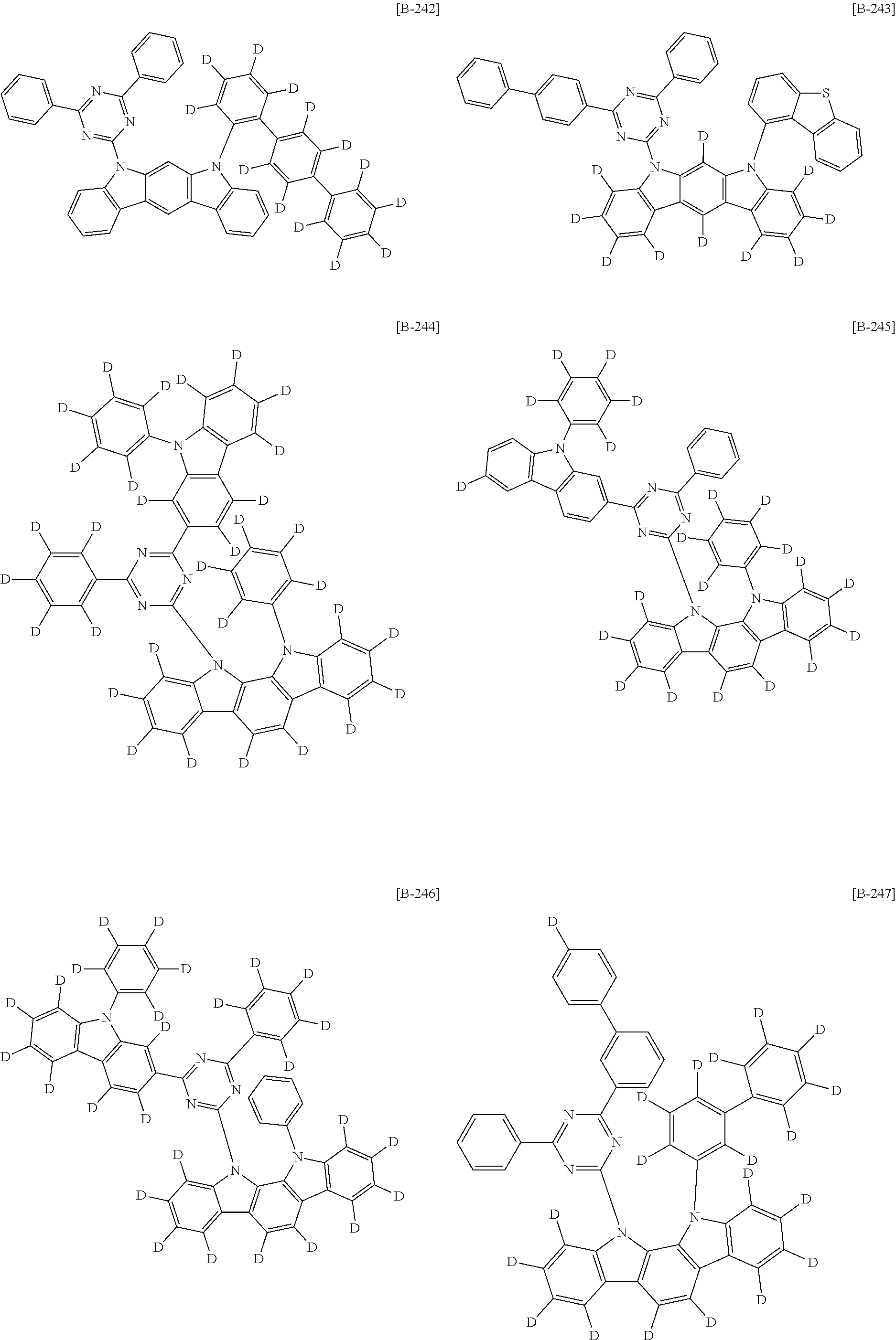

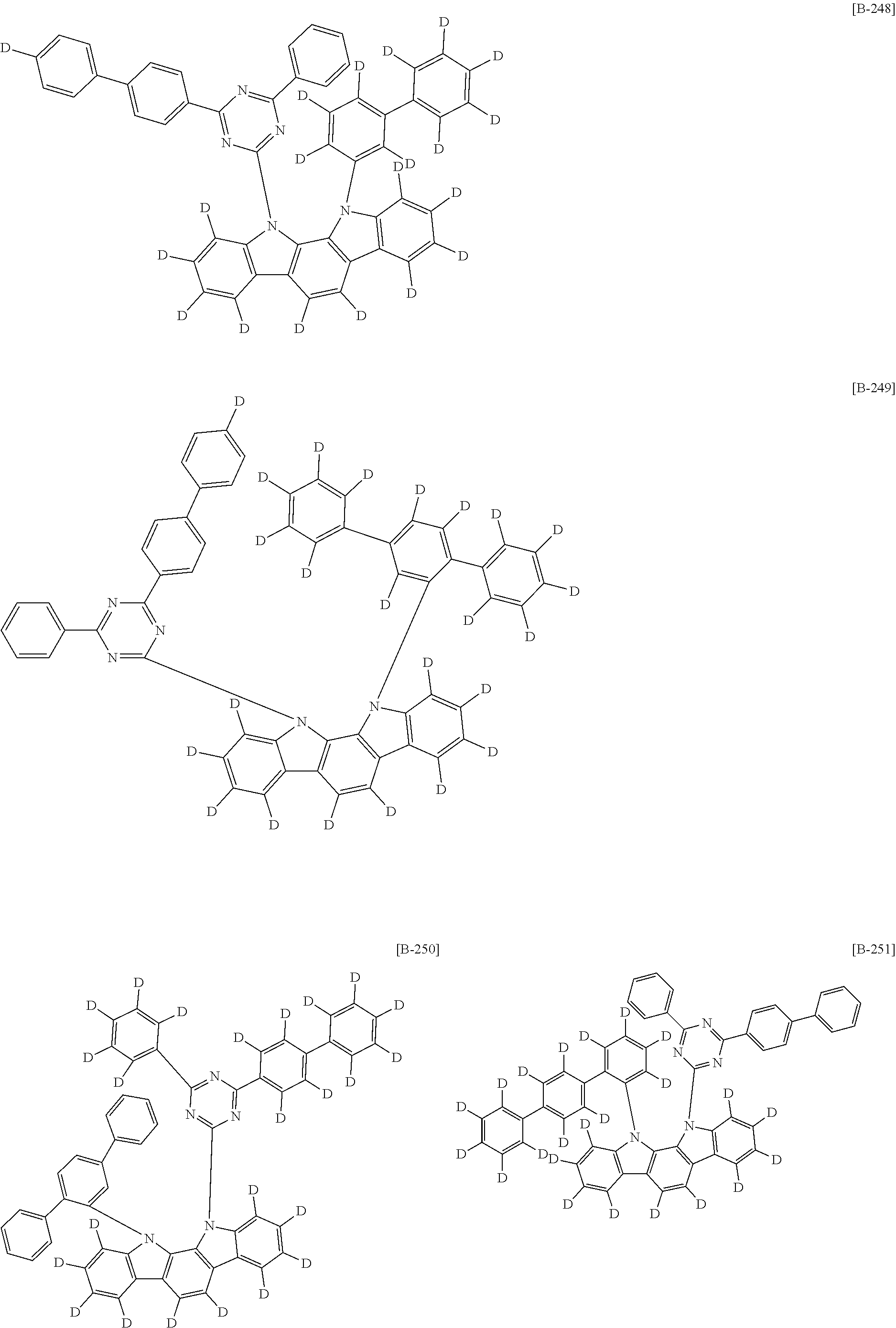

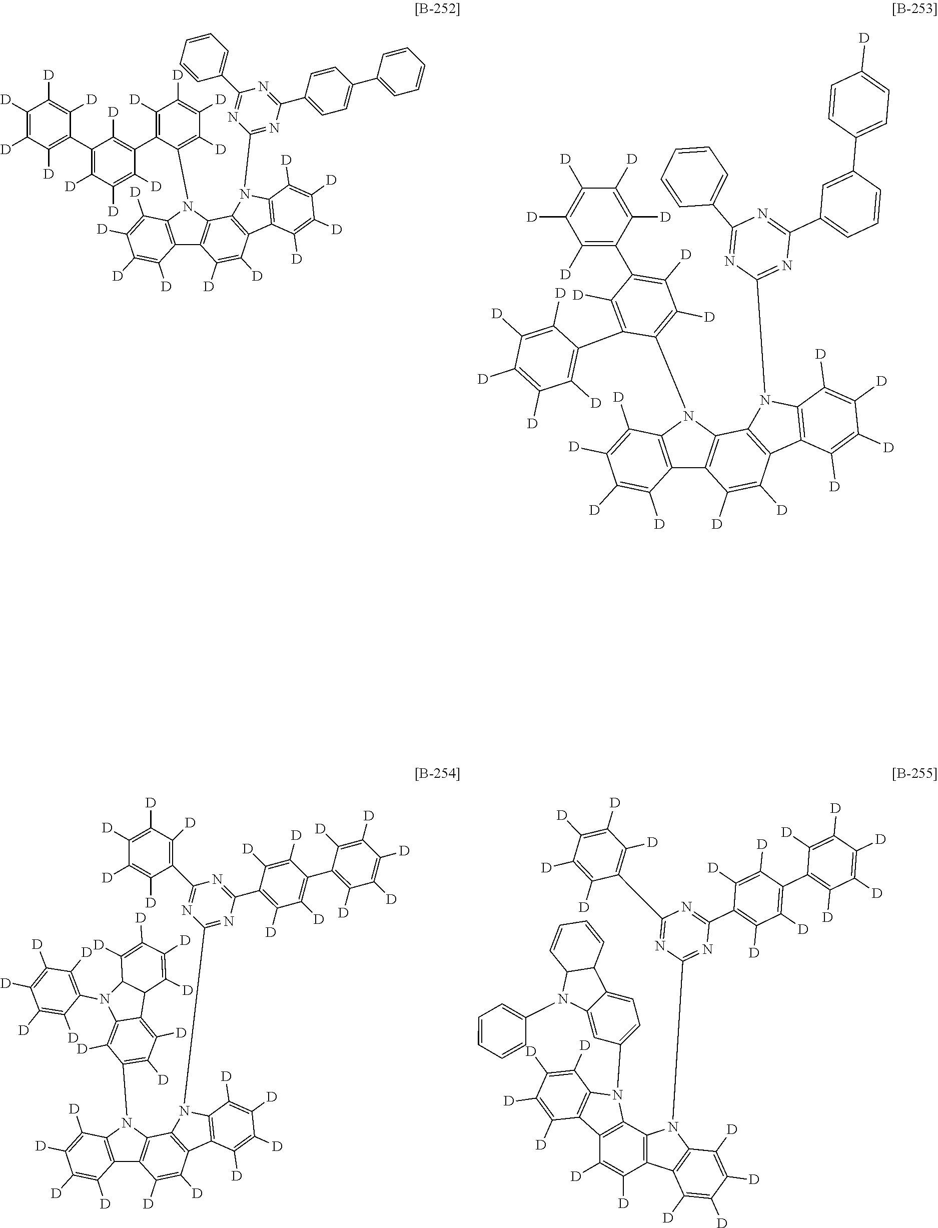

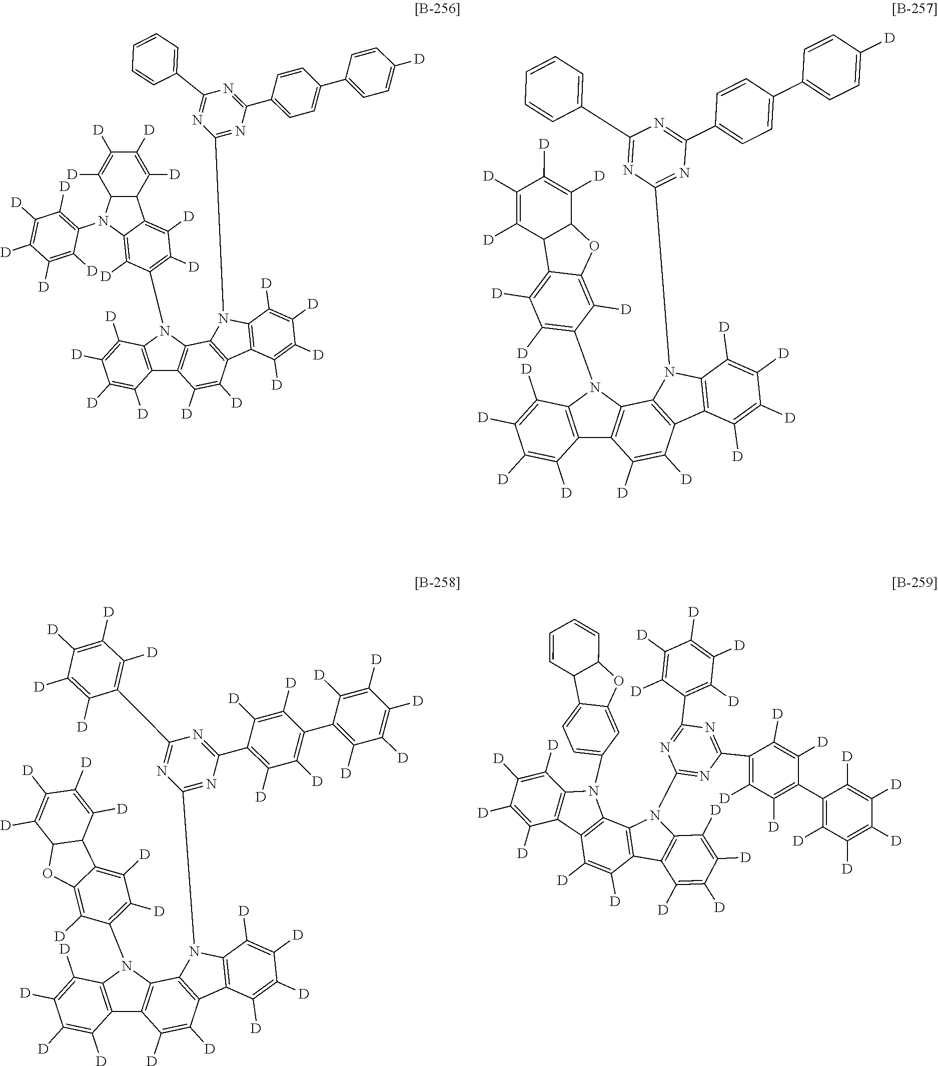

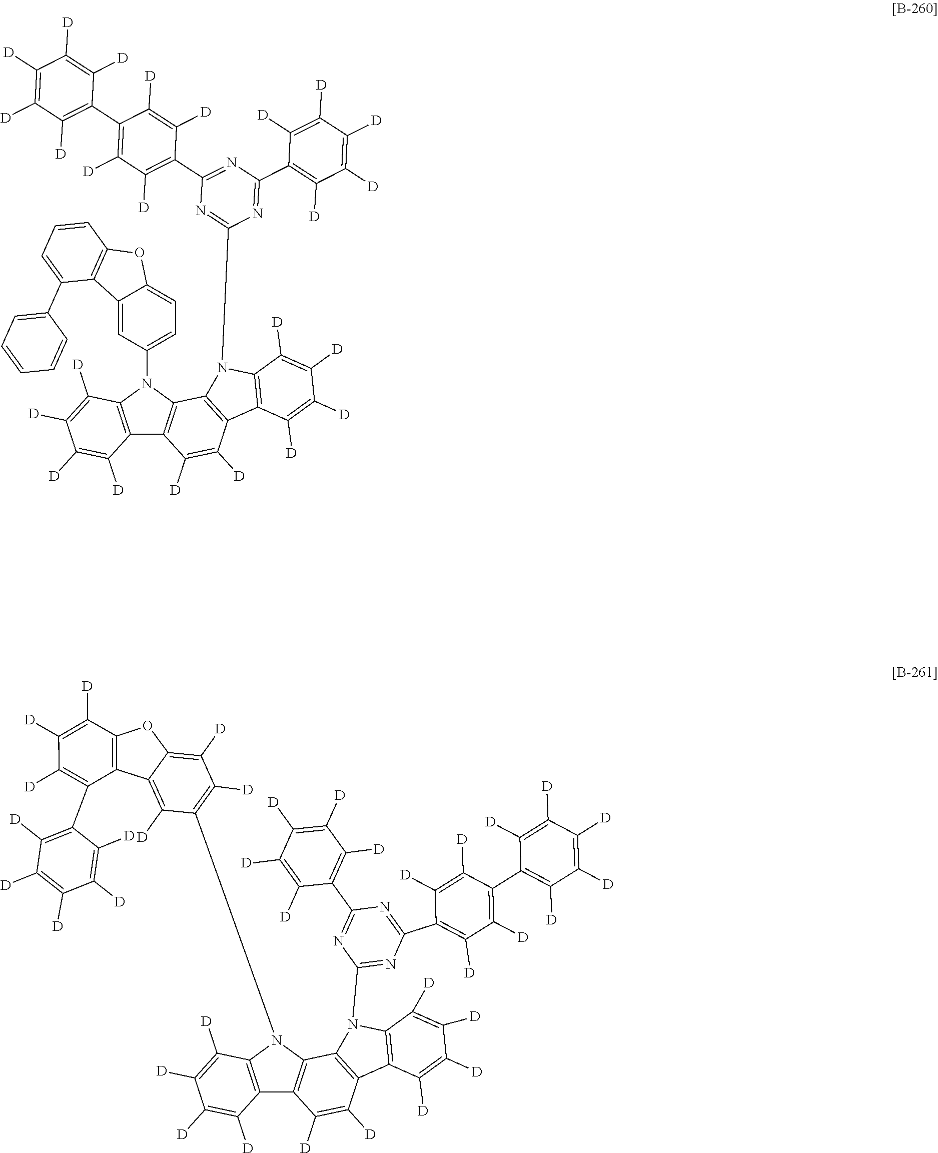

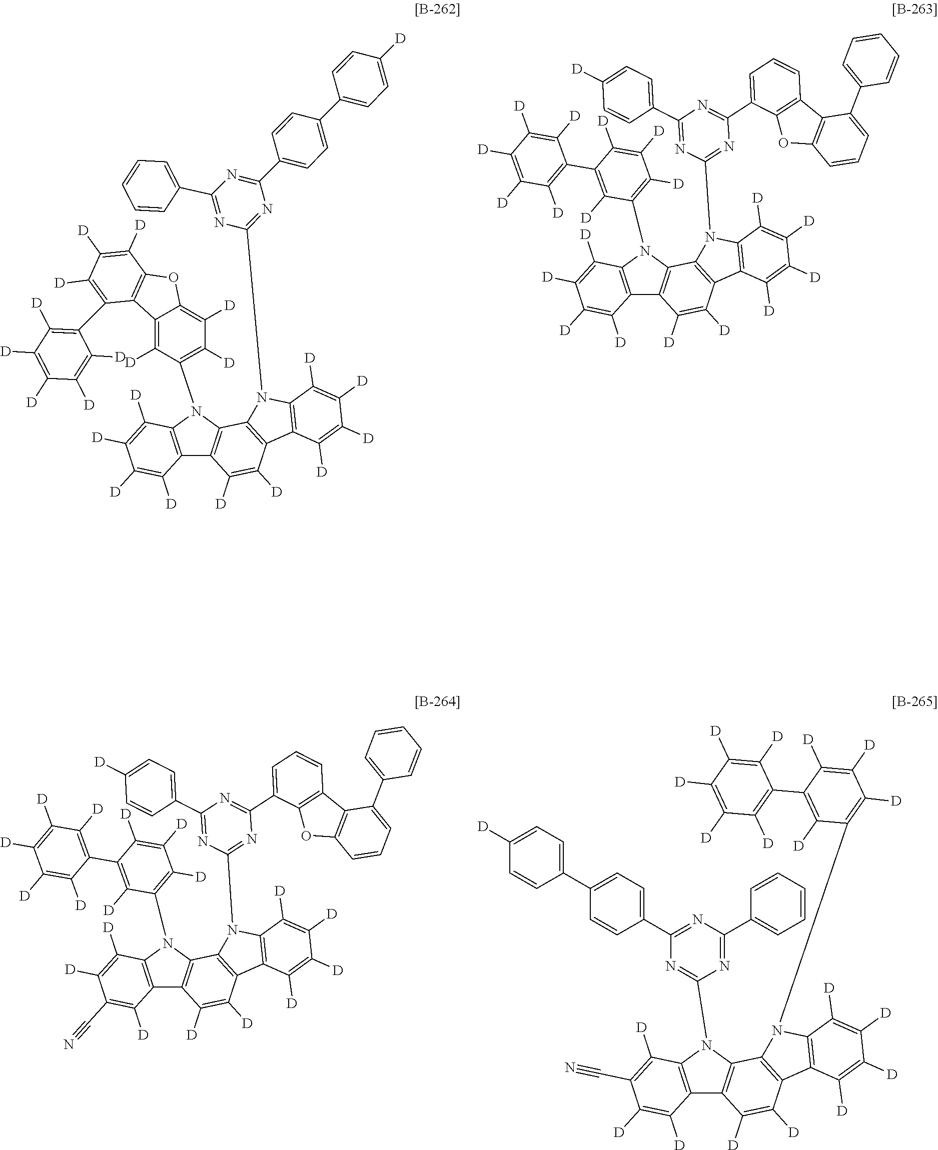

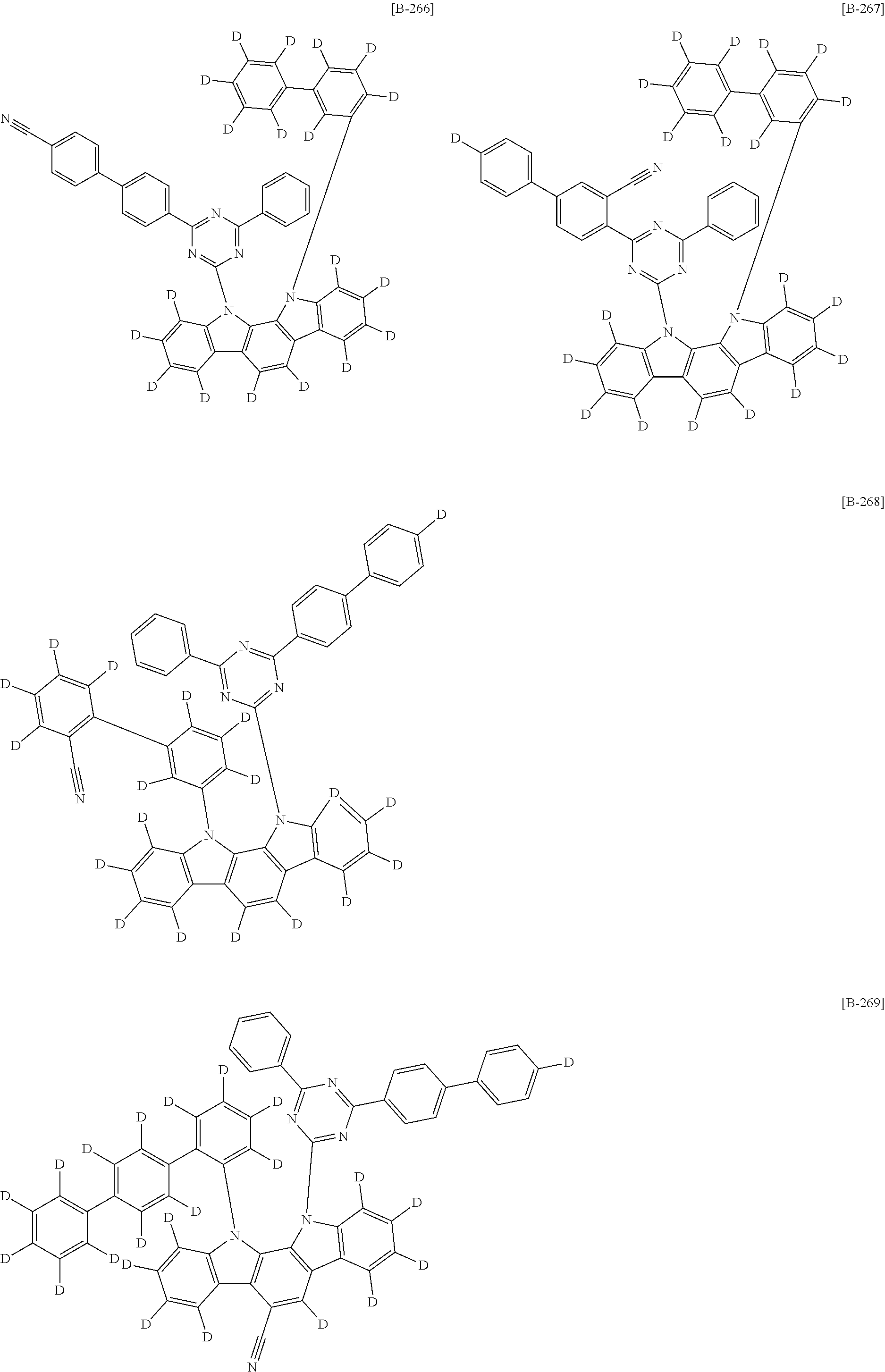

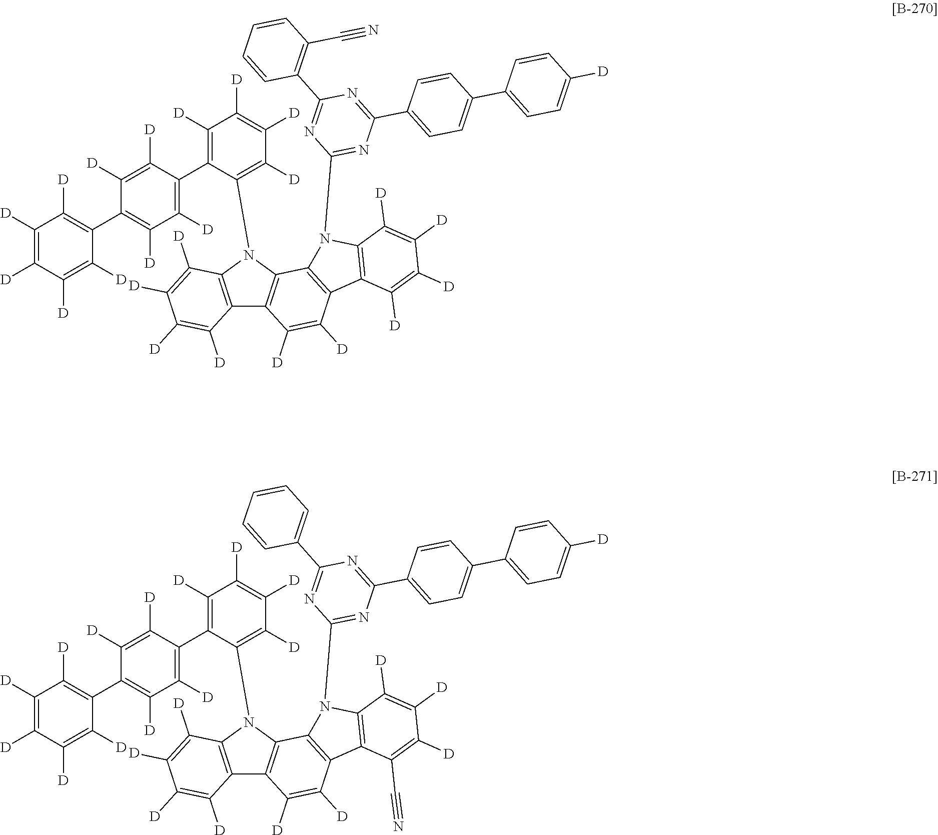 [SMILES: [2H]c1ccc(-c2ccc(-c3nc(-c4ccccc4C#N)nc(-n4c5c([2H])c([2H])c([2H])c([2H])c5c5c([2H])c([2H])c6c7c([2H])c([2H])c([2H])c([2H])c7n(-c7c([2H])c([2H])c([2H])c([2H])c7-c7c([2H])c([2H])c(-c8c([2H])c([2H])c([2H])c([2H])c8[2H])c([2H])c7[2H])c6c54)n3)cc2)cc1 ; [2H]c1ccc(-c2ccc(-c3nc(-c4ccccc4)nc(-n4c5c([2H])c([2H])c([2H])c(C#N)c5c5c([2H])c([2H])c6c7c([2H])c([2H])c([2H])c([2H])c7n(-c7c([2H])c([2H])c([2H])c([2H])c7-c7c([2H])c([2H])c(-c8c([2H])c([2H])c([2H])c([2H])c8[2H])c([2H])c7[2H])c6c54)n3)cc2)cc1]

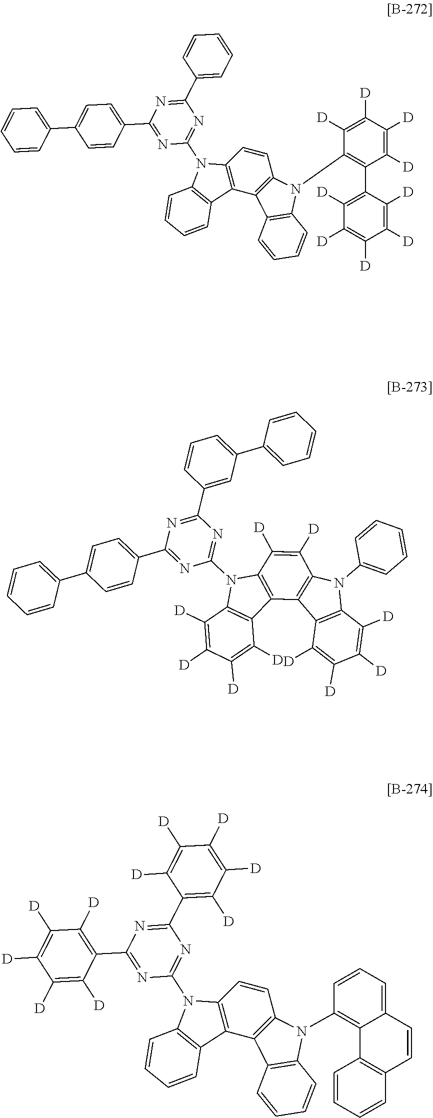 [SMILES: [2H]c1c([2H])c([2H])c(-c2c([2H])c([2H])c([2H])c([2H])c2-n2c3ccccc3c3c4c5ccccc5n(-c5nc(-c6ccccc6)nc(-c6ccc(-c7ccccc7)cc6)n5)c4ccc32)c([2H])c1[2H] ; [2H]c1c([2H])c([2H])c2c(c1[2H])c1c3c4c([2H])c([2H])c([2H])c([2H])c4n(-c4nc(-c5ccc(-c6ccccc6)cc5)nc(-c5cccc(-c6ccccc6)c5)n4)c3c([2H])c([2H])c1n2-c1ccccc1 ; [2H]c1c([2H])c([2H])c(-c2nc(-c3c([2H])c([2H])c([2H])c([2H])c3[2H])nc(-n3c4ccccc4c4c5c6ccccc6n(-c6cccc7ccc8ccccc8c67)c5ccc43)n2)c([2H])c1[2H]]

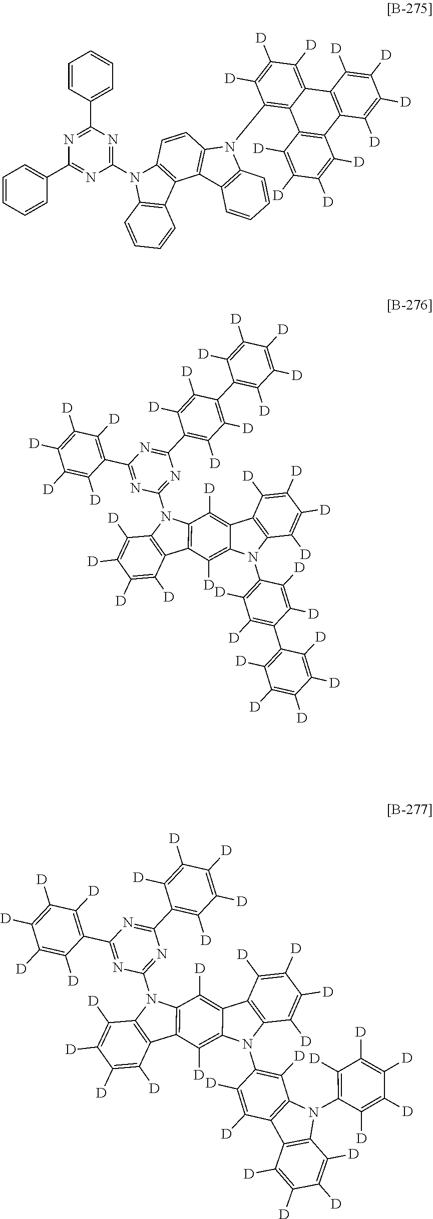

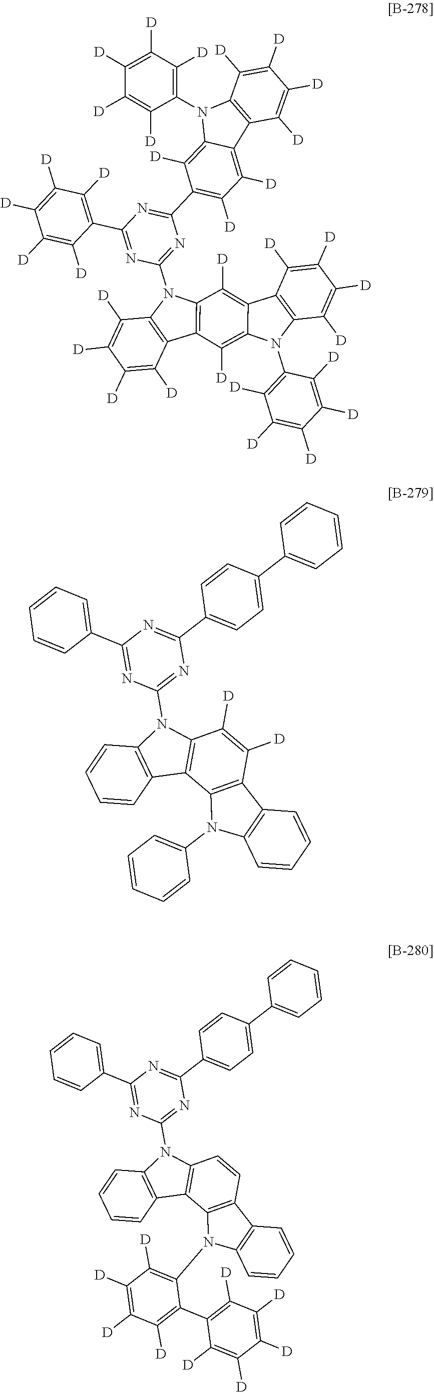

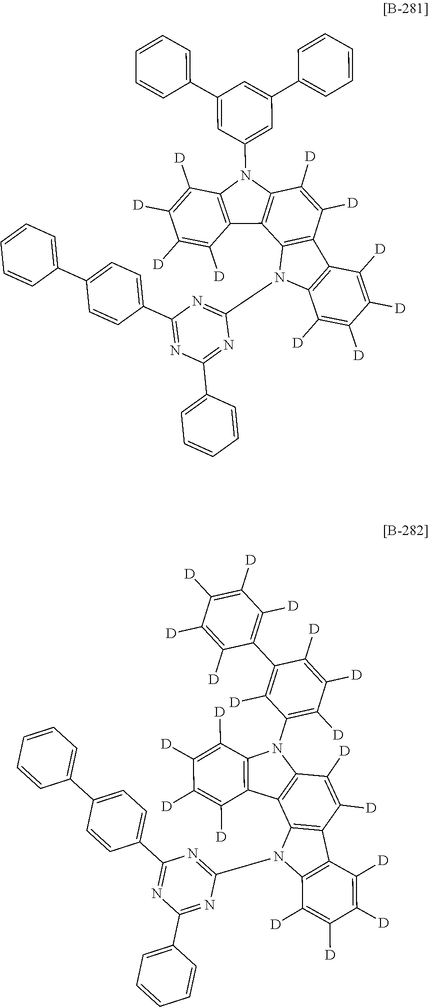 [SMILES: [2H]c1c([2H])c([2H])c2c(c1[2H])c1c(c([2H])c([2H])c3c4c([2H])c([2H])c([2H])c([2H])c4n(-c4nc(-c5ccccc5)nc(-c5ccc(-c6ccccc6)cc5)n4)c31)n2-c1cc(-c2ccccc2)cc(-c2ccccc2)c1 ; [2H]c1c([2H])c([2H])c(-c2c([2H])c([2H])c([2H])c(-n3c4c([2H])c([2H])c([2H])c([2H])c4c4c3c([2H])c([2H])c3c5c([2H])c([2H])c([2H])c([2H])c5n(-c5nc(-c6ccccc6)nc(-c6ccc(-c7ccccc7)cc6)n5)c34)c2[2H])c([2H])c1[2H]]

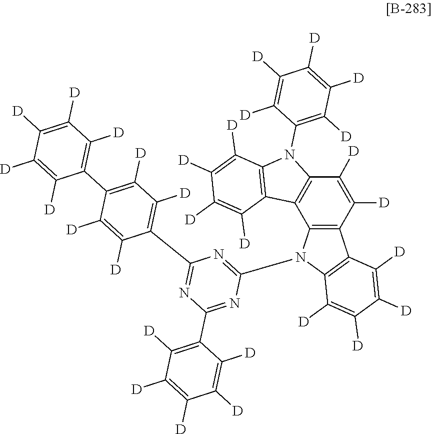 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-n3c4c([2H])c([2H])c([2H])c([2H])c4c4c([2H])c([2H])c5c(c6c([2H])c([2H])c([2H])c([2H])c6n5-c5c([2H])c([2H])c([2H])c([2H])c5[2H])c43)n2)c([2H])c1[2H]]

The first compound and the second compound may be included, for example, in a weight ratio of about 1:99 to about 99:1. Within the range, a desirable weight ratio may be adjusted using an electron transport capability of the first compound and a hole transport capability of the second compound to realize bipolar characteristics and thus to improve efficiency and life-span. Within the range, they may be for example included in a weight ratio of about 10:90 to 90:10, about 20:80 to 80:20, for example, about 20:80 to about 70:30, about 20:80 to about 60:40, and about 30:70 to about 60:40. As a specific example, they may be included in a weight ratio of 40:60, 50:50, or 60:40.

In addition to the aforementioned first and second compounds, one or more compounds may be further included.

The aforementioned composition for an organic optoelectronic device may further include a dopant.

The dopant may for example be a phosphorescent dopant, for example a red, green or blue phosphorescent dopant, for example a red or green phosphorescent dopant.

The dopant is a material mixed with the compound for the organic optoelectronic device in a small amount to cause light emission and may be generally a material such as a metal complex that emits light by multiple excitation into a triplet or more. The dopant may be, for example an inorganic, organic, or organic-inorganic compound, and one or more types thereof may be used.

Examples of the dopant may be a phosphorescent dopant and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, for example, a compound represented by Chemical Formula Z, but is not limited thereto.

L⁷MX [Chemical Formula Z]

In Chemical Formula Z, Mis a metal, and L′ and X are the same as or different from each other, and are ligands forming a complex compound with M.

M may be, for example Ir, Pt, Os, Ti, Zr, Hf, Eu, Tb, Tm, Fe, Co, Ni, Ru, Rh, Pd, or a combination thereof, and L⁷ and X may be, for example a bidentate ligand.

Examples of the ligands represented by L⁷ and X may be selected from the chemical formulas of Group A, but are not limited thereto.

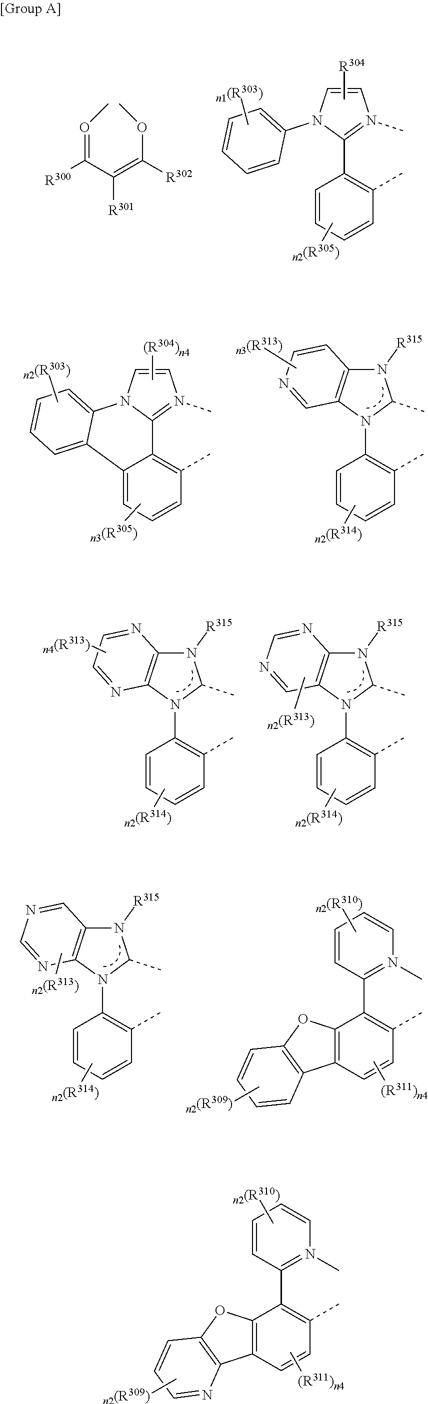

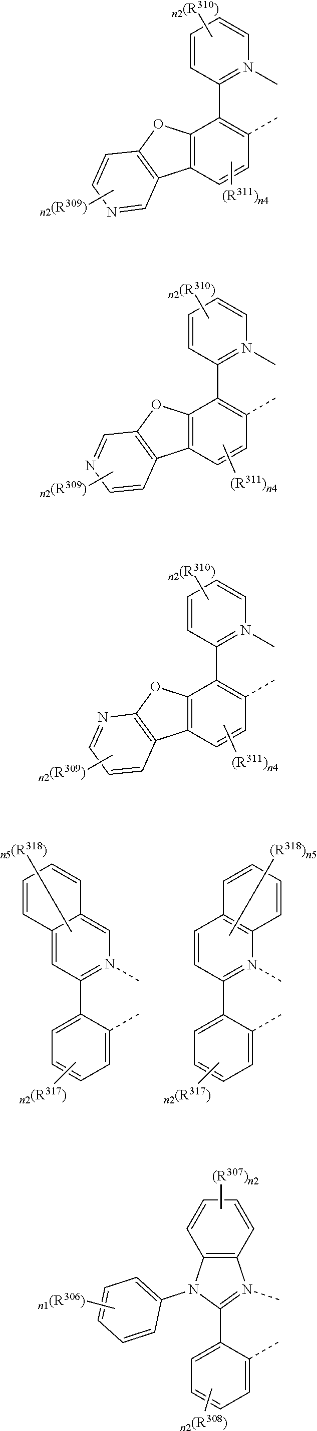

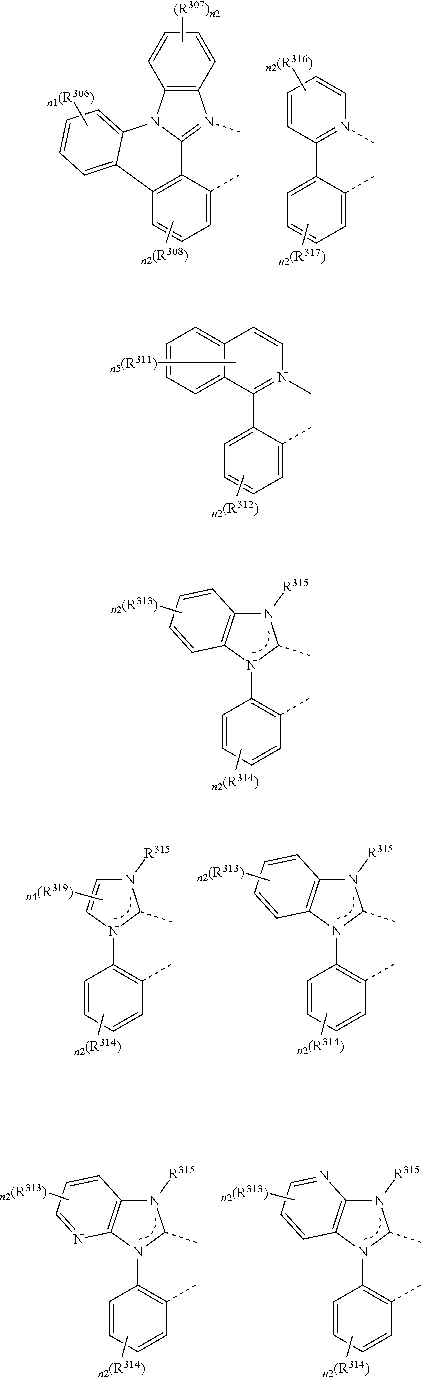

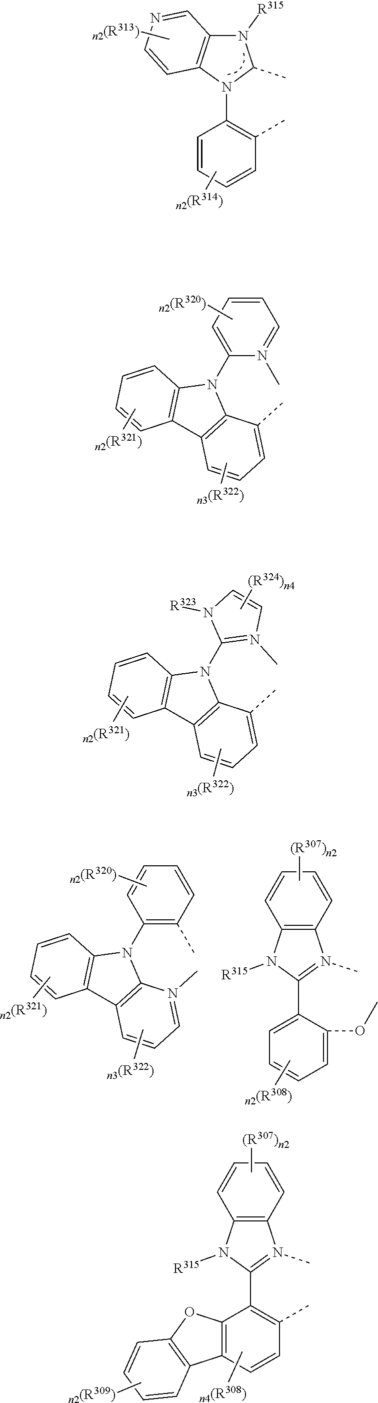

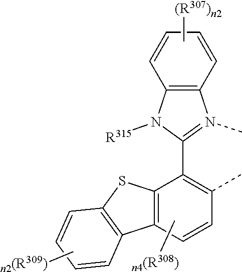

In Group A,
R³⁰⁰ to R³⁰² are each independently hydrogen, deuterium, a C1 to C30 alkyl group with or without halogen substitution, a C6 to C30 aryl group with or without C1 to C30 alkyl substitution, or a halogen, and
R³⁰³ to R³²⁴ are each independently hydrogen, deuterium, a halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and a C6 to C30 aryl group, or a triarylsilyl group having a substituted or unsubstituted C6 to C30 aryl group.

The dopant according to an embodiment may be an iridium complex, for example, represented by Chemical Formula 4-1 or Chemical Formula 4-2.

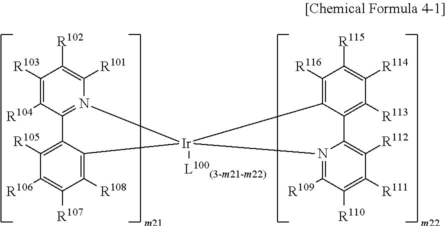

In Chemical Formula 4-1,
R¹⁰¹ to R¹¹⁶ are each independently hydrogen, deuterium, a substituted or unsubstituted C1 to C10 alkyl group, a substituted or unsubstituted C6 to C20 aryl group, or —SiR¹³²R¹³³R¹³⁴,
R¹³² to R¹³⁴ are each independently a substituted or unsubstituted C1 to C6 alkyl group,
at least one of R¹⁰¹ to R¹¹⁶ is a functional group represented by Chemical Formula V-1,
L¹⁰⁰ is a bidentate ligand of a monovalent anion, and is a ligand that coordinates to iridium through a lone pair of carbons or heteroatoms, and
m21 and m22 are each independently any one of integers of 0 to 3 and m21+m22 is any one of integers of 1 to 3,

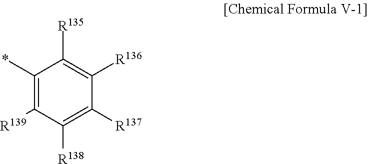

wherein, in Chemical Formula V-1,
R¹³⁵ to R¹³⁹ are each independently hydrogen, deuterium, a substituted or unsubstituted C1 to C10 alkyl group, a substituted or unsubstituted C6 to C20 aryl group, or —SiR¹³²R¹³³R¹³⁴, and
* refers to a portion linked to the carbon atom.

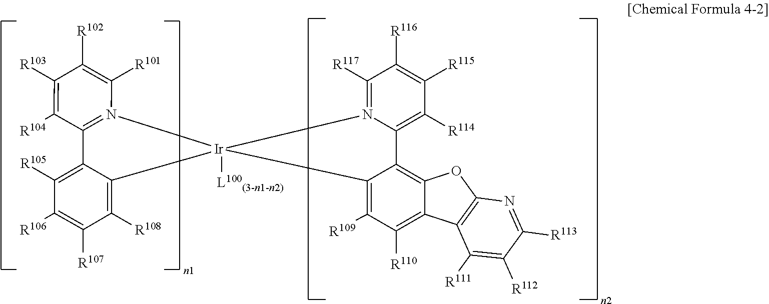

In Chemical Formula 4-2,
R¹⁰¹ to R¹¹⁷ are each independently hydrogen, deuterium, a substituted or unsubstituted C1 to C10 alkyl group, a substituted or unsubstituted C6 to C20 aryl group, or —Si R¹³³R¹³⁴R¹³⁵,
R¹³³ to R¹³⁵ are each independently a substituted or unsubstituted C1 to C6 alkyl group,
L¹⁰⁰ is a bidentate ligand of a monovalent anion, and is a ligand that coordinates to iridium through a lone pair of carbons or heteroatoms, and
n1 and n2 are each independently any one of integers of 0 to 3 and n1+n2 is any one of integers of 1 to 3.

In another embodiment, the dopant may be a platinum complex, for example represented by Chemical Formula Z-1.

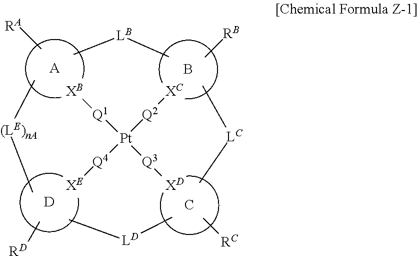

In Chemical Formula Z-1, rings A, B, C, and D each independently represent a 5-membered or 6-membered carbocyclic or heterocyclic ring;
R^{A}, R^{B}, R^{C}, and R^{D} each independently represent mono-, di-, tri-, or tetra-substitution, or unsubstitution;
L^{B}, L^{C}, and L^{D} are each independent selected from a direct bond, BR, NR, PR, O, S, Se, C═O, S═O, SO₂, CRR′, SiRR′, GeRR′, and a combination thereof;
when nA is 1, L^{E} is selected from a direct bond, BR, NR, PR, O, S, Se, C═O, S═O, SO₂, CRR′, SiRR′, GeRR′, and a combination thereof; when nA is 0, L^{E} does not exist; and
R^{A}, R^{B}, R^{C}, R^{D}, R, and R′ are each independently selected from hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, and a combination thereof; any adjacent R^{A}, R^{B}, R^{C}, R^{D}, R, and R′ are optionally linked to each other to provide a ring; X^{B}, X^{C}, X^{D}, and X^{E} are each independently selected from carbon and nitrogen; and Q¹, Q², Q³, and Q⁴ each represent oxygen or a direct bond.

The platinum complex may be represented, for example, by Chemical Formula 5-1 or Chemical Formula 5-2.

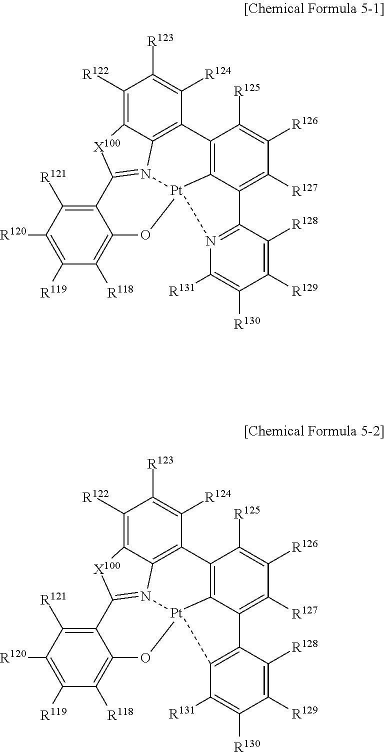

In Chemical Formula 5-1 and Chemical Formula 5-2,
X¹⁰⁰ is selected from O, S, and NR¹³²,
R¹¹⁸ to R¹³² are each independently hydrogen, deuterium, a substituted or unsubstituted C1 to C10 alkyl group, a substituted or unsubstituted C6 to C20 aryl group, or —SiR¹³³R¹³⁴R¹³⁵,
R¹³³ to R¹³⁵ are each independently a substituted or unsubstituted C1 to C6 alkyl group, at least one of R¹¹⁸ to R¹³² is —SiR¹³³R¹³⁴R¹³⁵ or a tert-butyl group, and
R¹³³ to R¹³⁵ are each independently a substituted or unsubstituted C1 to C6 alkyl group.

Hereinafter, an organic optoelectronic device including the aforementioned composition for an organic optoelectronic device is described.

The organic optoelectronic device may be any device to convert electrical energy into photoenergy and vice versa without particular limitation, and may be, for example an organic photoelectric device, an organic light emitting diode, an organic solar cell, and an organic photo-conductor drum.

Here, an organic light emitting diode as one example of an organic optoelectronic device is described referring to drawings.

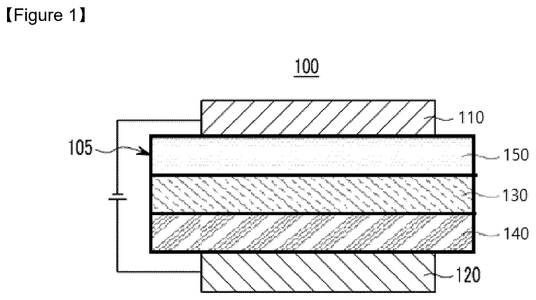

FIG. 1 is a cross-sectional view illustrating an organic light emitting diode according to some embodiments.

Referring to FIG. 1, an organic light emitting diode 100 according to some embodiments includes an anode 120 and a cathode 110 facing each other and an organic layer 105 between the anode 120 and cathode 110.

The anode 120 may be made of a conductor having a large work function to help hole injection, and may be for example a metal, a metal oxide and/or a conductive polymer. The anode 120 may be, for example a metal such as nickel, platinum, vanadium, chromium, copper, zinc, gold, and the like or an alloy thereof, a metal oxide such as zinc oxide, indium oxide, indium tin oxide (ITO), indium zinc oxide (IZO), and the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, and polyaniline, but is not limited thereto.

The cathode 110 may be made of a conductor having a small work function to help electron injection, and may be for example a metal, a metal oxide and/or a conductive polymer. The cathode 110 may be for example a metal such as magnesium, calcium, sodium, potassium, titanium, indium, yttrium, lithium, gadolinium, aluminum silver, tin, lead, cesium, barium, and the like, or an alloy thereof; a multi-layer structure material such as LiF/Al, LiO₂/Al, LiF/Ca, LiF/Al, and BaF₂/Ca, but is not limited thereto.

The organic layer 105 may include the aforementioned composition for an organic optoelectronic device.

The organic layer 105 includes a light emitting layer 130, and the light emitting layer 130 may include the aforementioned composition for an organic optoelectronic device.

The composition for an organic optoelectronic device further including a dopant may be, for example, a green light emitting composition.

The light emitting layer 130 may include, for example, the aforementioned composition for an organic optoelectronic device as a phosphorescent host.

The organic layer may further include a charge transport region in addition to the light emitting layer.

The charge transport region may be, for example, a hole transport region 140.

The hole transport region 140 may further increase hole injection and/or hole mobility and block electrons between the anode 120 and the light emitting layer 130.

Specifically, the hole transport region 140 may include a hole transport layer between the anode 120 and the light emitting layer 130, and a hole transport auxiliary layer between the light emitting layer 130 and the hole transport layer.

The aforementioned composition for an organic optoelectronic device is included in the light emitting layer, and at least one of the compounds listed in Group B may be included in at least one of the hole transport layer and the hole transport auxiliary layer.

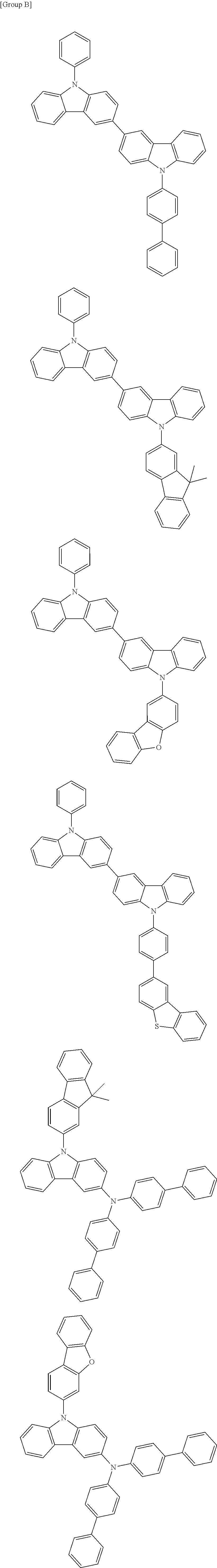

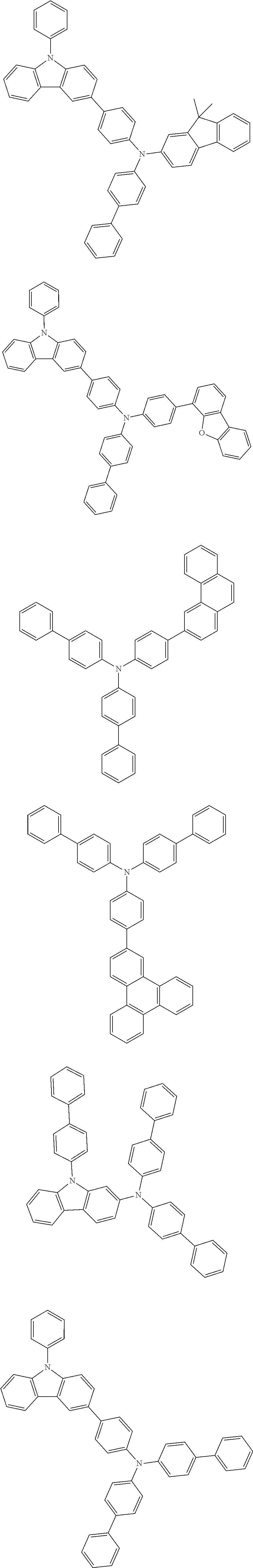

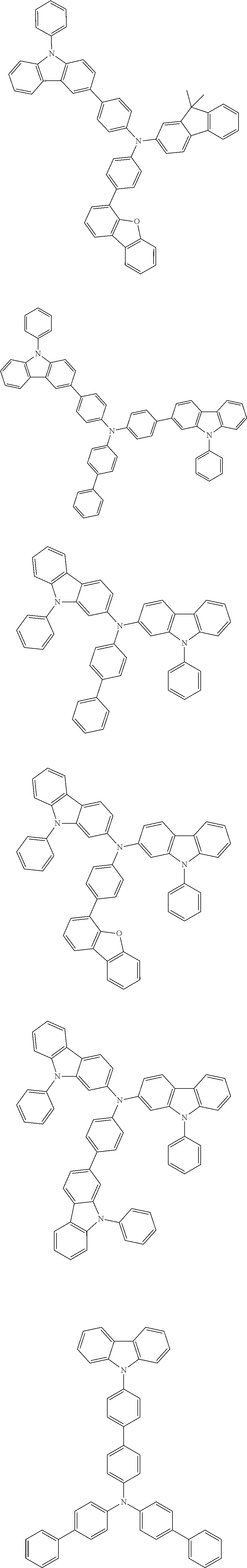

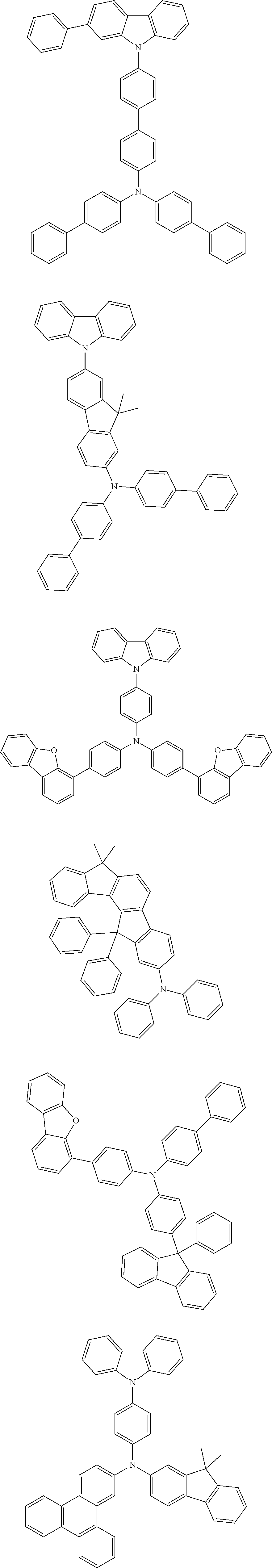

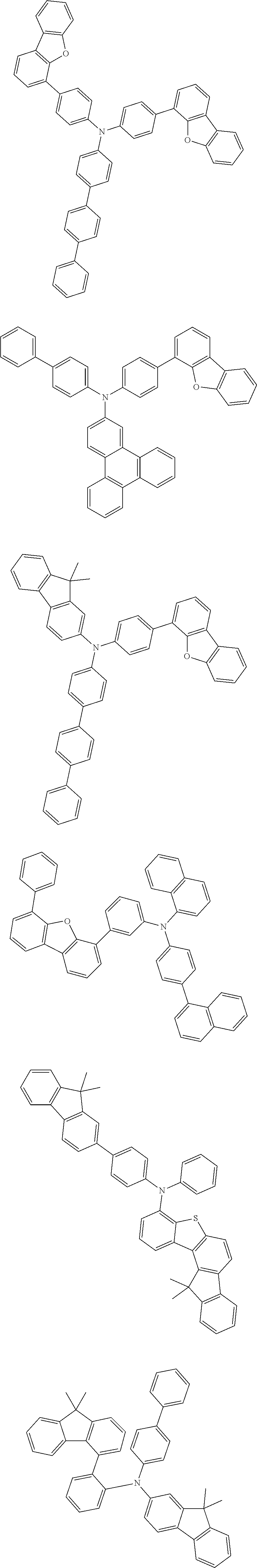

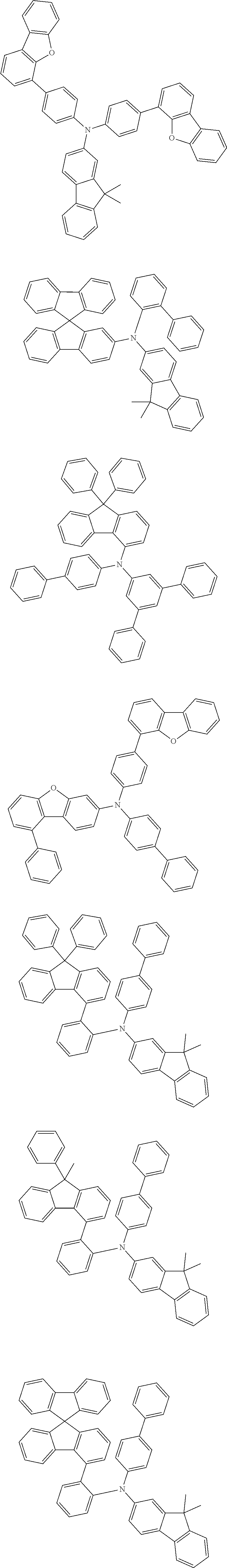

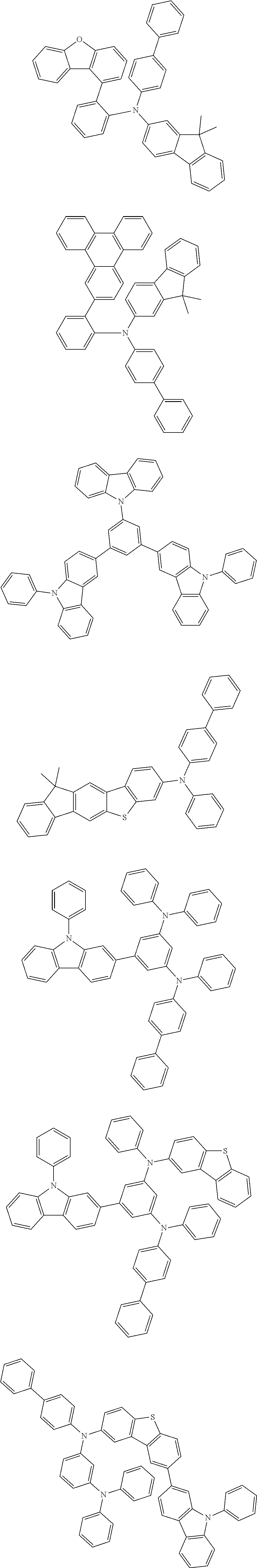

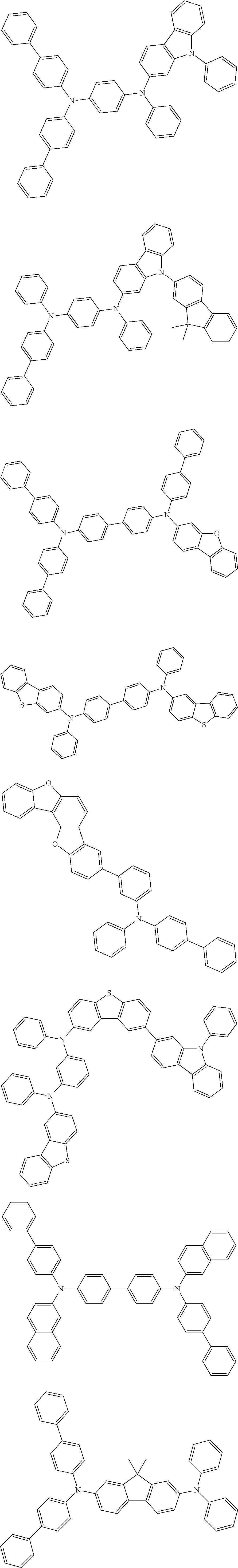

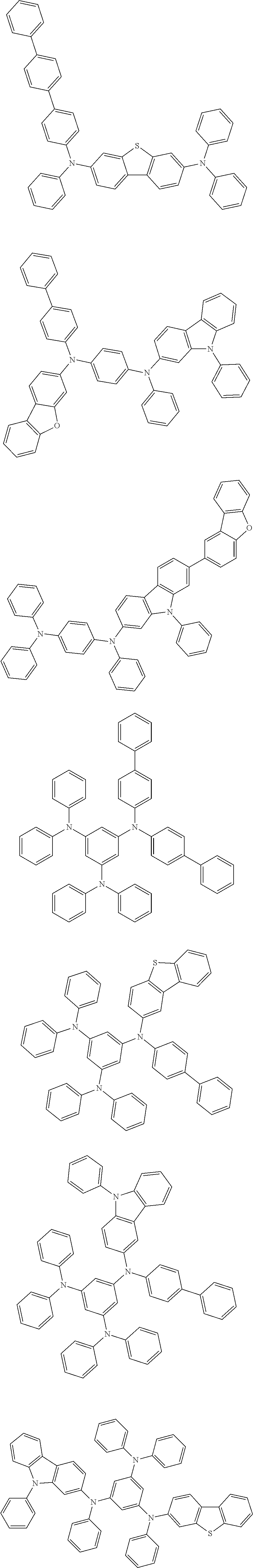

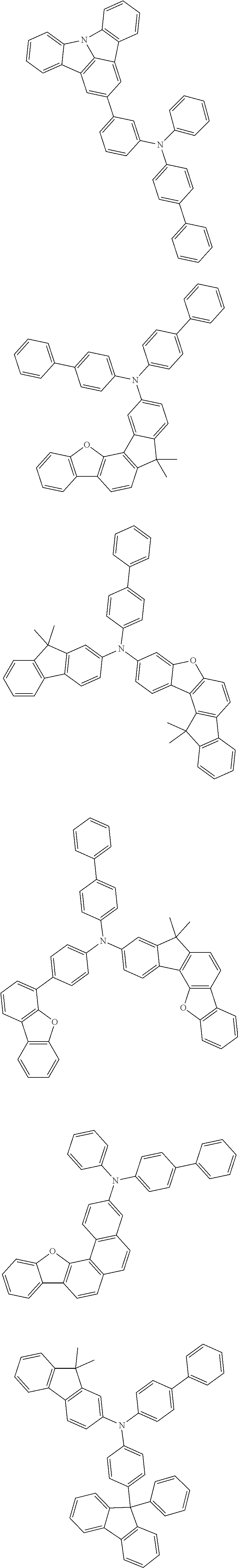

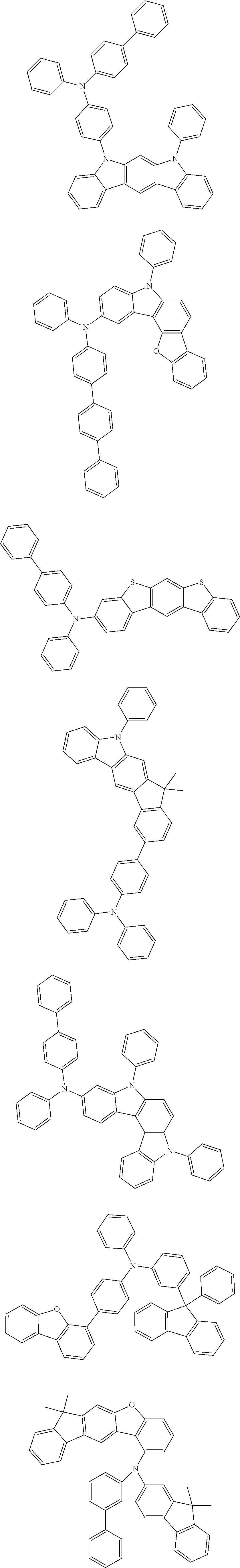

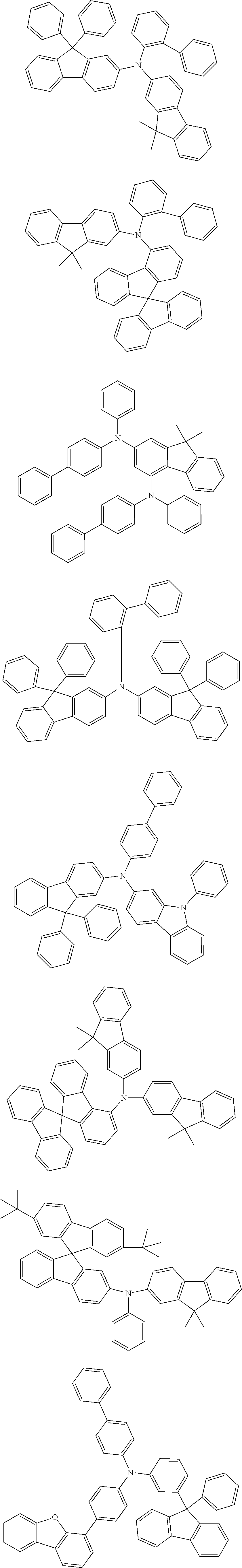

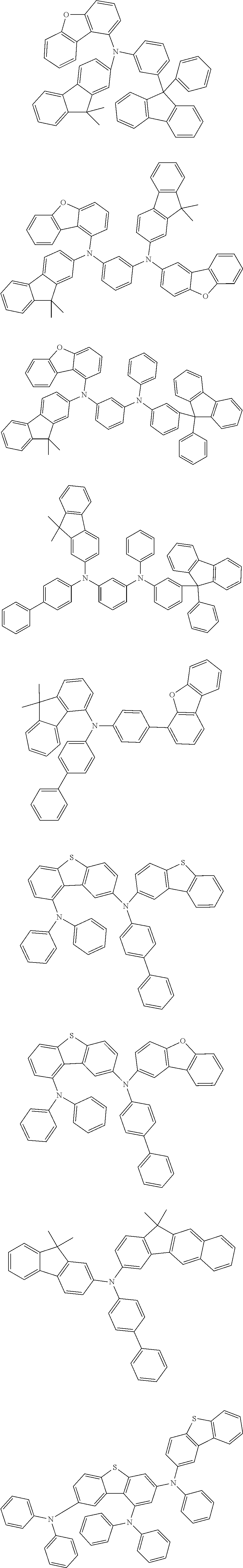

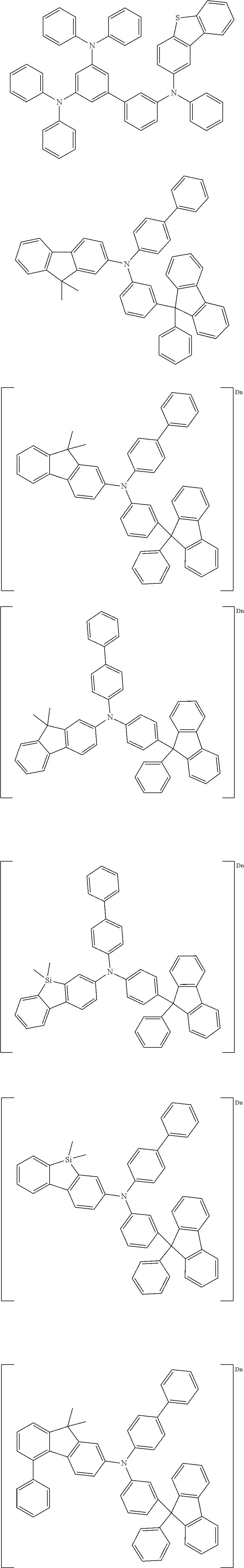

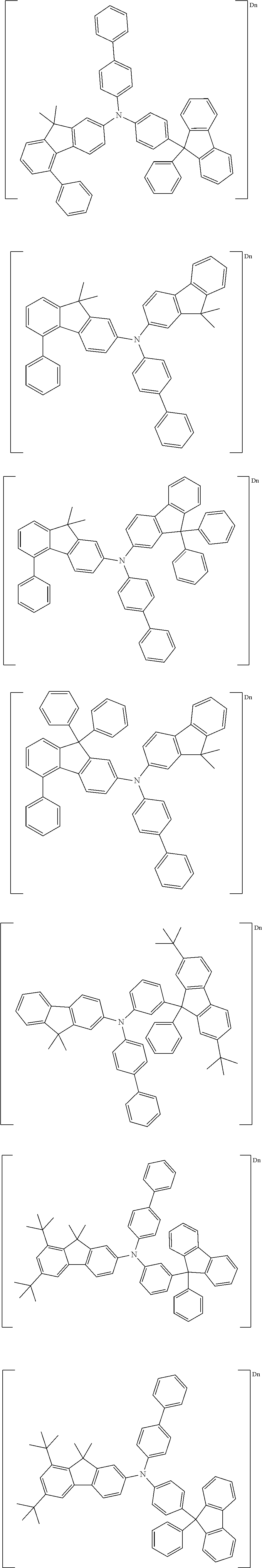

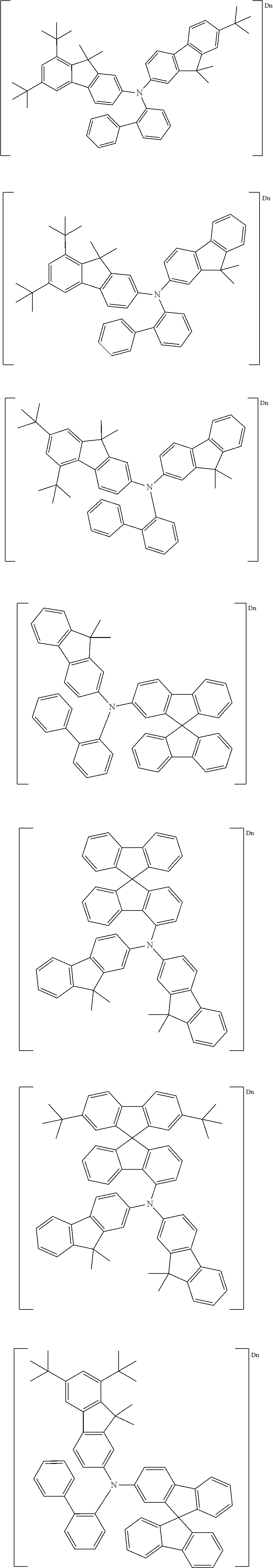

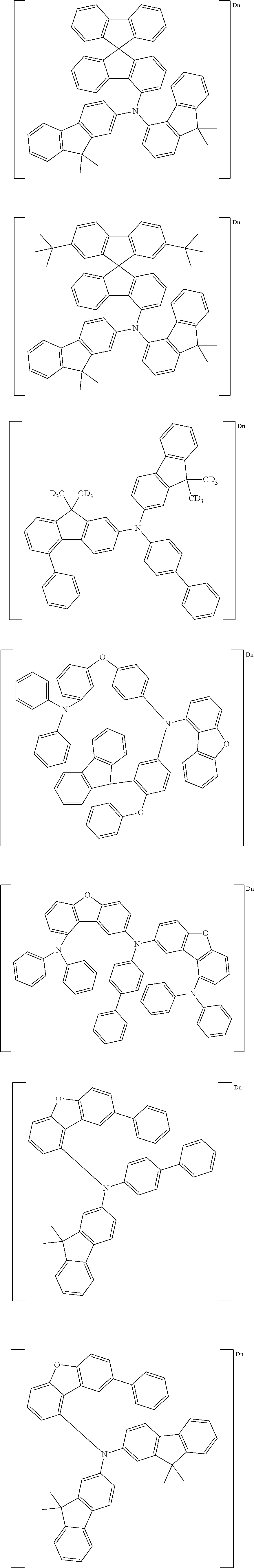

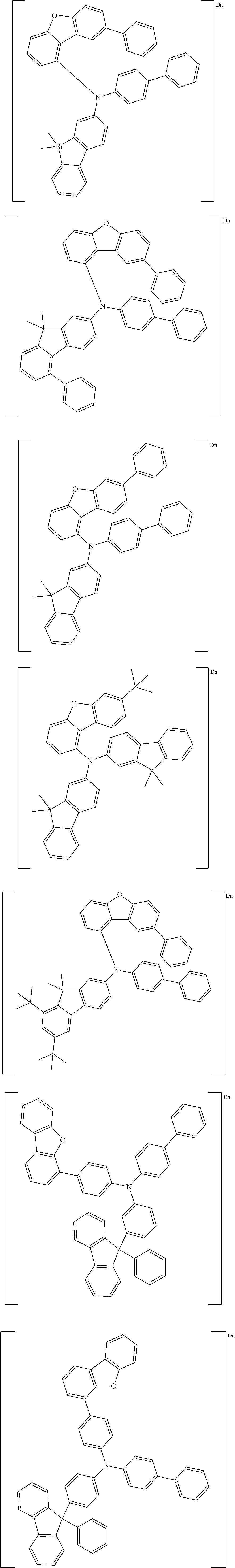

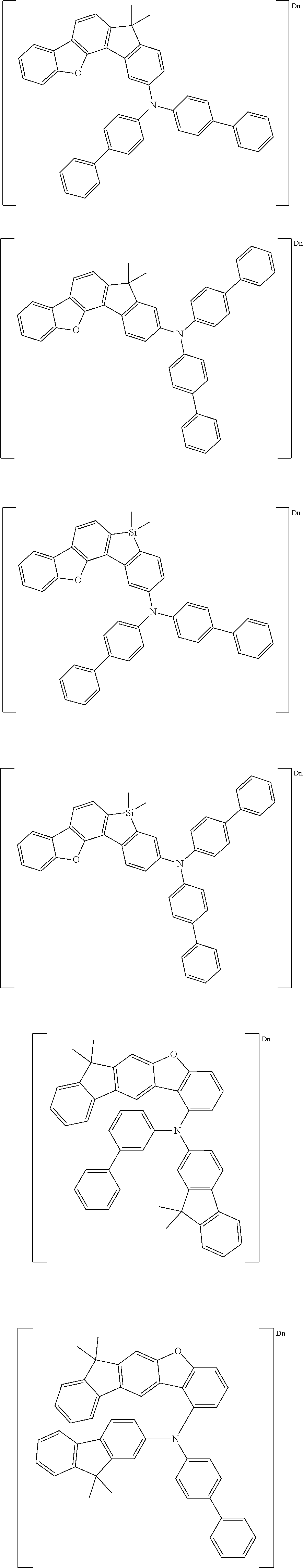

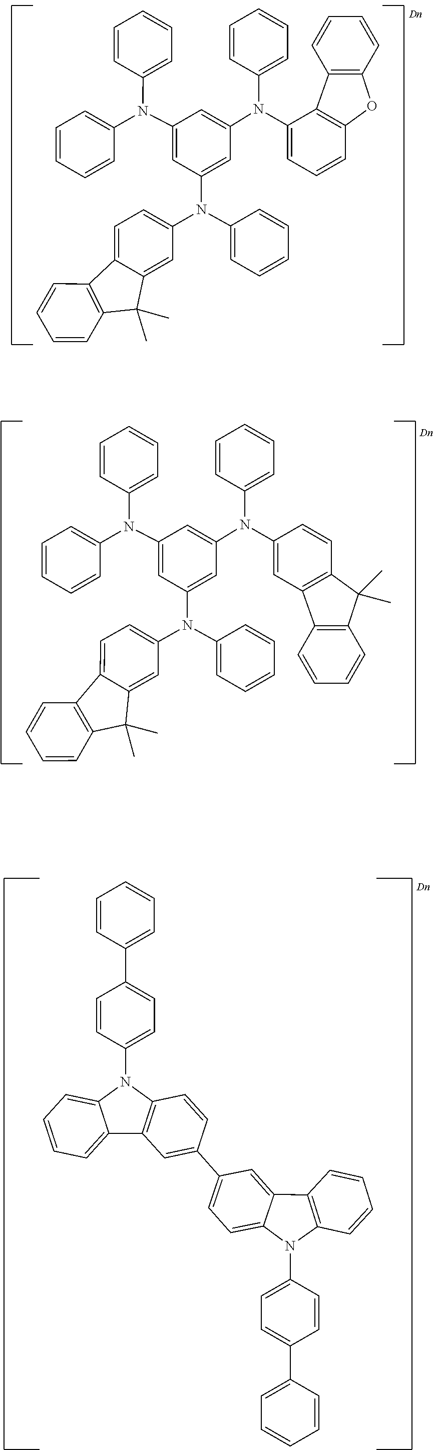

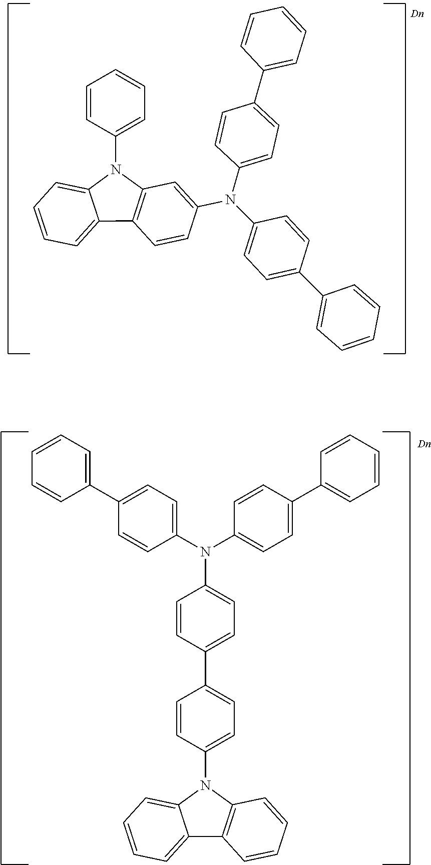

Here, Dn refers to the number of deuterium atoms substituted, and indicates a structure substituted with one or more deuterium atoms.

In the hole transport region 140, known compounds disclosed in U.S. Pat. No. 5,061,569A, JP1993-009471A, WO1995-009147A1, JP1995-126615A, JP1998-095973A, and the like and compounds similar thereto may be used in addition to the aforementioned compounds.

In addition, the charge transport region may be for example an electron transport region 150.

The electron transport region 150 may further increase electron injection and/or electron mobility and block holes between the cathode 110 and the light emitting layer 130.

Specifically, the electron transport region 150 may include an electron transport layer between the cathode 110 and the light emitting layer 130, and an electron transport auxiliary layer between the light emitting layer 130 and the electron transport layer, and at least one of the compounds of Group C may be included in at least one of the electron transport layer and the electron transport auxiliary layer.

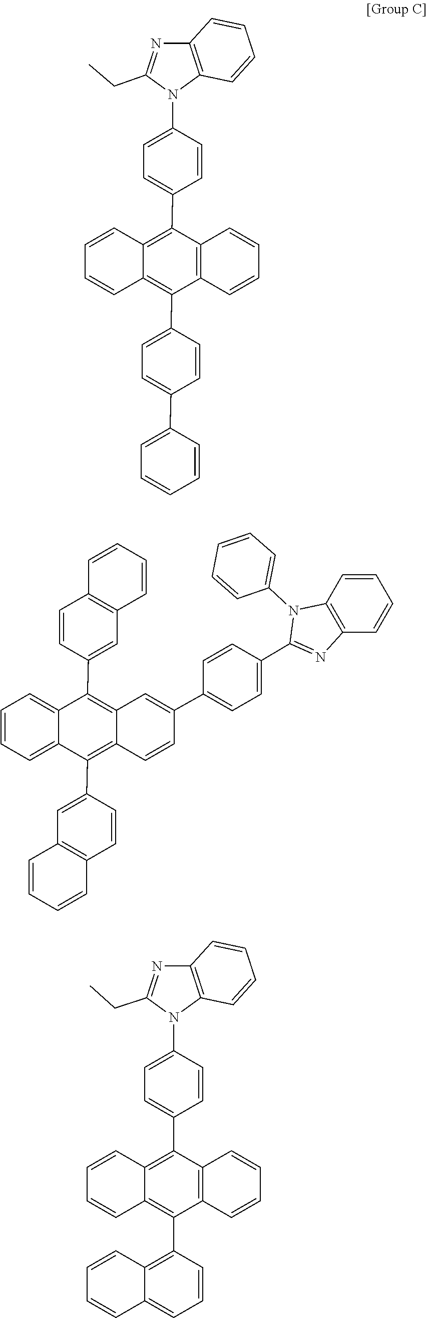 [SMILES: CCc1nc2ccccc2n1-c1ccc(-c2c3ccccc3c(-c3ccc(-c4ccccc4)cc3)c3ccccc23)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; CCc1nc2ccccc2n1-c1ccc(-c2c3ccccc3c(-c3cccc4ccccc34)c3ccccc23)cc1]

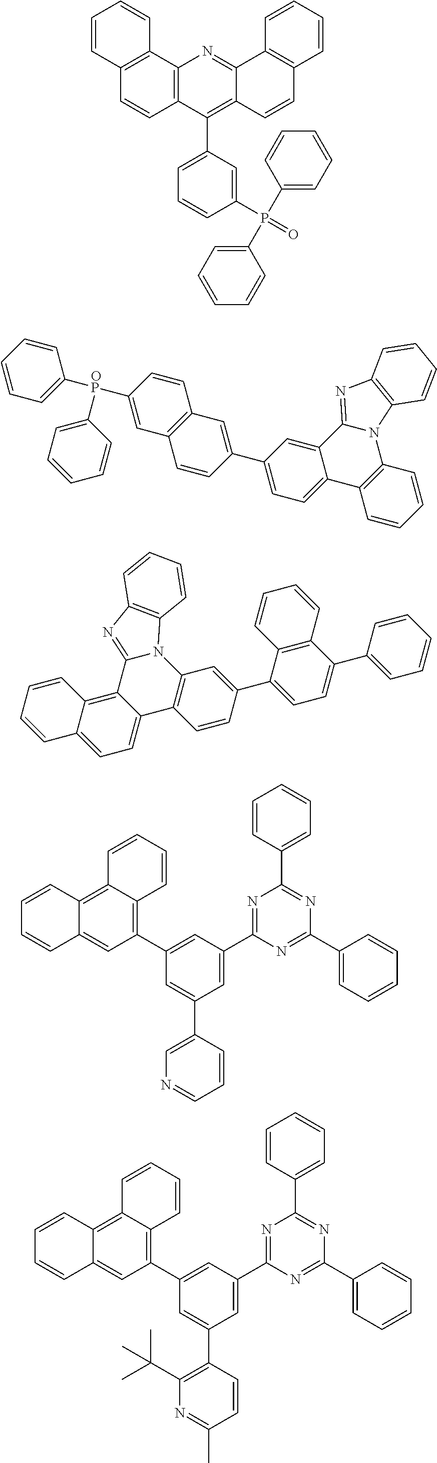

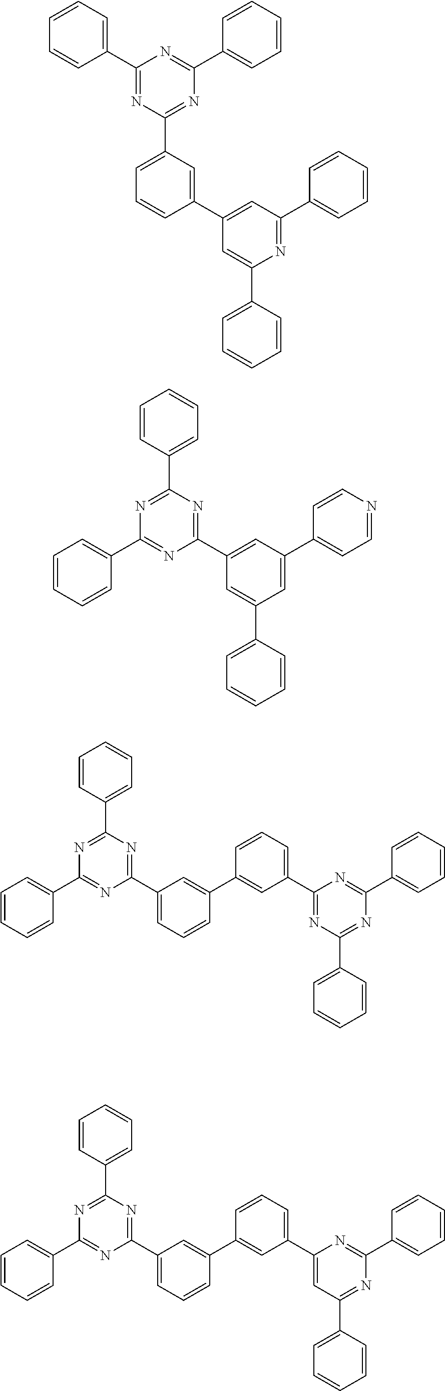 [SMILES: c1ccc(-c2cc(-c3cccc(-c4nc(-c5ccccc5)nc(-c5ccccc5)n4)c3)cc(-c3ccccc3)n2)cc1 ; c1ccc(-c2cc(-c3ccncc3)cc(-c3nc(-c4ccccc4)nc(-c4ccccc4)n3)c2)cc1 ; c1ccc(-c2nc(-c3ccccc3)nc(-c3cccc(-c4cccc(-c5nc(-c6ccccc6)nc(-c6ccccc6)n5)c4)c3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1]

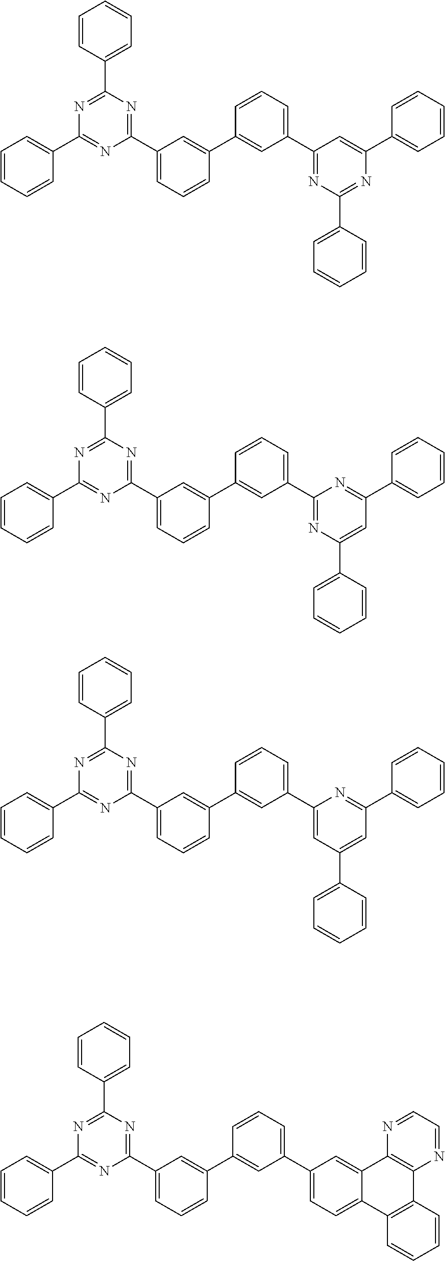 [SMILES: c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1]

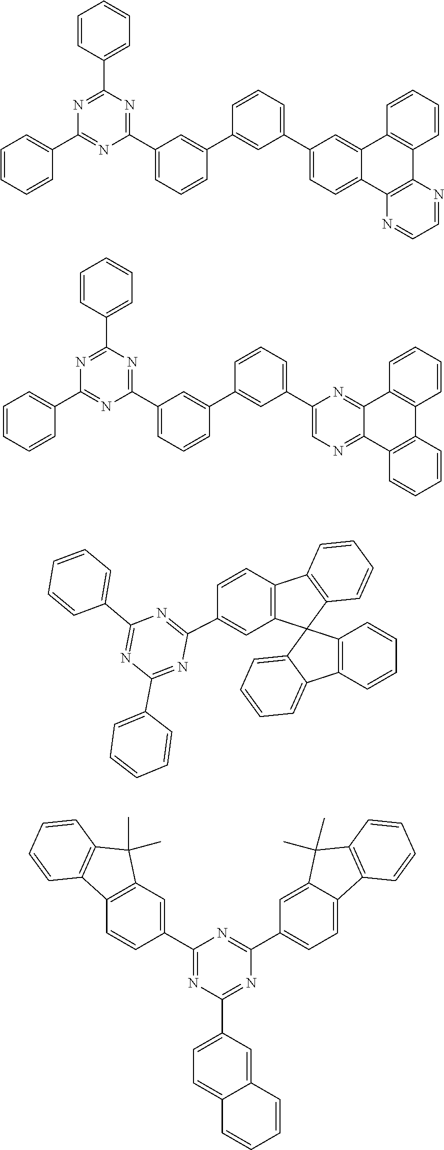

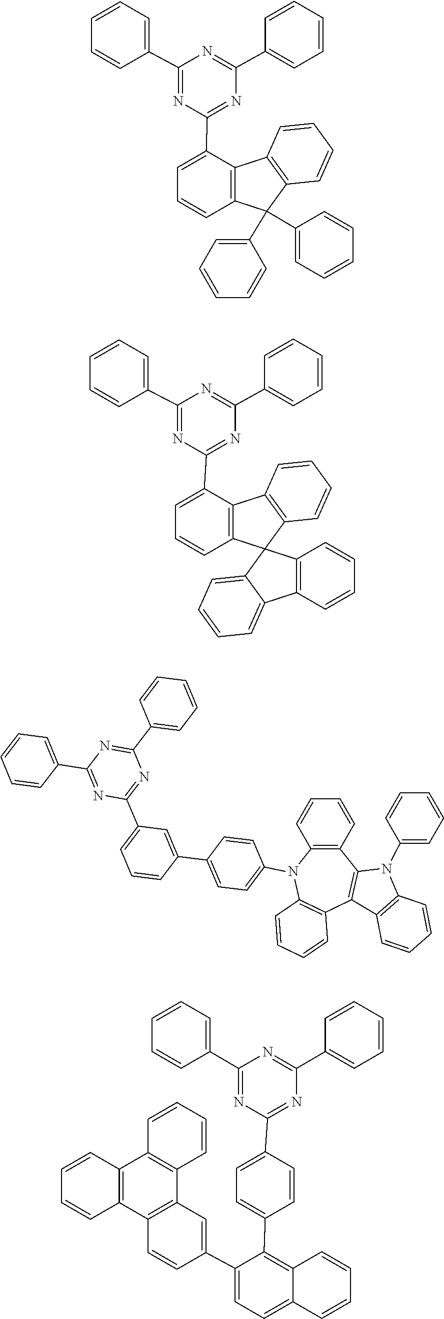 [SMILES: c1ccc(-c2nc(-c3ccccc3)nc(-c3cccc4c3-c3ccccc3C4(c3ccccc3)c3ccccc3)n2)cc1 ; c1ccc(-c2nc(-c3ccccc3)nc(-c3cccc4c3-c3ccccc3C43c4ccccc4-c4ccccc43)n2)cc1 ; c1ccc(-c2nc(-c3ccccc3)nc(-c3cccc(-c4ccc(N5c6ccccc6-c6c(n(-c7ccccc7)c7ccccc67)-c6ccccc65)cc4)c3)n2)cc1 ; c1ccc(-c2nc(-c3ccccc3)nc(-c3ccc(-c4c(-c5ccc6c7ccccc7c7ccccc7c6c5)ccc5ccccc45)cc3)n2)cc1]

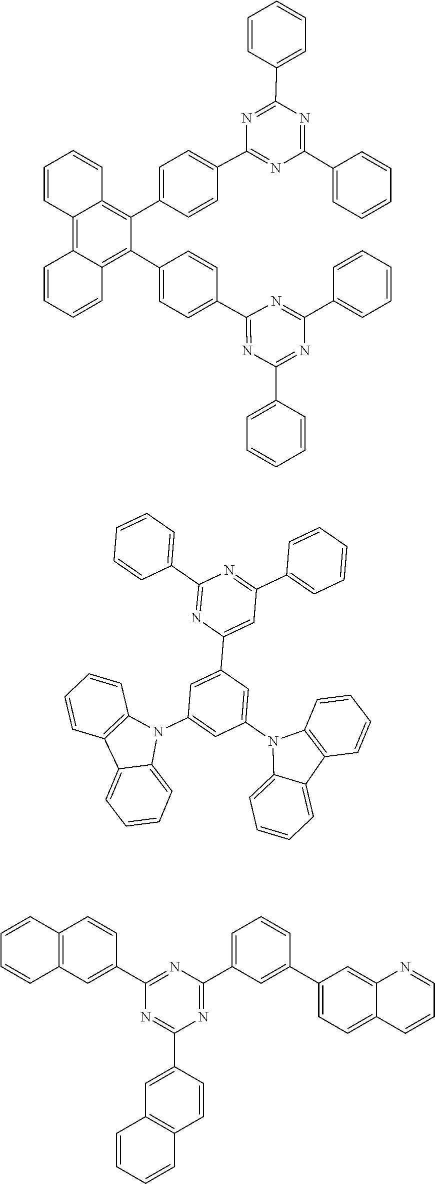 [SMILES: c1ccc(-c2nc(-c3ccccc3)nc(-c3ccc(-c4c(-c5ccc(-c6nc(-c7ccccc7)nc(-c7ccccc7)n6)cc5)c5ccccc5c5ccccc45)cc3)n2)cc1 ; c1ccc(-c2cc(-c3cc(-n4c5ccccc5c5ccccc54)cc(-n4c5ccccc5c5ccccc54)c3)nc(-c3ccccc3)n2)cc1 ; c1cc(-c2ccc3cccnc3c2)cc(-c2nc(-c3ccc4ccccc4c3)nc(-c3ccc4ccccc4c3)n2)c1]

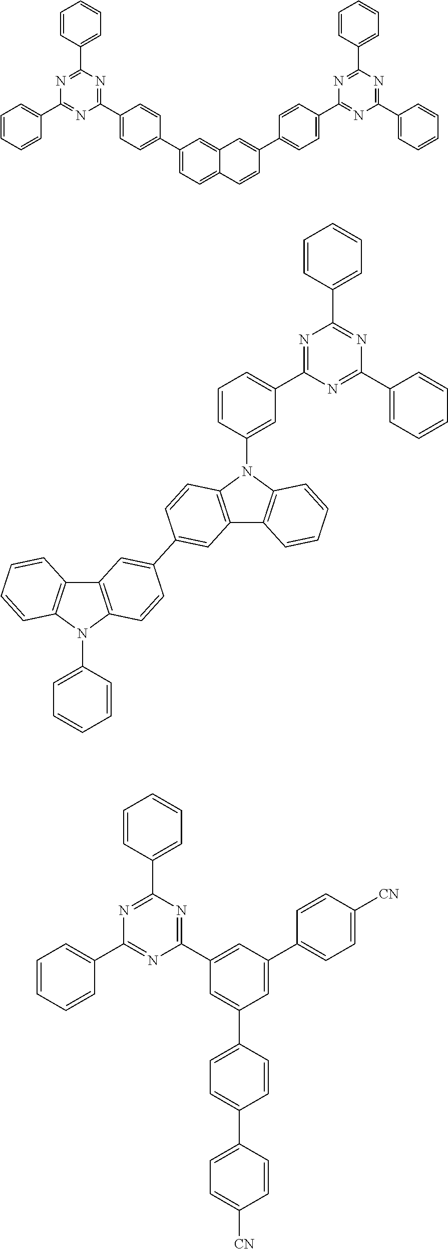 [SMILES: c1ccc(-c2nc(-c3ccccc3)nc(-c3ccc(-c4ccc5ccc(-c6ccc(-c7nc(-c8ccccc8)nc(-c8ccccc8)n7)cc6)cc5c4)cc3)n2)cc1 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; N#Cc1ccc(-c2ccc(-c3cc(-c4ccc(C#N)cc4)cc(-c4nc(-c5ccccc5)nc(-c5ccccc5)n4)c3)cc2)cc1]

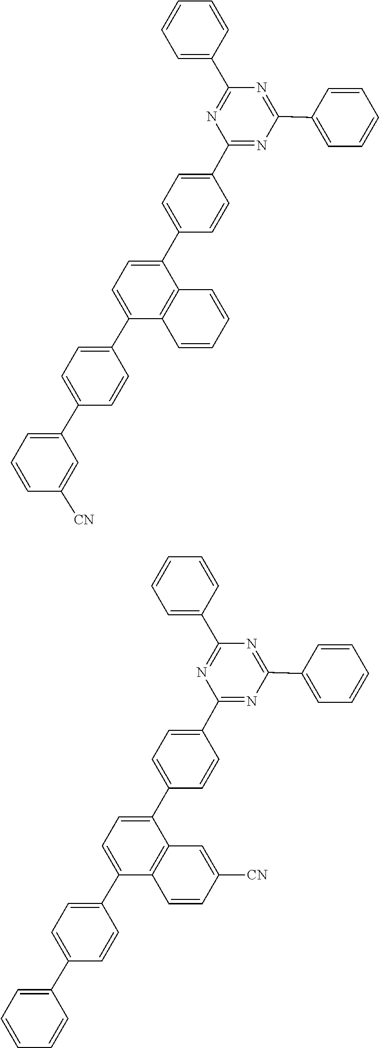 [SMILES: N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1]

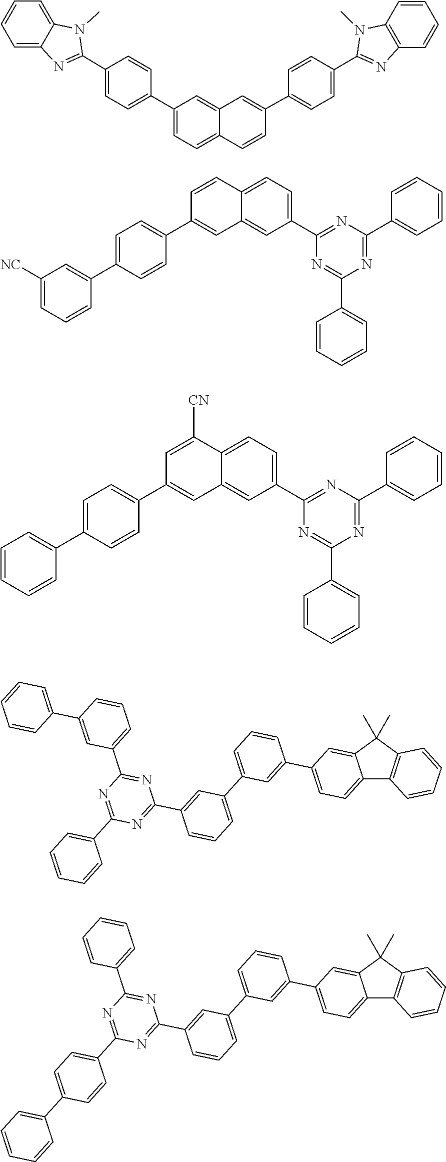

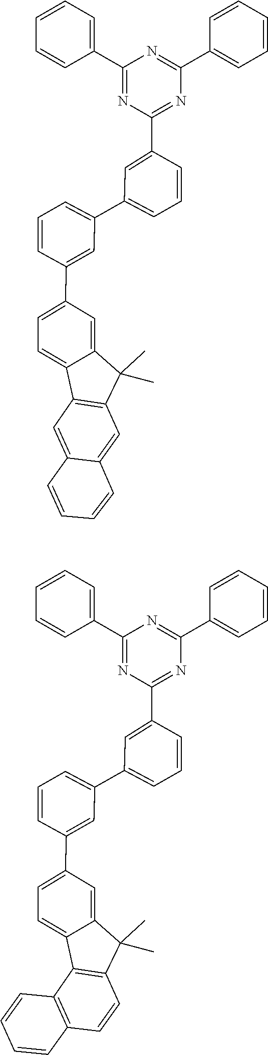 [SMILES: CC1(C)c2cc(-c3cccc(-c4cccc(-c5nc(-c6ccccc6)nc(-c6ccccc6)n5)c4)c3)ccc2-c2cc3ccccc3cc21 ; CC1(C)c2cc(-c3cccc(-c4cccc(-c5nc(-c6ccccc6)nc(-c6ccccc6)n5)c4)c3)ccc2-c2c1ccc1ccccc21]

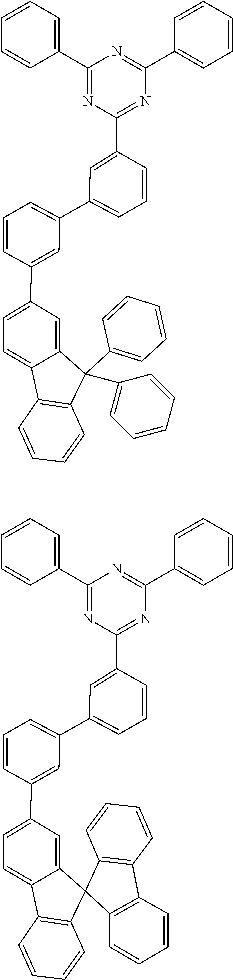 [SMILES: c1ccc(-c2nc(-c3ccccc3)nc(-c3cccc(-c4cccc(-c5ccc6c(c5)C(c5ccccc5)(c5ccccc5)c5ccccc5-6)c4)c3)n2)cc1 ; c1ccc(-c2nc(-c3ccccc3)nc(-c3cccc(-c4cccc(-c5ccc6c(c5)C5(c7ccccc7-c7ccccc75)c5ccccc5-6)c4)c3)n2)cc1]

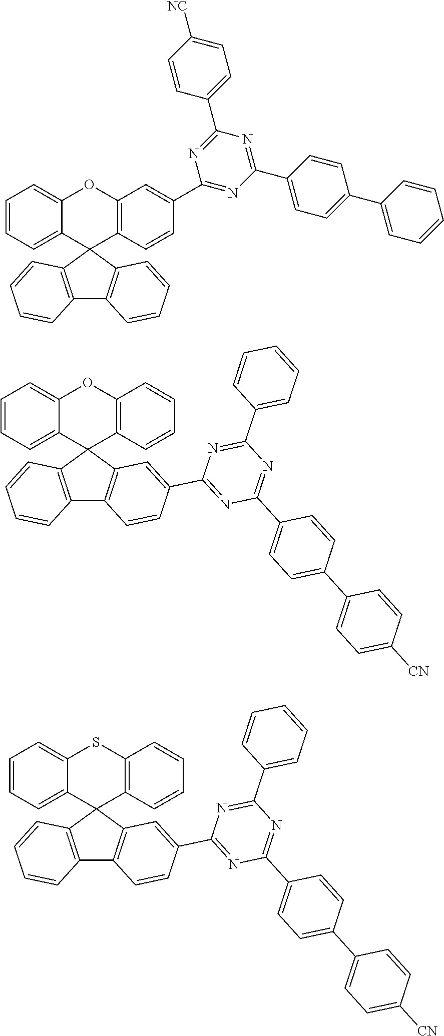 [SMILES: N#Cc1ccc(-c2nc(-c3ccc(-c4ccccc4)cc3)nc(-c3ccc4c(c3)Oc3ccccc3C43c4ccccc4-c4ccccc43)n2)cc1 ; N#Cc1ccc(-c2ccc(-c3nc(-c4ccccc4)nc(-c4ccc5c(c4)C4(c6ccccc6Oc6ccccc64)c4ccccc4-5)n3)cc2)cc1 ; N#Cc1ccc(-c2ccc(-c3nc(-c4ccccc4)nc(-c4ccc5c(c4)C4(c6ccccc6Sc6ccccc64)c4ccccc4-5)n3)cc2)cc1]

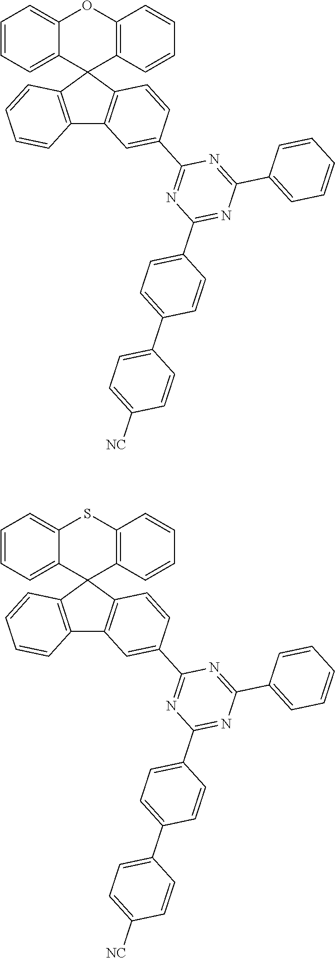 [SMILES: N#Cc1ccc(-c2ccc(-c3nc(-c4ccccc4)nc(-c4ccc5c(c4)-c4ccccc4C54c5ccccc5Oc5ccccc54)n3)cc2)cc1 ; N#Cc1ccc(-c2ccc(-c3nc(-c4ccccc4)nc(-c4ccc5c(c4)-c4ccccc4C54c5ccccc5Sc5ccccc54)n3)cc2)cc1]

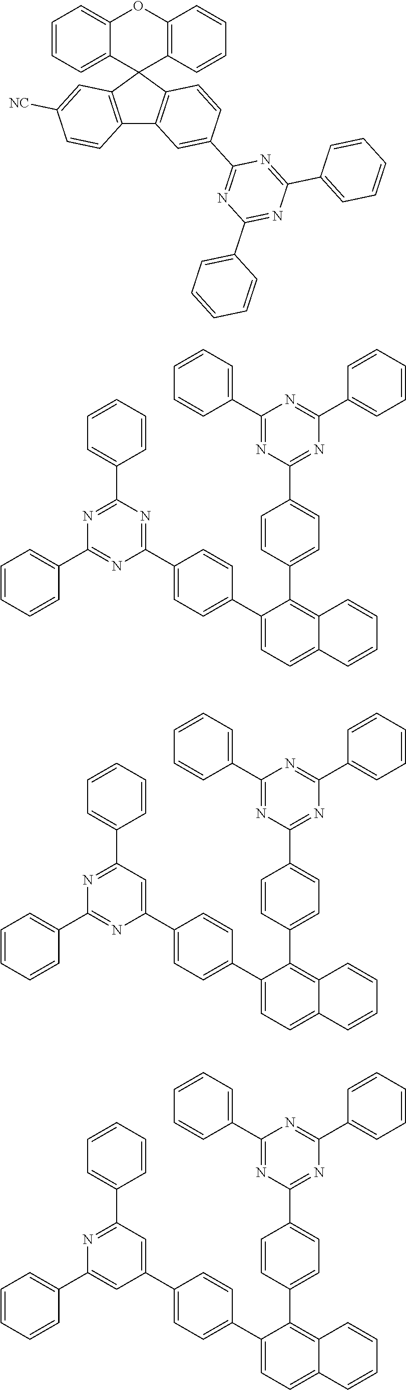

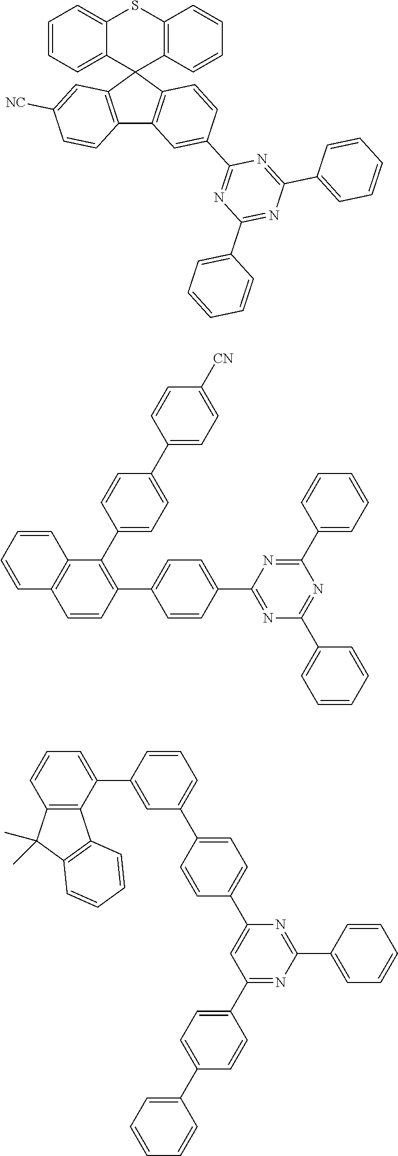

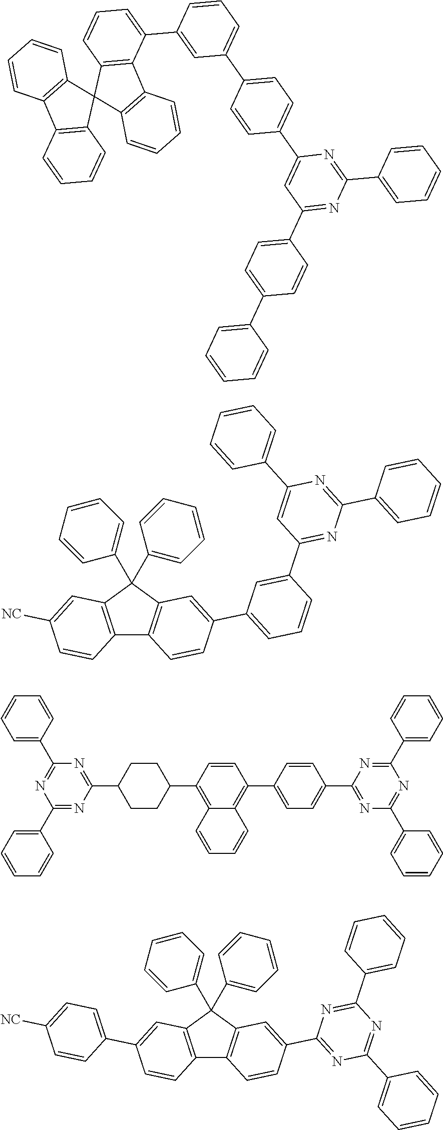

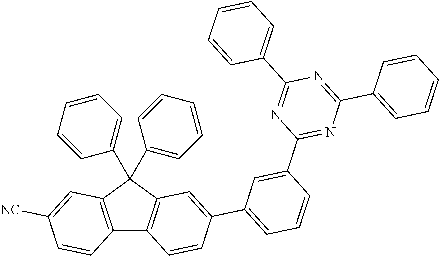 [SMILES: N#Cc1ccc2c(c1)C(c1ccccc1)(c1ccccc1)c1cc(-c3cccc(-c4nc(-c5ccccc5)nc(-c5ccccc5)n4)c3)ccc1-2]

An embodiment may be an organic light emitting diode including a light emitting layer as an organic layer.

Another embodiment may be an organic light emitting diode including a light emitting layer and a hole transport region as an organic layer.

Another embodiment may be an organic light emitting diode including a light emitting layer and an electron transport region as an organic layer.

As shown in FIG. 1, the organic light emitting diode according to the embodiment of the present invention may include a hole transport region 140 and an electron transport region 150 in addition to the light emitting layer 130 as the organic layer 105.

On the other hand, the organic light emitting diode may further include an electron injection layer (not shown), a hole injection layer (not shown), etc. in addition to the light emitting layer as the aforementioned organic layer.

The organic light emitting diode 100 may be produced by forming an anode or a cathode on a substrate, forming an organic layer using a dry film formation method such as a vacuum deposition method (evaporation), sputtering, plasma plating, and ion plating, and forming a cathode or an anode thereon.

The organic light emitting diode may be applied to an organic light emitting display device.

### MODE FOR INVENTION

Hereinafter, the embodiments are illustrated in more detail with reference to examples. However, these examples are exemplary, and the scope of claims is not limited thereto.

Hereinafter, starting materials and reactants used in examples and synthesis examples were purchased from Sigma-Aldrich Co. Ltd., TCI Inc., Tokyo chemical industry, or P&H tech as far as there is no particular comment or were synthesized by known methods.

### Synthesis of First Compound

### Synthesis Example 1: Synthesis of Compound A-4

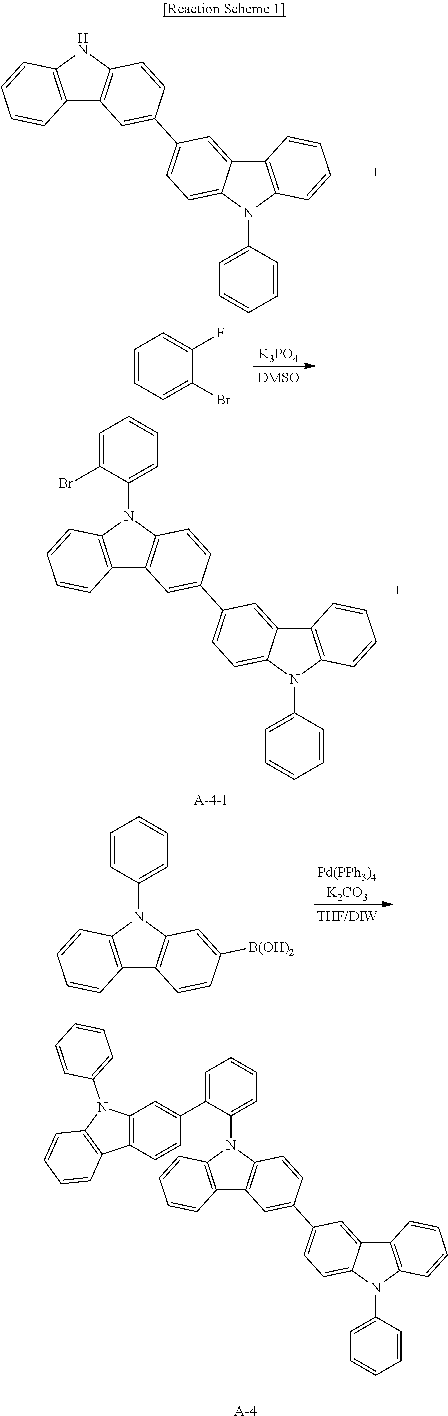

### 1st Step: Synthesis of Intermediate A-4-1

9-phenyl-9H,9′H-3,3′-bicarbazole (50 g, 122.4 mmol), 1-bromo-2-fluorobenzene (22.5 g, 128.5 mmol), and potassium phosphate (77.9 g, 367.2 mmol) were dissolved in 600 mL of dimethylsulfoxide and then, stirred under reflux at 180° C. for 12 hours. When a reaction was completed, purification through column chromatography (dichloromethane:n-hexane) was performed to obtain 46.9 g (a yield: 68.0%) of Intermediate A-4-1.

### 2^{nd} Step: Synthesis of Compound A-4

Intermediate A-1 (10.0 g, 17.7 mmol), (9-phenyl-9H-carbazol-2-yl) boronic acid (5.4 g, 18.6 mmol), tetrakis(triphenylphosphine)palladium (0) (1.0 g, 0.9 mmol), and potassium carbonate (7.4 g, 53.2 mmol) were dissolved in 90 mL of a mixed solvent of tetrahydrofuran: distilled water in a volume ratio of 2:1 and then, stirred under reflux at 80° C. for 12 hours. When a reaction was completed, purification through column chromatography (dichloromethane:n-hexane) was performed to obtain 9.7 g (a yield: 75.2%) of Compound A-4.

### Synthesis Examples 2 and 3

Reactant 1 and Reactant 2 as starting materials were used for the 2^{nd} step reaction (Suzuki rxn) of Synthesis Example 1 to synthesize each final compound.

TABLE 1

| Synthesis | | | | |
|---|---|---|---|---|
| Example | Reactant 1 | Reactant 2 | Product | Yield |
| Synthesis Example 2 | 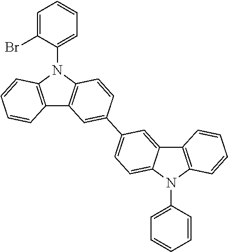 [SMILES: Brc1ccccc1-n1c2ccccc2c2cc(-c3ccc4c(c3)c3ccccc3n4-c3ccccc3)ccc21] | 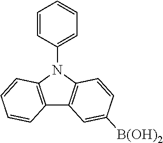 [SMILES: OB(O)c1ccc2c(c1)c1ccccc1n2-c1ccccc1] | 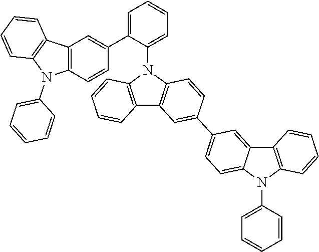 [SMILES: c1ccc(-n2c3ccccc3c3cc(-c4ccc5c(c4)c4ccccc4n5-c4ccccc4-c4ccc5c(c4)c4ccccc4n5-c4ccccc4)ccc32)cc1] | 80.1% |
| | A-4-1 | | A-5 | |
| Synthesis Example 3 | 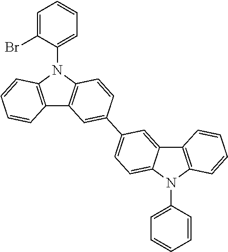 [SMILES: Brc1ccccc1-n1c2ccccc2c2cc(-c3ccc4c(c3)c3ccccc3n4-c3ccccc3)ccc21] | 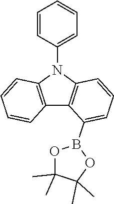 [SMILES: CC1(C)OB(c2cccc3c2c2ccccc2n3-c2ccccc2)OC1(C)C] | 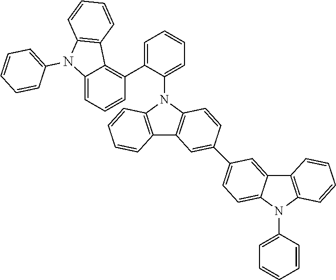 | 55.4% |
| | A-4-1 | | A-6 | |

### Comparative Synthesis Example 1: Synthesis of Compound C-1

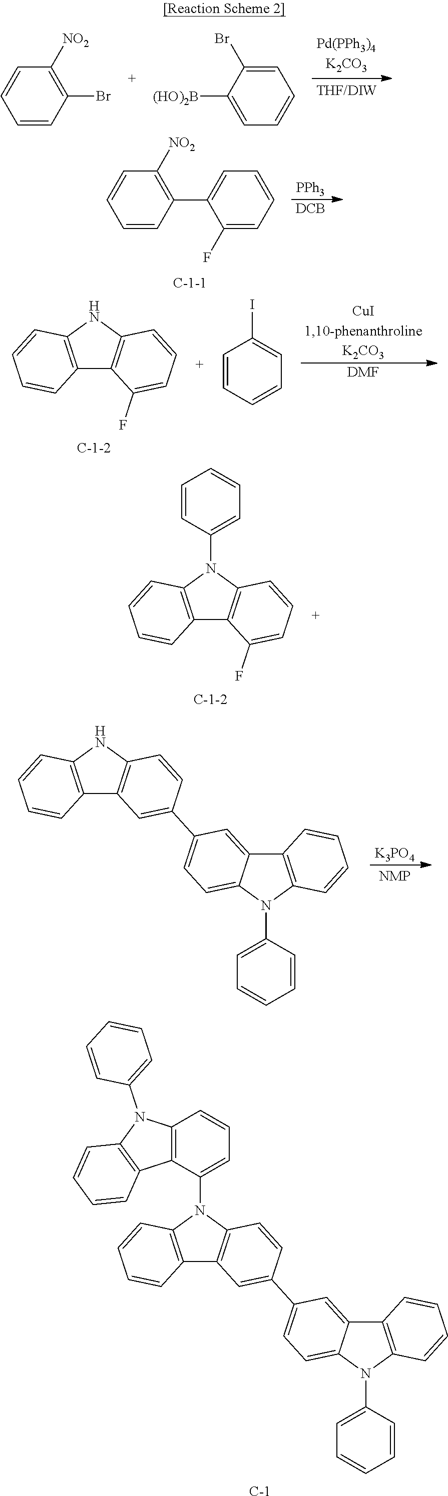

### 1 st Step: Synthesis of Intermediate C-1-1

45.2 g (a yield: 77.6%) of Intermediate C-1-1 was obtained in the same manner as in the 2^{nd} step of Synthesis Example 1 by using 1-bromo-2-nitrobenzene and 2-fluorophenylboronic acid as starting materials.

### 2^{nd} Step: Synthesis of Intermediate C-1-2

Intermediate C-1-1 (45.0 g, 207.2 mmol) and triphenylphosphine (163.0 g, 621.5 mmol) were added to 1 L of 1,2-dichlorobenzene and then, stirred under reflux for 12 hours under a nitrogen flow. When a reaction was completed, after removing the solvent, purification through column chromatography (dichloromethane:n-hexane) was performed to obtain 22.9 g (a yield: 59.8%) of Intermediate C-1-2.

### 3^{rd} Step: Synthesis of Intermediate C-1-3

Intermediate C-1-2 (22.9 g, 123.7 mmol), copper iodide (4.7 g, 24.7 mmol), potassium carbonate (25.6 g, 185.5 mmol), and 1,10-phenanthroline (2.7 g, 14.8 mmol) were added to 600 mL of N,N-dimethylformamide and then, stirred under reflux by heating for 12 hours under a nitrogen flow. When a reaction was completed, after removing the solvent, purification through column chromatography (dichloromethane:n-hexane) was performed to obtain 25.7 g (a yield: 79.5%) of Intermediate C-1-3.

### 4^{th} Step: Synthesis of Compound C-1

5.8 g (a yield: 63.7%) of Compound C-1 was obtained in the same manner as the synthesis method of Intermediate A-4-1 of Synthesis Example 1 except that Intermediate C-1-3 and 9-phenyl-9H,9′H-3,3′-bicarbazole as starting materials and N-Methyl-2-pyrrolidone as a solvent were used.

### Comparative Synthesis Example 2: Synthesis of Compound C-2

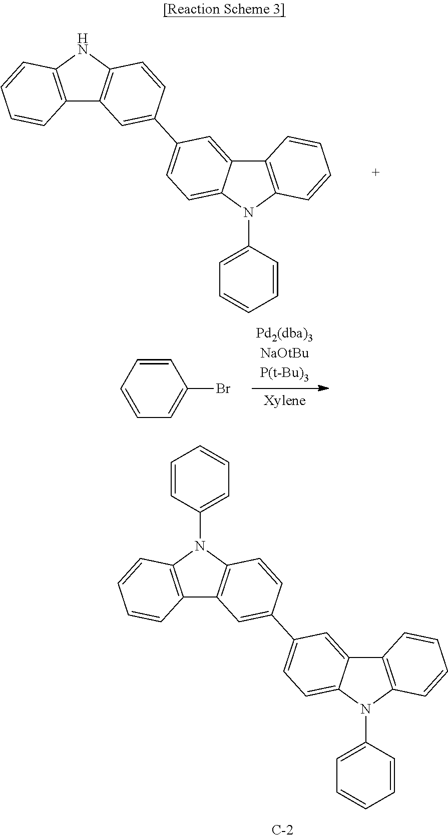

9-phenyl-9H,9′H-3,3′-bicarbazole (10.0 g, 24.5 mmol), bromobenzene (4.2 g, 26.9 mmol), sodium t-butoxide (3.5 g, 36.7 mmol), tris(dibenzylideneacetone)dipalladium (0) (1.1 g, 1.2 mmol), and a tri-tert-butylphosphine solution (0.7 g, 3.7 mmol) were added to 120 mL of xylene and then, stirred under reflux by heating for 12 hours under a nitrogen flow. After removing the solvent, purification through column chromatography (dichloromethane:n-hexane) was performed to obtain 9.0 g (a yield: 75.5%) of Compound C-2.

### Comparative Synthesis Example 3: Synthesis of Compound C-3

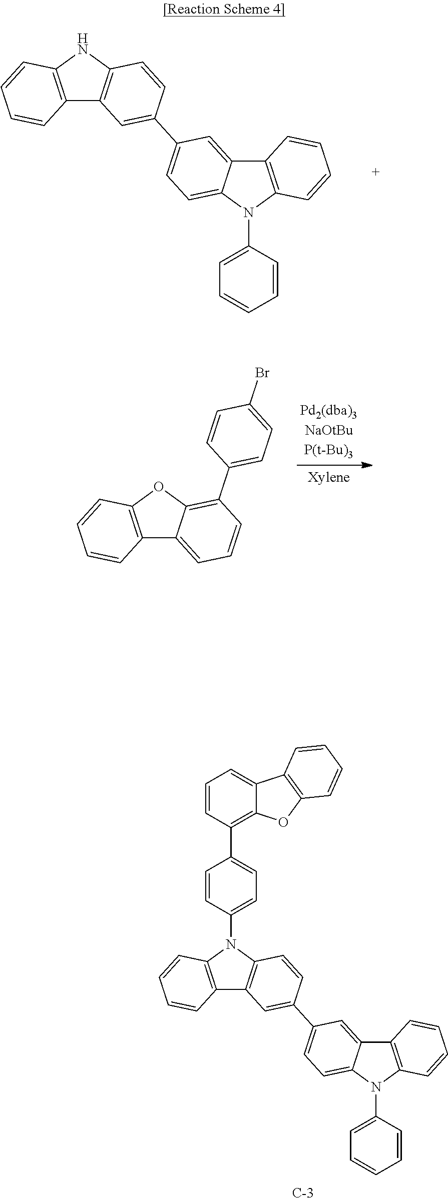

3.7 g (a yield: 52.3%) of Compound C-3 was obtained in the same manner as the synthesis method of Compound C-2 of Comparative Synthesis Example 2 except that 9-phenyl-9H,9′H-3,3′-bicarbazole and 4-(4-bromophenyl)dibenzo[b,d]furan as starting materials were used.

### Synthesis of Second Compound

### Synthesis Example 4: Synthesis of Compound B-42

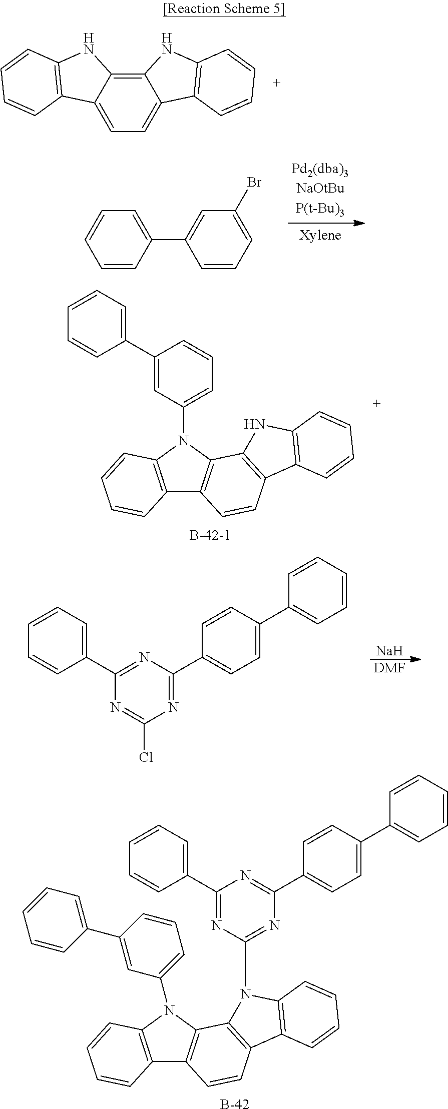

### 1^{st} Step: Synthesis of Intermediate B-42-1

22.5 g (a yield: 42.3%) of Intermediate B-42-1 was obtained in the same manner as the synthesis method of Compound C-2 of Comparative Synthesis Example 2 except that 11,12-dihydroindolo[2,3-a]carbazole and 3-bromobiphenyl as starting materials were used.

### 2^{nd} Step: Synthesis of Compound B-42

Intermediate B-42-1 (10 g, 24.5 mmol) was dissolved in 120 mL of N,N-dimethylformamide, and sodium hydride (0.9 g, 36.7 mmol) was slowly added thereto and then, stirred for 30 minutes. Subsequently, 2-([1,1′-biphenyl]-4-yl)-4-chloro-6-phenyl-1,3,5-triazine (8.8 g, 25.7 mmol) was slowly added thereto and then, stirred for 12 hours at room temperature. When a reaction was completed, purification by recrystallization with a mixed solution of dichloromethane:n-hexane was performed to obtain 12.3 g (a yield: 70.2%) of Compound B-42.

### Synthesis Example 5: Synthesis of Compound B-76

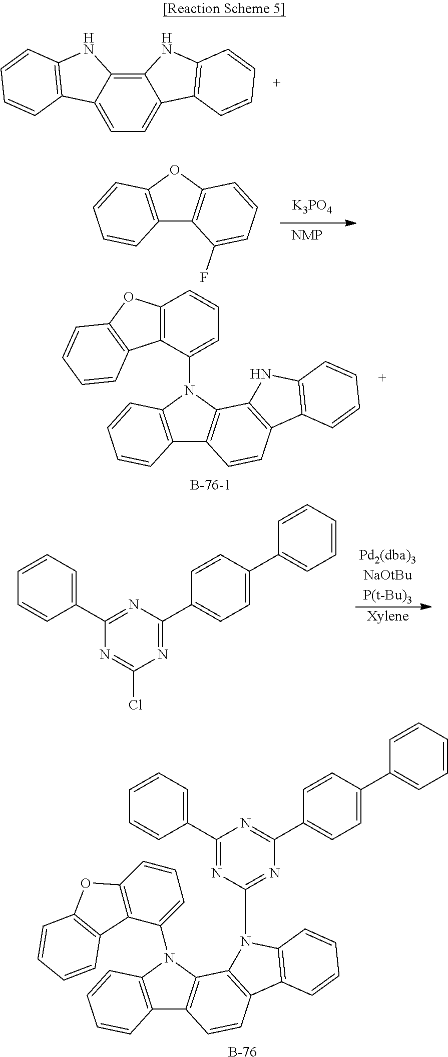

### 1^{st} Step: Synthesis of Intermediate B-76-1

16.4 g (a yield: 79.2%) of Intermediate B-76-1 was obtained in the same manner as the synthesis method of Compound C-1 of Comparative Synthesis Example 1 except that 11,12-dihydroindolo[2,3-a]carbazole and 1-fluorodibenzofuran as starting materials were used.

### 2^{nd} Step: Synthesis of Compound B-76

5.2 g (a yield: 45.1%) of Compound B-76 was obtained in the same manner as the synthesis method of Compound C-2 of Comparative Synthesis Example 2 except that Intermediate B-76-1 and 2-([1,1′-biphenyl]-4-yl)-4-chloro-6-phenyl-1,3,5-triazine as starting materials were used.

### (Manufacturing of Organic Light Emitting Diode)

### Example 1

A glass substrate coated with a thin film of ITO (indium tin oxide) was ultrasonically cleaned with distilled water. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. This obtained ITO transparent electrode was used as an anode, Compound A doped with 3% NDP-9 (available from Novaled) was vacuum-deposited on the ITO substrate to form a 100 Å-thick hole injection layer, and Compound A was deposited to on the hole injection layer to form a 1350 Å-thick hole transport layer. On the hole transport layer, 350 Å-thick hole transport auxiliary layer was formed by depositing Compound B. On the hole transport auxiliary layer, Compound A-4 obtained in Synthesis Example 1 and Compound B-42 obtained in Synthesis Example 4 were used simultaneously as hosts, and 10 wt % of PhGD was doped as a dopant to form a 330 Å-thick light emitting layer by vacuum deposition. Subsequently, Compound C was deposited on the light emitting layer to form a 50 Å-thick electron transport auxiliary layer, and Compound D and LiQ were simultaneously vacuum-deposited at a weight ratio of 1:1 to form a 300 Å-thick electron transport layer. LiQ (15 Å) and Al (1200 Å) were sequentially vacuum-deposited on the electron transport layer to form a cathode, thereby manufacturing an organic light emitting diode.

ITO/Compound A (3% NDP-9 doping, 100 Å)/Compound A (1350 Å)/Compound B (350 Å)/EML [Host (Compound A-4: Compound B-42=4:6 w/w): 10 wt % of PhGD] (330 Å)/Compound C (50 Å)/Compound D:LiQ (300 Å)/LiQ (15 Å)/Al (1200 Å).
Compound A: N-(biphenyl-4-yl)-9,9-dimethyl-N-(4-(9-phenyl-9H-carbazol-3-yl)phenyl)-9H-fluoren-2-amine
Compound B: N,N-bis(9,9-dimethyl-9H-fluoren-4-yl)-9,9-spirobi(fluorene)-2-amine Compound C: 2-[3′-(9,9-Dimethyl-9H-fluoren-2-yl) [1,1′-biphenyl]-3-yl]-4,6-diphenyl-1,3,5-triazine
Compound D: 2-[4-[4-(4′-Cyano-1,1′-biphenyl-4-yl)-1-naphthyl]phenyl]-4,6-diphenyl-1,3,5-triazine

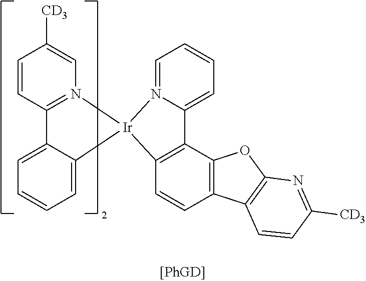

### Examples 2 to 6 and Comparative Examples 1 to 6

Diodes of Examples 2 to 6 and Comparative Examples 1 to 6 were manufactured in the same manner as in Example 1, except that the hosts were changed as described in Table 2.

### Example 7 and Comparative Example 7

Diodes of Example 7 and Comparative Example 7 were manufactured in the same manner as in Example 1, except that the hosts were changed as described in Table 2 and the mixing ratios of the first host and the second host were changed to 3:7.

### Example 8 and Comparative Example 8

Diodes of Example 8 and Comparative Example 8 were manufactured in the same manner as in Example 1, except that the hosts were changed as described in Table 2 and the mixing ratios of the first host and the second host were changed to 2:8.

### Evaluation

### (1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta Cs-1000A), while the voltage of the organic light emitting diodes was increased from 0 V to 10 V.

### (3) Measurement of Luminous Efficiency

Luminous efficiency (cd/A) at the same current density (10 mA/cm²) were calculated by using the luminance and current density from the items (1) and (2).

Relative values based on the luminous efficiency of Comparative Example 1 are shown in Table 2.

### (4) Measurement of Life-Span

T95 life-spans of the organic light emitting diodes were measured as a time when their luminance decreased down to 95% relative to the initial luminance (cd/m2) after emitting light with the initial luminance of 2,4000 cd/m² and measuring their luminance decrease depending on time with a Polanonix life-span measurement system.

Relative values based on the T95 life-span of Comparative Example 1 are shown in Table 2.

TABLE 2

| | First | Second | Luminous | Life-span |
|---|---|---|---|---|
| | host | host | efficiency (%) | T95 (%) |
| Example 1 | A-4 | B-42 | 104 | 190 |
| Example 2 | A-5 | B-42 | 104 | 220 |
| Example 3 | A-6 | B-42 | 106 | 150 |
| Example 4 | A-4 | B-76 | 106 | 180 |
| Example 5 | A-5 | B-76 | 106 | 220 |
| Example 6 | A-6 | B-76 | 107 | 145 |
| Example 7 | A-6 | B-42 | 105 | 170 |
| Example 8 | A-6 | B-42 | 102 | 160 |
| Comparative Example 1 | C-1 | B-42 | 100 | 100 |
| Comparative Example 2 | C-1 | B-76 | 101 | 90 |
| Comparative Example 3 | C-2 | B-42 | 102 | 130 |
| Comparative Example 4 | C-2 | B-76 | 102 | 120 |
| Comparative Example 5 | C-3 | B-42 | 102 | 80 |
| Comparative Example 6 | C-3 | B-76 | 102 | 70 |
| Comparative Example 7 | C-1 | B-42 | 98 | 110 |
| Comparative Example 8 | C-1 | B-42 | 96 | 90 |

Referring to Table 2, the organic light emitting diode to which the compounds according to Examples of the present invention are applied exhibited significantly improved efficiency, and life-span characteristics compared to the organic light emitting diodes according to Comparative Examples.

While this invention has been described in connection with what is presently considered to be practical embodiments, it is to be understood that the invention is not limited to the disclosed embodiments, but, on the contrary, is intended to cover various modifications and equivalent arrangements included within the spirit and scope of the appended claims.

1. A composition for an organic optoelectronic device, the composition comprising:
a first compound represented by Chemical Formula 1, and
a second compound represented by a combination of Chemical Formula 2 and Chemical Formula 3: 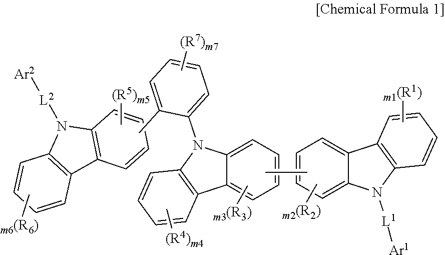
wherein, in Chemical Formula 1,
R¹ to R⁶ are each independently hydrogen, deuterium, a cyano group, a halogen, a substituted or unsubstituted C1 to C30 alkyl group, a substituted or unsubstituted C6 to C30 aryl group, a substituted or unsubstituted C2 to C30 heterocyclic group, or a combination thereof,
each R⁷ is independently hydrogen, deuterium, or a substituted or unsubstituted C6 to C12 aryl group,
L¹ and L² are each independently a single bond, a substituted or unsubstituted C6 to C20 arylene group, a substituted or unsubstituted C2 to C20 heterocyclic group, or a combination thereof,
Ar¹ and Ar² are each independently a substituted or unsubstituted C6 to C30 aryl group or a substituted or unsubstituted C2 to C30 heterocyclic group,
m¹, m⁴, m⁶, and m⁷ are each independently an integer of 1 to 4, and
m², m³, and m⁵ are each independently an integer of 1 to 3; 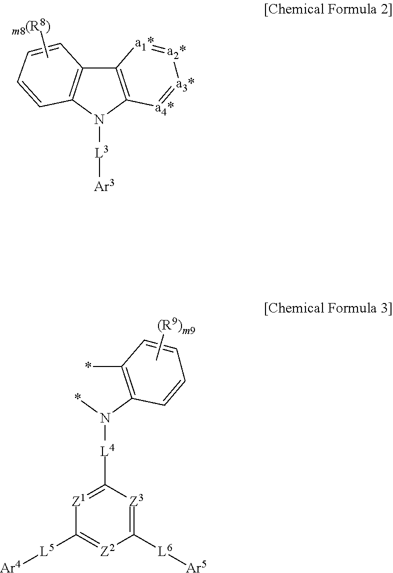
wherein, in Chemical Formula 2 and Chemical Formula 3,
Ar³ to Ar⁵ are each independently a substituted or unsubstituted C6 to C30 aryl group, or a substituted or unsubstituted C2 to C30 heterocyclic group,
two adjacent ones of a1* to a4* are linking carbons linked at * of Chemical Formula 3,
the remaining two of a1* to a4* of Chemical Formula 2, not linked at * of Chemical Formula 3, are each independently C-L^{a}-R^{a},
Z¹ to Z³ are each independently N or C-L^{b}-R^{b},
at least two of Z¹ to Z³ are N,
L^{a}, L^{b}, and L³ to L⁶ are each independently a single bond or a substituted or unsubstituted C6 to C20 arylene group,
R^{a}, R^{b}, R⁸ and R⁹ are each independently hydrogen, deuterium, a cyano group, a halogen, a substituted or unsubstituted amine group, a substituted or unsubstituted C1 to C30 alkyl group, a substituted or unsubstituted silyl group, a substituted or unsubstituted C6 to C30 aryl group, or a substituted or unsubstituted C2 to C30 heterocyclic group, and
m⁸ and m⁹ are each independently an integer of 1 to 4.

2. The composition for an organic optoelectronic device as claimed in claim 1, wherein:
the first compound is represented by one of Chemical Formula 1-1 to Chemical Formula 1-8: 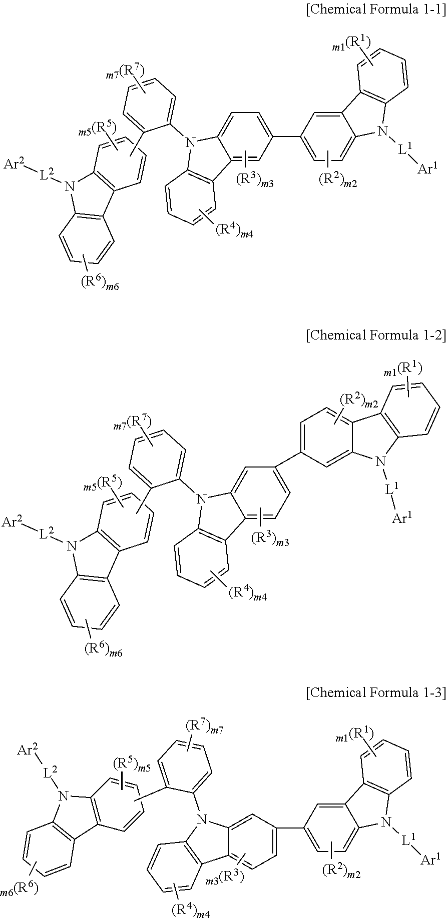 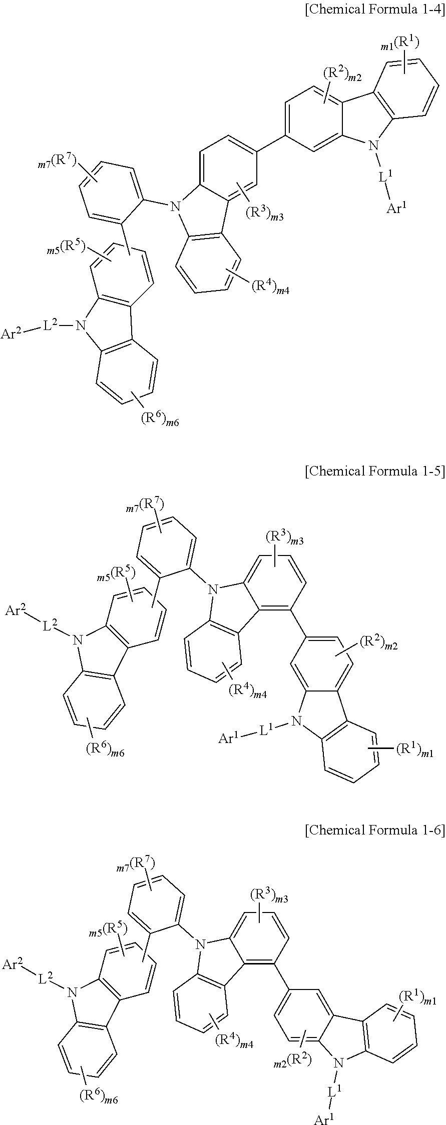 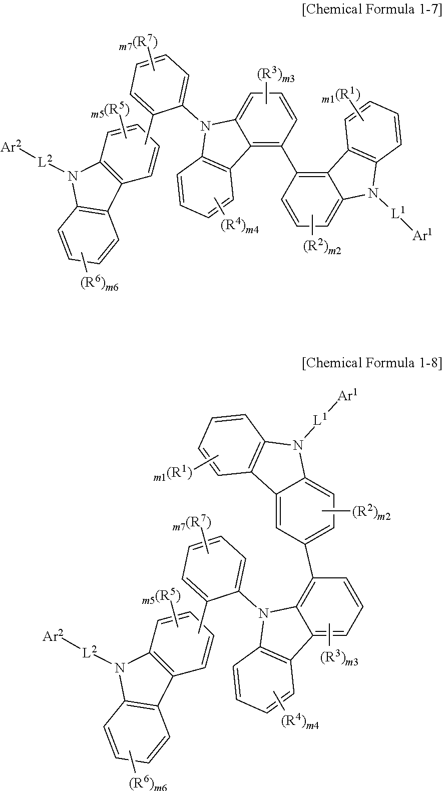
in Chemical Formula 1-1 to Chemical Formula 1-8, R¹ to R⁷, L¹ and L², Ar¹ and Ar², and m¹ to m⁷ are defined the same as those of Chemical Formula 1.

3. The composition for an organic optoelectronic device as claimed in claim 1, wherein in Chemical Formula 1, Ar¹ and Ar² are each independently a substituted or unsubstituted phenyl group, a substituted or unsubstituted biphenyl group, a substituted or unsubstituted terphenyl group, a substituted or unsubstituted fluorenyl group, a substituted or unsubstituted dibenzofuranyl group, a substituted or unsubstituted dibenzothiophenyl group, or a substituted or unsubstituted dibenzosilolyl group.

4. The composition for an organic optoelectronic device as claimed in claim 1, wherein:
moieties *-L¹-Ar¹ and *-L²-Ar² of Chemical Formula 1 are each independently a moiety of Group I: 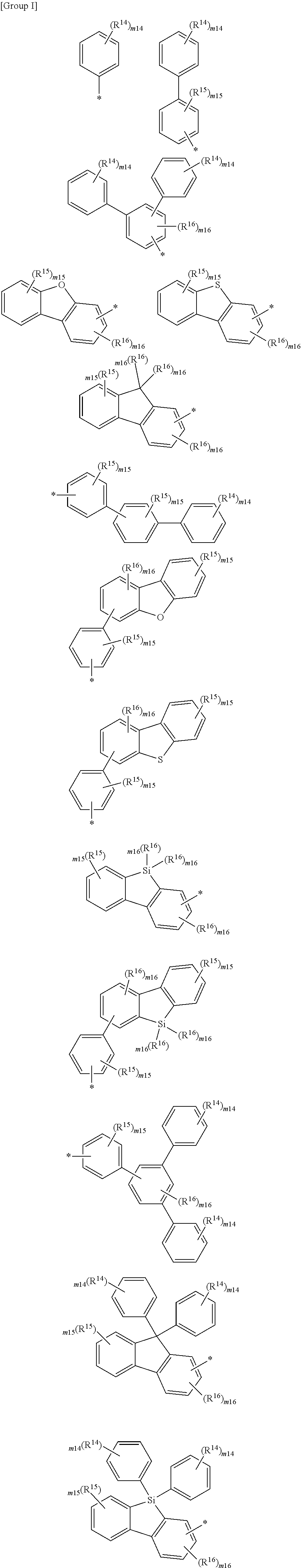
in Group I,
R¹⁴ to R¹⁶ are each independently hydrogen, deuterium, a substituted or unsubstituted C1 to C4 alkyl group, a substituted or unsubstituted C6 to C18 aryl group, or a substituted or unsubstituted C2 to C30 heteroaryl group,
m¹⁴ is an integer of 1 to 5,
m¹⁵ is an integer of 1 to 4,
m¹⁶ is an integer of 1 to 3, and
* is a linking point.

5. The composition for an organic optoelectronic device as claimed in claim 1, wherein the first compound is a compound of Group 1:

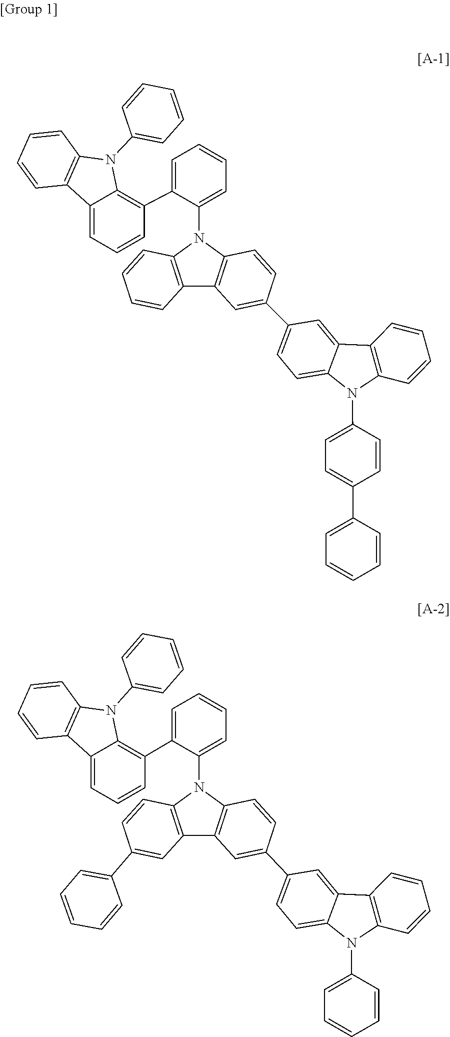 [SMILES: c1ccc(-c2ccc(-n3c4ccccc4c4cc(-c5ccc6c(c5)c5ccccc5n6-c5ccccc5-c5cccc6c7ccccc7n(-c7ccccc7)c56)ccc43)cc2)cc1 ; c1ccc(-c2ccc3c(c2)c2cc(-c4ccc5c(c4)c4ccccc4n5-c4ccccc4)ccc2n3-c2ccccc2-c2cccc3c4ccccc4n(-c4ccccc4)c23)cc1]

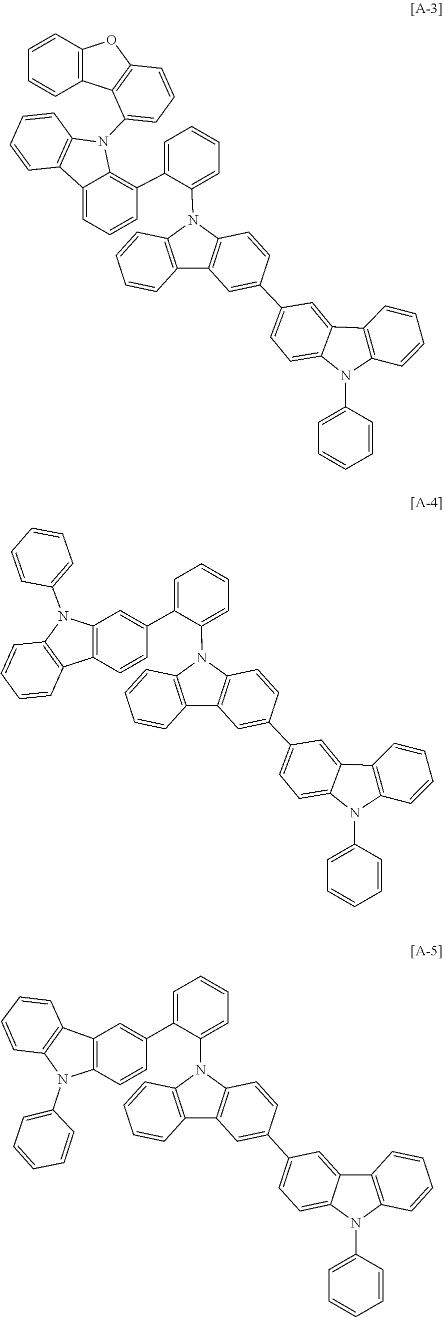 [SMILES: c1ccc(-n2c3ccccc3c3cc(-c4ccc5c(c4)c4ccccc4n5-c4ccccc4-c4cccc5c6ccccc6n(-c6cccc7oc8ccccc8c67)c45)ccc32)cc1 ; c1ccc(-n2c3ccccc3c3cc(-c4ccc5c(c4)c4ccccc4n5-c4ccccc4-c4ccc5c6ccccc6n(-c6ccccc6)c5c4)ccc32)cc1 ; c1ccc(-n2c3ccccc3c3cc(-c4ccc5c(c4)c4ccccc4n5-c4ccccc4-c4ccc5c(c4)c4ccccc4n5-c4ccccc4)ccc32)cc1]

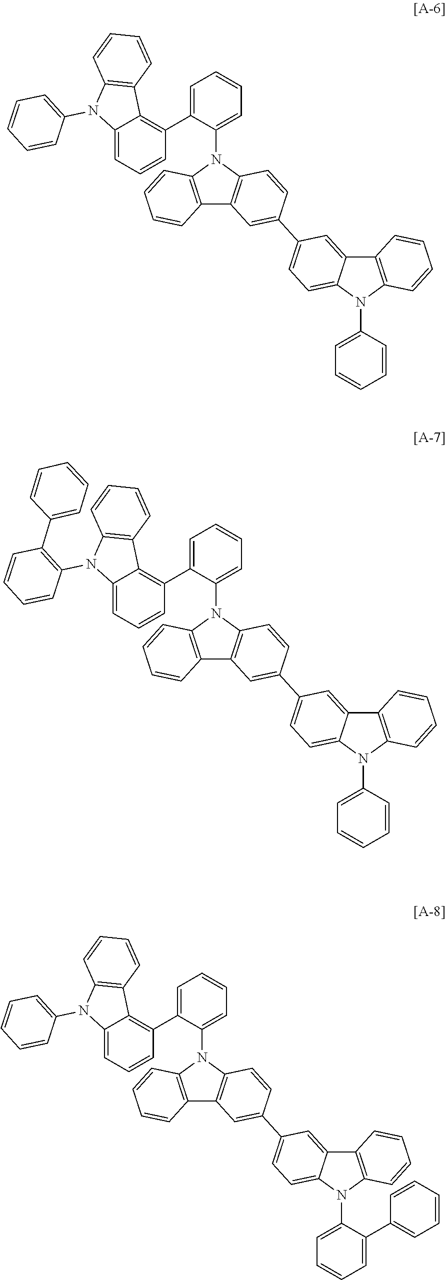 [SMILES: c1ccc(-n2c3ccccc3c3cc(-c4ccc5c(c4)c4ccccc4n5-c4ccccc4-c4cccc5c4c4ccccc4n5-c4ccccc4)ccc32)cc1 ; c1ccc(-c2ccccc2-n2c3ccccc3c3c(-c4ccccc4-n4c5ccccc5c5cc(-c6ccc7c(c6)c6ccccc6n7-c6ccccc6)ccc54)cccc32)cc1 ; c1ccc(-c2ccccc2-n2c3ccccc3c3cc(-c4ccc5c(c4)c4ccccc4n5-c4ccccc4-c4cccc5c4c4ccccc4n5-c4ccccc4)ccc32)cc1]

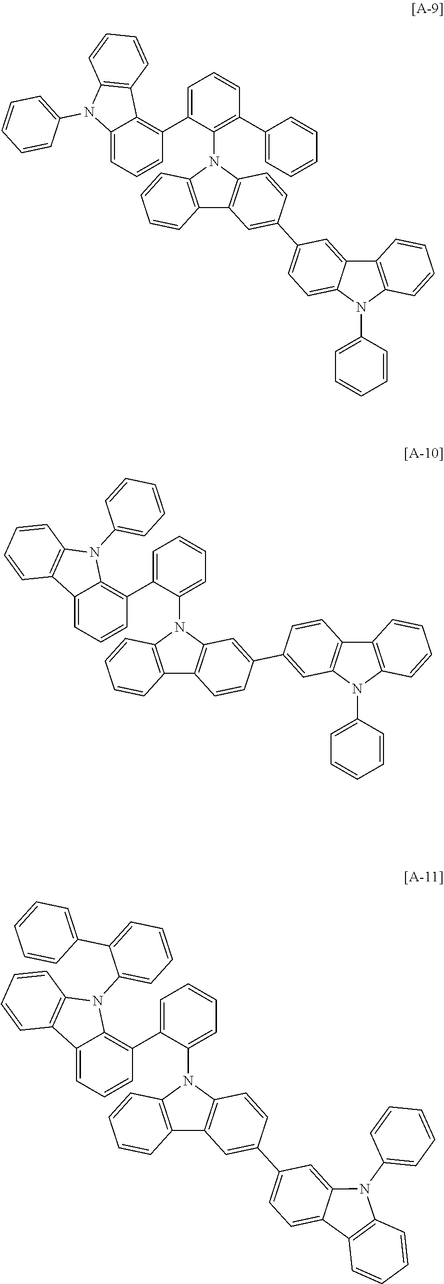

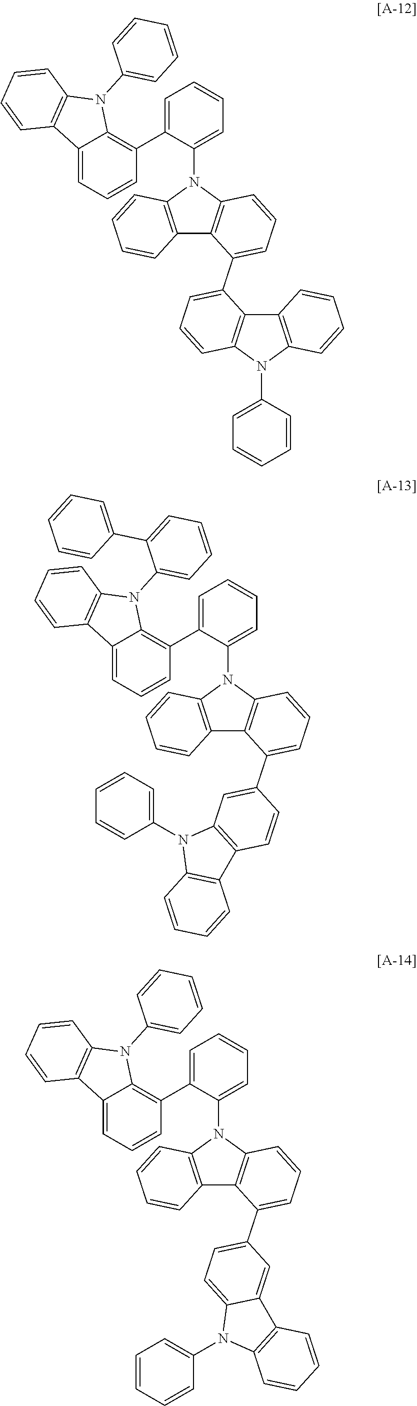

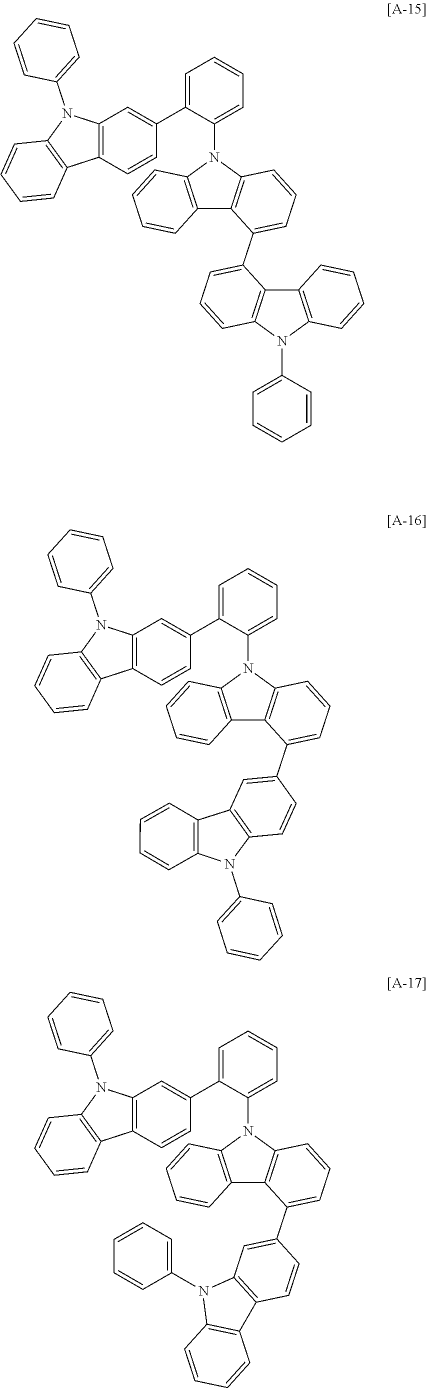 [SMILES: c1ccc(-n2c3ccccc3c3ccc(-c4ccccc4-n4c5ccccc5c5c(-c6cccc7c6c6ccccc6n7-c6ccccc6)cccc54)cc32)cc1 ; c1ccc(-n2c3ccccc3c3cc(-c4cccc5c4c4ccccc4n5-c4ccccc4-c4ccc5c6ccccc6n(-c6ccccc6)c5c4)ccc32)cc1 ; c1ccc(-n2c3ccccc3c3ccc(-c4ccccc4-n4c5ccccc5c5c(-c6ccc7c8ccccc8n(-c8ccccc8)c7c6)cccc54)cc32)cc1]

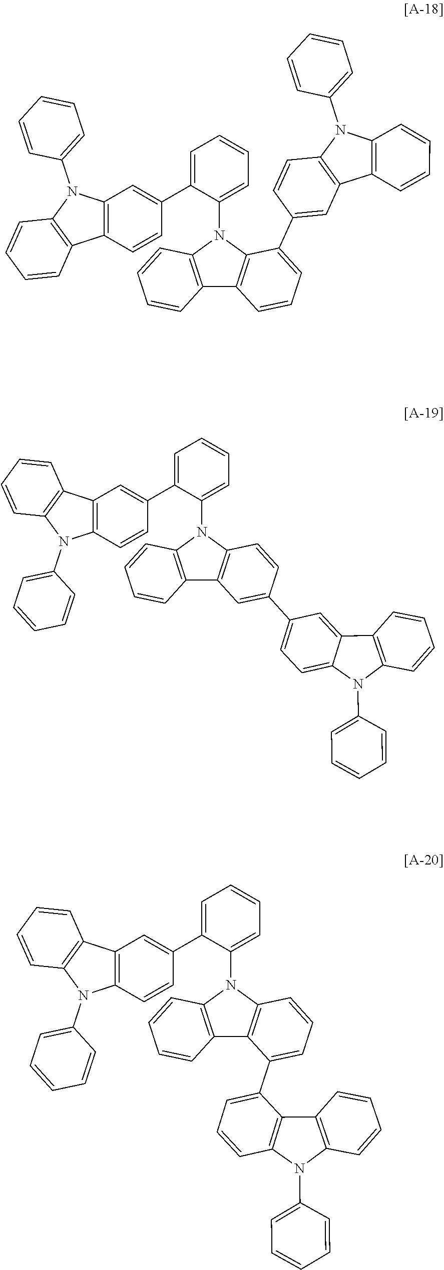 [SMILES: c1ccc(-n2c3ccccc3c3cc(-c4cccc5c6ccccc6n(-c6ccccc6-c6ccc7c8ccccc8n(-c8ccccc8)c7c6)c45)ccc32)cc1 ; c1ccc(-n2c3ccccc3c3cc(-c4ccc5c(c4)c4ccccc4n5-c4ccccc4-c4ccc5c(c4)c4ccccc4n5-c4ccccc4)ccc32)cc1 ; c1ccc(-n2c3ccccc3c3cc(-c4ccccc4-n4c5ccccc5c5c(-c6cccc7c6c6ccccc6n7-c6ccccc6)cccc54)ccc32)cc1]

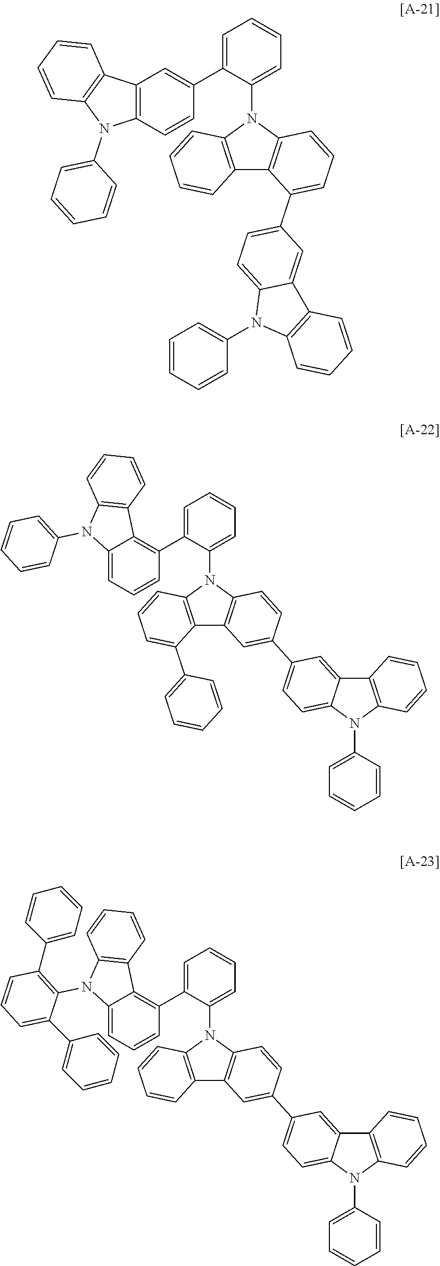 [SMILES: c1ccc(-n2c3ccccc3c3cc(-c4ccccc4-n4c5ccccc5c5c(-c6ccc7c(c6)c6ccccc6n7-c6ccccc6)cccc54)ccc32)cc1 ; c1ccc(-c2cccc3c2c2cc(-c4ccc5c(c4)c4ccccc4n5-c4ccccc4)ccc2n3-c2ccccc2-c2cccc3c2c2ccccc2n3-c2ccccc2)cc1 ; c1ccc(-c2cccc(-c3ccccc3)c2-n2c3ccccc3c3c(-c4ccccc4-n4c5ccccc5c5cc(-c6ccc7c(c6)c6ccccc6n7-c6ccccc6)ccc54)cccc32)cc1]

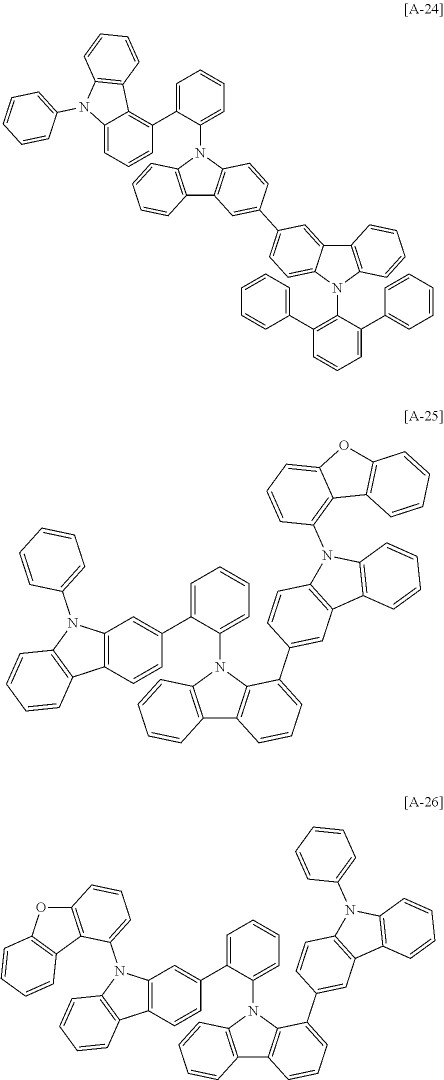 [SMILES: c1ccc(-c2cccc(-c3ccccc3)c2-n2c3ccccc3c3cc(-c4ccc5c(c4)c4ccccc4n5-c4ccccc4-c4cccc5c4c4ccccc4n5-c4ccccc4)ccc32)cc1 ; c1ccc(-n2c3ccccc3c3ccc(-c4ccccc4-n4c5ccccc5c5cccc(-c6ccc7c(c6)c6ccccc6n7-c6cccc7oc8ccccc8c67)c54)cc32)cc1 ; c1ccc(-n2c3ccccc3c3cc(-c4cccc5c6ccccc6n(-c6ccccc6-c6ccc7c8ccccc8n(-c8cccc9oc%10ccccc%10c89)c7c6)c45)ccc32)cc1]

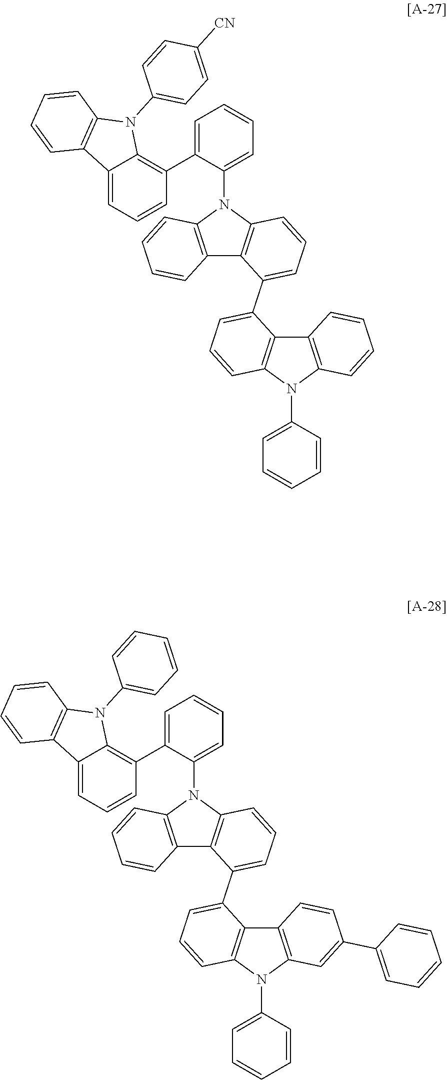 [SMILES: N#Cc1ccc(-n2c3ccccc3c3cccc(-c4ccccc4-n4c5ccccc5c5c(-c6cccc7c6c6ccccc6n7-c6ccccc6)cccc54)c32)cc1 ; c1ccc(-c2ccc3c4c(-c5cccc6c5c5ccccc5n6-c5ccccc5-c5cccc6c7ccccc7n(-c7ccccc7)c56)cccc4n(-c4ccccc4)c3c2)cc1]

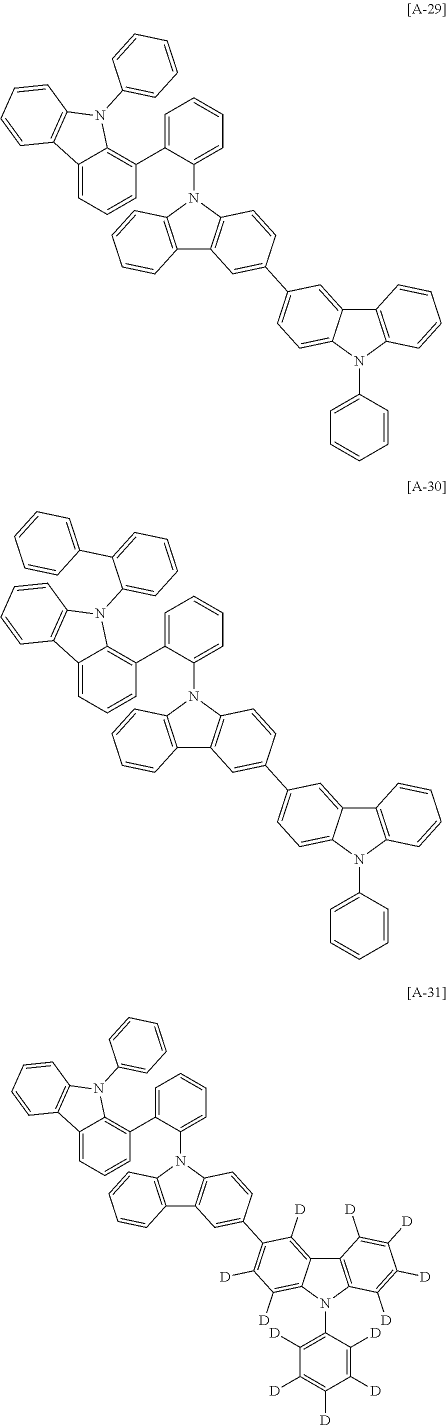 [SMILES: c1ccc(-n2c3ccccc3c3cc(-c4ccc5c(c4)c4ccccc4n5-c4ccccc4-c4cccc5c6ccccc6n(-c6ccccc6)c45)ccc32)cc1 ; c1ccc(-c2ccccc2-n2c3ccccc3c3cccc(-c4ccccc4-n4c5ccccc5c5cc(-c6ccc7c(c6)c6ccccc6n7-c6ccccc6)ccc54)c32)cc1 ; [2H]c1c([2H])c([2H])c(-n2c3c([2H])c([2H])c([2H])c([2H])c3c3c([2H])c(-c4ccc5c(c4)c4ccccc4n5-c4ccccc4-c4cccc5c6ccccc6n(-c6ccccc6)c45)c([2H])c([2H])c32)c([2H])c1[2H]]

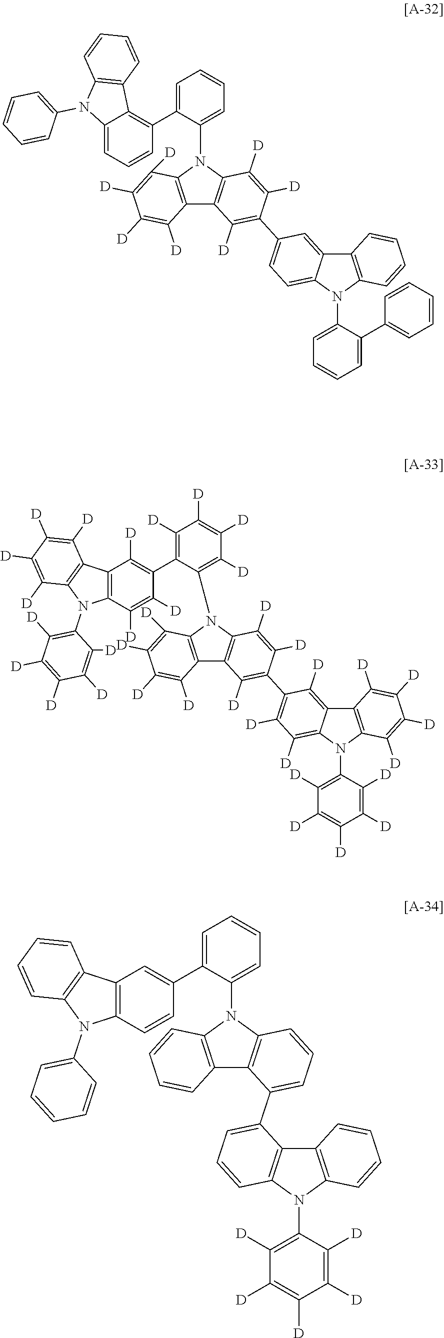

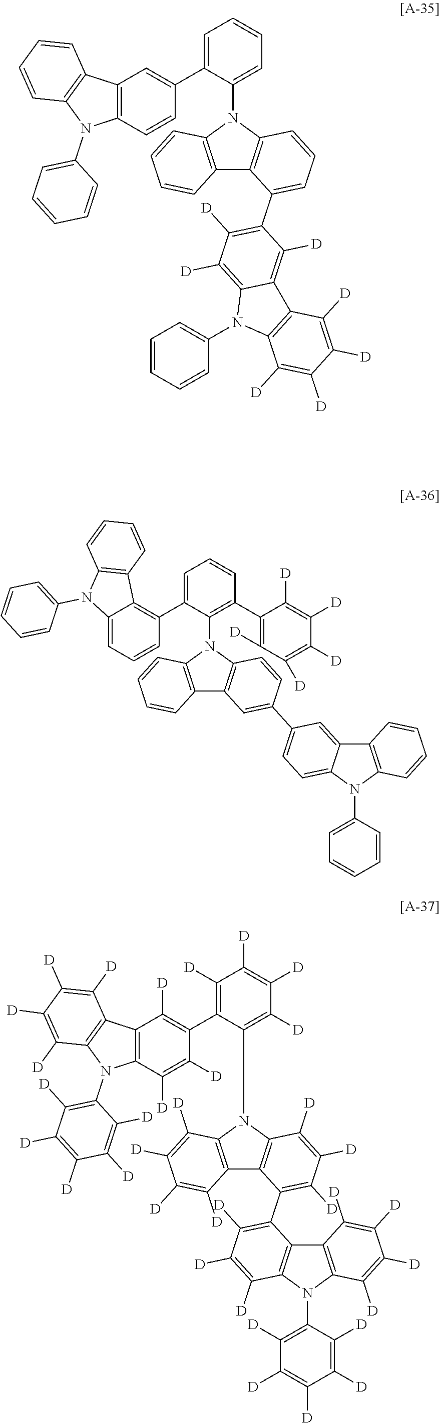

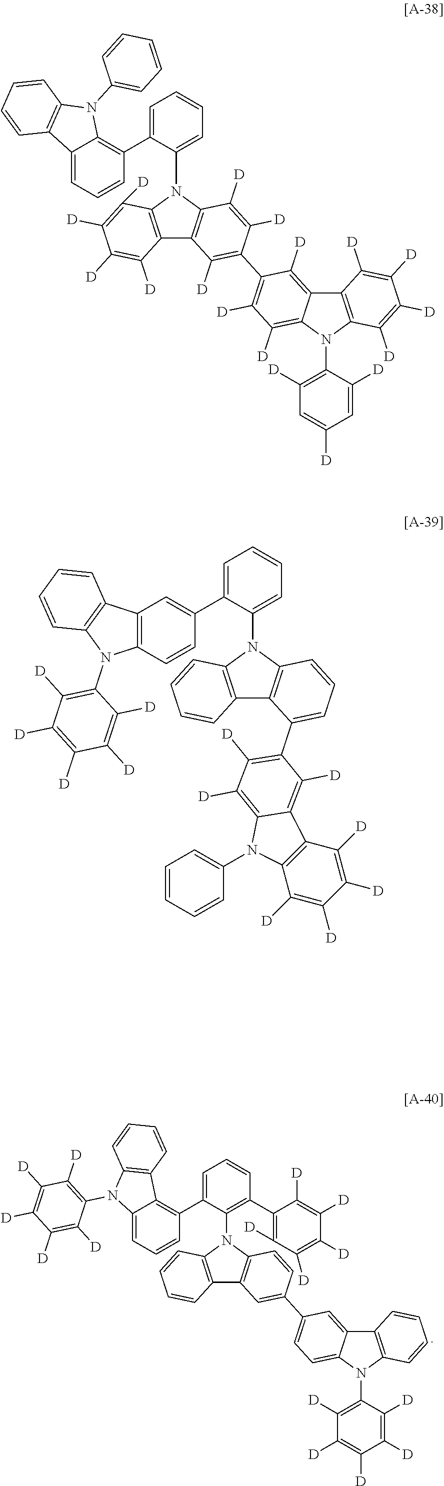

6. The composition for an organic optoelectronic device as claimed in claim 1, wherein;
the second compound is represented by one of Chemical Formula 2A to Chemical Formula 2F: 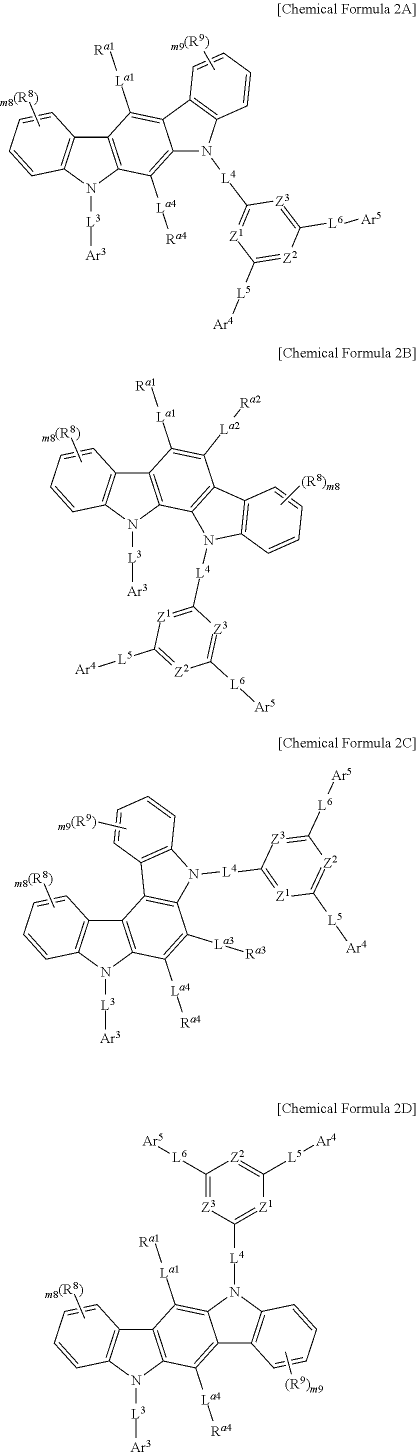 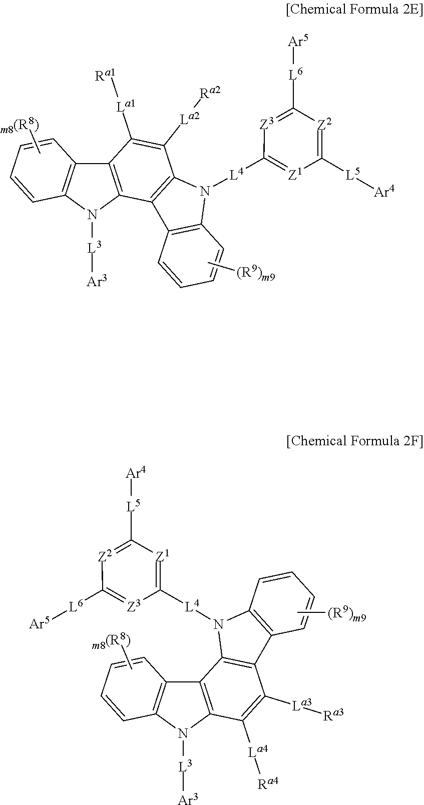
in Chemical Formula 2A to Chemical Formula 2F,
Z¹ to Z³, R⁸, R⁹, L³ to L⁶, Ar³ to Ar⁵, m⁸, and m⁹ are defined the same as those of Chemical Formula 2 and Chemical Formula 3,
R^{a1} to R^{a4} are defined the same as R⁸ and R⁹, and
L^{a1} to L^{a4} are defined the same as L³ to L⁶.

7. The composition for an organic optoelectronic device as claimed in claim 1, wherein:
Ar³ is a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, a substituted or unsubstituted dibenzofuranyl group, a substituted or unsubstituted dibenzothiophenyl group, a substituted or unsubstituted carbazolyl group, or a substituted or unsubstituted dibenzosilolyl group, and
Ar⁴ and Ar⁵ are each independently a substituted or unsubstituted phenyl group, a substituted or unsubstituted biphenyl group, a substituted or unsubstituted terphenyl group, a substituted or unsubstituted fluorenyl group, a substituted or unsubstituted dibenzofuranyl group, a substituted or unsubstituted dibenzothiophenyl group, a substituted or unsubstituted carbazolyl group, or a substituted or unsubstituted dibenzosilolyl group.

8. The composition for an organic optoelectronic device as claimed in claim 1, wherein:
moieties *-L⁵-Ar⁴ and *-L⁶-Ar⁵ are each independently a moiety of Group II: 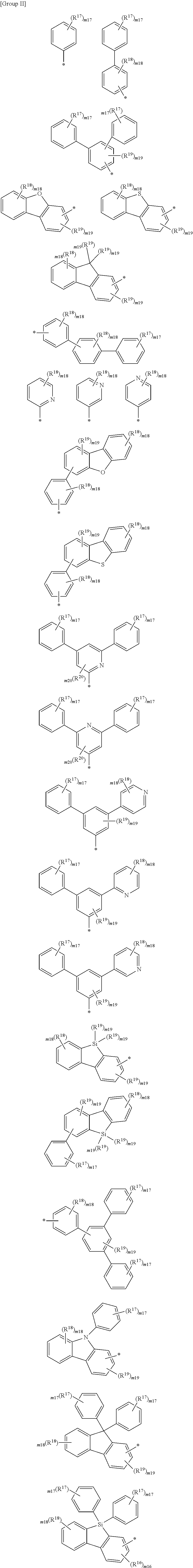
in Group II,
R¹⁷ to R²⁰ are each independently hydrogen, deuterium, a substituted or unsubstituted C1 to C4 alkyl group, a substituted or unsubstituted C6 to C18 aryl group, or a substituted or unsubstituted C2 to C30 heteroaryl group,
m¹⁷ is an integer of 1 to 5,
m¹⁸ is an integer of 1 to 4,
m¹⁹ is an integer of 1 to 3,
m²⁰ is an integer of 1 or 2, and
* is a linking point.

9. The composition for an organic optoelectronic device as claimed in claim 1, wherein the second compound is a compound of Group 2:

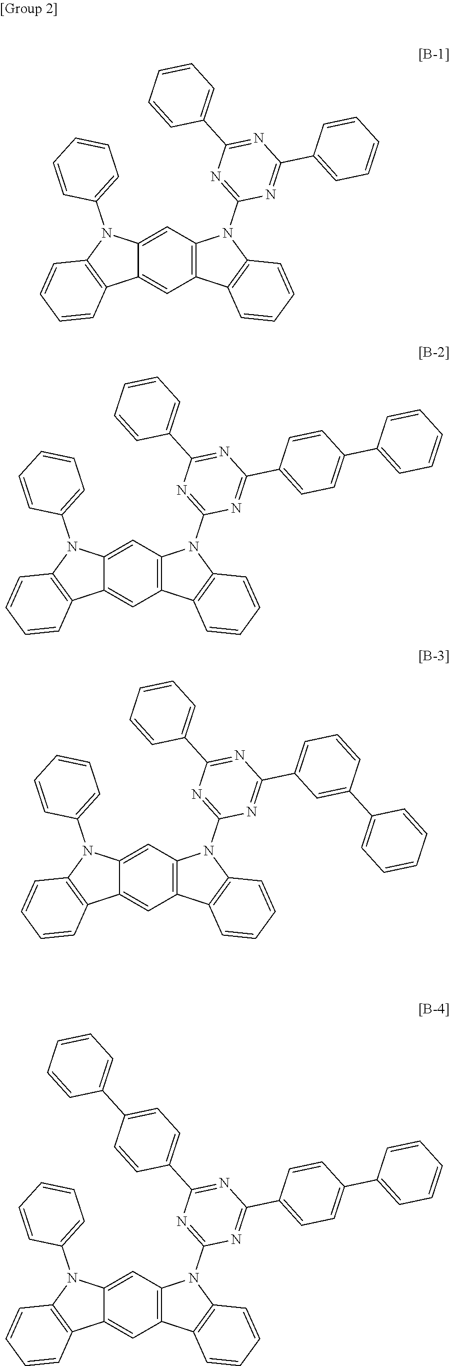 [SMILES: c1ccc(-c2nc(-c3ccccc3)nc(-n3c4ccccc4c4cc5c6ccccc6n(-c6ccccc6)c5cc43)n2)cc1 ; c1ccc(-c2ccc(-c3nc(-c4ccccc4)nc(-n4c5ccccc5c5cc6c7ccccc7n(-c7ccccc7)c6cc54)n3)cc2)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc(-c2ccc(-c3nc(-c4ccc(-c5ccccc5)cc4)nc(-n4c5ccccc5c5cc6c7ccccc7n(-c7ccccc7)c6cc54)n3)cc2)cc1]

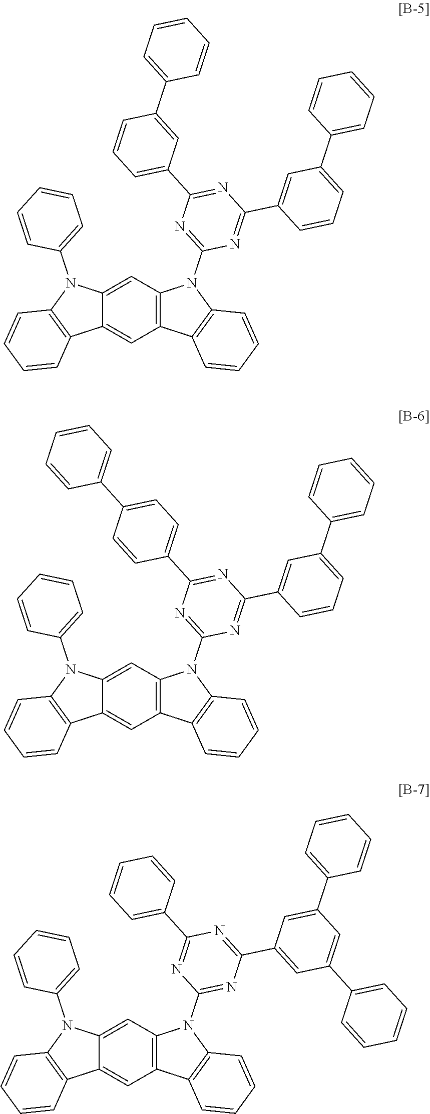 [SMILES: c1ccc(-c2cccc(-c3nc(-c4cccc(-c5ccccc5)c4)nc(-n4c5ccccc5c5cc6c7ccccc7n(-c7ccccc7)c6cc54)n3)c2)cc1 ; c1ccc(-c2ccc(-c3nc(-c4cccc(-c5ccccc5)c4)nc(-n4c5ccccc5c5cc6c7ccccc7n(-c7ccccc7)c6cc54)n3)cc2)cc1 ; c1ccc(-c2cc(-c3ccccc3)cc(-c3nc(-c4ccccc4)nc(-n4c5ccccc5c5cc6c7ccccc7n(-c7ccccc7)c6cc54)n3)c2)cc1]

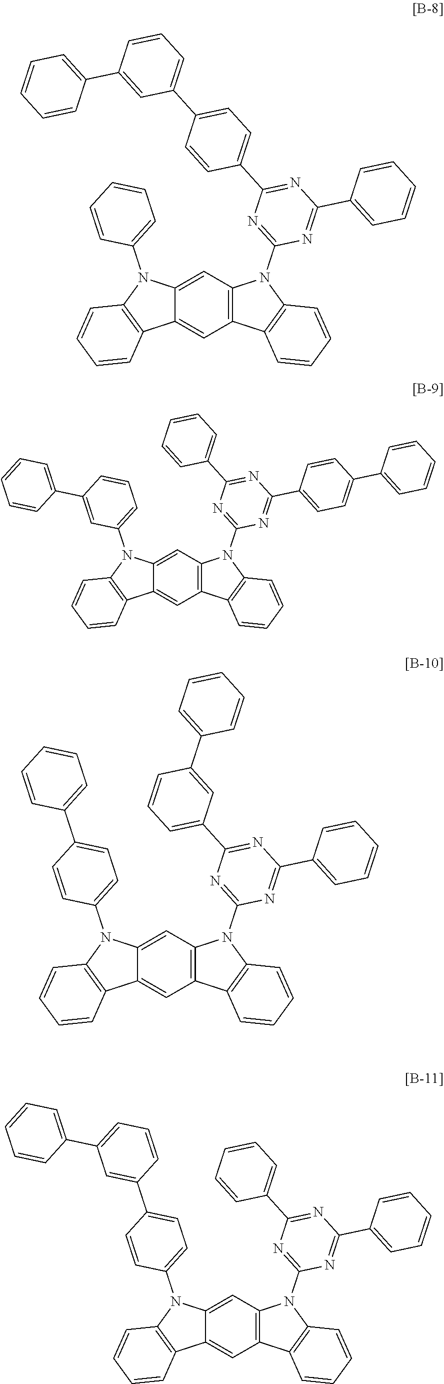 [SMILES: c1ccc(-c2cccc(-c3ccc(-c4nc(-c5ccccc5)nc(-n5c6ccccc6c6cc7c8ccccc8n(-c8ccccc8)c7cc65)n4)cc3)c2)cc1 ; c1ccc(-c2ccc(-c3nc(-c4ccccc4)nc(-n4c5ccccc5c5cc6c7ccccc7n(-c7cccc(-c8ccccc8)c7)c6cc54)n3)cc2)cc1 ; c1ccc(-c2ccc(-n3c4ccccc4c4cc5c6ccccc6n(-c6nc(-c7ccccc7)nc(-c7cccc(-c8ccccc8)c7)n6)c5cc43)cc2)cc1 ; c1ccc(-c2cccc(-c3ccc(-n4c5ccccc5c5cc6c7ccccc7n(-c7nc(-c8ccccc8)nc(-c8ccccc8)n7)c6cc54)cc3)c2)cc1]

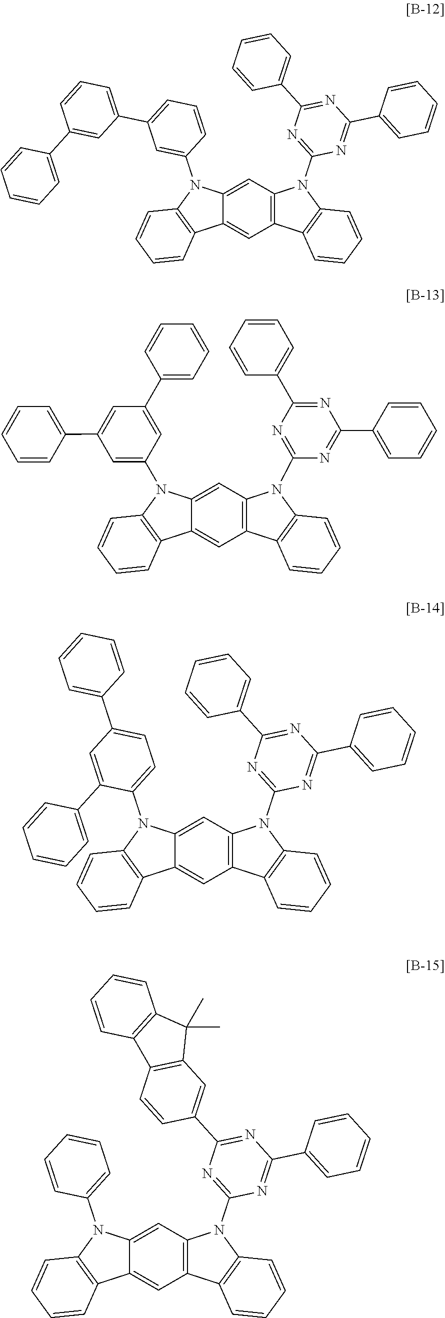 [SMILES: c1ccc(-c2cccc(-c3cccc(-n4c5ccccc5c5cc6c7ccccc7n(-c7nc(-c8ccccc8)nc(-c8ccccc8)n7)c6cc54)c3)c2)cc1 ; c1ccc(-c2cc(-c3ccccc3)cc(-n3c4ccccc4c4cc5c6ccccc6n(-c6nc(-c7ccccc7)nc(-c7ccccc7)n6)c5cc43)c2)cc1 ; c1ccc(-c2ccc(-n3c4ccccc4c4cc5c6ccccc6n(-c6nc(-c7ccccc7)nc(-c7ccccc7)n6)c5cc43)c(-c3ccccc3)c2)cc1 ; CC1(C)c2ccccc2-c2ccc(-c3nc(-c4ccccc4)nc(-n4c5ccccc5c5cc6c7ccccc7n(-c7ccccc7)c6cc54)n3)cc21]

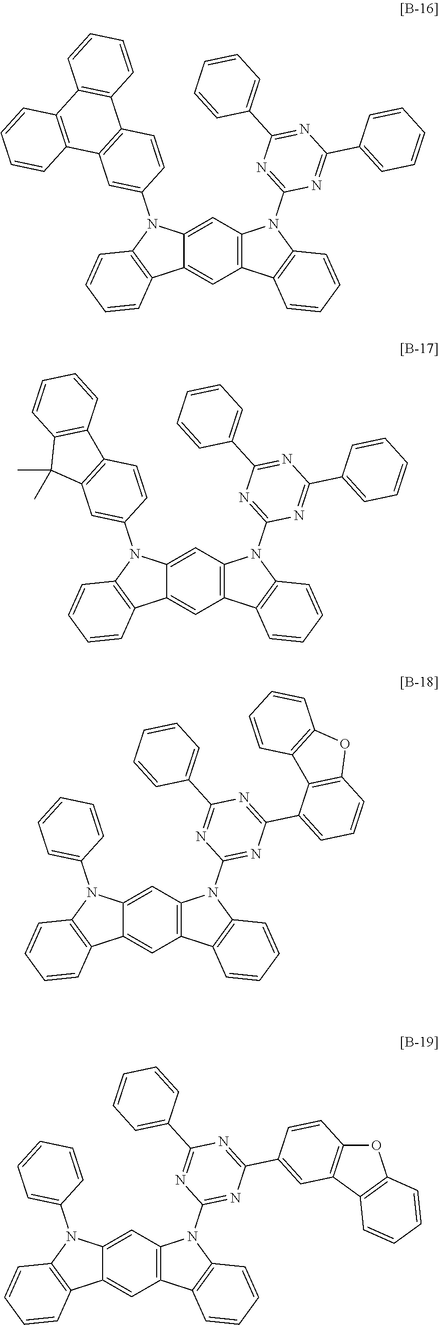 [SMILES: c1ccc(-c2nc(-c3ccccc3)nc(-n3c4ccccc4c4cc5c6ccccc6n(-c6ccc7c8ccccc8c8ccccc8c7c6)c5cc43)n2)cc1 ; CC1(C)c2ccccc2-c2ccc(-n3c4ccccc4c4cc5c6ccccc6n(-c6nc(-c7ccccc7)nc(-c7ccccc7)n6)c5cc43)cc21 ; c1ccc(-c2nc(-c3cccc4oc5ccccc5c34)nc(-n3c4ccccc4c4cc5c6ccccc6n(-c6ccccc6)c5cc43)n2)cc1 ; c1ccc(-c2nc(-c3ccc4oc5ccccc5c4c3)nc(-n3c4ccccc4c4cc5c6ccccc6n(-c6ccccc6)c5cc43)n2)cc1]

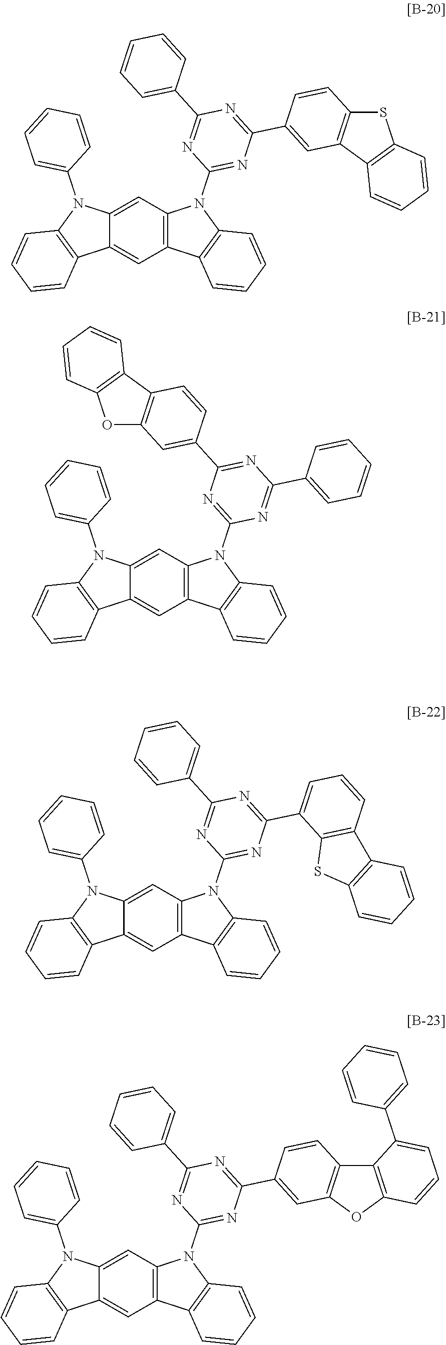

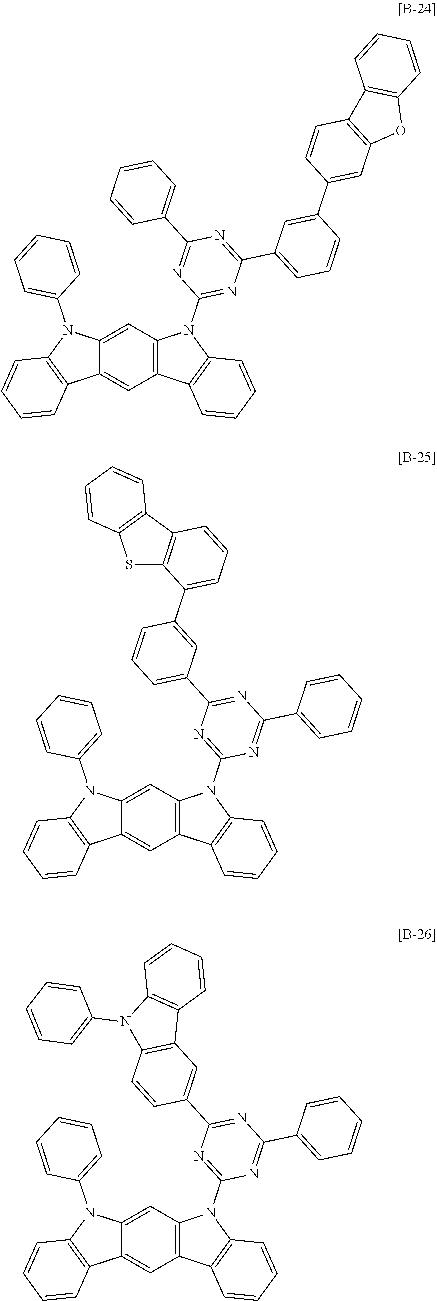 [SMILES: c1ccc(-c2nc(-c3cccc(-c4ccc5c(c4)oc4ccccc45)c3)nc(-n3c4ccccc4c4cc5c6ccccc6n(-c6ccccc6)c5cc43)n2)cc1 ; c1ccc(-c2nc(-c3cccc(-c4cccc5c4sc4ccccc45)c3)nc(-n3c4ccccc4c4cc5c6ccccc6n(-c6ccccc6)c5cc43)n2)cc1 ; c1ccc(-c2nc(-c3ccc4c(c3)c3ccccc3n4-c3ccccc3)nc(-n3c4ccccc4c4cc5c6ccccc6n(-c6ccccc6)c5cc43)n2)cc1]

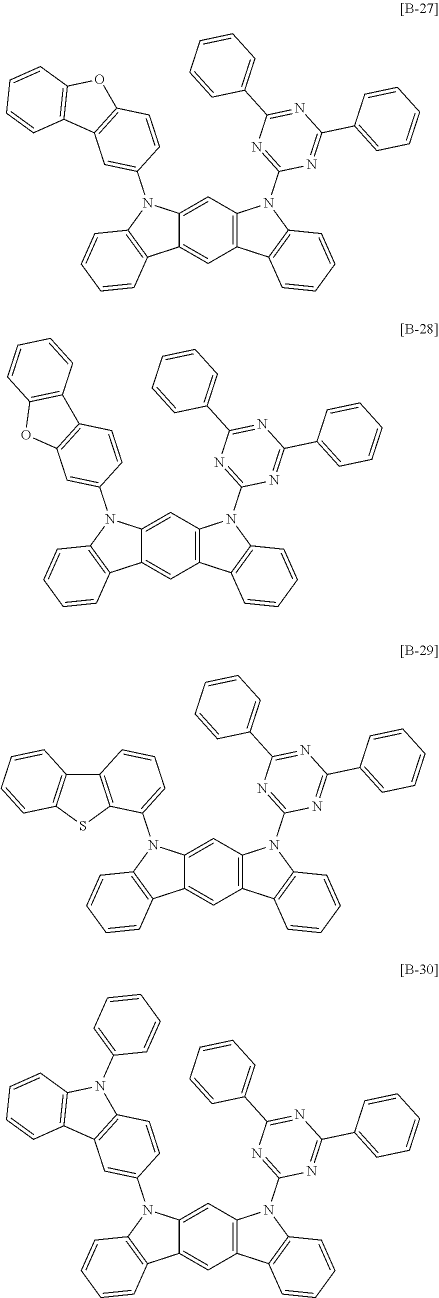 [SMILES: c1ccc(-c2nc(-c3ccccc3)nc(-n3c4ccccc4c4cc5c6ccccc6n(-c6ccc7oc8ccccc8c7c6)c5cc43)n2)cc1 ; c1ccc(-c2nc(-c3ccccc3)nc(-n3c4ccccc4c4cc5c6ccccc6n(-c6ccc7c(c6)oc6ccccc67)c5cc43)n2)cc1 ; c1ccc(-c2nc(-c3ccccc3)nc(-n3c4ccccc4c4cc5c6ccccc6n(-c6cccc7c6sc6ccccc67)c5cc43)n2)cc1 ; c1ccc(-c2nc(-c3ccccc3)nc(-n3c4ccccc4c4cc5c6ccccc6n(-c6ccc7c(c6)c6ccccc6n7-c6ccccc6)c5cc43)n2)cc1]

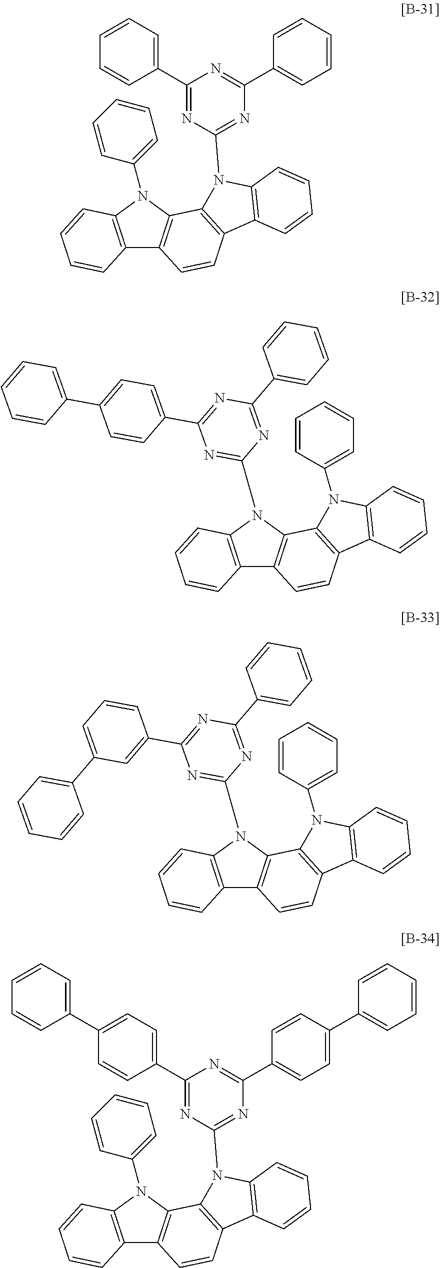 [SMILES: c1ccc(-c2nc(-c3ccccc3)nc(-n3c4ccccc4c4ccc5c6ccccc6n(-c6ccccc6)c5c43)n2)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-c2cccc(-c3nc(-c4ccccc4)nc(-n4c5ccccc5c5ccc6c7ccccc7n(-c7ccccc7)c6c54)n3)c2)cc1 ; c1ccc(-c2ccc(-c3nc(-c4ccc(-c5ccccc5)cc4)nc(-n4c5ccccc5c5ccc6c7ccccc7n(-c7ccccc7)c6c54)n3)cc2)cc1]

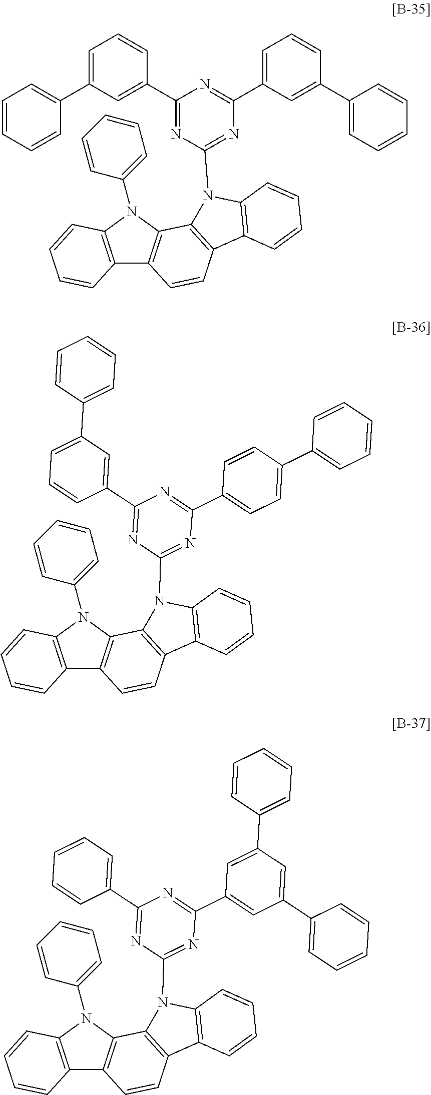 [SMILES: c1ccc(-c2cccc(-c3nc(-c4cccc(-c5ccccc5)c4)nc(-n4c5ccccc5c5ccc6c7ccccc7n(-c7ccccc7)c6c54)n3)c2)cc1 ; c1ccc(-c2ccc(-c3nc(-c4cccc(-c5ccccc5)c4)nc(-n4c5ccccc5c5ccc6c7ccccc7n(-c7ccccc7)c6c54)n3)cc2)cc1 ; c1ccc(-c2cc(-c3ccccc3)cc(-c3nc(-c4ccccc4)nc(-n4c5ccccc5c5ccc6c7ccccc7n(-c7ccccc7)c6c54)n3)c2)cc1]

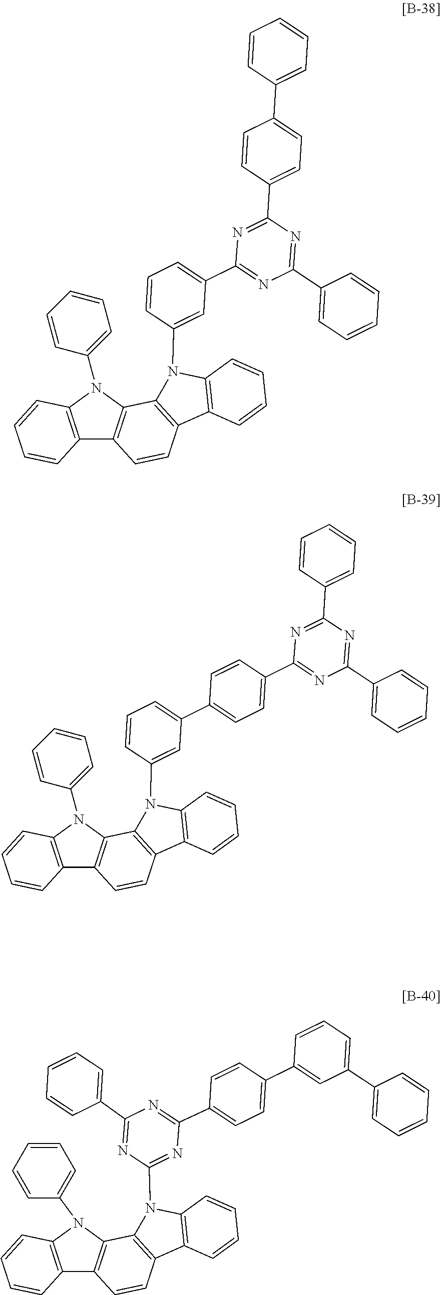 [SMILES: c1ccc(-c2ccc(-c3nc(-c4ccccc4)nc(-c4cccc(-n5c6ccccc6c6ccc7c8ccccc8n(-c8ccccc8)c7c65)c4)n3)cc2)cc1 ; c1ccc(-c2nc(-c3ccccc3)nc(-c3ccc(-c4cccc(-n5c6ccccc6c6ccc7c8ccccc8n(-c8ccccc8)c7c65)c4)cc3)n2)cc1 ; c1ccc(-c2cccc(-c3ccc(-c4nc(-c5ccccc5)nc(-n5c6ccccc6c6ccc7c8ccccc8n(-c8ccccc8)c7c65)n4)cc3)c2)cc1]

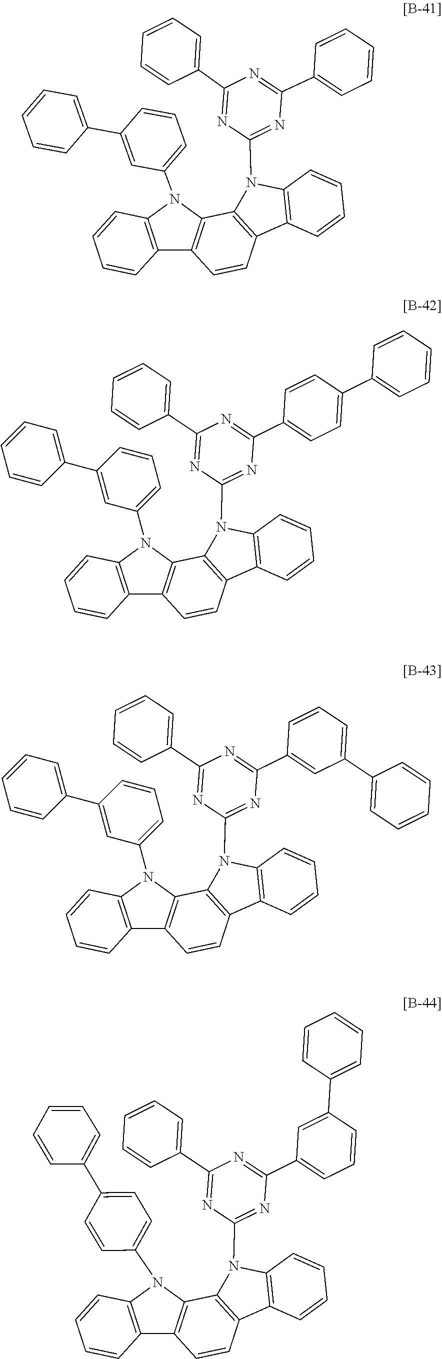 [SMILES: c1ccc(-c2cccc(-n3c4ccccc4c4ccc5c6ccccc6n(-c6nc(-c7ccccc7)nc(-c7ccccc7)n6)c5c43)c2)cc1 ; c1ccc(-c2ccc(-c3nc(-c4ccccc4)nc(-n4c5ccccc5c5ccc6c7ccccc7n(-c7cccc(-c8ccccc8)c7)c6c54)n3)cc2)cc1 ; c1ccc(-c2cccc(-c3nc(-c4ccccc4)nc(-n4c5ccccc5c5ccc6c7ccccc7n(-c7cccc(-c8ccccc8)c7)c6c54)n3)c2)cc1 ; c1ccc(-c2ccc(-n3c4ccccc4c4ccc5c6ccccc6n(-c6nc(-c7ccccc7)nc(-c7cccc(-c8ccccc8)c7)n6)c5c43)cc2)cc1]

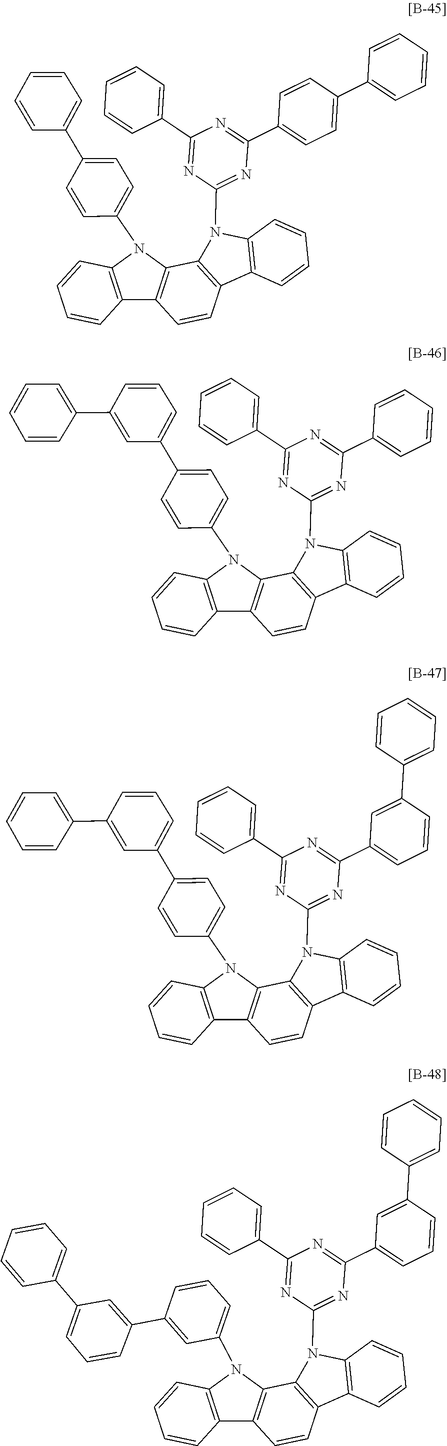 [SMILES: c1ccc(-c2ccc(-c3nc(-c4ccccc4)nc(-n4c5ccccc5c5ccc6c7ccccc7n(-c7ccc(-c8ccccc8)cc7)c6c54)n3)cc2)cc1 ; c1ccc(-c2cccc(-c3ccc(-n4c5ccccc5c5ccc6c7ccccc7n(-c7nc(-c8ccccc8)nc(-c8ccccc8)n7)c6c54)cc3)c2)cc1 ; c1ccc(-c2cccc(-c3ccc(-n4c5ccccc5c5ccc6c7ccccc7n(-c7nc(-c8ccccc8)nc(-c8cccc(-c9ccccc9)c8)n7)c6c54)cc3)c2)cc1 ; c1ccc(-c2cccc(-c3cccc(-n4c5ccccc5c5ccc6c7ccccc7n(-c7nc(-c8ccccc8)nc(-c8cccc(-c9ccccc9)c8)n7)c6c54)c3)c2)cc1]

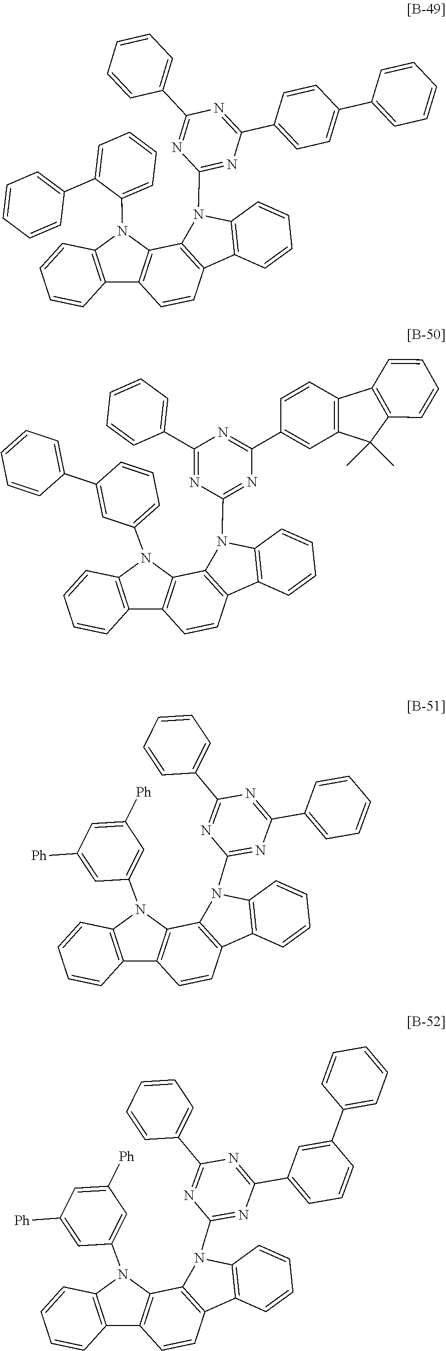 [SMILES: c1ccc(-c2ccc(-c3nc(-c4ccccc4)nc(-n4c5ccccc5c5ccc6c7ccccc7n(-c7ccccc7-c7ccccc7)c6c54)n3)cc2)cc1 ; CC1(C)c2ccccc2-c2ccc(-c3nc(-c4ccccc4)nc(-n4c5ccccc5c5ccc6c7ccccc7n(-c7cccc(-c8ccccc8)c7)c6c54)n3)cc21 ; c1ccc(-c2cc(-c3ccccc3)cc(-n3c4ccccc4c4ccc5c6ccccc6n(-c6nc(-c7ccccc7)nc(-c7ccccc7)n6)c5c43)c2)cc1 ; c1ccc(-c2cccc(-c3nc(-c4ccccc4)nc(-n4c5ccccc5c5ccc6c7ccccc7n(-c7cc(-c8ccccc8)cc(-c8ccccc8)c7)c6c54)n3)c2)cc1]

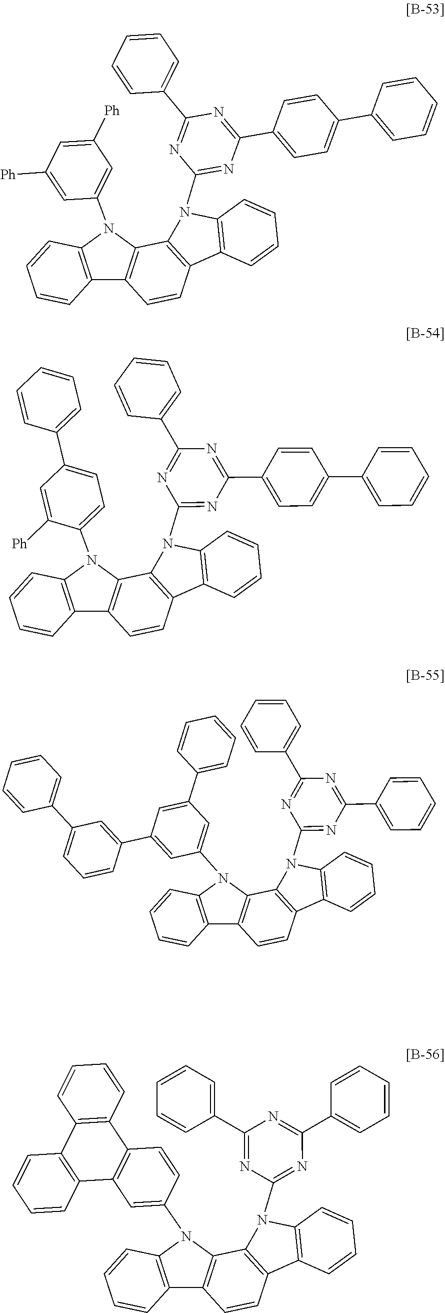 [SMILES: c1ccc(-c2ccc(-c3nc(-c4ccccc4)nc(-n4c5ccccc5c5ccc6c7ccccc7n(-c7cc(-c8ccccc8)cc(-c8ccccc8)c7)c6c54)n3)cc2)cc1 ; c1ccc(-c2ccc(-c3nc(-c4ccccc4)nc(-n4c5ccccc5c5ccc6c7ccccc7n(-c7ccc(-c8ccccc8)cc7-c7ccccc7)c6c54)n3)cc2)cc1 ; c1ccc(-c2cccc(-c3cc(-c4ccccc4)cc(-n4c5ccccc5c5ccc6c7ccccc7n(-c7nc(-c8ccccc8)nc(-c8ccccc8)n7)c6c54)c3)c2)cc1 ; c1ccc(-c2nc(-c3ccccc3)nc(-n3c4ccccc4c4ccc5c6ccccc6n(-c6ccc7c8ccccc8c8ccccc8c7c6)c5c43)n2)cc1]

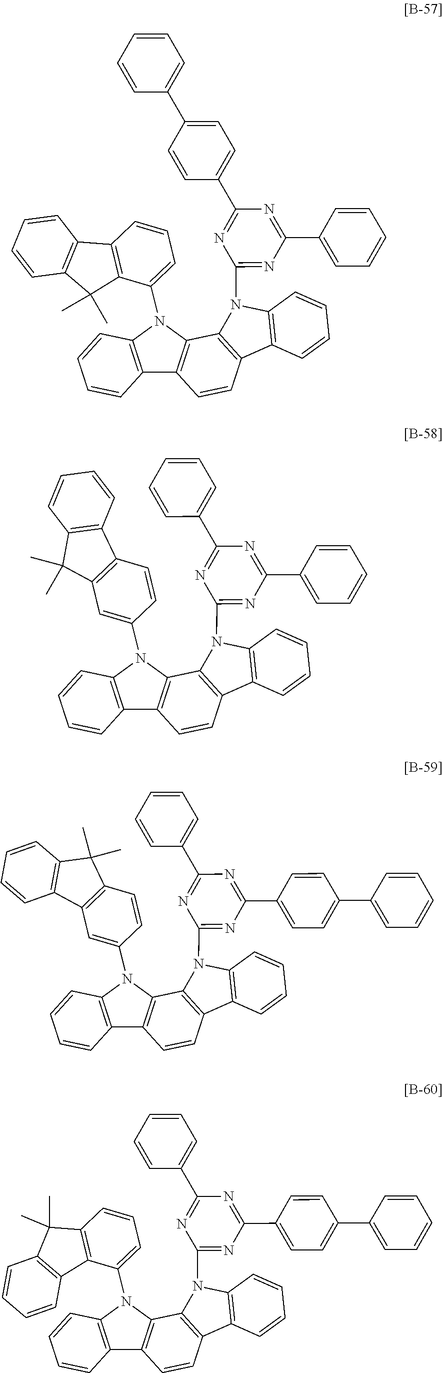 [SMILES: CC1(C)c2ccccc2-c2cccc(-n3c4ccccc4c4ccc5c6ccccc6n(-c6nc(-c7ccccc7)nc(-c7ccc(-c8ccccc8)cc7)n6)c5c43)c21 ; CC1(C)c2ccccc2-c2ccc(-n3c4ccccc4c4ccc5c6ccccc6n(-c6nc(-c7ccccc7)nc(-c7ccccc7)n6)c5c43)cc21 ; CC1(C)c2ccccc2-c2cc(-n3c4ccccc4c4ccc5c6ccccc6n(-c6nc(-c7ccccc7)nc(-c7ccc(-c8ccccc8)cc7)n6)c5c43)ccc21 ; CC1(C)c2ccccc2-c2c(-n3c4ccccc4c4ccc5c6ccccc6n(-c6nc(-c7ccccc7)nc(-c7ccc(-c8ccccc8)cc7)n6)c5c43)cccc21]

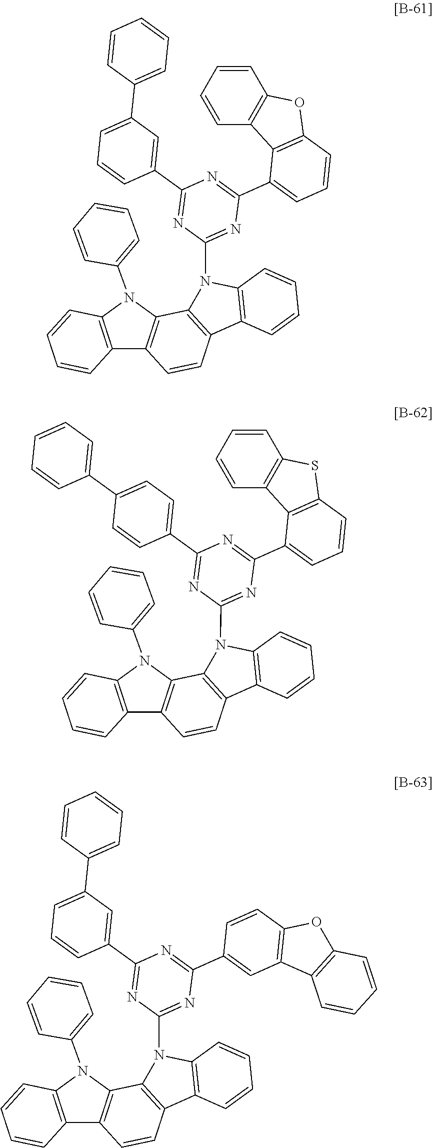 [SMILES: c1ccc(-c2cccc(-c3nc(-c4cccc5oc6ccccc6c45)nc(-n4c5ccccc5c5ccc6c7ccccc7n(-c7ccccc7)c6c54)n3)c2)cc1 ; c1ccc(-c2ccc(-c3nc(-c4cccc5sc6ccccc6c45)nc(-n4c5ccccc5c5ccc6c7ccccc7n(-c7ccccc7)c6c54)n3)cc2)cc1 ; c1ccc(-c2cccc(-c3nc(-c4ccc5oc6ccccc6c5c4)nc(-n4c5ccccc5c5ccc6c7ccccc7n(-c7ccccc7)c6c54)n3)c2)cc1]

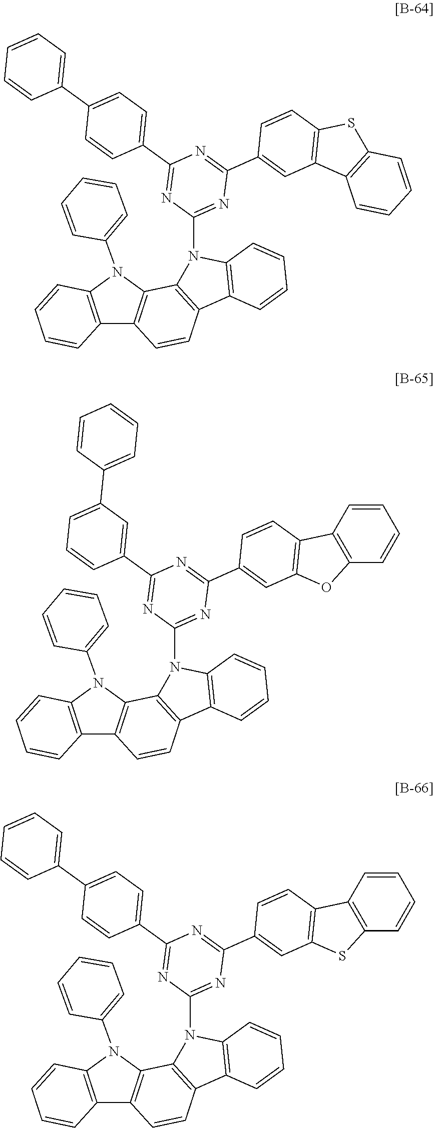 [SMILES: c1ccc(-c2ccc(-c3nc(-c4ccc5sc6ccccc6c5c4)nc(-n4c5ccccc5c5ccc6c7ccccc7n(-c7ccccc7)c6c54)n3)cc2)cc1 ; c1ccc(-c2cccc(-c3nc(-c4ccc5c(c4)oc4ccccc45)nc(-n4c5ccccc5c5ccc6c7ccccc7n(-c7ccccc7)c6c54)n3)c2)cc1 ; c1ccc(-c2ccc(-c3nc(-c4ccc5c(c4)sc4ccccc45)nc(-n4c5ccccc5c5ccc6c7ccccc7n(-c7ccccc7)c6c54)n3)cc2)cc1]

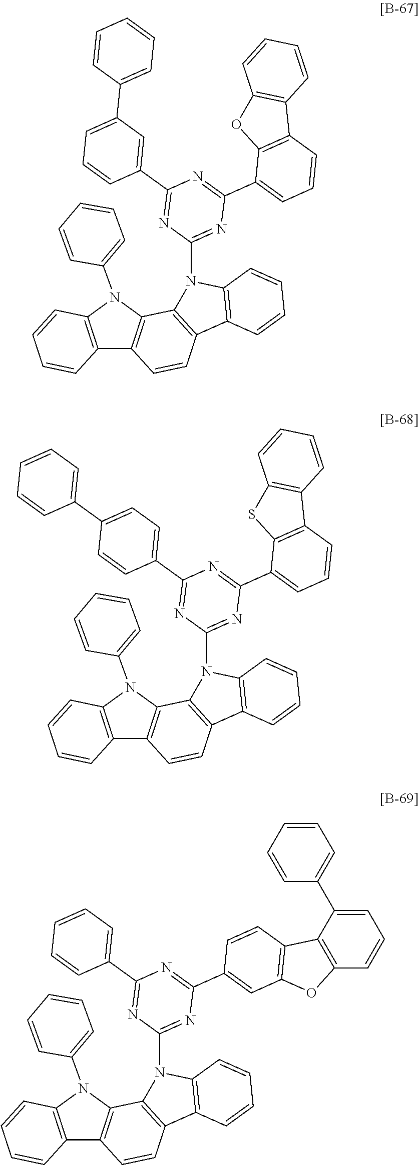 [SMILES: c1ccc(-c2cccc(-c3nc(-c4cccc5c4oc4ccccc45)nc(-n4c5ccccc5c5ccc6c7ccccc7n(-c7ccccc7)c6c54)n3)c2)cc1 ; c1ccc(-c2ccc(-c3nc(-c4cccc5c4sc4ccccc45)nc(-n4c5ccccc5c5ccc6c7ccccc7n(-c7ccccc7)c6c54)n3)cc2)cc1 ; c1ccc(-c2nc(-c3ccc4c(c3)oc3cccc(-c5ccccc5)c34)nc(-n3c4ccccc4c4ccc5c6ccccc6n(-c6ccccc6)c5c43)n2)cc1]

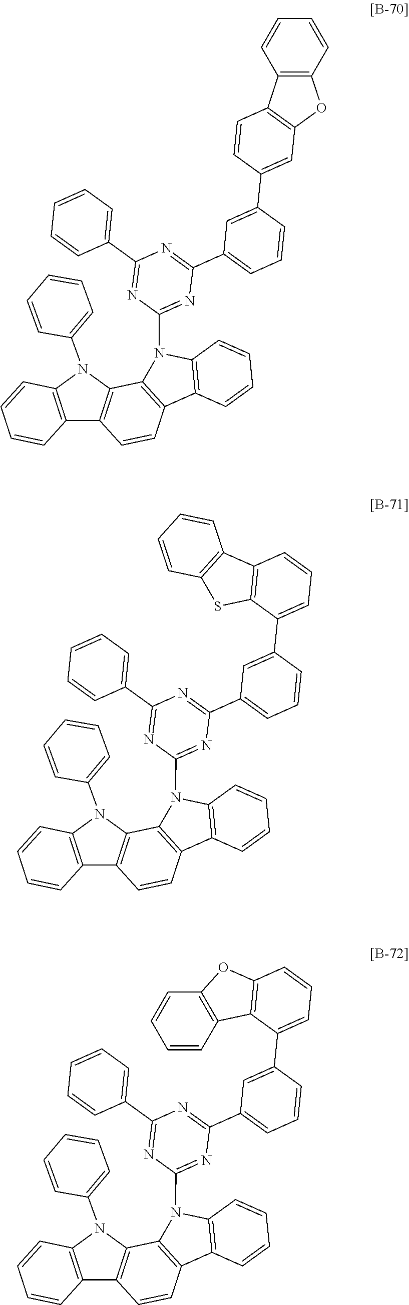 [SMILES: c1ccc(-c2nc(-c3cccc(-c4ccc5c(c4)oc4ccccc45)c3)nc(-n3c4ccccc4c4ccc5c6ccccc6n(-c6ccccc6)c5c43)n2)cc1 ; c1ccc(-c2nc(-c3cccc(-c4cccc5c4sc4ccccc45)c3)nc(-n3c4ccccc4c4ccc5c6ccccc6n(-c6ccccc6)c5c43)n2)cc1 ; c1ccc(-c2nc(-c3cccc(-c4cccc5oc6ccccc6c45)c3)nc(-n3c4ccccc4c4ccc5c6ccccc6n(-c6ccccc6)c5c43)n2)cc1]

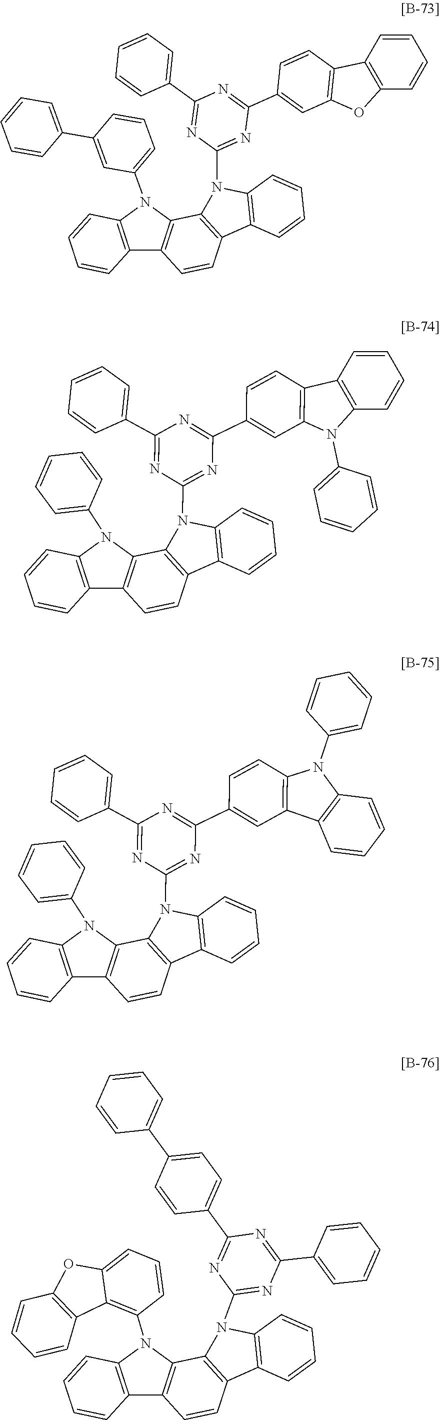 [SMILES: c1ccc(-c2cccc(-n3c4ccccc4c4ccc5c6ccccc6n(-c6nc(-c7ccccc7)nc(-c7ccc8c(c7)oc7ccccc78)n6)c5c43)c2)cc1 ; c1ccc(-c2nc(-c3ccc4c5ccccc5n(-c5ccccc5)c4c3)nc(-n3c4ccccc4c4ccc5c6ccccc6n(-c6ccccc6)c5c43)n2)cc1 ; c1ccc(-c2nc(-c3ccc4c(c3)c3ccccc3n4-c3ccccc3)nc(-n3c4ccccc4c4ccc5c6ccccc6n(-c6ccccc6)c5c43)n2)cc1 ; c1ccc(-c2ccc(-c3nc(-c4ccccc4)nc(-n4c5ccccc5c5ccc6c7ccccc7n(-c7cccc8oc9ccccc9c78)c6c54)n3)cc2)cc1]

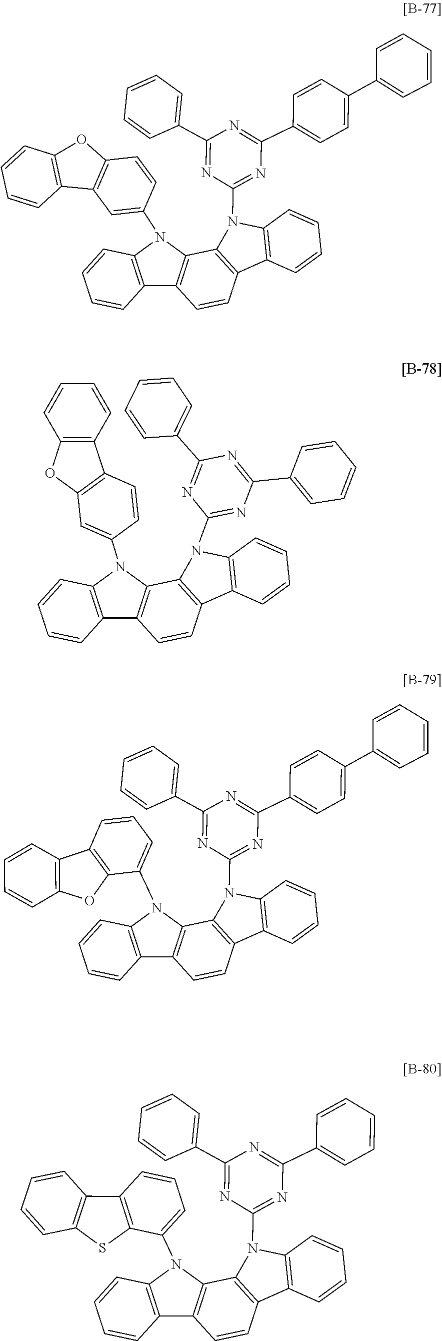 [SMILES: c1ccc(-c2ccc(-c3nc(-c4ccccc4)nc(-n4c5ccccc5c5ccc6c7ccccc7n(-c7ccc8oc9ccccc9c8c7)c6c54)n3)cc2)cc1 ; c1ccc(-c2nc(-c3ccccc3)nc(-n3c4ccccc4c4ccc5c6ccccc6n(-c6ccc7c(c6)oc6ccccc67)c5c43)n2)cc1 ; c1ccc(-c2ccc(-c3nc(-c4ccccc4)nc(-n4c5ccccc5c5ccc6c7ccccc7n(-c7cccc8c7oc7ccccc78)c6c54)n3)cc2)cc1 ; c1ccc(-c2nc(-c3ccccc3)nc(-n3c4ccccc4c4ccc5c6ccccc6n(-c6cccc7c6sc6ccccc67)c5c43)n2)cc1]

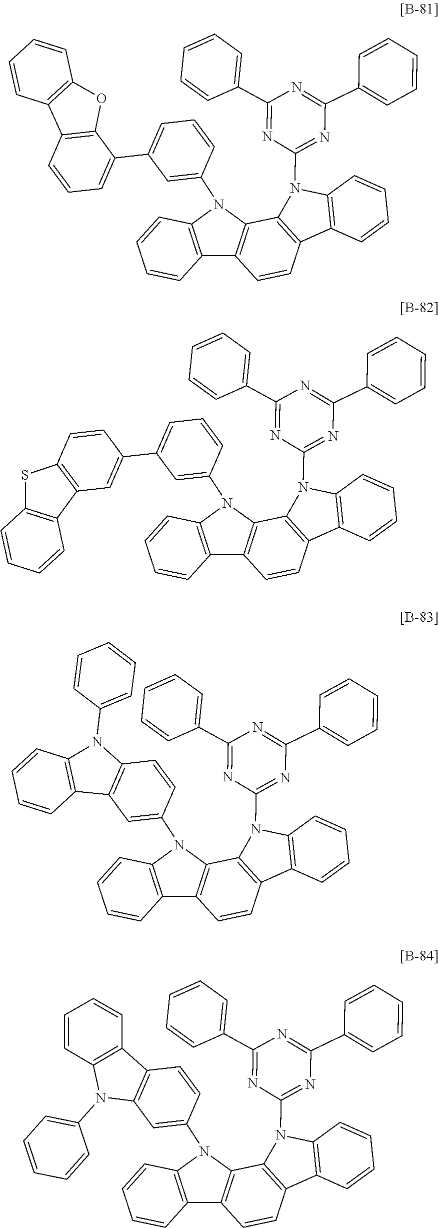 [SMILES: c1ccc(-c2nc(-c3ccccc3)nc(-n3c4ccccc4c4ccc5c6ccccc6n(-c6cccc(-c7cccc8c7oc7ccccc78)c6)c5c43)n2)cc1 ; c1ccc(-c2nc(-c3ccccc3)nc(-n3c4ccccc4c4ccc5c6ccccc6n(-c6cccc(-c7ccc8sc9ccccc9c8c7)c6)c5c43)n2)cc1 ; c1ccc(-c2nc(-c3ccccc3)nc(-n3c4ccccc4c4ccc5c6ccccc6n(-c6ccc7c(c6)c6ccccc6n7-c6ccccc6)c5c43)n2)cc1 ; c1ccc(-c2nc(-c3ccccc3)nc(-n3c4ccccc4c4ccc5c6ccccc6n(-c6ccc7c8ccccc8n(-c8ccccc8)c7c6)c5c43)n2)cc1]

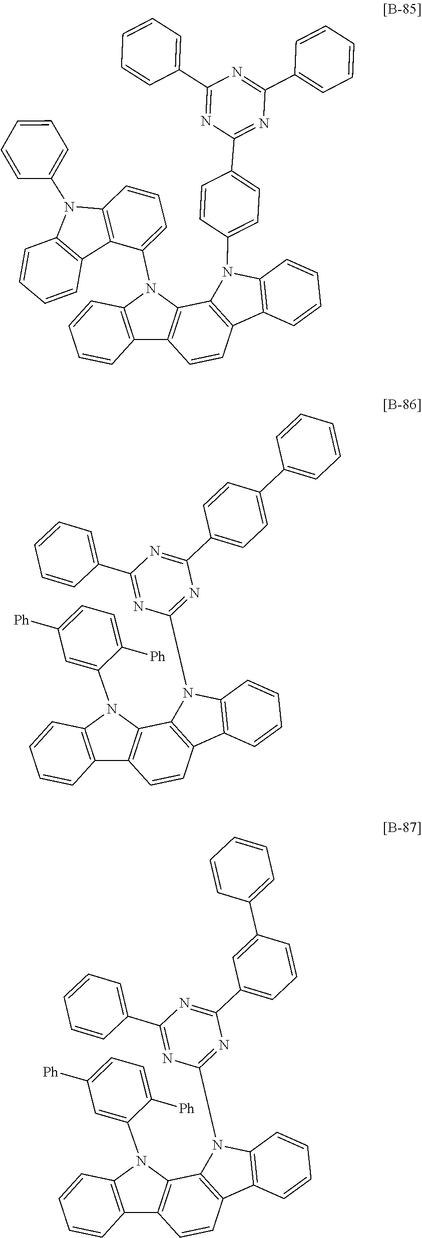 [SMILES: c1ccc(-c2nc(-c3ccccc3)nc(-c3ccc(-n4c5ccccc5c5ccc6c7ccccc7n(-c7cccc8c7c7ccccc7n8-c7ccccc7)c6c54)cc3)n2)cc1 ; c1ccc(-c2ccc(-c3nc(-c4ccccc4)nc(-n4c5ccccc5c5ccc6c7ccccc7n(-c7cc(-c8ccccc8)ccc7-c7ccccc7)c6c54)n3)cc2)cc1 ; c1ccc(-c2cccc(-c3nc(-c4ccccc4)nc(-n4c5ccccc5c5ccc6c7ccccc7n(-c7cc(-c8ccccc8)ccc7-c7ccccc7)c6c54)n3)c2)cc1]

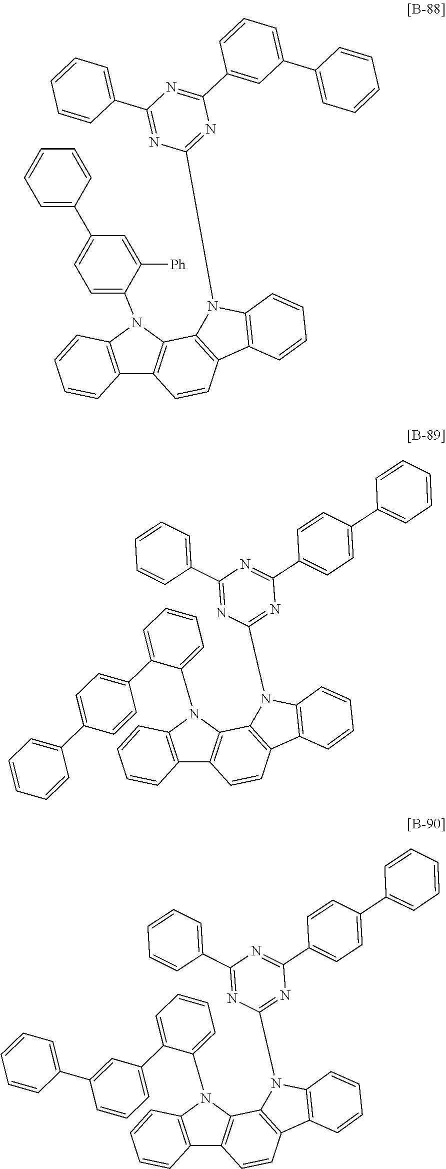 [SMILES: c1ccc(-c2cccc(-c3nc(-c4ccccc4)nc(-n4c5ccccc5c5ccc6c7ccccc7n(-c7ccc(-c8ccccc8)cc7-c7ccccc7)c6c54)n3)c2)cc1 ; c1ccc(-c2ccc(-c3nc(-c4ccccc4)nc(-n4c5ccccc5c5ccc6c7ccccc7n(-c7ccccc7-c7ccc(-c8ccccc8)cc7)c6c54)n3)cc2)cc1 ; c1ccc(-c2ccc(-c3nc(-c4ccccc4)nc(-n4c5ccccc5c5ccc6c7ccccc7n(-c7ccccc7-c7cccc(-c8ccccc8)c7)c6c54)n3)cc2)cc1]

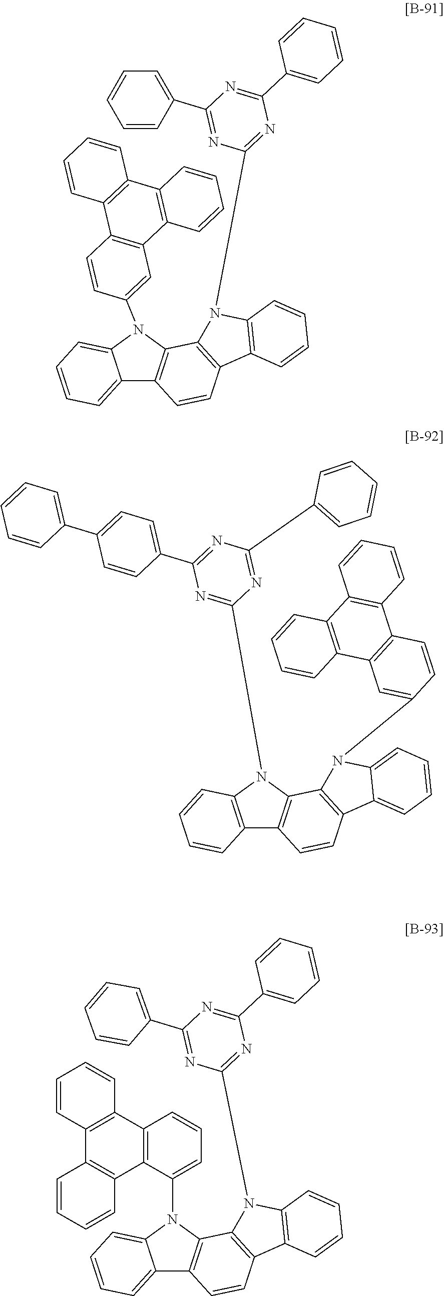 [SMILES: c1ccc(-c2nc(-c3ccccc3)nc(-n3c4ccccc4c4ccc5c6ccccc6n(-c6ccc7c8ccccc8c8ccccc8c7c6)c5c43)n2)cc1 ; c1ccc(-c2ccc(-c3nc(-c4ccccc4)nc(-n4c5ccccc5c5ccc6c7ccccc7n(-c7ccc8c9ccccc9c9ccccc9c8c7)c6c54)n3)cc2)cc1 ; c1ccc(-c2nc(-c3ccccc3)nc(-n3c4ccccc4c4ccc5c6ccccc6n(-c6cccc7c8ccccc8c8ccccc8c67)c5c43)n2)cc1]

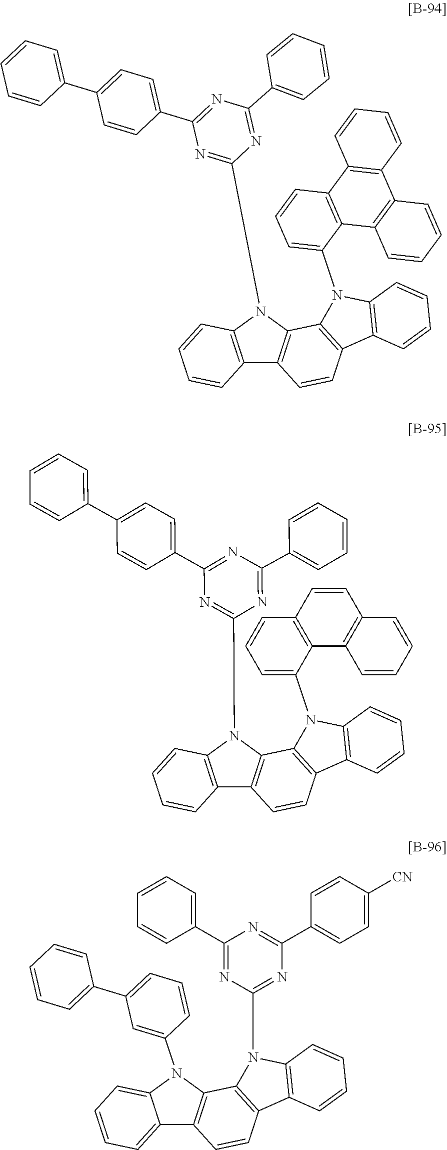 [SMILES: c1ccc(-c2ccc(-c3nc(-c4ccccc4)nc(-n4c5ccccc5c5ccc6c7ccccc7n(-c7cccc8c9ccccc9c9ccccc9c78)c6c54)n3)cc2)cc1 ; c1ccc(-c2ccc(-c3nc(-c4ccccc4)nc(-n4c5ccccc5c5ccc6c7ccccc7n(-c7cccc8ccc9ccccc9c78)c6c54)n3)cc2)cc1 ; N#Cc1ccc(-c2nc(-c3ccccc3)nc(-n3c4ccccc4c4ccc5c6ccccc6n(-c6cccc(-c7ccccc7)c6)c5c43)n2)cc1]

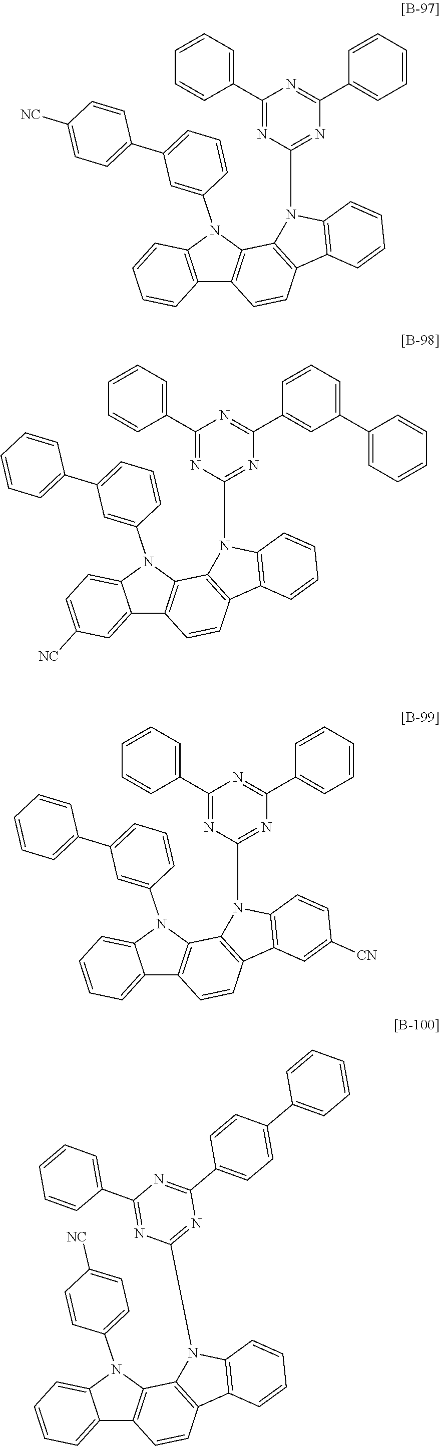 [SMILES: N#Cc1ccc(-c2cccc(-n3c4ccccc4c4ccc5c6ccccc6n(-c6nc(-c7ccccc7)nc(-c7ccccc7)n6)c5c43)c2)cc1 ; N#Cc1ccc2c(c1)c1ccc3c4ccccc4n(-c4nc(-c5ccccc5)nc(-c5cccc(-c6ccccc6)c5)n4)c3c1n2-c1cccc(-c2ccccc2)c1 ; N#Cc1ccc2c(c1)c1ccc3c4ccccc4n(-c4cccc(-c5ccccc5)c4)c3c1n2-c1nc(-c2ccccc2)nc(-c2ccccc2)n1 ; N#Cc1ccc(-n2c3ccccc3c3ccc4c5ccccc5n(-c5nc(-c6ccccc6)nc(-c6ccc(-c7ccccc7)cc6)n5)c4c32)cc1]

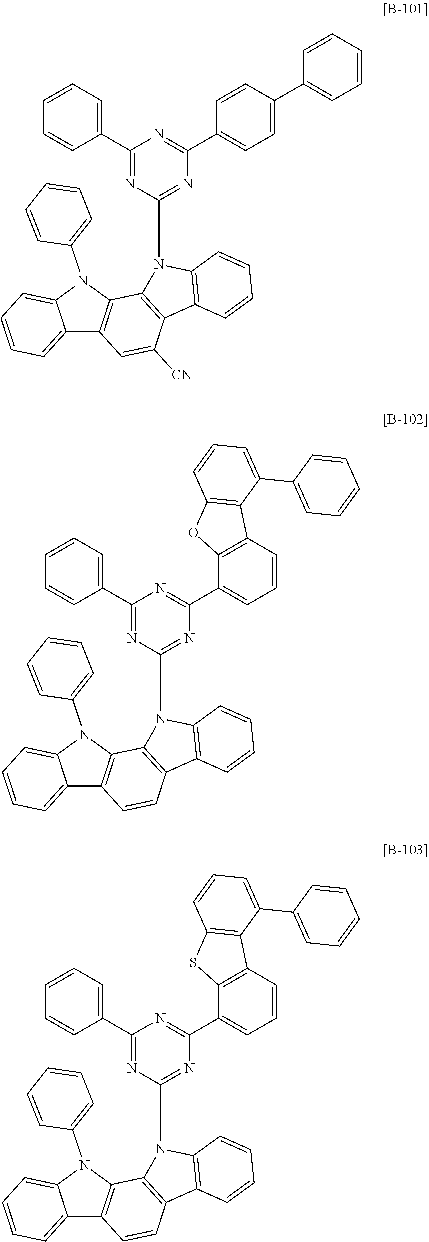 [SMILES: N#Cc1cc2c3ccccc3n(-c3ccccc3)c2c2c1c1ccccc1n2-c1nc(-c2ccccc2)nc(-c2ccc(-c3ccccc3)cc2)n1 ; c1ccc(-c2nc(-c3cccc4c3oc3cccc(-c5ccccc5)c34)nc(-n3c4ccccc4c4ccc5c6ccccc6n(-c6ccccc6)c5c43)n2)cc1 ; c1ccc(-c2nc(-c3cccc4c3sc3cccc(-c5ccccc5)c34)nc(-n3c4ccccc4c4ccc5c6ccccc6n(-c6ccccc6)c5c43)n2)cc1]

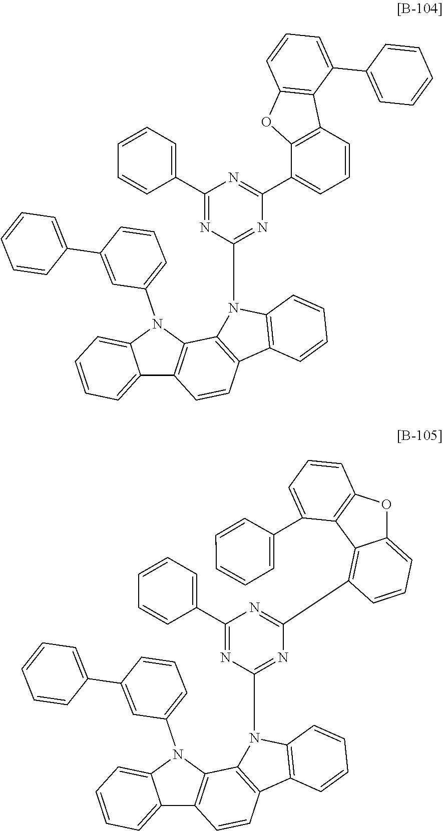 [SMILES: c1ccc(-c2cccc(-n3c4ccccc4c4ccc5c6ccccc6n(-c6nc(-c7ccccc7)nc(-c7cccc8c7oc7cccc(-c9ccccc9)c78)n6)c5c43)c2)cc1 ; c1ccc(-c2cccc(-n3c4ccccc4c4ccc5c6ccccc6n(-c6nc(-c7ccccc7)nc(-c7cccc8oc9cccc(-c%10ccccc%10)c9c78)n6)c5c43)c2)cc1]

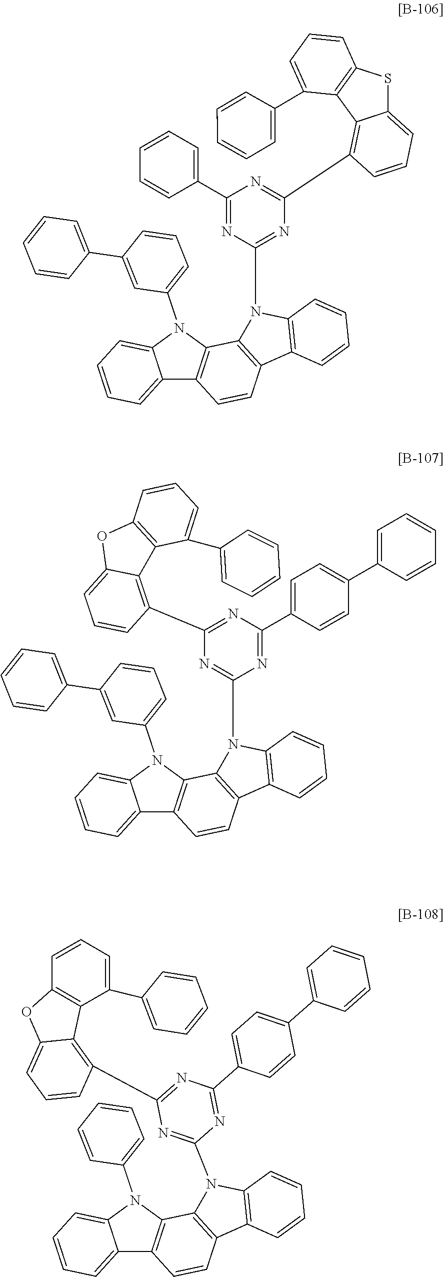 [SMILES: c1ccc(-c2cccc(-n3c4ccccc4c4ccc5c6ccccc6n(-c6nc(-c7ccccc7)nc(-c7cccc8sc9cccc(-c%10ccccc%10)c9c78)n6)c5c43)c2)cc1 ; c1ccc(-c2ccc(-c3nc(-c4cccc5oc6cccc(-c7ccccc7)c6c45)nc(-n4c5ccccc5c5ccc6c7ccccc7n(-c7cccc(-c8ccccc8)c7)c6c54)n3)cc2)cc1 ; c1ccc(-c2ccc(-c3nc(-c4cccc5oc6cccc(-c7ccccc7)c6c45)nc(-n4c5ccccc5c5ccc6c7ccccc7n(-c7ccccc7)c6c54)n3)cc2)cc1]

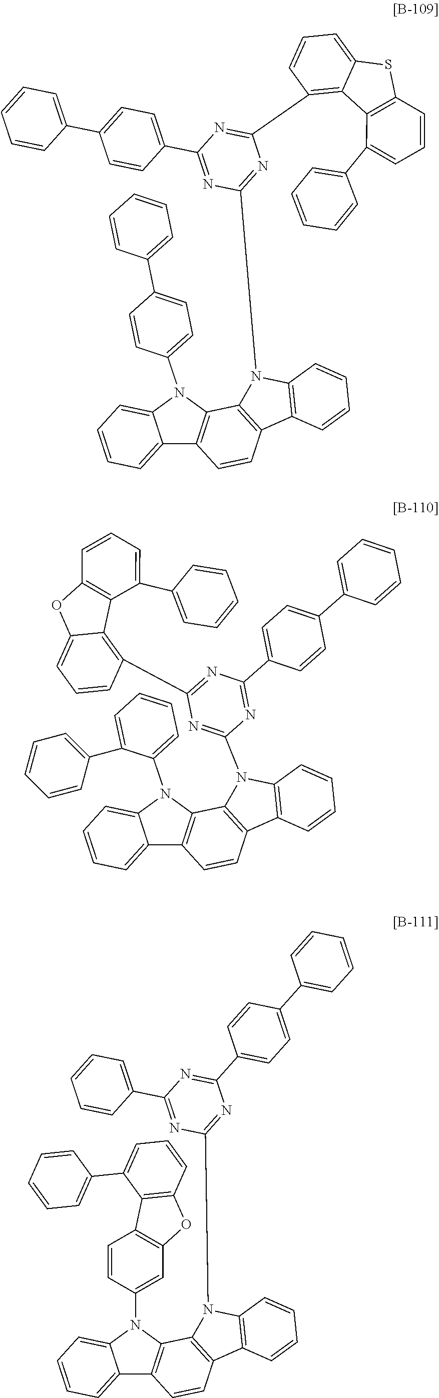 [SMILES: c1ccc(-c2ccc(-c3nc(-c4cccc5sc6cccc(-c7ccccc7)c6c45)nc(-n4c5ccccc5c5ccc6c7ccccc7n(-c7ccc(-c8ccccc8)cc7)c6c54)n3)cc2)cc1 ; c1ccc(-c2ccc(-c3nc(-c4cccc5oc6cccc(-c7ccccc7)c6c45)nc(-n4c5ccccc5c5ccc6c7ccccc7n(-c7ccccc7-c7ccccc7)c6c54)n3)cc2)cc1 ; c1ccc(-c2ccc(-c3nc(-c4ccccc4)nc(-n4c5ccccc5c5ccc6c7ccccc7n(-c7ccc8c(c7)oc7cccc(-c9ccccc9)c78)c6c54)n3)cc2)cc1]

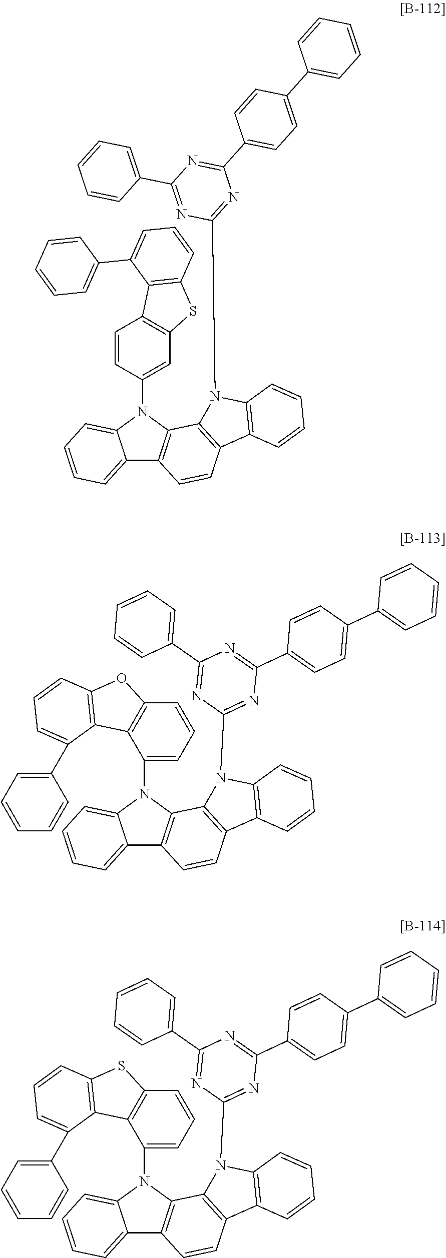 [SMILES: c1ccc(-c2ccc(-c3nc(-c4ccccc4)nc(-n4c5ccccc5c5ccc6c7ccccc7n(-c7ccc8c(c7)sc7cccc(-c9ccccc9)c78)c6c54)n3)cc2)cc1 ; c1ccc(-c2ccc(-c3nc(-c4ccccc4)nc(-n4c5ccccc5c5ccc6c7ccccc7n(-c7cccc8oc9cccc(-c%10ccccc%10)c9c78)c6c54)n3)cc2)cc1 ; c1ccc(-c2ccc(-c3nc(-c4ccccc4)nc(-n4c5ccccc5c5ccc6c7ccccc7n(-c7cccc8sc9cccc(-c%10ccccc%10)c9c78)c6c54)n3)cc2)cc1]

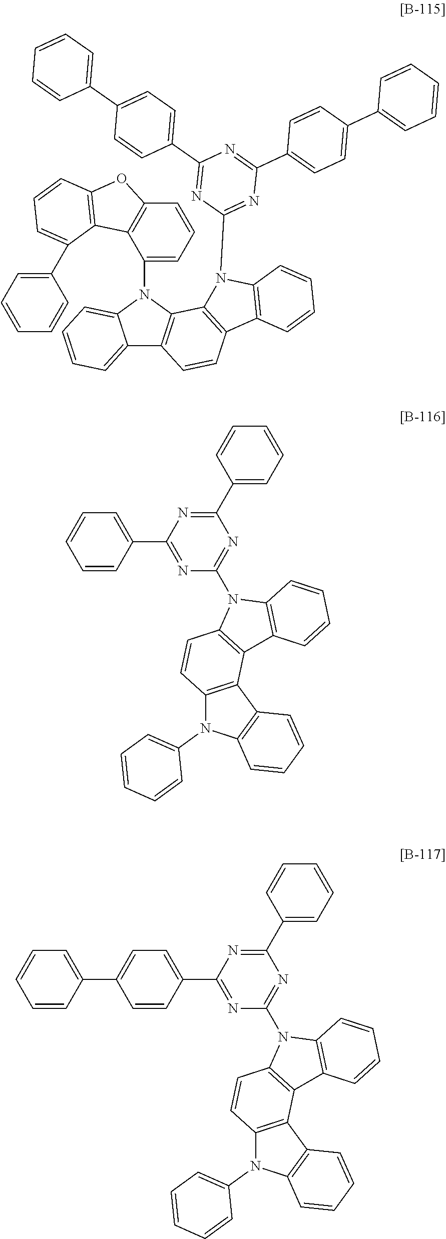 [SMILES: c1ccc(-c2ccc(-c3nc(-c4ccc(-c5ccccc5)cc4)nc(-n4c5ccccc5c5ccc6c7ccccc7n(-c7cccc8oc9cccc(-c%10ccccc%10)c9c78)c6c54)n3)cc2)cc1 ; c1ccc(-c2nc(-c3ccccc3)nc(-n3c4ccccc4c4c5c6ccccc6n(-c6ccccc6)c5ccc43)n2)cc1 ; c1ccc(-c2ccc(-c3nc(-c4ccccc4)nc(-n4c5ccccc5c5c6c7ccccc7n(-c7ccccc7)c6ccc54)n3)cc2)cc1]

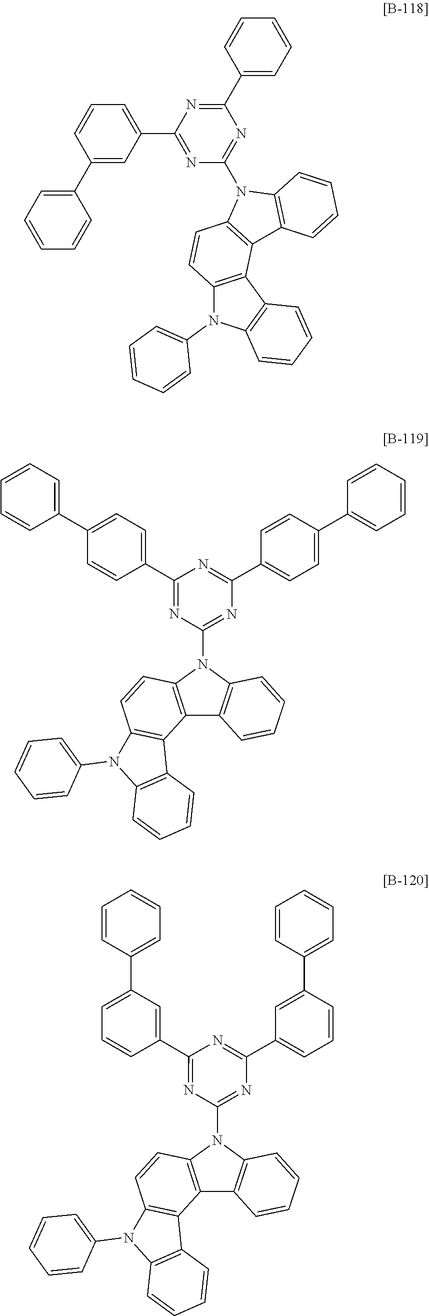 [SMILES: c1ccc(-c2cccc(-c3nc(-c4ccccc4)nc(-n4c5ccccc5c5c6c7ccccc7n(-c7ccccc7)c6ccc54)n3)c2)cc1 ; c1ccc(-c2ccc(-c3nc(-c4ccc(-c5ccccc5)cc4)nc(-n4c5ccccc5c5c6c7ccccc7n(-c7ccccc7)c6ccc54)n3)cc2)cc1 ; c1ccc(-c2cccc(-c3nc(-c4cccc(-c5ccccc5)c4)nc(-n4c5ccccc5c5c6c7ccccc7n(-c7ccccc7)c6ccc54)n3)c2)cc1]

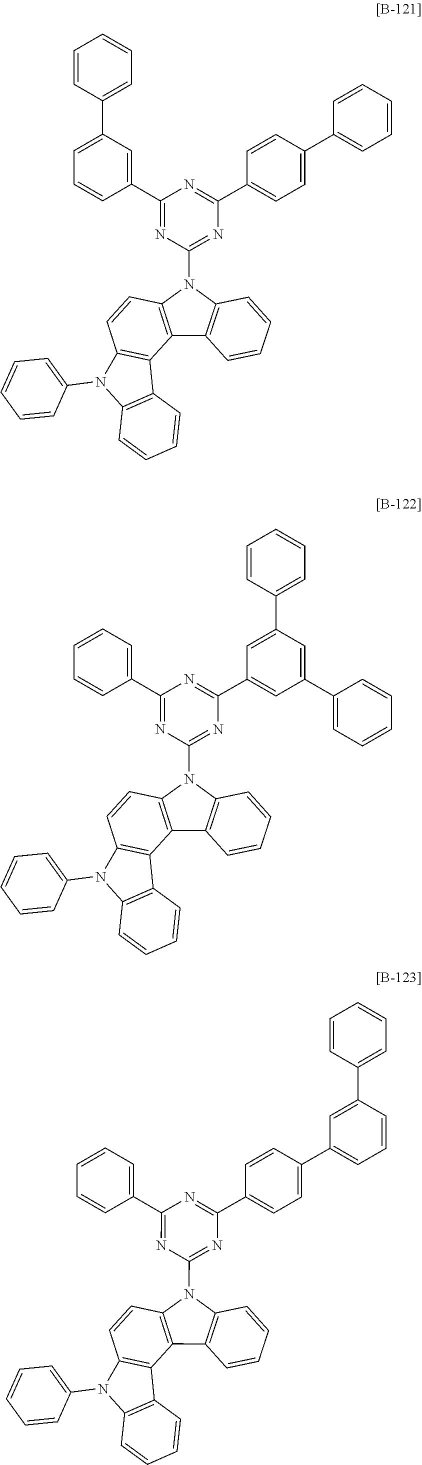 [SMILES: c1ccc(-c2ccc(-c3nc(-c4cccc(-c5ccccc5)c4)nc(-n4c5ccccc5c5c6c7ccccc7n(-c7ccccc7)c6ccc54)n3)cc2)cc1 ; c1ccc(-c2cc(-c3ccccc3)cc(-c3nc(-c4ccccc4)nc(-n4c5ccccc5c5c6c7ccccc7n(-c7ccccc7)c6ccc54)n3)c2)cc1 ; c1ccc(-c2cccc(-c3ccc(-c4nc(-c5ccccc5)nc(-n5c6ccccc6c6c7c8ccccc8n(-c8ccccc8)c7ccc65)n4)cc3)c2)cc1]

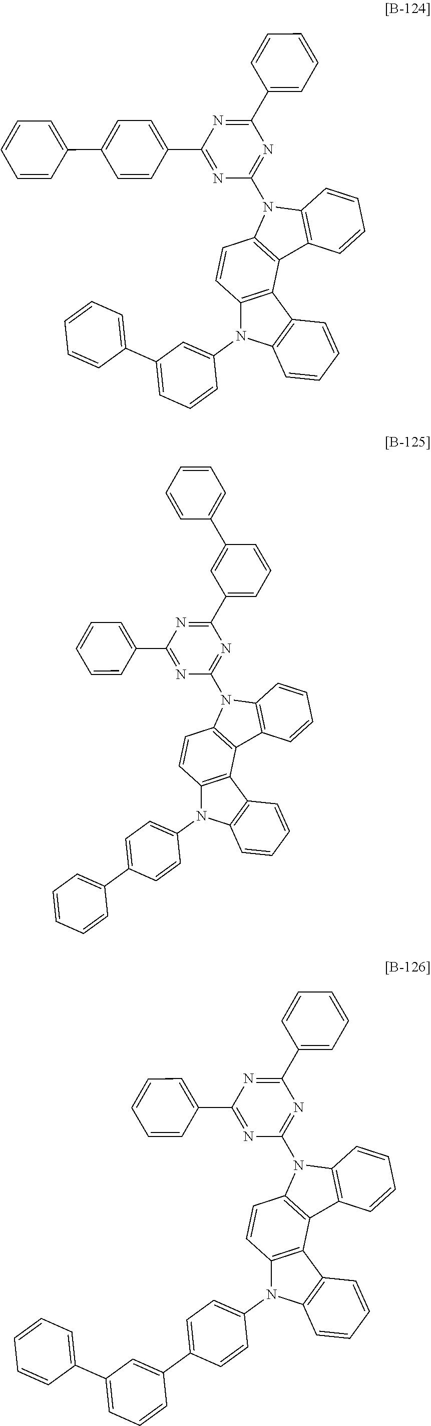 [SMILES: c1ccc(-c2ccc(-c3nc(-c4ccccc4)nc(-n4c5ccccc5c5c6c7ccccc7n(-c7cccc(-c8ccccc8)c7)c6ccc54)n3)cc2)cc1 ; c1ccc(-c2ccc(-n3c4ccccc4c4c5c6ccccc6n(-c6nc(-c7ccccc7)nc(-c7cccc(-c8ccccc8)c7)n6)c5ccc43)cc2)cc1 ; c1ccc(-c2cccc(-c3ccc(-n4c5ccccc5c5c6c7ccccc7n(-c7nc(-c8ccccc8)nc(-c8ccccc8)n7)c6ccc54)cc3)c2)cc1]

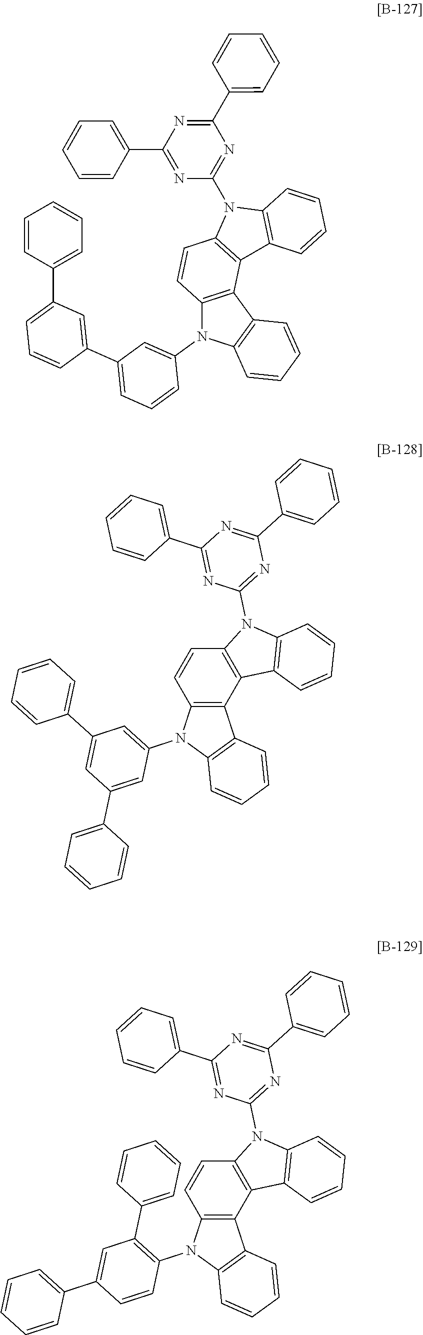 [SMILES: c1ccc(-c2cccc(-c3cccc(-n4c5ccccc5c5c6c7ccccc7n(-c7nc(-c8ccccc8)nc(-c8ccccc8)n7)c6ccc54)c3)c2)cc1 ; c1ccc(-c2cc(-c3ccccc3)cc(-n3c4ccccc4c4c5c6ccccc6n(-c6nc(-c7ccccc7)nc(-c7ccccc7)n6)c5ccc43)c2)cc1 ; c1ccc(-c2ccc(-n3c4ccccc4c4c5c6ccccc6n(-c6nc(-c7ccccc7)nc(-c7ccccc7)n6)c5ccc43)c(-c3ccccc3)c2)cc1]

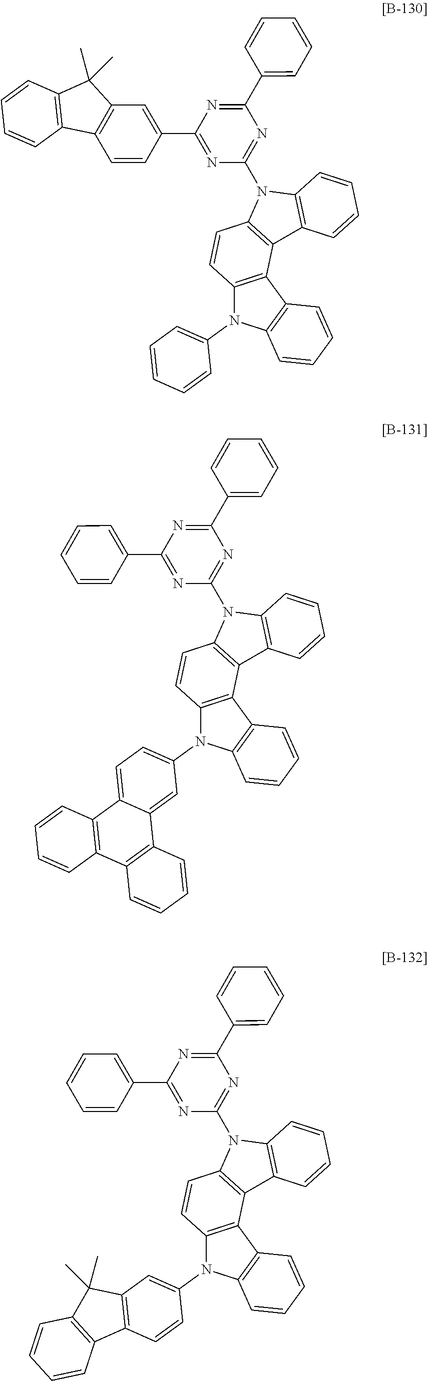 [SMILES: CC1(C)c2ccccc2-c2ccc(-c3nc(-c4ccccc4)nc(-n4c5ccccc5c5c6c7ccccc7n(-c7ccccc7)c6ccc54)n3)cc21 ; c1ccc(-c2nc(-c3ccccc3)nc(-n3c4ccccc4c4c5c6ccccc6n(-c6ccc7c8ccccc8c8ccccc8c7c6)c5ccc43)n2)cc1 ; CC1(C)c2ccccc2-c2ccc(-n3c4ccccc4c4c5c6ccccc6n(-c6nc(-c7ccccc7)nc(-c7ccccc7)n6)c5ccc43)cc21]

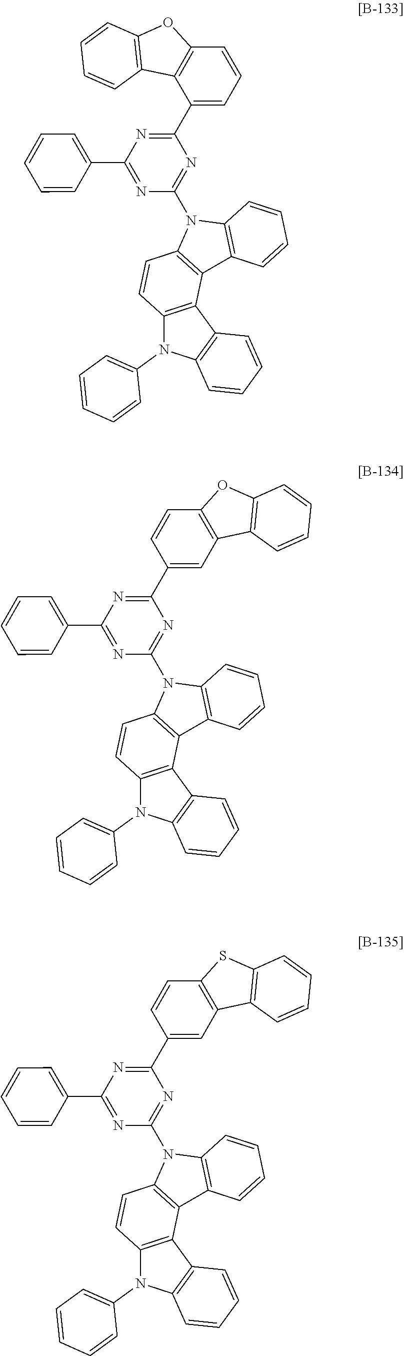 [SMILES: c1ccc(-c2nc(-c3cccc4oc5ccccc5c34)nc(-n3c4ccccc4c4c5c6ccccc6n(-c6ccccc6)c5ccc43)n2)cc1 ; c1ccc(-c2nc(-c3ccc4oc5ccccc5c4c3)nc(-n3c4ccccc4c4c5c6ccccc6n(-c6ccccc6)c5ccc43)n2)cc1 ; c1ccc(-c2nc(-c3ccc4sc5ccccc5c4c3)nc(-n3c4ccccc4c4c5c6ccccc6n(-c6ccccc6)c5ccc43)n2)cc1]

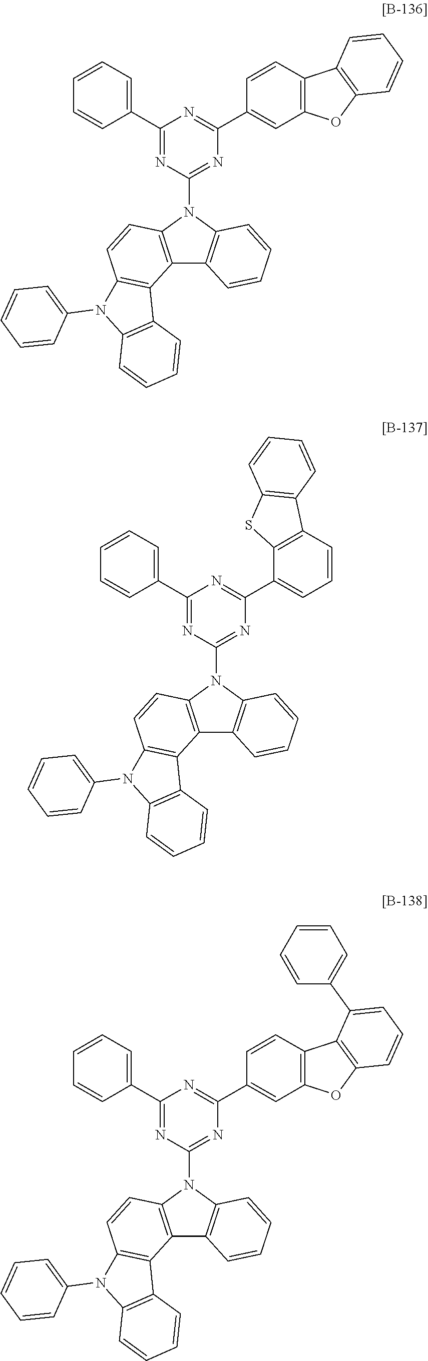 [SMILES: c1ccc(-c2nc(-c3ccc4c(c3)oc3ccccc34)nc(-n3c4ccccc4c4c5c6ccccc6n(-c6ccccc6)c5ccc43)n2)cc1 ; c1ccc(-c2nc(-c3cccc4c3sc3ccccc34)nc(-n3c4ccccc4c4c5c6ccccc6n(-c6ccccc6)c5ccc43)n2)cc1 ; c1ccc(-c2nc(-c3ccc4c(c3)oc3cccc(-c5ccccc5)c34)nc(-n3c4ccccc4c4c5c6ccccc6n(-c6ccccc6)c5ccc43)n2)cc1]

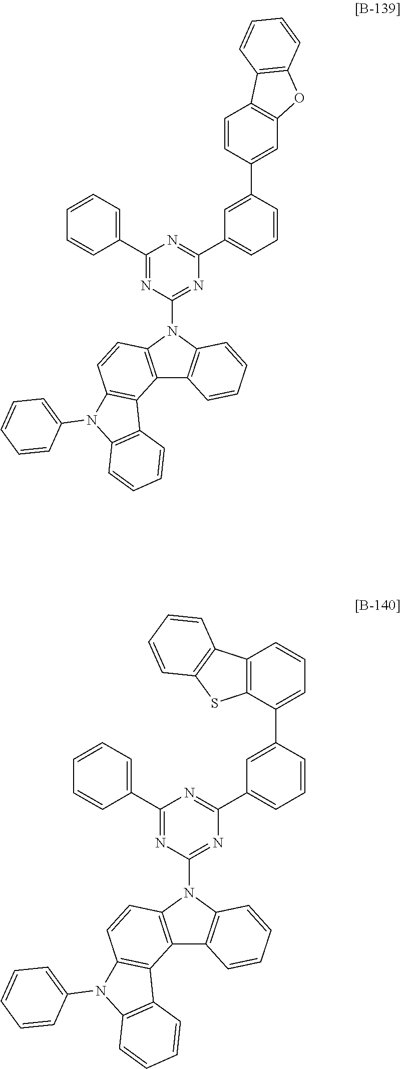 [SMILES: c1ccc(-c2nc(-c3cccc(-c4ccc5c(c4)oc4ccccc45)c3)nc(-n3c4ccccc4c4c5c6ccccc6n(-c6ccccc6)c5ccc43)n2)cc1 ; c1ccc(-c2nc(-c3cccc(-c4cccc5c4sc4ccccc45)c3)nc(-n3c4ccccc4c4c5c6ccccc6n(-c6ccccc6)c5ccc43)n2)cc1]

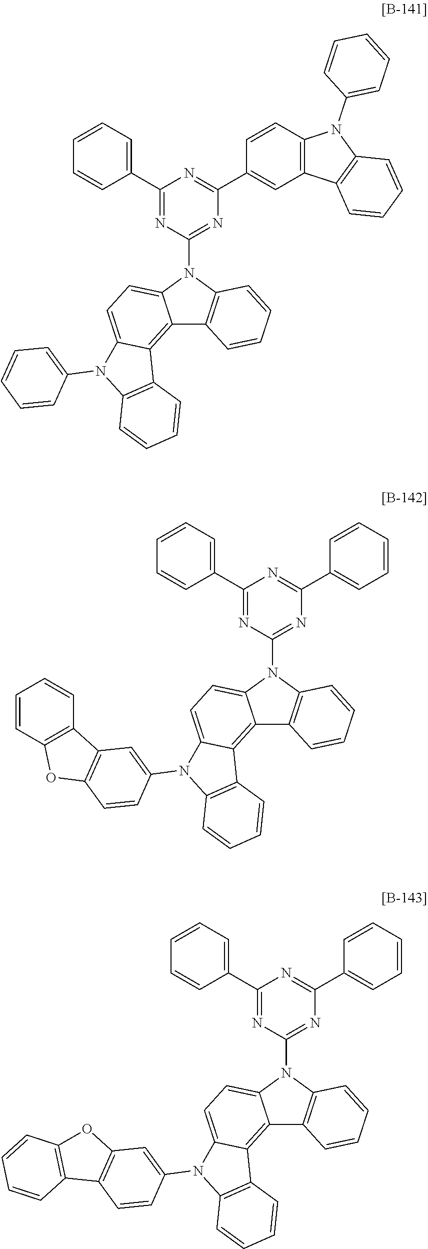 [SMILES: c1ccc(-c2nc(-c3ccc4c(c3)c3ccccc3n4-c3ccccc3)nc(-n3c4ccccc4c4c5c6ccccc6n(-c6ccccc6)c5ccc43)n2)cc1 ; c1ccc(-c2nc(-c3ccccc3)nc(-n3c4ccccc4c4c5c6ccccc6n(-c6ccc7oc8ccccc8c7c6)c5ccc43)n2)cc1 ; c1ccc(-c2nc(-c3ccccc3)nc(-n3c4ccccc4c4c5c6ccccc6n(-c6ccc7c(c6)oc6ccccc67)c5ccc43)n2)cc1]

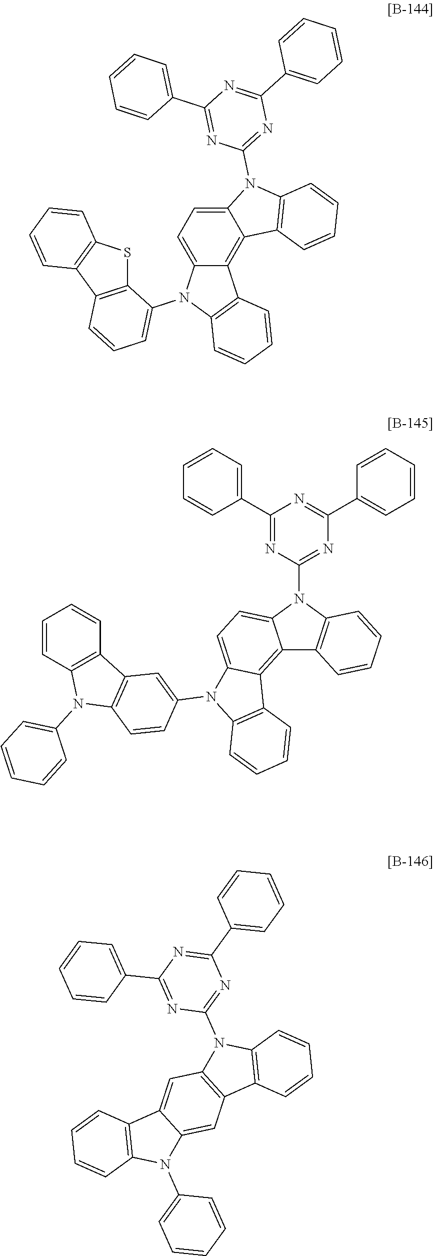 [SMILES: c1ccc(-c2nc(-c3ccccc3)nc(-n3c4ccccc4c4c5c6ccccc6n(-c6cccc7c6sc6ccccc67)c5ccc43)n2)cc1 ; c1ccc(-c2nc(-c3ccccc3)nc(-n3c4ccccc4c4c5c6ccccc6n(-c6ccc7c(c6)c6ccccc6n7-c6ccccc6)c5ccc43)n2)cc1 ; c1ccc(-c2nc(-c3ccccc3)nc(-n3c4ccccc4c4cc5c(cc43)c3ccccc3n5-c3ccccc3)n2)cc1]

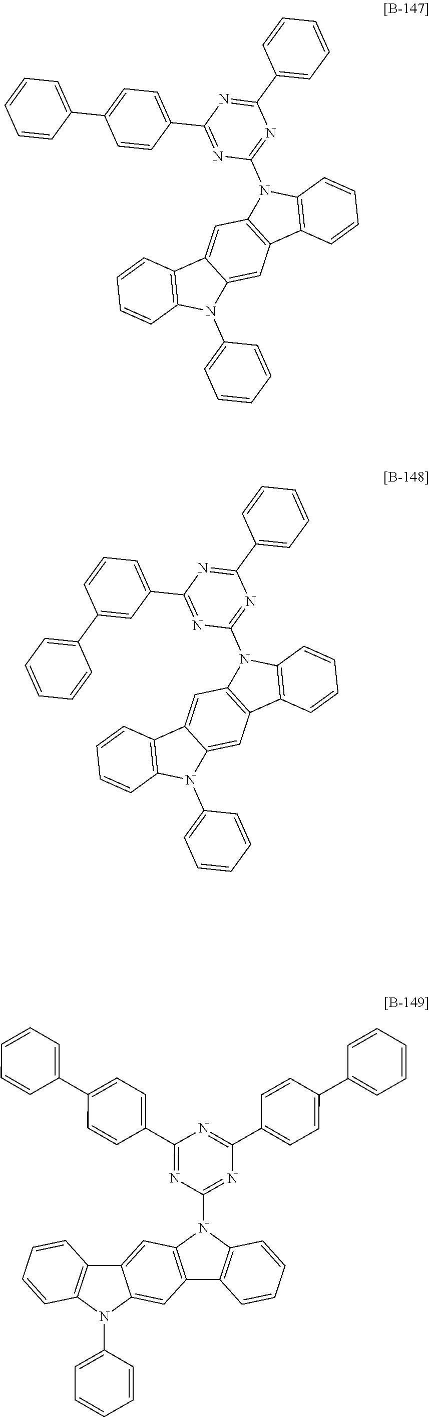 [SMILES: c1ccc(-c2ccc(-c3nc(-c4ccccc4)nc(-n4c5ccccc5c5cc6c(cc54)c4ccccc4n6-c4ccccc4)n3)cc2)cc1 ; c1ccc(-c2cccc(-c3nc(-c4ccccc4)nc(-n4c5ccccc5c5cc6c(cc54)c4ccccc4n6-c4ccccc4)n3)c2)cc1 ; c1ccc(-c2ccc(-c3nc(-c4ccc(-c5ccccc5)cc4)nc(-n4c5ccccc5c5cc6c(cc54)c4ccccc4n6-c4ccccc4)n3)cc2)cc1]

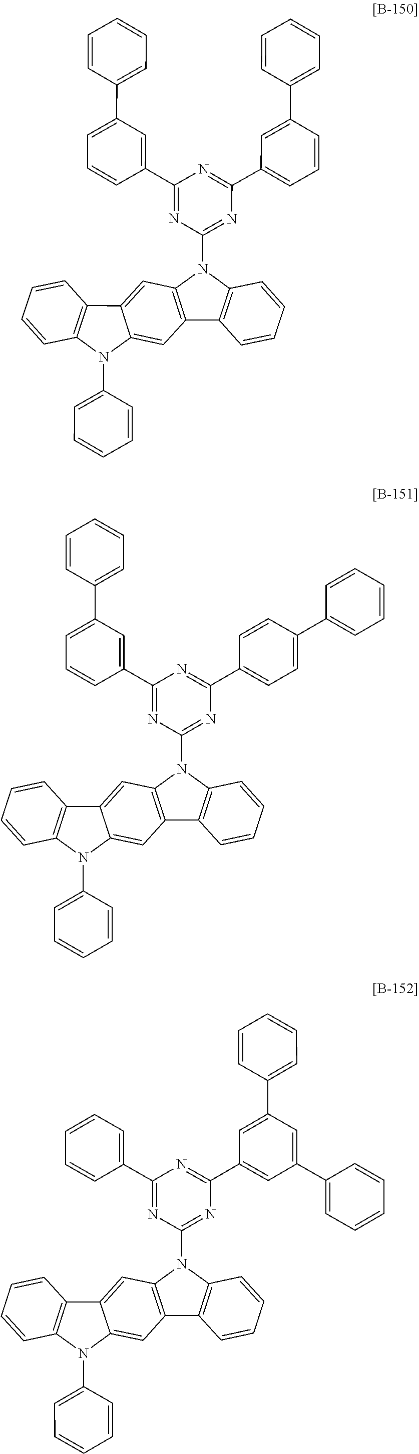 [SMILES: c1ccc(-c2cccc(-c3nc(-c4cccc(-c5ccccc5)c4)nc(-n4c5ccccc5c5cc6c(cc54)c4ccccc4n6-c4ccccc4)n3)c2)cc1 ; c1ccc(-c2ccc(-c3nc(-c4cccc(-c5ccccc5)c4)nc(-n4c5ccccc5c5cc6c(cc54)c4ccccc4n6-c4ccccc4)n3)cc2)cc1 ; c1ccc(-c2cc(-c3ccccc3)cc(-c3nc(-c4ccccc4)nc(-n4c5ccccc5c5cc6c(cc54)c4ccccc4n6-c4ccccc4)n3)c2)cc1]

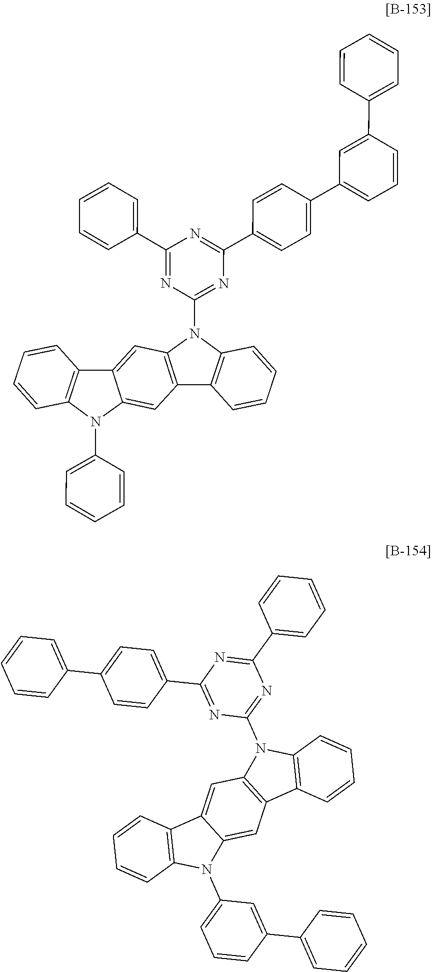 [SMILES: c1ccc(-c2cccc(-c3ccc(-c4nc(-c5ccccc5)nc(-n5c6ccccc6c6cc7c(cc65)c5ccccc5n7-c5ccccc5)n4)cc3)c2)cc1 ; c1ccc(-c2ccc(-c3nc(-c4ccccc4)nc(-n4c5ccccc5c5cc6c(cc54)c4ccccc4n6-c4cccc(-c5ccccc5)c4)n3)cc2)cc1]

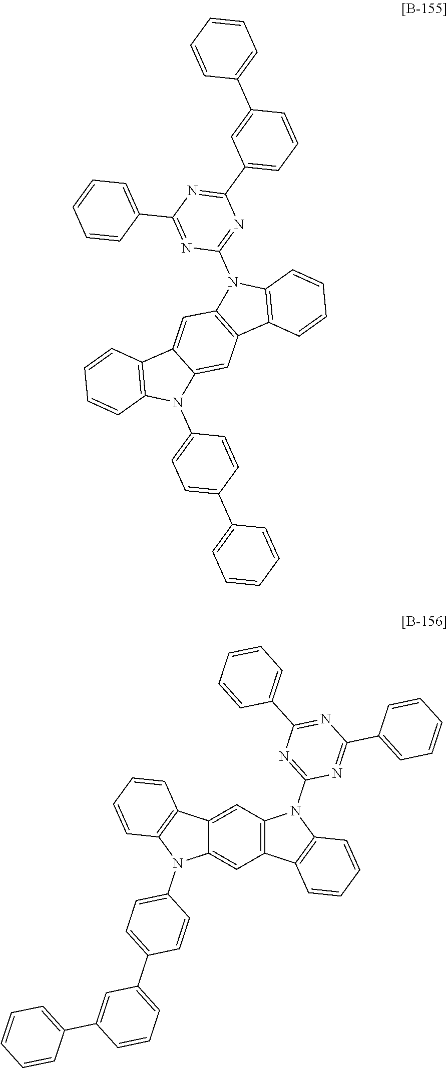 [SMILES: c1ccc(-c2ccc(-n3c4ccccc4c4cc5c(cc43)c3ccccc3n5-c3nc(-c4ccccc4)nc(-c4cccc(-c5ccccc5)c4)n3)cc2)cc1 ; c1ccc(-c2cccc(-c3ccc(-n4c5ccccc5c5cc6c(cc54)c4ccccc4n6-c4nc(-c5ccccc5)nc(-c5ccccc5)n4)cc3)c2)cc1]

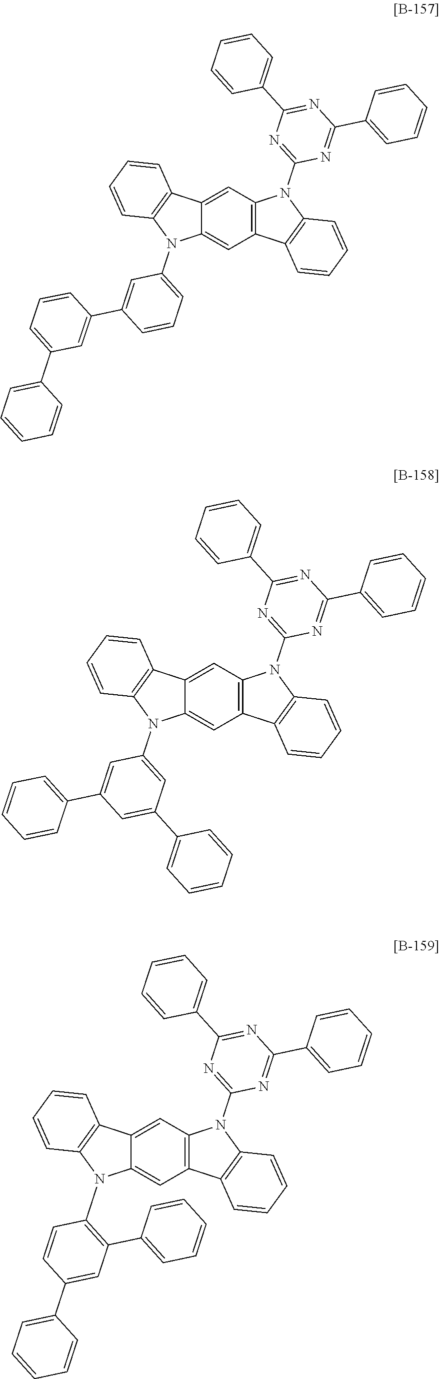 [SMILES: c1ccc(-c2cccc(-c3cccc(-n4c5ccccc5c5cc6c(cc54)c4ccccc4n6-c4nc(-c5ccccc5)nc(-c5ccccc5)n4)c3)c2)cc1 ; c1ccc(-c2cc(-c3ccccc3)cc(-n3c4ccccc4c4cc5c(cc43)c3ccccc3n5-c3nc(-c4ccccc4)nc(-c4ccccc4)n3)c2)cc1 ; c1ccc(-c2ccc(-n3c4ccccc4c4cc5c(cc43)c3ccccc3n5-c3nc(-c4ccccc4)nc(-c4ccccc4)n3)c(-c3ccccc3)c2)cc1]

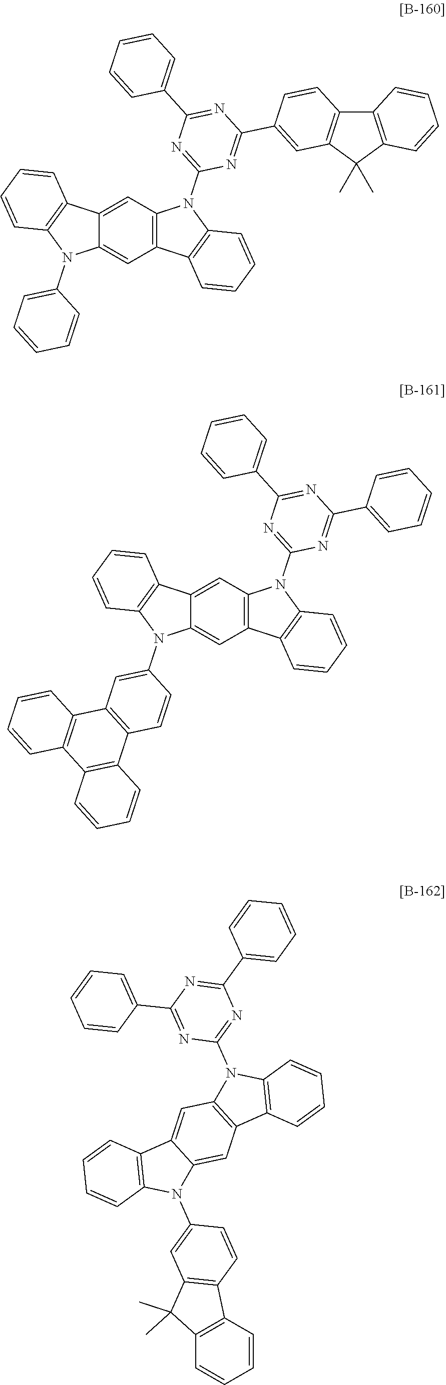 [SMILES: CC1(C)c2ccccc2-c2ccc(-c3nc(-c4ccccc4)nc(-n4c5ccccc5c5cc6c(cc54)c4ccccc4n6-c4ccccc4)n3)cc21 ; c1ccc(-c2nc(-c3ccccc3)nc(-n3c4ccccc4c4cc5c(cc43)c3ccccc3n5-c3ccc4c5ccccc5c5ccccc5c4c3)n2)cc1 ; CC1(C)c2ccccc2-c2ccc(-n3c4ccccc4c4cc5c(cc43)c3ccccc3n5-c3nc(-c4ccccc4)nc(-c4ccccc4)n3)cc21]

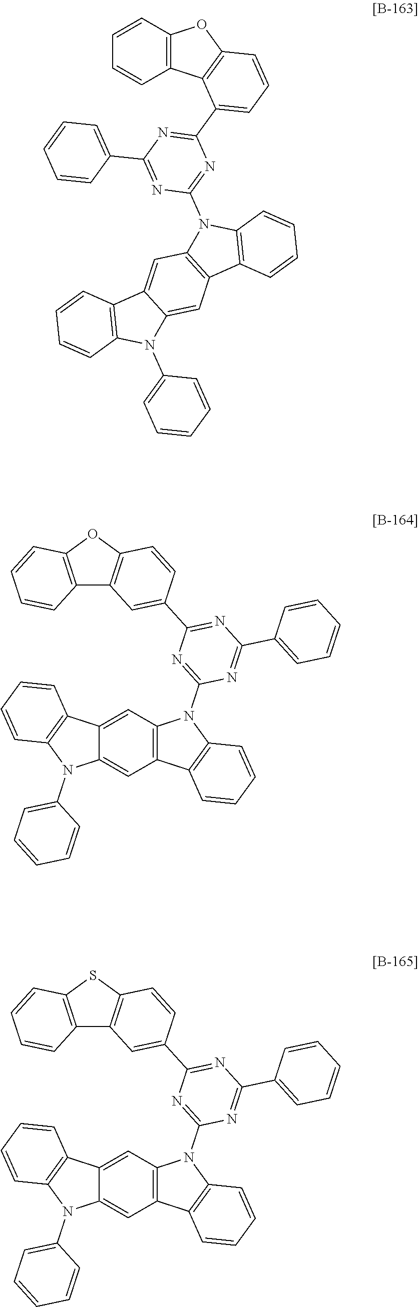 [SMILES: c1ccc(-c2nc(-c3cccc4oc5ccccc5c34)nc(-n3c4ccccc4c4cc5c(cc43)c3ccccc3n5-c3ccccc3)n2)cc1 ; c1ccc(-c2nc(-c3ccc4oc5ccccc5c4c3)nc(-n3c4ccccc4c4cc5c(cc43)c3ccccc3n5-c3ccccc3)n2)cc1 ; c1ccc(-c2nc(-c3ccc4sc5ccccc5c4c3)nc(-n3c4ccccc4c4cc5c(cc43)c3ccccc3n5-c3ccccc3)n2)cc1]

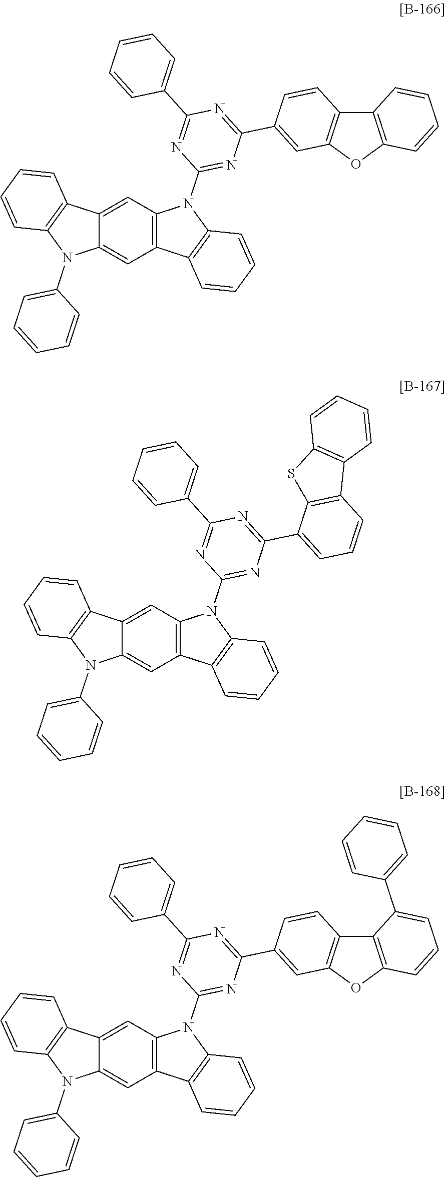 [SMILES: c1ccc(-c2nc(-c3ccc4c(c3)oc3ccccc34)nc(-n3c4ccccc4c4cc5c(cc43)c3ccccc3n5-c3ccccc3)n2)cc1 ; c1ccc(-c2nc(-c3cccc4c3sc3ccccc34)nc(-n3c4ccccc4c4cc5c(cc43)c3ccccc3n5-c3ccccc3)n2)cc1 ; c1ccc(-c2nc(-c3ccc4c(c3)oc3cccc(-c5ccccc5)c34)nc(-n3c4ccccc4c4cc5c(cc43)c3ccccc3n5-c3ccccc3)n2)cc1]

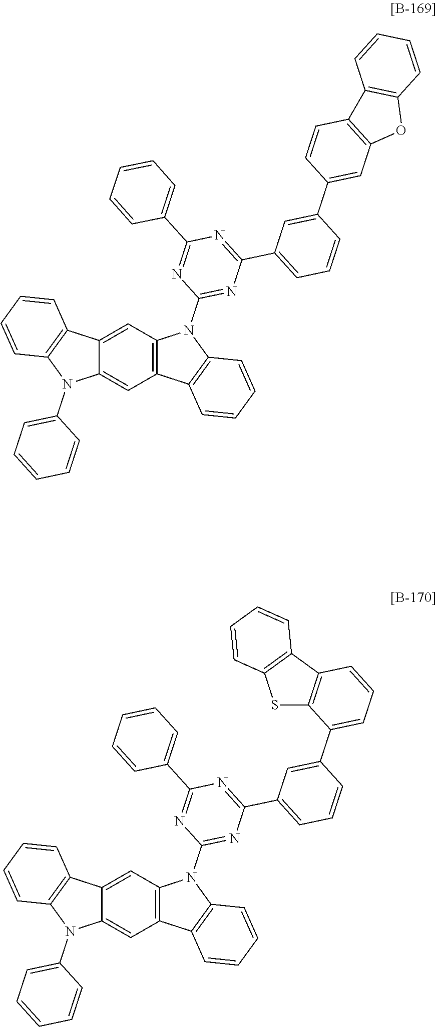 [SMILES: c1ccc(-c2nc(-c3cccc(-c4ccc5c(c4)oc4ccccc45)c3)nc(-n3c4ccccc4c4cc5c(cc43)c3ccccc3n5-c3ccccc3)n2)cc1 ; c1ccc(-c2nc(-c3cccc(-c4cccc5c4sc4ccccc45)c3)nc(-n3c4ccccc4c4cc5c(cc43)c3ccccc3n5-c3ccccc3)n2)cc1]

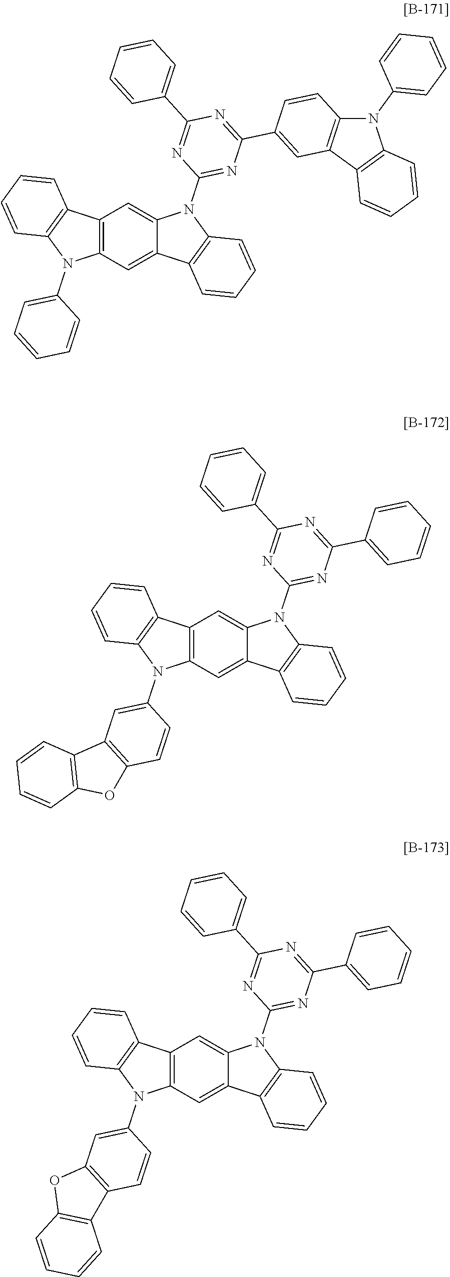 [SMILES: c1ccc(-c2nc(-c3ccc4c(c3)c3ccccc3n4-c3ccccc3)nc(-n3c4ccccc4c4cc5c(cc43)c3ccccc3n5-c3ccccc3)n2)cc1 ; c1ccc(-c2nc(-c3ccccc3)nc(-n3c4ccccc4c4cc5c(cc43)c3ccccc3n5-c3ccc4oc5ccccc5c4c3)n2)cc1 ; c1ccc(-c2nc(-c3ccccc3)nc(-n3c4ccccc4c4cc5c(cc43)c3ccccc3n5-c3ccc4c(c3)oc3ccccc34)n2)cc1]

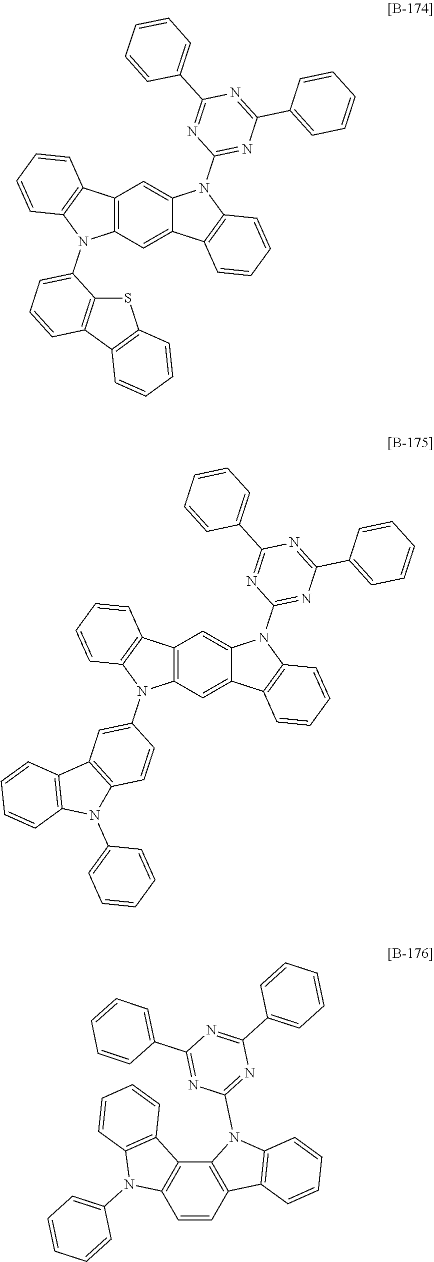 [SMILES: c1ccc(-c2nc(-c3ccccc3)nc(-n3c4ccccc4c4cc5c(cc43)c3ccccc3n5-c3cccc4c3sc3ccccc34)n2)cc1 ; c1ccc(-c2nc(-c3ccccc3)nc(-n3c4ccccc4c4cc5c(cc43)c3ccccc3n5-c3ccc4c(c3)c3ccccc3n4-c3ccccc3)n2)cc1 ; c1ccc(-c2nc(-c3ccccc3)nc(-n3c4ccccc4c4ccc5c(c6ccccc6n5-c5ccccc5)c43)n2)cc1]

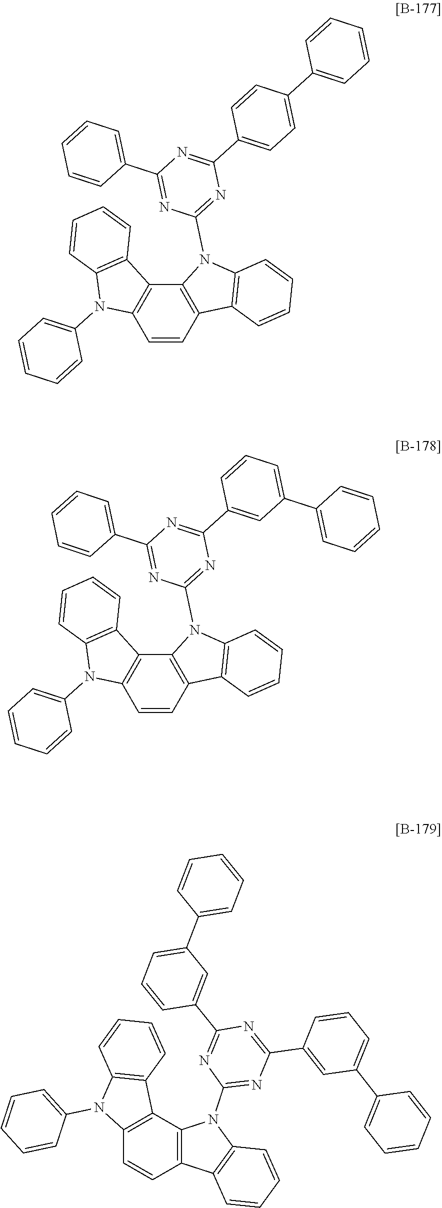 [SMILES: c1ccc(-c2ccc(-c3nc(-c4ccccc4)nc(-n4c5ccccc5c5ccc6c(c7ccccc7n6-c6ccccc6)c54)n3)cc2)cc1 ; c1ccc(-c2cccc(-c3nc(-c4ccccc4)nc(-n4c5ccccc5c5ccc6c(c7ccccc7n6-c6ccccc6)c54)n3)c2)cc1 ; c1ccc(-c2cccc(-c3nc(-c4cccc(-c5ccccc5)c4)nc(-n4c5ccccc5c5ccc6c(c7ccccc7n6-c6ccccc6)c54)n3)c2)cc1]

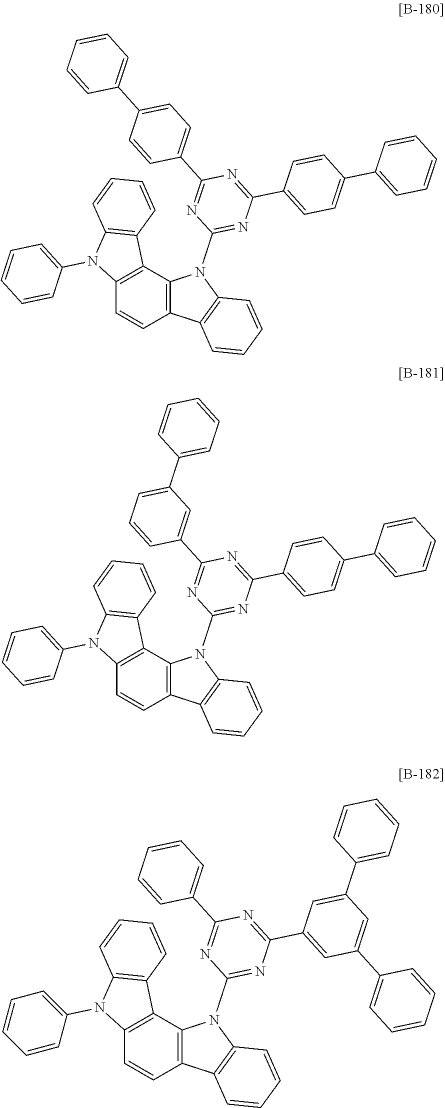 [SMILES: c1ccc(-c2ccc(-c3nc(-c4ccc(-c5ccccc5)cc4)nc(-n4c5ccccc5c5ccc6c(c7ccccc7n6-c6ccccc6)c54)n3)cc2)cc1 ; c1ccc(-c2ccc(-c3nc(-c4cccc(-c5ccccc5)c4)nc(-n4c5ccccc5c5ccc6c(c7ccccc7n6-c6ccccc6)c54)n3)cc2)cc1 ; c1ccc(-c2cc(-c3ccccc3)cc(-c3nc(-c4ccccc4)nc(-n4c5ccccc5c5ccc6c(c7ccccc7n6-c6ccccc6)c54)n3)c2)cc1]

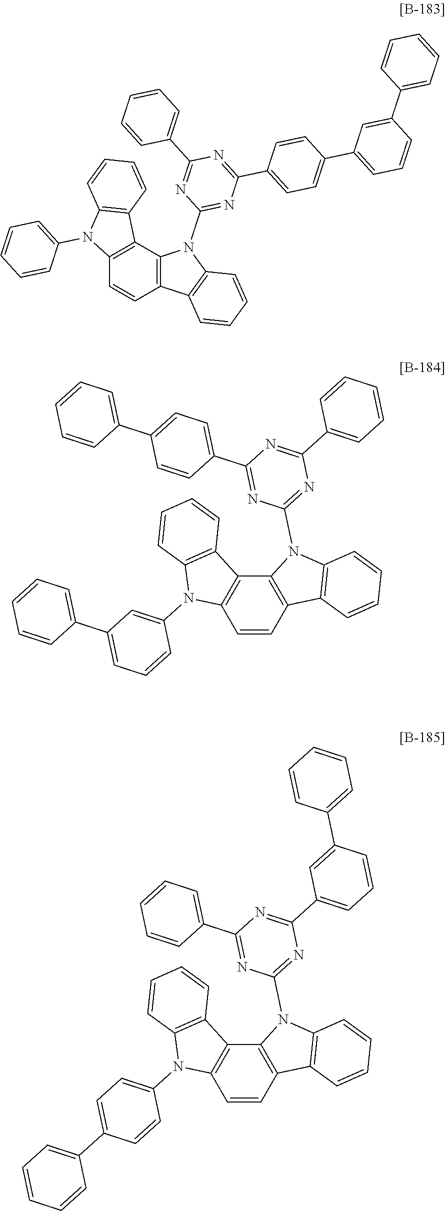

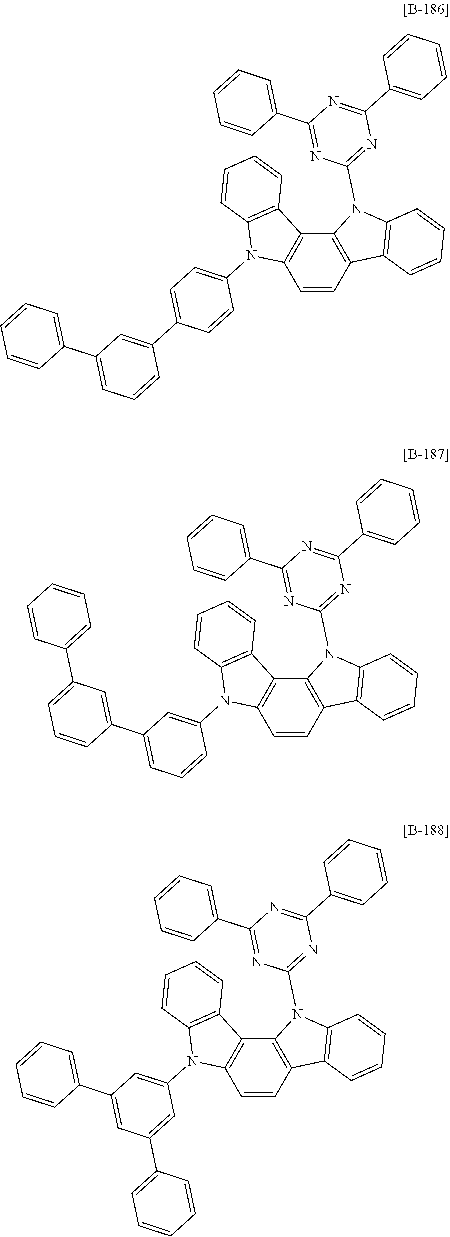 [SMILES: c1ccc(-c2cccc(-c3ccc(-n4c5ccccc5c5c4ccc4c6ccccc6n(-c6nc(-c7ccccc7)nc(-c7ccccc7)n6)c45)cc3)c2)cc1 ; c1ccc(-c2cccc(-c3cccc(-n4c5ccccc5c5c4ccc4c6ccccc6n(-c6nc(-c7ccccc7)nc(-c7ccccc7)n6)c45)c3)c2)cc1 ; c1ccc(-c2cc(-c3ccccc3)cc(-n3c4ccccc4c4c3ccc3c5ccccc5n(-c5nc(-c6ccccc6)nc(-c6ccccc6)n5)c34)c2)cc1]

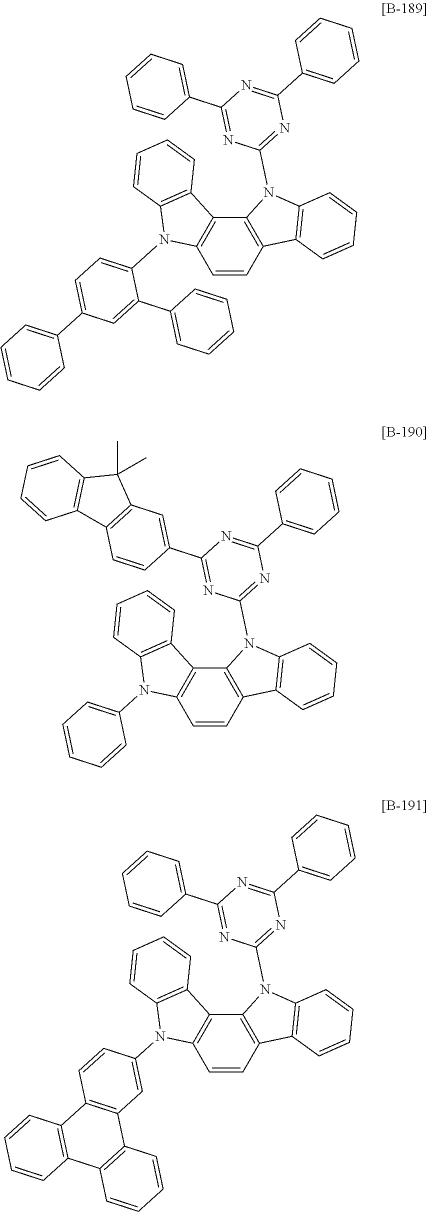 [SMILES: c1ccc(-c2ccc(-n3c4ccccc4c4c3ccc3c5ccccc5n(-c5nc(-c6ccccc6)nc(-c6ccccc6)n5)c34)c(-c3ccccc3)c2)cc1 ; CC1(C)c2ccccc2-c2ccc(-c3nc(-c4ccccc4)nc(-n4c5ccccc5c5ccc6c(c7ccccc7n6-c6ccccc6)c54)n3)cc21 ; c1ccc(-c2nc(-c3ccccc3)nc(-n3c4ccccc4c4ccc5c(c6ccccc6n5-c5ccc6c7ccccc7c7ccccc7c6c5)c43)n2)cc1]

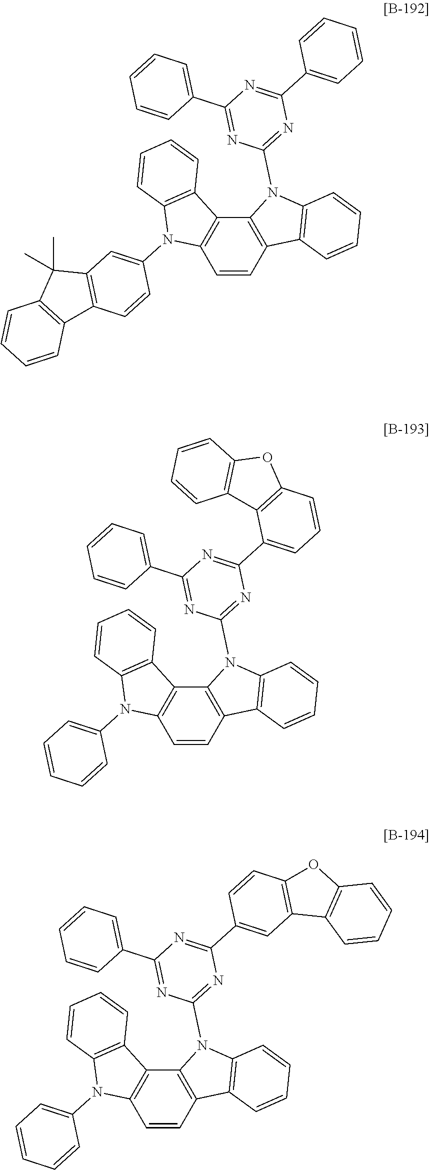 [SMILES: CC1(C)c2ccccc2-c2ccc(-n3c4ccccc4c4c3ccc3c5ccccc5n(-c5nc(-c6ccccc6)nc(-c6ccccc6)n5)c34)cc21 ; c1ccc(-c2nc(-c3cccc4oc5ccccc5c34)nc(-n3c4ccccc4c4ccc5c(c6ccccc6n5-c5ccccc5)c43)n2)cc1 ; c1ccc(-c2nc(-c3ccc4oc5ccccc5c4c3)nc(-n3c4ccccc4c4ccc5c(c6ccccc6n5-c5ccccc5)c43)n2)cc1]

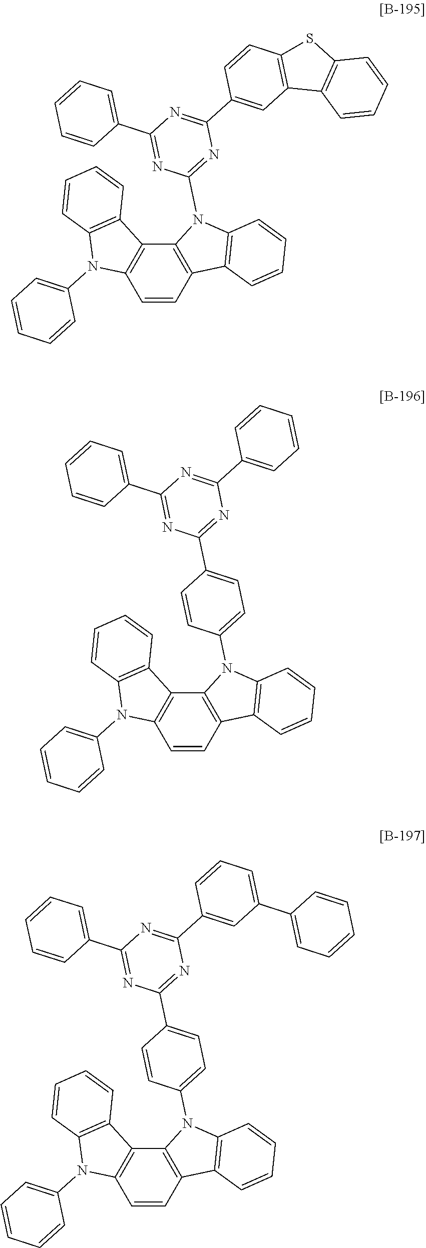 [SMILES: c1ccc(-c2nc(-c3ccc4sc5ccccc5c4c3)nc(-n3c4ccccc4c4ccc5c(c6ccccc6n5-c5ccccc5)c43)n2)cc1 ; c1ccc(-c2nc(-c3ccccc3)nc(-c3ccc(-n4c5ccccc5c5ccc6c(c7ccccc7n6-c6ccccc6)c54)cc3)n2)cc1 ; c1ccc(-c2cccc(-c3nc(-c4ccccc4)nc(-c4ccc(-n5c6ccccc6c6ccc7c(c8ccccc8n7-c7ccccc7)c65)cc4)n3)c2)cc1]

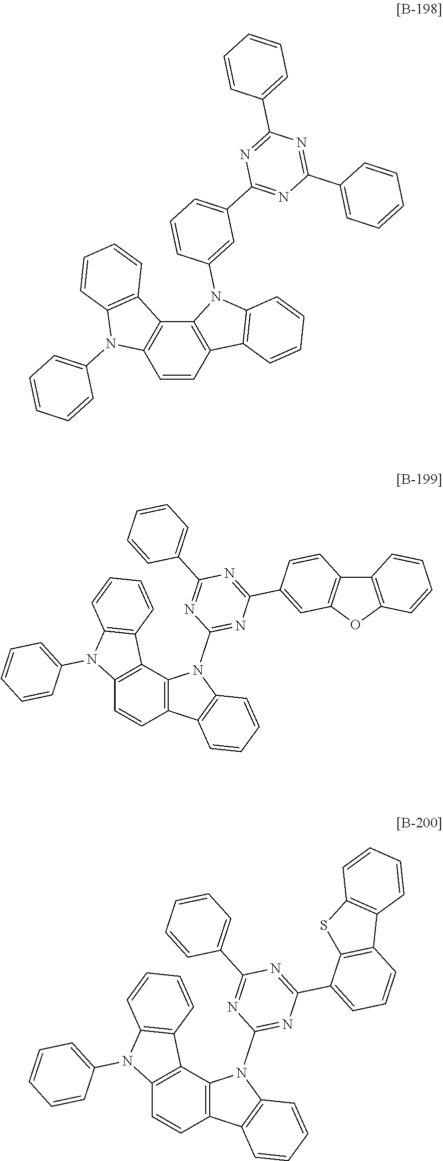 [SMILES: c1ccc(-c2nc(-c3ccccc3)nc(-c3cccc(-n4c5ccccc5c5ccc6c(c7ccccc7n6-c6ccccc6)c54)c3)n2)cc1 ; c1ccc(-c2nc(-c3ccc4c(c3)oc3ccccc34)nc(-n3c4ccccc4c4ccc5c(c6ccccc6n5-c5ccccc5)c43)n2)cc1 ; c1ccc(-c2nc(-c3cccc4c3sc3ccccc34)nc(-n3c4ccccc4c4ccc5c(c6ccccc6n5-c5ccccc5)c43)n2)cc1]

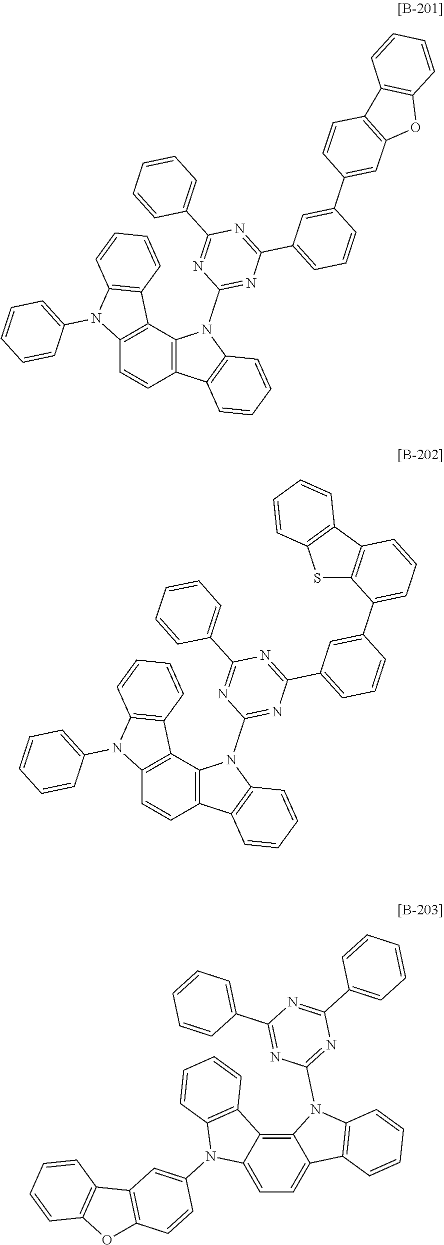 [SMILES: c1ccc(-c2nc(-c3cccc(-c4ccc5c(c4)oc4ccccc45)c3)nc(-n3c4ccccc4c4ccc5c(c6ccccc6n5-c5ccccc5)c43)n2)cc1 ; c1ccc(-c2nc(-c3cccc(-c4cccc5c4sc4ccccc45)c3)nc(-n3c4ccccc4c4ccc5c(c6ccccc6n5-c5ccccc5)c43)n2)cc1 ; c1ccc(-c2nc(-c3ccccc3)nc(-n3c4ccccc4c4ccc5c(c6ccccc6n5-c5ccc6oc7ccccc7c6c5)c43)n2)cc1]

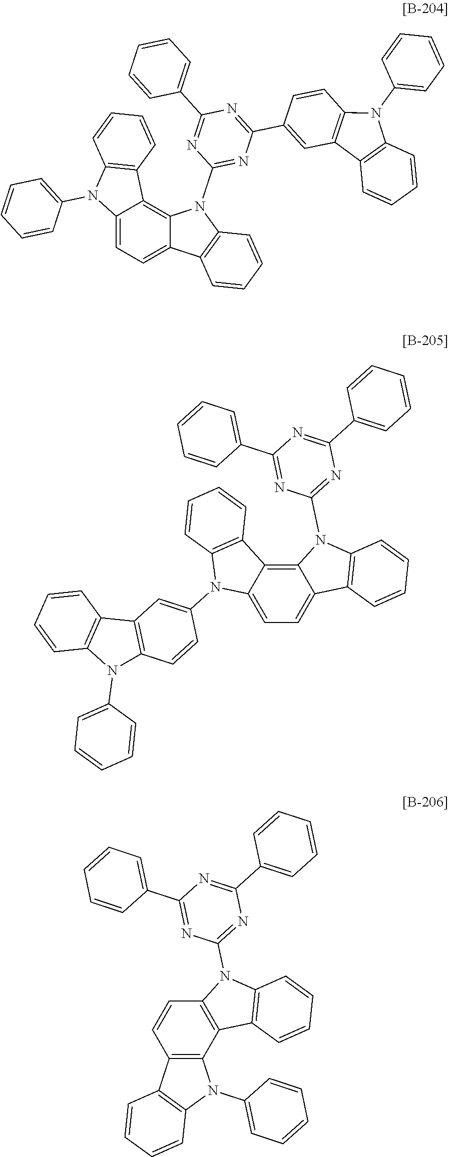 [SMILES: c1ccc(-c2nc(-c3ccc4c(c3)c3ccccc3n4-c3ccccc3)nc(-n3c4ccccc4c4ccc5c(c6ccccc6n5-c5ccccc5)c43)n2)cc1 ; c1ccc(-c2nc(-c3ccccc3)nc(-n3c4ccccc4c4ccc5c(c6ccccc6n5-c5ccc6c(c5)c5ccccc5n6-c5ccccc5)c43)n2)cc1 ; c1ccc(-c2nc(-c3ccccc3)nc(-n3c4ccccc4c4c3ccc3c5ccccc5n(-c5ccccc5)c34)n2)cc1]

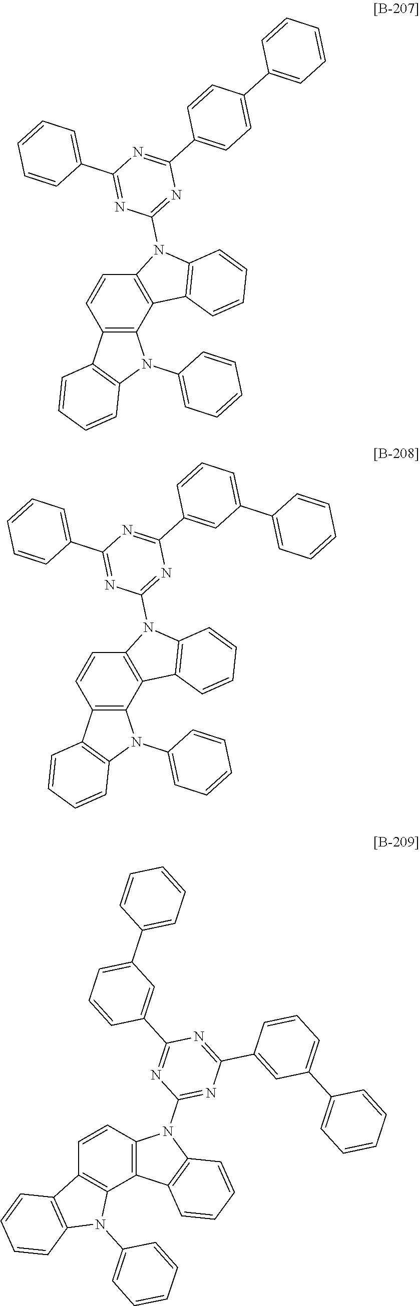 [SMILES: c1ccc(-c2ccc(-c3nc(-c4ccccc4)nc(-n4c5ccccc5c5c4ccc4c6ccccc6n(-c6ccccc6)c45)n3)cc2)cc1 ; c1ccc(-c2cccc(-c3nc(-c4ccccc4)nc(-n4c5ccccc5c5c4ccc4c6ccccc6n(-c6ccccc6)c45)n3)c2)cc1 ; c1ccc(-c2cccc(-c3nc(-c4cccc(-c5ccccc5)c4)nc(-n4c5ccccc5c5c4ccc4c6ccccc6n(-c6ccccc6)c45)n3)c2)cc1]

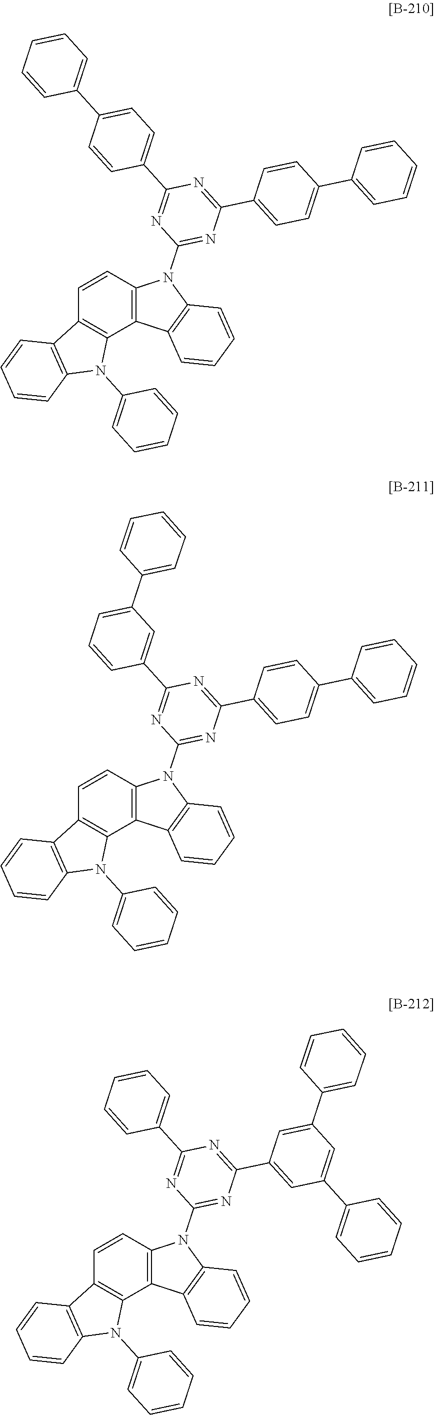 [SMILES: c1ccc(-c2ccc(-c3nc(-c4ccc(-c5ccccc5)cc4)nc(-n4c5ccccc5c5c4ccc4c6ccccc6n(-c6ccccc6)c45)n3)cc2)cc1 ; c1ccc(-c2ccc(-c3nc(-c4cccc(-c5ccccc5)c4)nc(-n4c5ccccc5c5c4ccc4c6ccccc6n(-c6ccccc6)c45)n3)cc2)cc1 ; c1ccc(-c2cc(-c3ccccc3)cc(-c3nc(-c4ccccc4)nc(-n4c5ccccc5c5c4ccc4c6ccccc6n(-c6ccccc6)c45)n3)c2)cc1]

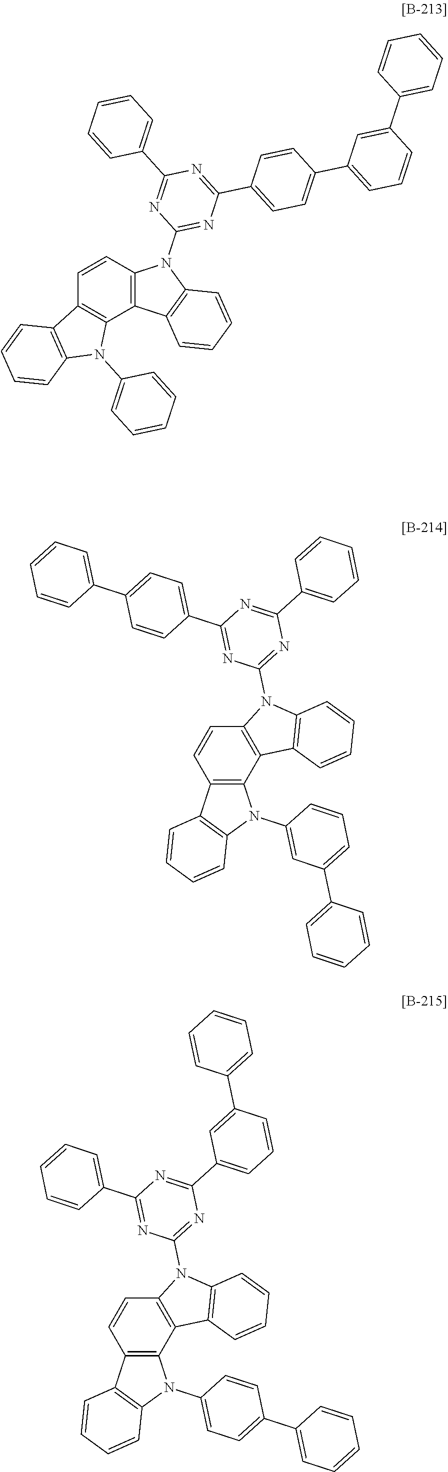 [SMILES: c1ccc(-c2cccc(-c3ccc(-c4nc(-c5ccccc5)nc(-n5c6ccccc6c6c5ccc5c7ccccc7n(-c7ccccc7)c56)n4)cc3)c2)cc1 ; c1ccc(-c2ccc(-c3nc(-c4ccccc4)nc(-n4c5ccccc5c5c4ccc4c6ccccc6n(-c6cccc(-c7ccccc7)c6)c45)n3)cc2)cc1 ; c1ccc(-c2ccc(-n3c4ccccc4c4ccc5c(c6ccccc6n5-c5nc(-c6ccccc6)nc(-c6cccc(-c7ccccc7)c6)n5)c43)cc2)cc1]

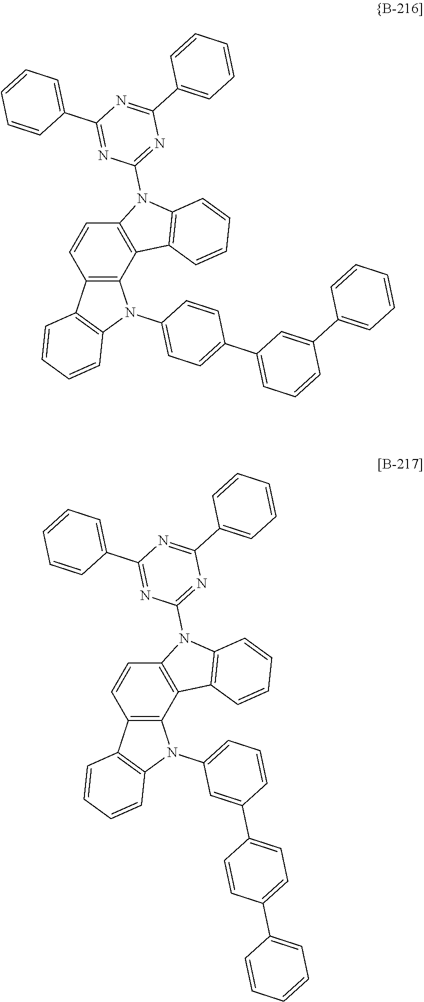 [SMILES: c1ccc(-c2cccc(-c3ccc(-n4c5ccccc5c5ccc6c(c7ccccc7n6-c6nc(-c7ccccc7)nc(-c7ccccc7)n6)c54)cc3)c2)cc1 ; c1ccc(-c2ccc(-c3cccc(-n4c5ccccc5c5ccc6c(c7ccccc7n6-c6nc(-c7ccccc7)nc(-c7ccccc7)n6)c54)c3)cc2)cc1]

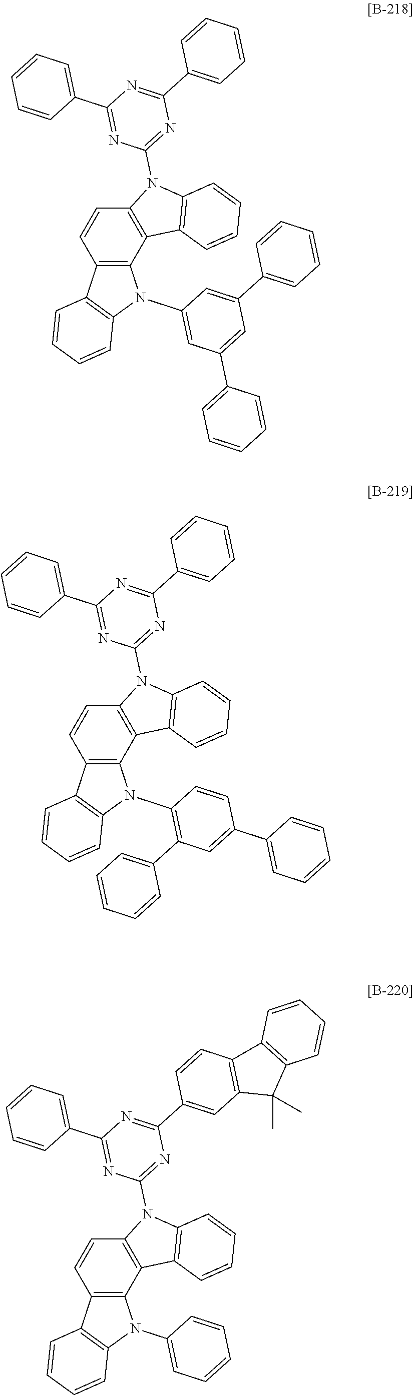 [SMILES: c1ccc(-c2cc(-c3ccccc3)cc(-n3c4ccccc4c4ccc5c(c6ccccc6n5-c5nc(-c6ccccc6)nc(-c6ccccc6)n5)c43)c2)cc1 ; c1ccc(-c2ccc(-n3c4ccccc4c4ccc5c(c6ccccc6n5-c5nc(-c6ccccc6)nc(-c6ccccc6)n5)c43)c(-c3ccccc3)c2)cc1 ; CC1(C)c2ccccc2-c2ccc(-c3nc(-c4ccccc4)nc(-n4c5ccccc5c5c4ccc4c6ccccc6n(-c6ccccc6)c45)n3)cc21]

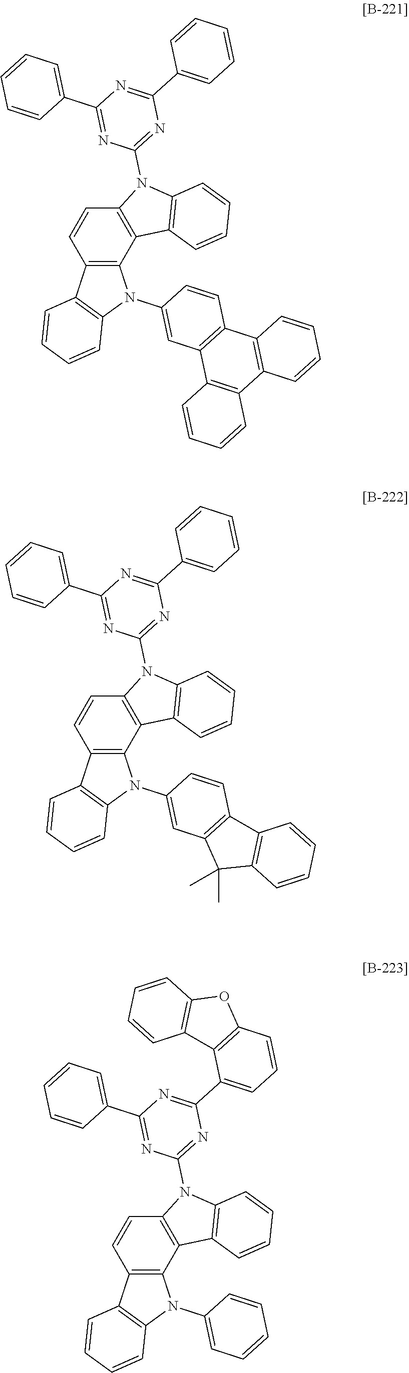 [SMILES: c1ccc(-c2nc(-c3ccccc3)nc(-n3c4ccccc4c4c3ccc3c5ccccc5n(-c5ccc6c7ccccc7c7ccccc7c6c5)c34)n2)cc1 ; CC1(C)c2ccccc2-c2ccc(-n3c4ccccc4c4ccc5c(c6ccccc6n5-c5nc(-c6ccccc6)nc(-c6ccccc6)n5)c43)cc21 ; c1ccc(-c2nc(-c3cccc4oc5ccccc5c34)nc(-n3c4ccccc4c4c3ccc3c5ccccc5n(-c5ccccc5)c34)n2)cc1]

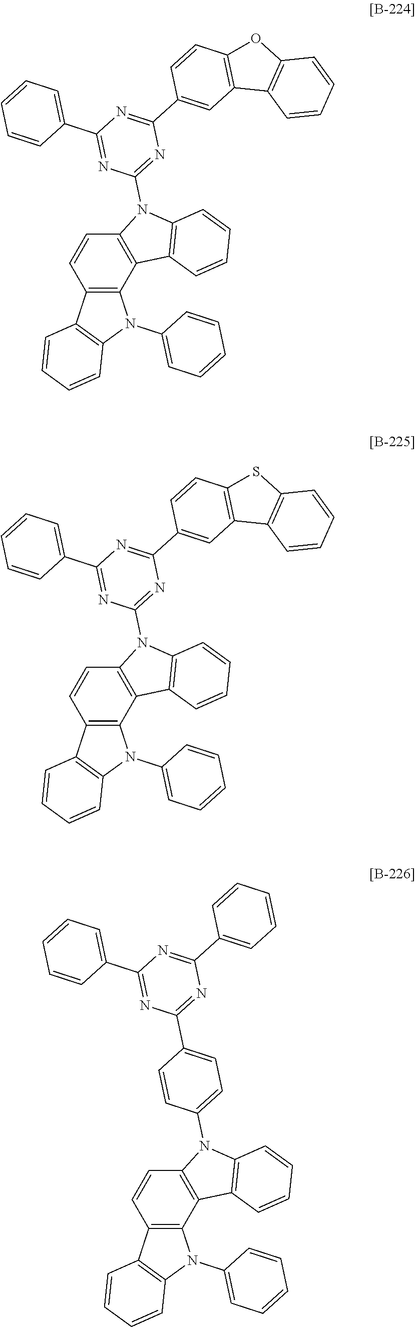 [SMILES: c1ccc(-c2nc(-c3ccc4oc5ccccc5c4c3)nc(-n3c4ccccc4c4c3ccc3c5ccccc5n(-c5ccccc5)c34)n2)cc1 ; c1ccc(-c2nc(-c3ccc4sc5ccccc5c4c3)nc(-n3c4ccccc4c4c3ccc3c5ccccc5n(-c5ccccc5)c34)n2)cc1 ; c1ccc(-c2nc(-c3ccccc3)nc(-c3ccc(-n4c5ccccc5c5c4ccc4c6ccccc6n(-c6ccccc6)c45)cc3)n2)cc1]

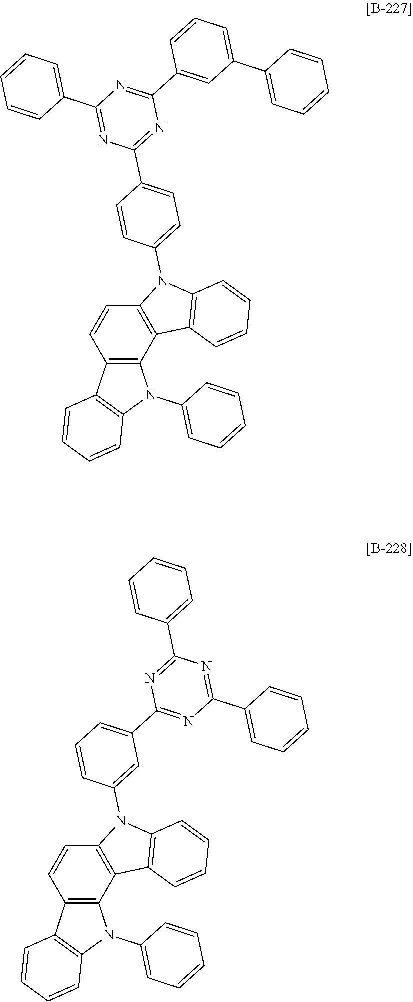 [SMILES: c1ccc(-c2cccc(-c3nc(-c4ccccc4)nc(-c4ccc(-n5c6ccccc6c6c5ccc5c7ccccc7n(-c7ccccc7)c56)cc4)n3)c2)cc1 ; c1ccc(-c2nc(-c3ccccc3)nc(-c3cccc(-n4c5ccccc5c5c4ccc4c6ccccc6n(-c6ccccc6)c45)c3)n2)cc1]

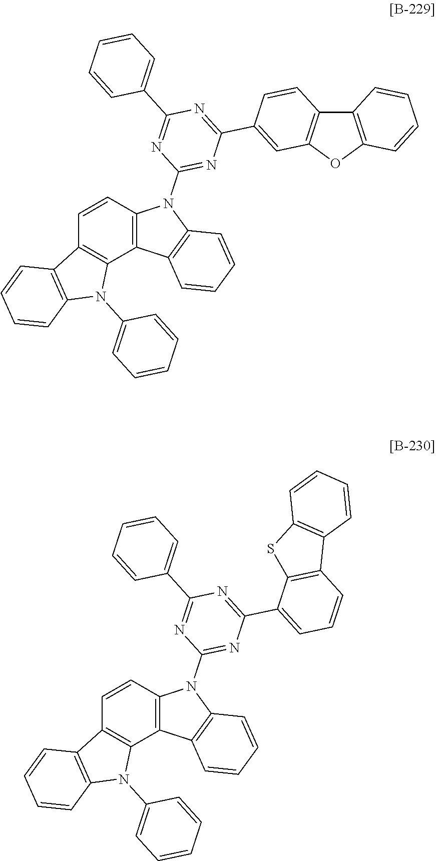 [SMILES: c1ccc(-c2nc(-c3ccc4c(c3)oc3ccccc34)nc(-n3c4ccccc4c4c3ccc3c5ccccc5n(-c5ccccc5)c34)n2)cc1 ; c1ccc(-c2nc(-c3cccc4c3sc3ccccc34)nc(-n3c4ccccc4c4c3ccc3c5ccccc5n(-c5ccccc5)c34)n2)cc1]

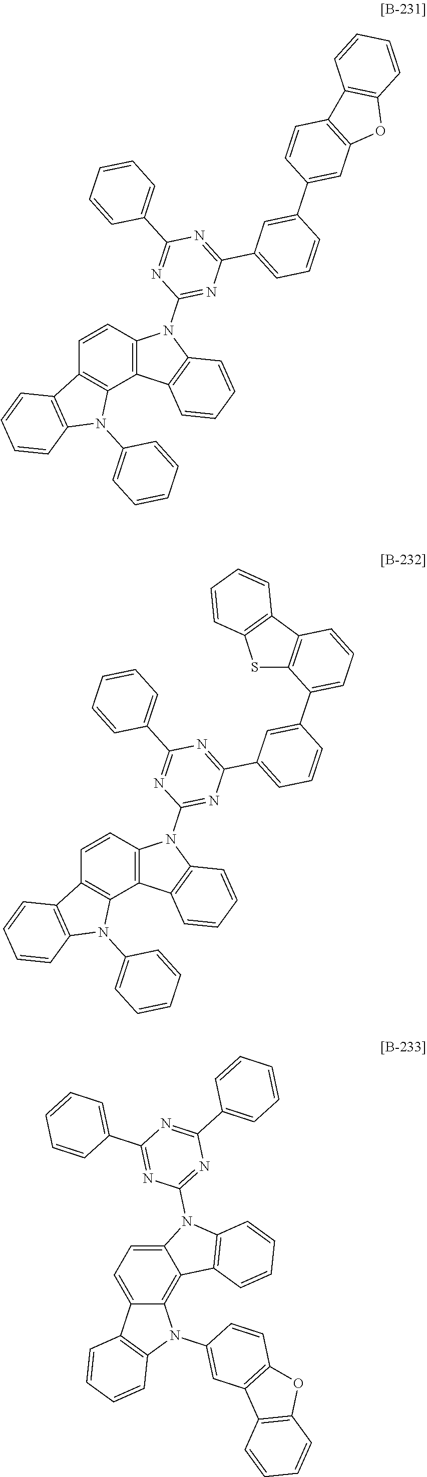 [SMILES: c1ccc(-c2nc(-c3cccc(-c4ccc5c(c4)oc4ccccc45)c3)nc(-n3c4ccccc4c4c3ccc3c5ccccc5n(-c5ccccc5)c34)n2)cc1 ; c1ccc(-c2nc(-c3cccc(-c4cccc5c4sc4ccccc45)c3)nc(-n3c4ccccc4c4c3ccc3c5ccccc5n(-c5ccccc5)c34)n2)cc1 ; c1ccc(-c2nc(-c3ccccc3)nc(-n3c4ccccc4c4c3ccc3c5ccccc5n(-c5ccc6oc7ccccc7c6c5)c34)n2)cc1]

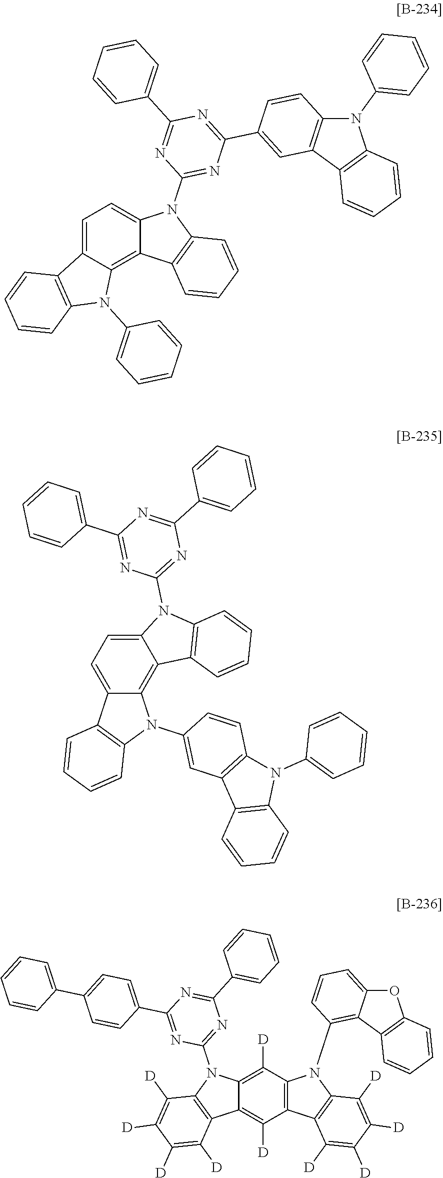 [SMILES: c1ccc(-c2nc(-c3ccc4c(c3)c3ccccc3n4-c3ccccc3)nc(-n3c4ccccc4c4c3ccc3c5ccccc5n(-c5ccccc5)c34)n2)cc1 ; c1ccc(-c2nc(-c3ccccc3)nc(-n3c4ccccc4c4c3ccc3c5ccccc5n(-c5ccc6c(c5)c5ccccc5n6-c5ccccc5)c34)n2)cc1 ; [2H]c1c([2H])c([2H])c2c(c1[2H])c1c([2H])c3c4c([2H])c([2H])c([2H])c([2H])c4n(-c4cccc5oc6ccccc6c45)c3c([2H])c1n2-c1nc(-c2ccccc2)nc(-c2ccc(-c3ccccc3)cc2)n1]

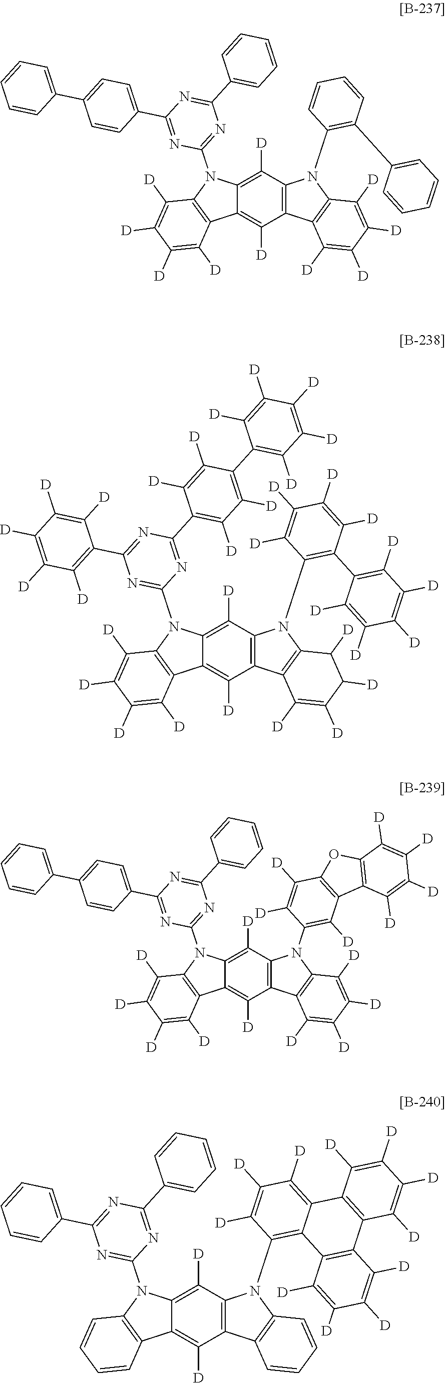

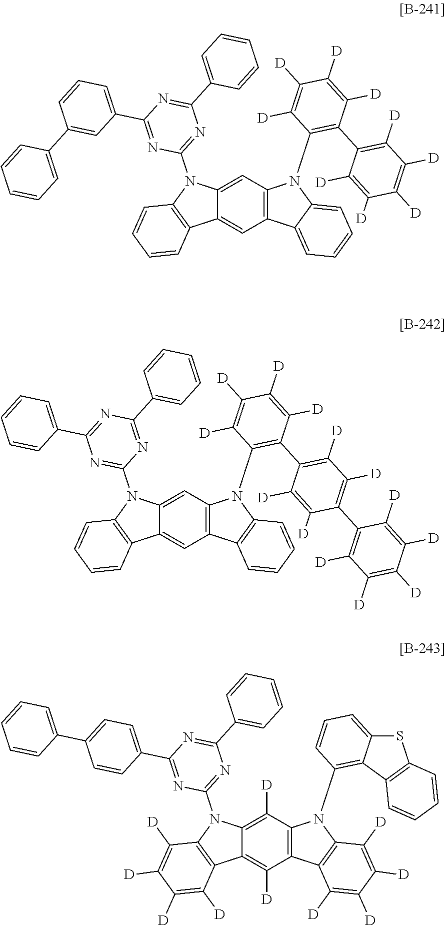 [SMILES: [2H]c1c([2H])c([2H])c(-c2c([2H])c([2H])c([2H])c([2H])c2-n2c3ccccc3c3cc4c5ccccc5n(-c5nc(-c6ccccc6)nc(-c6cccc(-c7ccccc7)c6)n5)c4cc32)c([2H])c1[2H] ; [2H]c1c([2H])c([2H])c(-c2c([2H])c([2H])c(-c3c([2H])c([2H])c([2H])c([2H])c3-n3c4ccccc4c4cc5c6ccccc6n(-c6nc(-c7ccccc7)nc(-c7ccccc7)n6)c5cc43)c([2H])c2[2H])c([2H])c1[2H] ; [2H]c1c([2H])c([2H])c2c(c1[2H])c1c([2H])c3c4c([2H])c([2H])c([2H])c([2H])c4n(-c4cccc5sc6ccccc6c45)c3c([2H])c1n2-c1nc(-c2ccccc2)nc(-c2ccc(-c3ccccc3)cc2)n1]

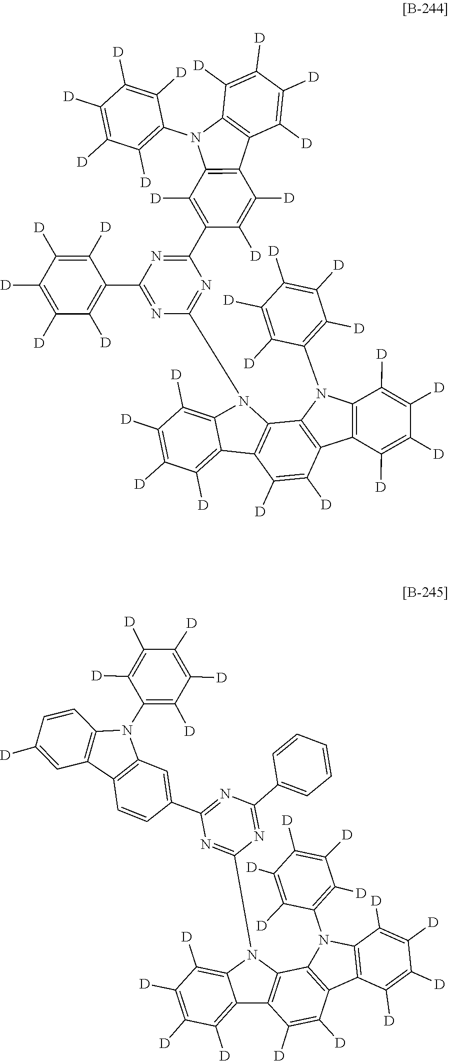 [SMILES: [2H]c1c([2H])c([2H])c(-c2nc(-c3c([2H])c([2H])c4c5c([2H])c([2H])c([2H])c([2H])c5n(-c5c([2H])c([2H])c([2H])c([2H])c5[2H])c4c3[2H])nc(-n3c4c([2H])c([2H])c([2H])c([2H])c4c4c([2H])c([2H])c5c6c([2H])c([2H])c([2H])c([2H])c6n(-c6c([2H])c([2H])c([2H])c([2H])c6[2H])c5c43)n2)c([2H])c1[2H] ; [2H]c1ccc2c(c1)c1ccc(-c3nc(-c4ccccc4)nc(-n4c5c([2H])c([2H])c([2H])c([2H])c5c5c([2H])c([2H])c6c7c([2H])c([2H])c([2H])c([2H])c7n(-c7c([2H])c([2H])c([2H])c([2H])c7[2H])c6c54)n3)cc1n2-c1c([2H])c([2H])c([2H])c([2H])c1[2H]]

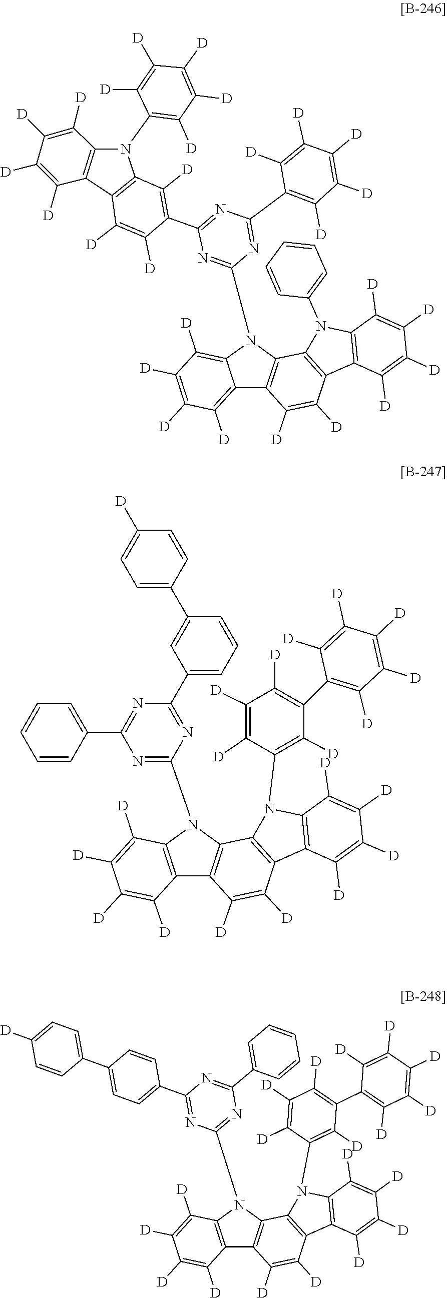

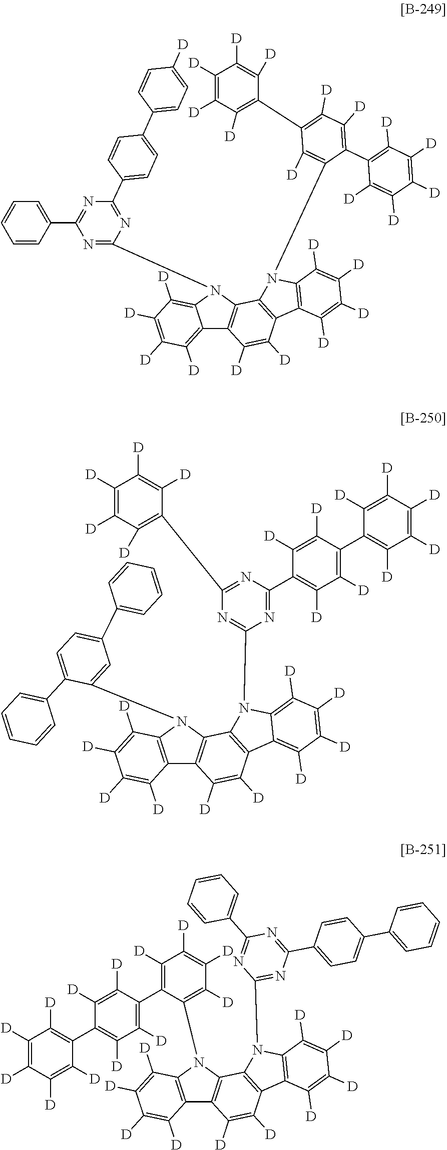

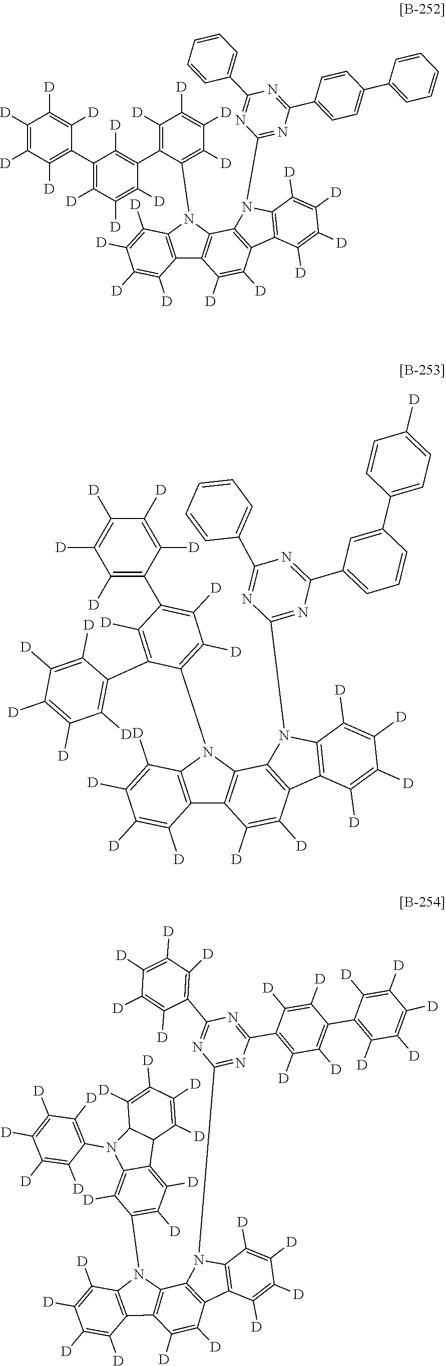

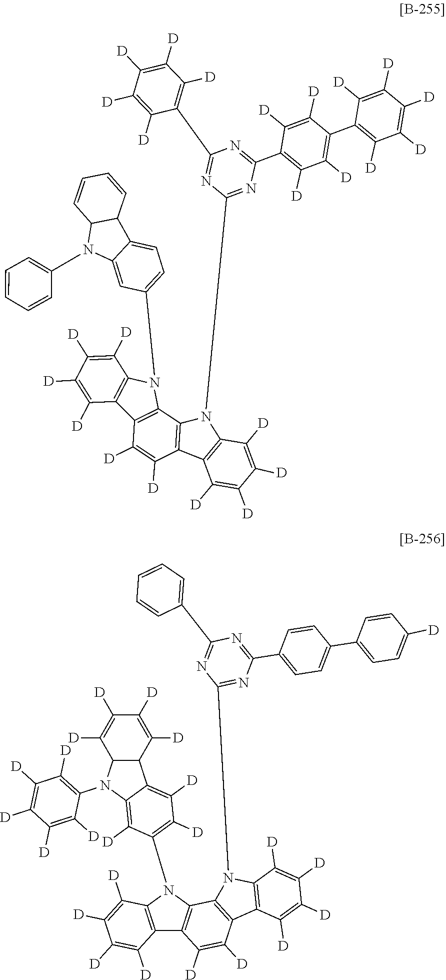 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-n3c4c([2H])c([2H])c([2H])c([2H])c4c4c([2H])c([2H])c5c6c([2H])c([2H])c([2H])c([2H])c6n(-c6ccc7c(c6)N(c6ccccc6)C6C=CC=CC76)c5c43)n2)c([2H])c1[2H] ; [2H]C1=C([2H])C2c3c([2H])c([2H])c(-n4c5c([2H])c([2H])c([2H])c([2H])c5c5c([2H])c([2H])c6c7c([2H])c([2H])c([2H])c([2H])c7n(-c7nc(-c8ccccc8)nc(-c8ccc(-c9ccc([2H])cc9)cc8)n7)c6c54)c([2H])c3N(c3c([2H])c([2H])c([2H])c([2H])c3[2H])C2C([2H])=C1[2H]]

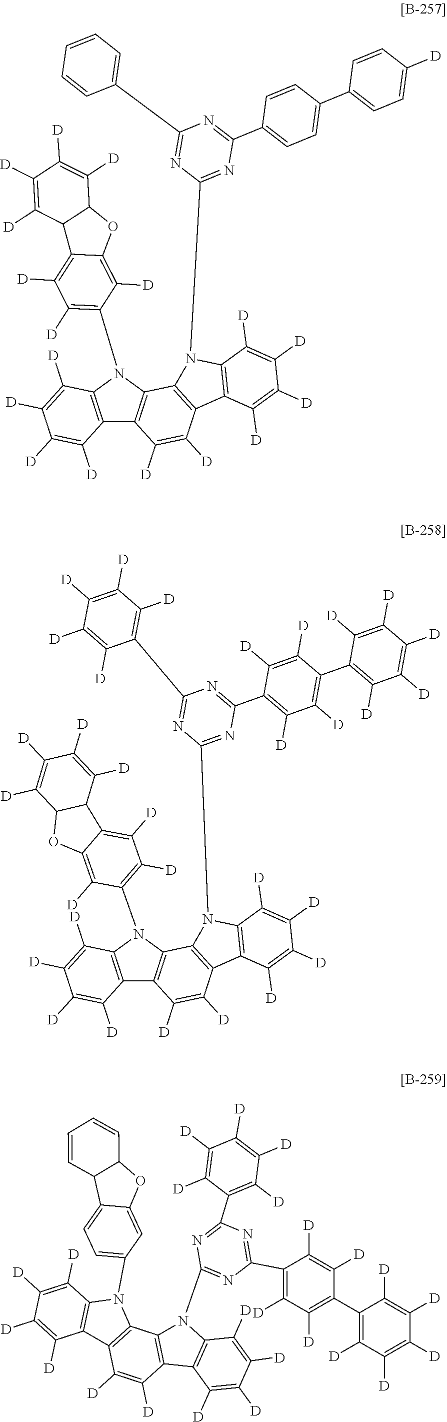

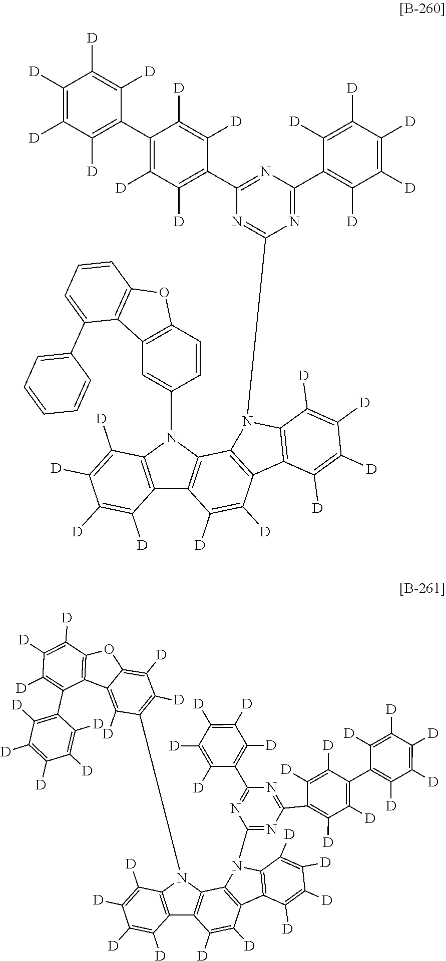

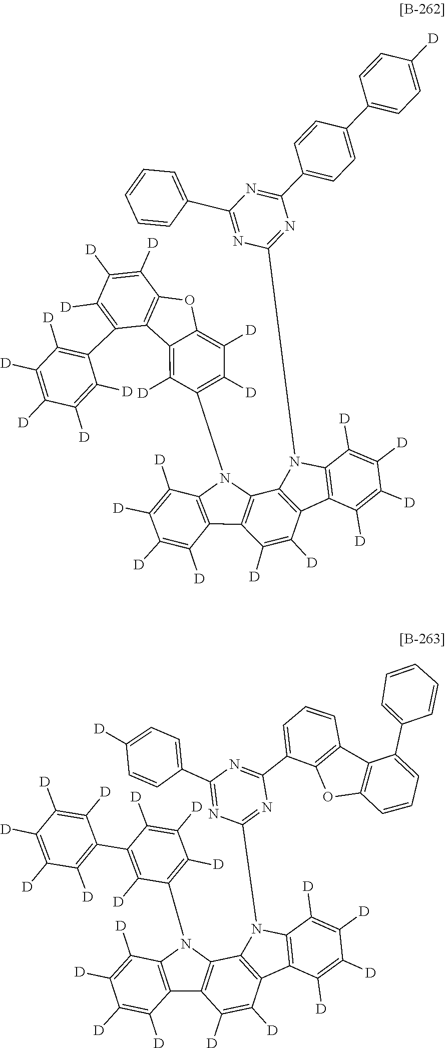 [SMILES: [2H]c1ccc(-c2ccc(-c3nc(-c4ccccc4)nc(-n4c5c([2H])c([2H])c([2H])c([2H])c5c5c([2H])c([2H])c6c7c([2H])c([2H])c([2H])c([2H])c7n(-c7c([2H])c([2H])c8oc9c([2H])c([2H])c([2H])c(-c%10c([2H])c([2H])c([2H])c([2H])c%10[2H])c9c8c7[2H])c6c54)n3)cc2)cc1 ; [2H]c1ccc(-c2nc(-c3cccc4c3oc3cccc(-c5ccccc5)c34)nc(-n3c4c([2H])c([2H])c([2H])c([2H])c4c4c([2H])c([2H])c5c6c([2H])c([2H])c([2H])c([2H])c6n(-c6c([2H])c([2H])c([2H])c(-c7c([2H])c([2H])c([2H])c([2H])c7[2H])c6[2H])c5c43)n2)cc1]

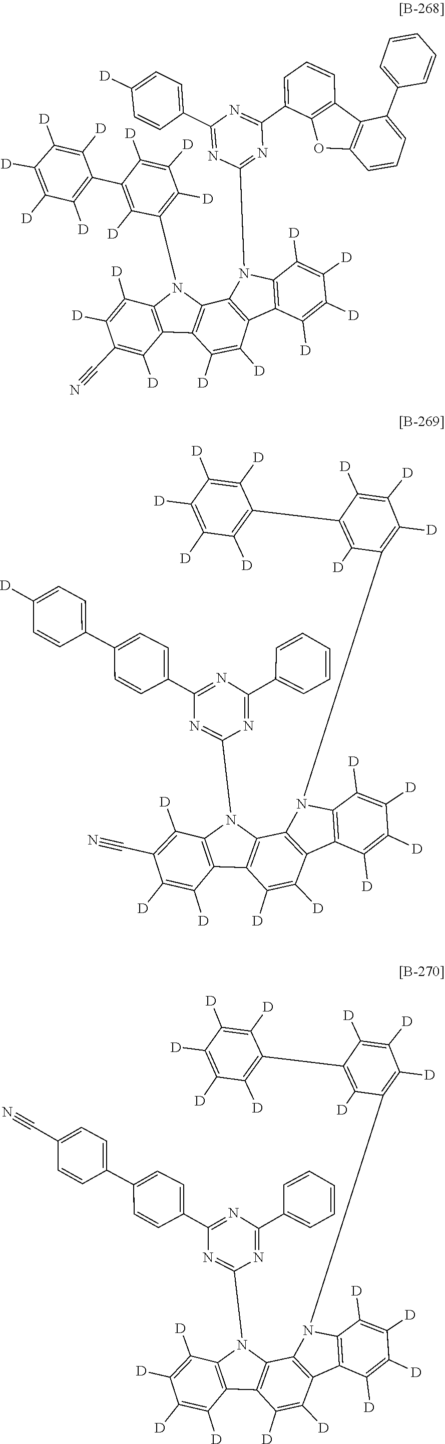

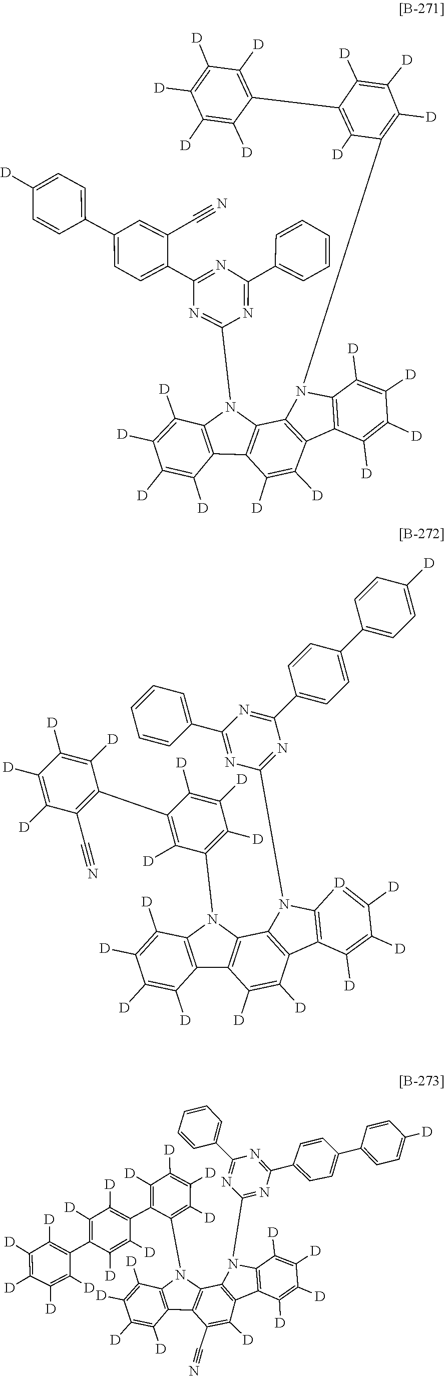

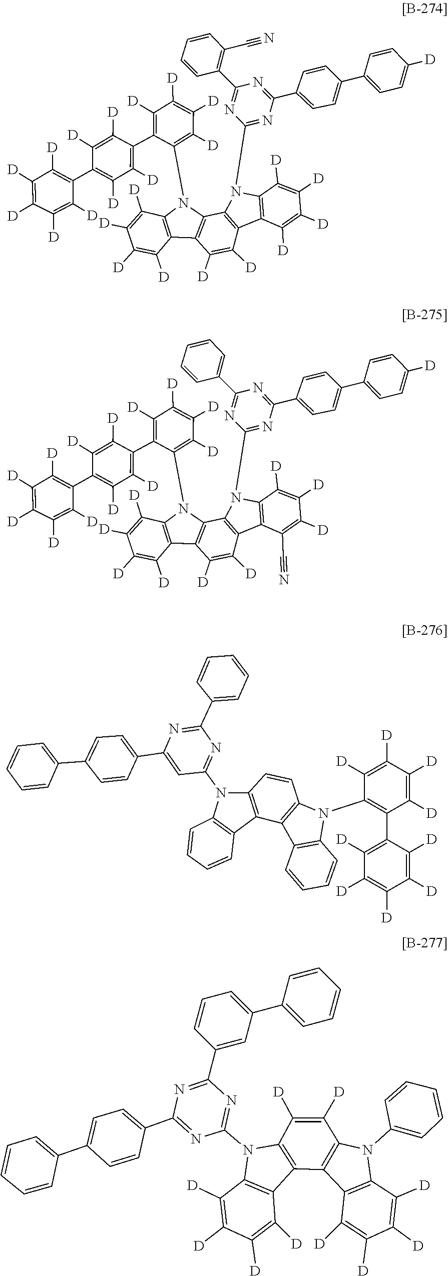

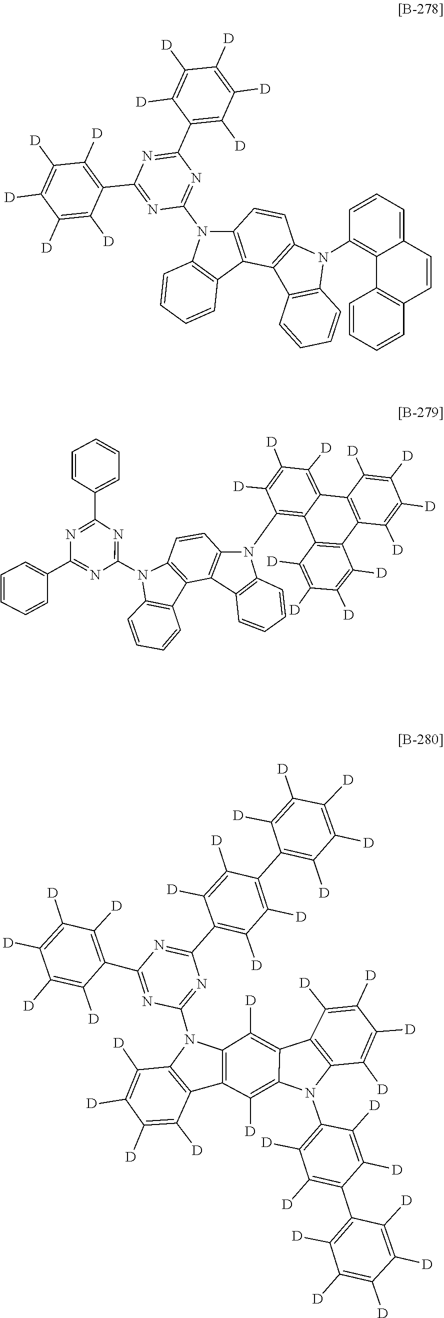

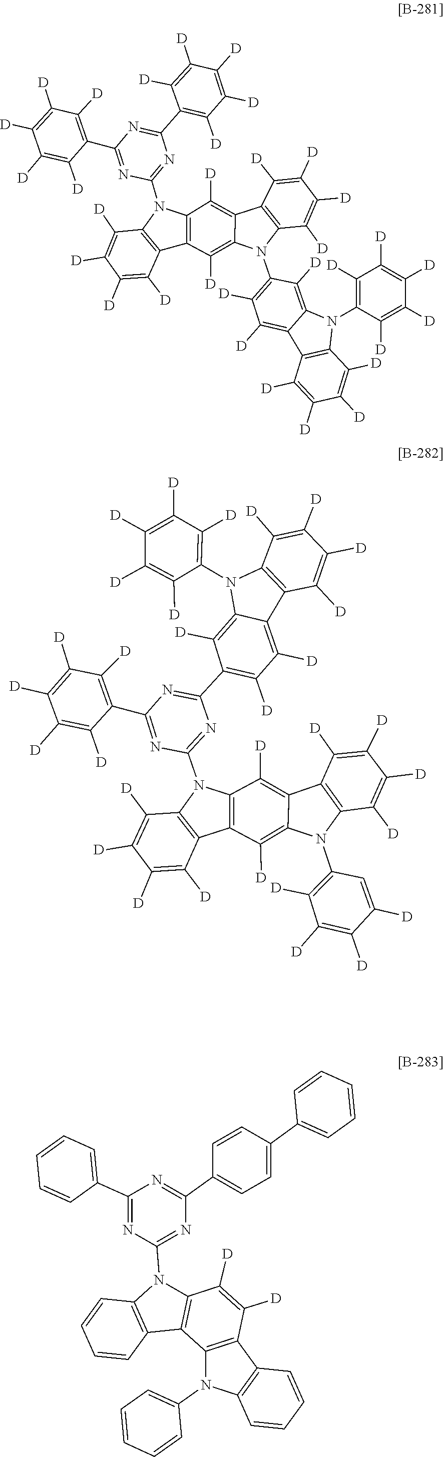

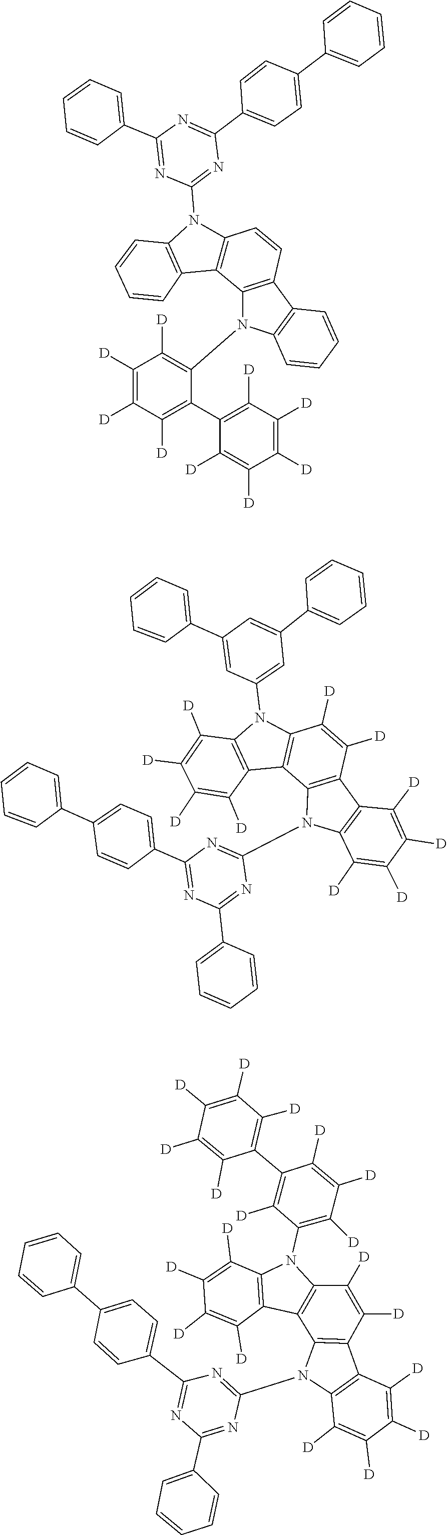

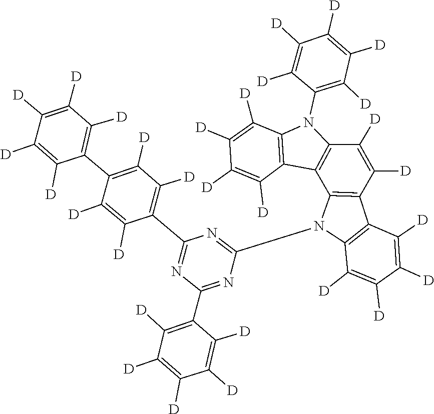 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-n3c4c([2H])c([2H])c([2H])c([2H])c4c4c([2H])c([2H])c5c(c6c([2H])c([2H])c([2H])c([2H])c6n5-c5c([2H])c([2H])c([2H])c([2H])c5[2H])c43)n2)c([2H])c1[2H]]

10. An organic photoelectronic device, comprising:
an anode and a cathode facing each other, and
at least one organic layer between the anode and the cathode,
wherein the at least one organic layer includes the composition for an organic optoelectronic device as claimed in claim 1.

11. The organic photoelectronic device as claimed in claim 10, wherein:
the at least one organic layer includes a light emitting layer, and
the light emitting layer includes the composition.

12. A display device comprising the organic photoelectronic device as claimed in claim 10.
